(19) United States
(12) Patent Application Publication
(10) Pub. No.: US 2020/0181284 A1
(43) Pub. Date: Jun. 11, 2020

(54) EPIGENETIC INHIBITORS FOR SENSITIZING HEMATOLOGIC OR OTHER MALIGNANCIES TO GLUCOCORTICOID THERAPY

(71) Applicants: UNIVERSITY OF SOUTHERN CALIFORNIA, Los Angeles, CA (US); UNIVERSITY OF IOWA RESEARCH FOUNDATION, Iowa City, IA (US)

(72) Inventors: Michael R. STALLCUP, Los Angeles, CA (US); Coralie POULARD, Los Angeles, CA (US); Miles A. PUFALL, Iowa City, IA (US)

(73) Assignees: UNIVERSITY OF SOUTHERN CALIFORNIA, Los Angeles, CA (US); UNIVERSITY OF IOWA RESEARCH FOUNDATION, Iowa City, IA (US)

(21) Appl. No.: 16/614,511

(22) PCT Filed: May 18, 2018
(86) PCT No.: PCT/US2018/033412

§ 371 Date: Nov. 18, 2019

Related U.S. Application Data
(60) Provisional application No. 62/508,233, filed on May 18, 2017.

(51) Int. Cl. C07K 16/40; A61K 31/573; A61K 31/713; A61K 31/7105; A61K 31/517; A61K 31/5355; A61K 31/496; A61K 31/506; A61K 31/7068; A61K 31/444; A61K 31/135; A61P 35/02; A61P 35/04

(52) U.S. Cl. CPC C07K 16/40; A61K 31/573; A61K 31/713; A61K 31/7105; A61K 31/517; A61K 31/5355; A61K 45/06; A61K 31/506; A61K 31/7068; A61K 31/444; A61K 31/135; A61P 35/02; A61P 35/04; A61K 31/496

(57) ABSTRACT

The present disclosure as disclosed in various embodiments is related to glueocorticoid compositions and glucocorticoid therapies for treating hematologic or other malignancies, methods and compositions for enhancing the chemotherapeutic effect of glucocorticoids, methods for determining early relapse of a hematologic or other malignancy in a subject, and methods for treating relapse of a hematologic or other malignancy in a subject.

### CROSS-REFERENCE TO RELATED APPLICATIONS

This application claims the benefit of U.S. Provisional Application No. 62/508,233 filed May 18, 2017, the disclosure of which is incorporated in its entirety by reference herein.

### STATEMENT REGARDING FEDERALLY SPONSORED RESEARCH OR DEVELOPMENT

The invention was made with Government support under Contract Nos. CA149088 and DK055274 awarded by the National Institutes of Health. The Government has certain rights to the invention.

### SEQUENCE LISTING

The text file Sequences_001_ST25.txt of size 17 KB created May 17, 2018, filed herewith, is incorporated in its entirety by reference herein.

### TECHNICAL FIELD

The present disclosure as disclosed in various embodiments is related to glucocorticoid compositions and glucocorticoid therapies for treating hematologic or other malignancies, methods and compositions for enhancing the chemotherapeutic effect of glucocorticoids, methods for determining early relapse of a hematologic or other malignancy in a subject, and methods for treating relapse of a hematologic or other malignancy in a subject.

### BACKGROUND

Although glucocorticoids (GCs or GC) have been used to treat lymphoid malignancies for over half a century^{1a}, the mechanism of their cytotoxicity is still not clear. Nonetheless, GC-based combination chemotherapy protocols are effective, particularly in children with B-cell precursor acute lymphoblastic leukemia (B-ALL). Although ˜90% of children on these protocols are cured, there are few effective treatments for the 10% who do not respond to this therapy^{1a}. Importantly, response to GCs alone is a good predictor of overall response to chemotherapy, indicating a central role for GCs in overall treatment efficacy and suggesting that the outcomes for resistant patients may be improved by enhancing GC potency^{1a}. Unfortunately, simply enhancing GC potency runs the risk of proportional increases in debilitating side effects, such as avascular necrosis and diabetes mellitus.

### SUMMARY

Synthetic glucocorticoid (GC) analogues are first-line drugs used to treat many hematologic cancers because they induce cell death by a mechanism shown in the lymphoid cell lineage. While many patients respond favorably to these drugs, the cancers for many patients are resistant to these drugs or develop resistance. In addition, long-term, high dose GC treatments cause serious adverse side-effects. The current application describes various methods, systems, and compositions of various embodiments to address these issues including, for example: 1) methods to increase sensitivity to GC-induced cell death at lower concentrations of GC for sensitive leukemias; 2) methods to increase GC sensitivity for resistant leukemias; and 3) methods to identify causes of GC resistance in hematologic cancers of individual patients and to predict which patients are likely to respond to GC. Facilitating the use of lower concentrations of GC may also help to reduce adverse side-effects.

The present disclosure as disclosed in various embodiments is related to glucocorticoid compositions and glucocorticoid therapies for treating hematologic or other malignancies, methods and compositions for enhancing the chemotherapeutic effect of glucocorticoids, methods for determining early relapse of a hematologic or other malignancy in a subject, and methods for treating relapse of a hematologic or other malignancy in a subject.

In various embodiments are disclosed methods or systems of treating a hematologic or other malignancy including administering to a subject a glucocorticoid and an Aurora Kinase B inhibitor. The administering of various embodiments can further include administering a demethylase inhibitor to the subject.

In various embodiments are disclosed compositions of treating a hematologic or other malignancy including therapeutically effective amounts of a glucocorticoid and an Aurora Kinase B inhibitor. The composition of various embodiments can further include therapeutically effective amounts of a demethylase inhibitor.

In various embodiments are disclosed methods or systems of enhancing chemotherapeutic effects of a glucocorticoid in a subject undergoing chemotherapy with the glucocorticoid for a hematologic or other malignancy including administering to the subject an amount of an Aurora Kinase B inhibitor effective to enhance chemotherapeutic effects of the glucocorticoid. The administering of various embodiments can further include administering a demethylase inhibitor to the subject to enhance chemotherapeutic effects of the glucocorticoid.

In various embodiments are disclosed methods or systems of treating a hematologic or other malignancy including administering to a subject a glucocorticoid and a demethylase inhibitor.

In various embodiments are disclosed compositions of treating a hematologic or other malignancy including therapeutically effective amounts of a glucocorticoid and a demethylase inhibitor.

In various embodiments are disclosed methods or systems of enhancing chemotherapeutic effects of a glucocorticoid in a subject undergoing chemotherapy with the glucocorticoid for a hematologic or other malignancy including administering to the subject an amount of a demethylase inhibitor effective to enhance the chemotherapeutic effect of a glucocorticoid.

In various embodiments are disclosed methods or systems of determining early relapse of hematologic or other malignancies in a subject including: quantifying a concentration or level of expression of Aurora Kinase B in a sample from a subject; comparing the concentration or level of expression of Aurora Kinase B in the sample to an Aurora Kinase B control; and identifying the subject as likely to have early relapse of a hematologic and other malignancy when the concentration or level of expression of Aurora Kinase B in the sample is greater than the Aurora Kinase B control.

In various embodiments are disclosed methods or systems of determining early relapse of hematologic or other malignancies in a subject and treating relapse of the hematologic or other malignancies in the subject including: quantifying a concentration or level of expression of Aurora Kinase B in a sample from a subject; comparing the concentration or level of expression of Aurora Kinase B in the sample to an Aurora Kinase B control; identifying the subject as likely to have early relapse of a hematologic and other malignancy when the concentration or level of expression of Aurora Kinase B in the sample is greater than the Aurora Kinase B control; and administering a glucocorticoid and an Aurora Kinase B inhibitor to the subject identified as likely to have early relapse of the hematologic and other malignancy when relapse of the hematologic and other malignancy occurs.

In various embodiments are disclosed methods or systems of determining early relapse of hematologic or other malignancies in a subject including: quantifying a concentration or level of expression of Aurora Kinase B in a sample from a subject; comparing the concentration or level of expression of Aurora Kinase B in the sample to an Aurora Kinase B control; quantifying a concentration or level of expression of demethylase in the sample; comparing the concentration or level of expression of demethylase in the sample to a demethylase control; and identifying the subject as likely to have early relapse of a hematologic and other malignancy when the concentration or expression of Aurora Kinase B and demethylase in the sample is greater than the Aurora Kinase B and demethylase controls.

In various embodiments are disclosed methods or systems of determining early relapse of hematologic or other malignancies in a subject and treating relapse of the hematologic or other malignancies in the subject including: quantifying a concentration or level of expression of Aurora Kinase B in a sample from a subject; comparing the concentration or level of expression of Aurora Kinase B in the sample to an Aurora Kinase B control; quantifying a concentration or level of expression demethylase in the sample; comparing the concentration or level of expression of demethylase in the sample to a demethylase control; identifying the subject as likely to have early relapse of a hematologic and other malignancy when the concentration or level of expression of Aurora Kinase B and demethylase in the sample is greater than the Aurora Kinase B and demethylase controls; and administering a glucocorticoid, an Aurora Kinase B inhibitor, and a demethylase inhibitor to the subject identified as likely to have early relapse of the hematologic and other malignancy when relapse of the hematologic and other malignancy occurs.

In various embodiments are disclosed methods or systems of determining early relapse of hematologic or other malignancies in a subject including: quantifying a concentration or level of expression of demethylase in a sample from a subject; comparing the concentration or level of expression of demethylase in the sample to a demethylase control; and identifying the subject as likely to have early relapse of a hematologic and other malignancy when the concentration or level of expression of demethylase in the sample is greater than the demethylase control.

In various embodiments are disclosed methods or systems of determining early relapse of hematologic or other malignancies in a subject and treating relapse of the hematologic or other malignancies in the subject including: quantifying a concentration or level of expression of demethylase in a sample form a subject; comparing the concentration or level of expression of demethylase in the sample to a demethylase control; identifying the subject as likely to have early relapse of a hematologic and other malignancy when the concentration or level of expression of demethylase in the sample is greater than the demethylase; and administering a glucocorticoid and a demethylase inhibitor to the subject identified as likely to have early relapse of the hematologic and other malignancy when relapse of the hematologic and other malignancy occurs.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein

FIGS. 1A, 1B, 1C, 1D, 1E, 1F, 2A-1, 2A-2, 2B, 2C, 2D, 3A, 3B, 4A, 4B, 4C, 4D, 4E-1, 4E-2, 4F, 4G, 4H, 4I, 5A-1, 5A-2, 5A-3, 5B-1, 5B-2, 5B-3, 5C-1, 5C-2, 5C-3, 6, 7, 8A, 8B, 8C, 8D, 9A, 9B, 9C, 9D, 10A, 10B, 10C, 10D, 11A, 11B, 11C, 11D, 11E, 11F, 12A, 12B, 12C, 12D, 13A-1, 13A-2, 13B, 13C, 13D, 14A-1, 14A-2, 14B-1, 14B-2, 15A, 15B, 15C, 15D, 15E, 16A, 16B, 16C, and 16D show various embodiments of the present disclosure.

FIGS. 17A, 17B-1, 17B-2, 17C, 17D, 17E-1, 17E-2, 17E-3, 18A, 18B, 18C-1, 18C-2, 18C-3, 18D-1, 18D-2, 18D-3, 18D-4, 18D-5, 18E-1, 18E-2, 18E-3, 19A, 19B, 20A, 20B, 20C, 20D, 21A, 21B, 21C, 21D-1, 21D-2, 21D-3, 21D-4, 21D-5, 21D-6, 21D-7, 21D-8, 21E-1, 21E-2, 21E-3, 21E-4, 21E-5, 21E-6, 21E-7, 21E-8, 21F-1, 21F-2, 21F-3, 21F-4, 21F-5, 21F-6, 21-7, 21F-8, 22A-1, 22A-2, 22B, 22C, 22D-1, 22D-2, 22D-3, 22D-4, 22D-5, 22D-6, 22E-1, 22E-2, 22E-3, 22E-4, 23A-1, 23A-2, 23B-1, 23B-2, 23B-3, 23C, 23D-1, 23D-2, 23D-3, 23E, 24A, 24B, 24C, 24D, 24E-1, 24E-2, 25A, 25B, 25C, 25D-1, 25D-2, 25E-1, 25E-2, 25F-1, 25F-2, 26A-1, 26A-2, 26B-1, 26B-2, 26C-1, 26C-2, 26C-3, 26D-1, 26D-2, 26D-3, 27A, 27B, 27C, 27D, 27E, 27F, 27G, 28A, 28B, 28C, 29A, 29B, 29C, 29D-1, 29D-2, 29D-3, 29D-4, 29D-5, 29E-1, 29E-2, 29E-3, 29E-4, 29E-5, 30A-1, 30A-2, 30A-3, 30A-4, 30A-5, 30A-6, 30B, 30C-1, 30C-2, 30C-3, 30C-4, 30D, 30E-1, 30E-2, 30E-1, 30E-2, 30G, 30H, 30I, 30J, 31A, 31B, 31C, 31D-1, 31D-2, 31D-3, 31D-4, 31D-5, 31D-6, 31D-7, 31D-8, 31E-1, 31E-2, 31E-3, 31E-4, 31E-5, 31E-6, 31E-7, 31E-8, 31F-1, 31F-2, 31F-3, 31F-4, 31F-5, 31F-6, 31F-7, 31F-8, 32A, 32B, 32C, 32D-1, 32D-2, 32D-3, 32D-4, 32E-1, 32E-2, 32E-3, 32F-1, 32F-2, and 33 show various embodiments of the present disclosure.

FIGS. 34A, 34B, 34C, 34D, 34E, 35A, 35B, 35C, 35D, 36A, 36B, 36C, 36D, 36E, 36F, 37A, 37B, 37C, 37D, 37E, 37F, 38A-1, 38A-2, 38A-3, 38A-4, 38B, 38C-1, 38C-2, 38C-3, 38D, 38E, 39A, 39B, 39C, 39D, 40A, 40B, 40C, 40D-1, 40D-2, 40D-3, 40D-4, 41A-1, 41A-2, 41B-1, 41B-2, 42A, 42B, 42C, 42D, 42E, 42F, 43A, 43B-1, 43B-2, 43C-1, 43C-2, 43D-1, 43D-2, 43D-3, 43D-4, 44A, 44B, 44C, 45A, 45B, 45C, 45D, 46A, 46B, 46C, 46D, 47A, 47B, 48A, 48B, 48C, 48D, 48E, and 48F show various embodiments of the present disclosure.

FIGS. 49, 50A, 50B, 50C-1, 50C-2, 51, 52A, 52B, 53, 54A, 54B, 54C, 54D, 55A, 55B, and 55C show various embodiments of the present disclosure.

### DETAILED DESCRIPTION

As required, detailed embodiments of the present disclosure are disclosed herein; however, it is to be understood that the disclosed embodiments are merely exemplary and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It is also noted that, as used in the specification and the appended claims, the singular form “a,” “an,” and “the” comprise plural referents unless the context clearly indicates otherwise. For example, reference to a component in the singular is intended to comprise a plurality of components.

The term “or” can be understood to mean “at least one of”. The term “and” can also be understood to mean “at least one of” or “all”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The terms “polynucleotide”, “nucleotide”, “nucleotide sequence”, “nucleic acid” and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “amino acid sequence” or “amino acid” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

The terms “peptide” or “protein” as used herein refers to any peptide, oligopeptide, polypeptide, gene product, expression product, or protein. A peptide is comprised of consecutive amino acids. The term “peptide” encompasses naturally occurring or synthetic molecules.

The term “subject(s)” refers a subject with a hematologic or other malignancy and can include any mammalian subject(s) of any mammalian species such as, but not limited to, humans, dogs, cats, horses, rodents, any domesticated animal, or any wild animal.

The term “inhibit or “inhibitition” refers to inhibiting a biological activity of a biological molecule or expression of a biological molecule. The biological molecule can, for example, be a biological molecule associated with various cancers at any stage of oncogenesis (i.e. epithelial-mesenchymal transition, metastisis, etc.).

The term “hematologic malignancy” can refer to hematopoietic precancerous (e.g., benign), malignant, pre-metastatic, metastatic, and non-metastatic cancers. Examples of hematologic malignanciues can include leukemias, lymphomas (Hodgkins and non-Hodgkins), myelomas, or myeloproliferative disorders.

The term “other malignancy” can refer to solid precancerous (e.g., benign), malignant, pre-metastatic, metastatic, and non-metastatic cancers. Examples of other malignancies can include breast cancers, skin cancers, esophageal cancers, liver cancers, pancreatic cancers, prostate cancers, uterine cancers, cervical cancers, lung cancers, bladder cancers, ovarian cancers, or melanomas.

The term “effective amount” of drug, compound, or pharmaceutical composition is an amount sufficient to effect beneficial or desired results. For example, an effective amount can include amounts used for treating cancers or amounts used for enhancing the chemotherapeutic effects of glutocorticoids and glutocorticoid therapies.

The term “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion (such as domain antibodies), and any other modified configuration of the immunoglobulin molecule that comprises an antigen recognition site. An antibody includes an antibody of any class, such as IgG, IgA, or IgM (or sub-class thereof), and the antibody need not be of any particular class.

The terms “siRNA oligonucleotides”, “RNAi oligonucleotides”, “short interfering RNA”, or “siRNA” are used interchangeably and refer to oligonucleotides that work through post-transcriptional gene silencing, also known as RNA interference (RNAi). The terms refer to a double stranded nucleic acid molecule capable of RNA interference “RNAi”, (PCT Publication No. WO 00/44895; WO 01/36646; WO 99/32619; WO 01/29058 that are all incorporated in their entirety by reference). SiRNA molecules are generally RNA molecules but further encompass chemically modified nucleotides and non-nucleotides. SiRNA gene-targeting experiments have been carried out by transient siRNA transfer into cells (achieved by such classic methods as liposome-mediated transfection, electroporation, or microinjection). Molecules of siRNA are 21- to 23-nucleotide RNAs, with characteristic 2- to 3-nucleotide 3 ′-overhanging ends resembling the RNase III processing products of long double-stranded RNAs (dsRNAs) that normally initiate RNAi. One method for efficient intracellular delivery of siRNA is the use of short hairpin RNAs, or “shRNAs”. shRNAs are single stranded RNA molecules that include two complementary sequences joined by a non-complementary region. In vivo, the complementary sequences anneal to create a double-stranded helix with an unpaired loop at one end. The resulting lollypop-shaped shaped structure is called a stem loop and can be recognized by the RNAi machinery and processed intracellularly into short duplex RNAs having siRNA-like properties.

Unless expressly stated to the contrary: all R groups (e.g. Rᵢ where i is an integer) include H or hydrogen, alkyl, lower alkyl, C₁₋₆ alkyl, C₆₋₁₀ aryl, or C₆₋₁₀ heteroaryl; single letters (e.g., “n” or “o”) are 1, 2, 3, 4,or 5; percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” as used herein means C120, linear, branched, rings, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Lower alkyl” refers to an alkyl group having 1 to about 8 carbon atoms (i.e., a C₁₋₈ alkyl), e.g., 1, 2, 3, 4, 5, 6, 7, or 8 carbon atoms. Lower alkyl can also refer to a range between any two numbers of carbon atoms listed above. “Higher alkyl” refers to an alkyl group having about 10 to about 20 carbon atoms, e.g., 10, 11, 12, 13, 14, 15, 16, 17, 18, 19, or 20 carbon atoms. Higher alkyl can also refer to a range between any two number of carbon atoms listed above.

The term “aryl” as used herein means an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether. Examples of aryl include, but are not limited to, phenyl, naphthyl, biphenyl, and diphenylether, and the like. Aryl groups include heteroaryl groups, wherein the aromatic ring or rings include a heteroatom (e.g., N, O, S, or Se). Exemplary heteroaryl groups include, but are not limited to, furanyl, pyridyl, pyrimidinyl, imidazoyl, benzimidazolyl, benzofuranyl, benzothiophenyl, quinolinyl, isoquinolinyl, thiophenyl, and the like. The aryl group can be optionally substituted (a “substituted aryl”) with one or more aryl group substituents, which can be the same or different, wherein “aryl group substituent” includes alkyl (saturated or unsaturated), substituted alkyl (e.g., haloalkyl and perhaloalkyl, such as but not limited to —CF₃), cylcoalkyl, aryl, substituted aryl, aralkyl, halo, nitro, hydroxyl, acyl, carboxyl, alkoxyl (e.g., methoxy), aryloxyl, aralkyloxyl, thioalkyl, thioaryl, thioaralkyl, amino (e.g., aminoalkyl, aminodialkyl, aminoaryl, etc.), sulfonyl, and sulfinyl.

The present disclosure as disclosed in various embodiments is related to glucocorticoid compositions and glucocorticoid therapies for treating hematologic or other malignancies, methods, systems, and compositions for enhancing the chemotherapeutic effect of glucocorticoids, methods and systems for determining early relapse of a hematologic or other malignancy in a subject, and methods for treating relapse of a hematologic or other malignancy in a subject.

In various embodiments are disclosed methods or systems of treating a hematologic or other malignancy including administering to a subject a glucocorticoid and an Aurora Kinase B inhibitor. The administering of various embodiments can further include administering a demethylase inhibitor to the subject.

In various embodiments are disclosed compositions of treating a hematologic or other malignancy including therapeutically effective amounts of a glucocorticoid and an Aurora Kinase B inhibitor. The composition of various embodiments can further include therapeutically effective amounts of a demethylase inhibitor.

In various embodiments are disclosed methods or systems of enhancing chemotherapeutic effects of a glucocorticoid in a subject undergoing chemotherapy with the glucocorticoid for a hematologic or other malignancy including administering to the subject an amount of an Aurora Kinase B inhibitor effective to enhance the chemotherapeutic effect of a glucocorticoid. The amount of an Aurora Kinase B inhibitor of various embodiments is an effective amount of Aurora Kinase B inhibitor to enhance the chemotherapeutic effect of the glucocorticoid.

In various embodiments are disclosed methods or systems of treating a hematologic or other malignancy including administering to a subject a glucocorticoid and a demethylase inhibitor.

In various embodiments are disclosed compositions of treating a hematologic or other malignancy including therapeutically effective amounts of a glucocorticoid and a demethylase inhibitor.

In various embodiments are disclosed methods or systems of enhancing chemotherapeutic effects of a glucocorticoid in a subject undergoing chemotherapy with the glucocorticoid for a hematologic or other malignancy including administering to the subject an amount of a demethylase inhibitor effective to enhance the chemotherapeutic effect of a glucocorticoid. The amount of demethylase inhibitor of various embodiments is an effective amount of demethylase inhibitor to enhance the chemotherapeutic effect of the glucocorticoid.

In various embodiments, the subject is a mammalian subject such as a human subject. The subject of various embodiments has a hematologic or other malignancy. Also, the Aurora Kinase B or demethylase of various embodiments can include any mammalian derived Aurora Kinase B or demethylase.

In various embodiments, the hematologic malignancy is a hematopoietic malignancy of a lymphoid lineage that can include, for example, adult or childhood malignant lymphoid cancers such as acute lymphoblastic leukemia, chronic lymphocytic leukemia, multiple myeloma, Hodgkin's lymphoma, or non-Hodgkin's lymphoma. The adult or childhood malignant lymphoid cancers of various embodiments is of a B-cell lineage such as, for example, B-lineage lymphoblastic leukemia, childhood B-lineage lymphoblastic leukemia, or childhood B-lineage acute lymphoblastic leukemia or of a T-cell lineage, such as, for example, peripheral T-cell lymphoma, anaplastic large cell lymphoma, angioimmunoblastic lymphoma, or cutaneous T-cell lymphoma.

In various embodiments, the other malignancy includes solid tumors including, for example, lung cancer. The glucocorticoid and Aurora Kinase B inhibitor or demethylase inhibitor of the methods and compositions for treating other malignancies of various embodiments prevents metastasis of the other malignancy. The glucocorticoid and Aurora Kinase B inhibitor or demethylase inhibitor of the methods and compositions for treating hematologic or other malignancies of various embodiments inhibits epithelial-mesenchymal transition(s), such as by enhancing E-cadherin expression in the other malignancy.

In various embodiments, the hematologic or other malignancies is resistant to glucocorticoid therapy. The hematologic or other malignancies of various embodiments is resistant to glucocorticoid-mediated cell death.

In various embodiment, the glucocorticoid can include any glucocorticoid such as synthetic glucocorticoids or glucocorticoid drugs such as, for example: beclomethasone, betamethasone, budesonide, cortisone, dexamethasone, hydrocortisone, methylprednisolone, prednisolone, prednisone, and triamcinolone.

In various embodiments, the dosage of the glucocorticoid is at least 10 nM or ranges from about 10 nM to about 1000 nM. In various embodiments, the dosage of the glucocorticoid is 10 nM, 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1000 nM. In various embodiments, the dosage of the glucocorticoid is a range between any two dosages listed above.

In various embodiments, the dosage of the Aurora Kinase B inhibitor ranges from about 0.1 nM to about 1000 nM. In other embodiments, the Aurora Kinase B inhibitor ranges from about 10 nM to about 50 nM. In various embodiments, the dosage of Aurora Kinase B inhibitor is about 0.1 nM, 0.2 nM, 0.3 nM, 0.4 nM, 0.5 nM, 0.6 nM, 0.7 nM, 0.8 nM, 0.9 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, 20 nM, 20.5 nM, 21 nM, 21.5 nM, 22 nM, 22.5 nM, 23 nM, 23.5 nM, 24 nM, 24.5 nM, 25 nM, 25.5 nM, 26 nM, 26.5 nM, 27 nM, 27.5 nM, 28 nM, 28.5 nM, 29 nM, 29.5 nM, 30 nM, 30.5 nM, 31 nM, 31.5 nM, 32 nM, 32.5 nM, 33 nM, 33.5 nM, 34 nM, 34.5 nM, 35 nM, 35.5 nM, 36 nM, 36.5 nM, 37 nM, 37.5 nM, 38 nM, 38.5 nM, 39 nM, 39.5 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, and 1000 nM. In various embodiments, the dosage of the Aurora Kinase B inhibitor is between any two concentrations from above.

In various embodiments, the Aurora Kinase B inhibitor is a plurality of Aurora Kinase B inhibitors and can include various types of competitive, non-competitive, uncompetitive, reversible, or irreversible inhibitors. In various embodiments, the plurality of Aurora Kinase B inhibitors is 1, 2, 3, 4, 5, 6, 7, 8, 9, or 10 different Aurora Kinase B inhibitors. In various embodiments, the plurality of Aurora Kinase B inhibitors is a range between any number of different Aurora Kinase B inhibitors listed above.

The Aurora Kinase B inhibitor of various embodiments can include various compounds, antibodies, sense or anti-sense nucleic acid molecules, or combinations thereof that inhibit the function of or expression of Aurora Kinase B. In various embodiments, the Aurora Kinase B inhibitor binds to at least one of Aurora Kinase B and antagonizes the activity of the Aurora Kinase B related nucleic acid or protein.

In various embodiments, Aurora Kinase B inhibitor includes compounds having a half maximal inhibitory concentration (IC₅₀) or inhibitory constant (Kᵢ) for inhibiting of Aurora Kinase B of less than about 1 μM. The Aurora Kinase B inhibitor of various embodiments includes compounds having an IC₅₀ or Kᵢ for inhibiting of Aurora Kinase B of about 0.1 nM, 0.2 nM, 0.3 nM, 0.4 nM, 0.5 nM, 0.6 nM, 0.7 nM, 0.8 nM, 0.9 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, 20 nM, 20.5 nM, 21 nM, 21.5 nM, 22 nM, 22.5 nM, 23 nM, 23.5 nM, 24 nM, 24.5 nM, 25 nM, 25.5 nM, 26 nM, 26.5 nM, 27 nM, 27.5 nM, 28 nM, 28.5 nM, 29 nM, 29.5 nM, 30 nM, 30.5 nM, 31 nM, 31.5 nM, 32 nM, 32.5 nM, 33 nM, 33.5 nM, 34 nM, 34.5 nM, 35 nM, 35.5 nM, 36 nM, 36.5 nM, 37 nM, 37.5 nM, 38 nM, 38.5 nM, 39 nM, 39.5 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, and 1000 nM. In various embodiments, the Aurora Kinase B inhibitor includes compounds having an IC₅₀ or Kᵢ for inhibiting of Aurora Kinase B between any two concentrations from above.

In various embodiments, the Aurora Kinase B inhibitor includes compounds such as, for example, Barasertib (AZD1152, AZD1152-HQPA, or AZD2811; CAS No. 722543-31-9), ZM 447439 (CAS No. 331771-20-1), Danusertib (PHA-739358; CAS No. 827318-97-8), AT9283 (CAS No. 896466-04-9), PF-03814735 (CAS No. 942487-16-3), AMG 900 (CAS No. 945595-80-2), and Cytarabine (CAS No. 147-94-4).

For example, the Aurora Kinase B inhibitor can be a compound having Formula I

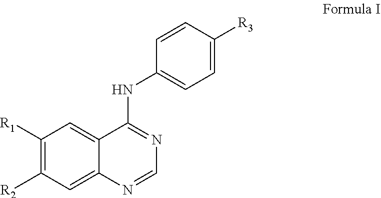

or a pharmaceutically acceptable salt thereof, wherein each of R₁ and R₂ is selected from the group consisting of: R₄—O—, H, and

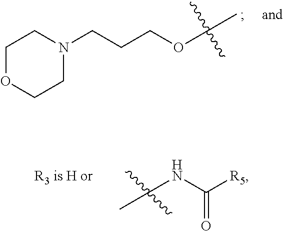

wherein R₄ is an alkyl (e.g. C₁-C₆ alkyl), or H;
R₅ is H, an alkyl or aryl (e.g. C₃-C₈ cycloalkyl such as cyclopropyl, benzyl),

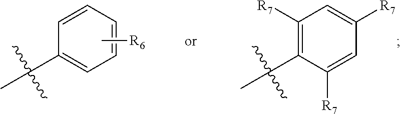

and

### R₆ is H, F, CL, or OMe; and

R₇ is a C₁-C₃ alkyl or H.

In other examples, the Aurora Kinase B inhibitor can be N-[4-[[6-methoxy-7-(3-morpholin-4-ylpropoxy)quinazolin-4-yl]amino]phenyl]benzamide (ZM 447439; CAS No. 331771-20-1), a compound having Formula II, or a pharmaceutically acceptable salt thereof.

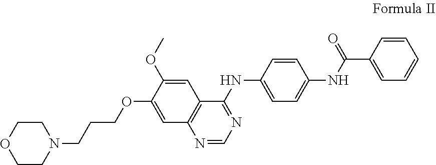 [SMILES: COc1cc2c(Nc3ccc(NC(=O)c4ccccc4)cc3)ncnc2cc1OCCCN1CCOCC1]

In various embodiments, the Aurora Kinase B inhibitor is an inhibitor disclosed in the following patents, patent application publications, and publications that are all incorporated in their entirety by reference herein: U.S. Pat. Nos. 7,563,787; 8,114,870; 8,624,027; U.S. Patent Application Publication No. 2015/0250824; 2016/0287602; 2016/0250175; 2014/0349969; 2013/0252924; 2016/0002222; 2015/0329828; 2014/0336073; 2014/0163028; 2016/0153052; and 2010/00196907.

In other examples, the Aurora Kinase B inhibitor can be 2-[ethyl-[3-[4-[[5-[2-(3-fluoroanilino)-2-oxoethyl]-1H-pyrazol-3-yl]amino]quinazolin-7-yl] oxypropyl]amino]ethyl dihydrogen phosphate, a compound having Formula III, or a pharmaceutically acceptable salt thereof.

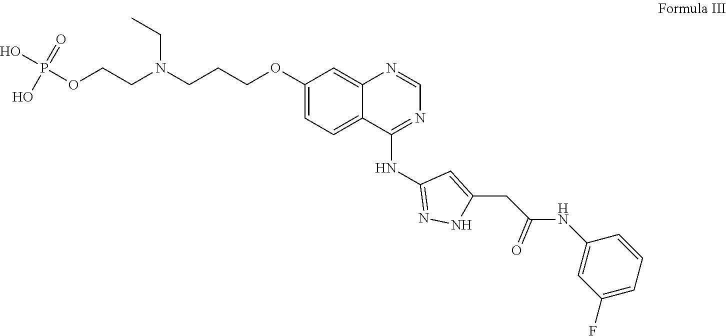 [SMILES: CCN(CCCOc1ccc2c(Nc3cc(CC(=O)Nc4cccc(F)c4)[nH]n3)ncnc2c1)CCOP(=O)(O)O]

In various embodiments, the Aurora Kinase B inhibitor is an inhibitor disclosed in the following patents, patent application publications, and publications that are all incorporated in their entirety by reference herein: U.S. Pat. Nos. 8,921,354; 8,933,069; 8,772,277; 8,877,445; 8,697,874; 8,324,395; 8,445,509; 8,399,449; 8,273,741; 8,344,135; 8,907,089; 8,927,718; 8,268,841; 8,691,828; 8,034,812; 8,304,557; 8,044,049; 7,528,121; 9,655,900; 8,722,660; 8,624,027; 8,486,965; 8,614,208; 7,625,910; 9,714,241; 9,718,814; 9,682,925; 9,745,325; 9,487,511; 9,567,358; 9,388,195; 9,447,092; 9,018,191; 9,278,931; 8,497,274; 8,143,258; 8,217,176; 8,063,066; 8,063,210; 9,568,483; U.S. Patent Application Publication No. 2011/0034469; 2009/0246198; 2009/0137580; 2017/0209452; 2015/0141380; 2010/0004247; 2017/0001994; 2017/044132; 2017/0029417; 2017/0015654; 2010/0168424; 2015/0160246; 2014/0336073; 2015/0140104; 2009/0253616; and 2010/0196907. For the compound of Formula III or compound of any formula, F is a halogen including fluorine.

In various embodiments, the Aurora Kinase B inhibitor is Danusertib (PHA-739358; CAS No. 827318-97-8; N-[5-[(2R)-2-methoxy-2-phenylacetyl]-4,6-dihydro-1H-pyrrolo[3,4-c]pyrazol-3-yl]-4-(4-methylpiperazin-1-yl)benzamide) or an inhibitor disclosed in the following patents, patent application publications, and publications that are all incorporated in their entirety by reference herein: U.S. Pat. Nos 7,141,568; 7,582,628; 8,084,455; 8,669,289; 9,016,221; 9,073,916; 9,1331,62; 9,447,092; 9,574,178; 9,801,851; U.S. Patent Application Publication No. 2011/0129467; 2012/0028917; 2012/0130144; 2012/0219506; 2012/0225057; 2013/0210771; 2014/0336073; 2015/0328193; 2015/0366866; 2016/0002222; 2016/0009785; and 2017/0121321.

In various embodiments, the Aurora Kinase B inhibitor is AT9283 (CAS No. 896466-04-9; 1-Cyclopropyl-3-(3-(5-(morpholinomethyl)-1H-benzo[d]imidazol-2-yl)-1H-pyrazol-4-yl)urea) or an inhibitor disclosed in the following patents, patent application publications, and publications that are all incorporated in their entirety by reference herein: U.S. Pat. Nos. 8,669,289; 8,110,573; 8,778,936; 8,883,790; 8,435,970; 8,399,442; 9,568,483; U.S. Patent Application Publication No. 2011/0159111; and 2013/0289014.

In various embodiments, the Aurora Kinase B inhibitor is PF-03814735 (CAS No. 942487-16-3; N-(2-((1S,4R)-6-((4-(Cyclobutylamino)-5-(trifluoromethyl)pyrimidin-2-yl)amino)-1,2,3,4-tetrahydro-1,4-epiminonaphthalen-9-yl)-2-oxoethyl)acetamide) or an inhibitor disclosed in the following patents, patent application publications, and publications that are all incorporated in their entirety by reference herein: U.S. Pat. No. 7,820,648.

In various embodiments, the Aurora Kinase B inhibitor is AMG 900 (CAS No. 945595-80-2; N-[4-[3-(2-aminopyrimidin-4-yl)pyridin-2-yl]oxyphenyl]-4-(4-methylthiophen-2-yl)phthalazin-1-amine) or an inhibitor disclosed in the following patents, patent application publications, and publications that are all incorporated in their entirety by reference herein: U.S. Pat. Nos. 7,560,551; 8,022,221; 8,623,885; 8,686,155; 8,921,367; 9,242,961; 9,359,355; 9,447,092; U.S. Patent Application Publication No. 2012/0028917; 2014/0113879; 2014/0114051; 2014/0127271; 2015/0072988; 2015/0079022; 2016/0008316; 2016/0009785; 2016/0129132; 2016/0213669; 2016/0264732; 2016/0298119; 2016/0304504; 2016/0346408; and 2016/0368933.

In various embodiments, the Aurora Kinase B inhibitor is Cytarabine (CAS No. 147-94-4; 4-amino-1-[(2R,3S,4S,5R)-3,4-dihydroxy-5-(hydroxymethyl)oxolan-2-yl]pyrimidin-2-one) or an inhibitor disclosed in the following patents, patent application publications, and publications that are all incorporated in their entirety by reference herein: U.S. Pat. Nos. 9,175,017; 9,233,115; 8,962,630; 9,512,107; and 8,975,282.

In various embodiments, the Aurora Kinase B inhibitor is an isolated antibody which specifically binds to Aurora Kinase B. The isolated antibody of various embodiments can have a complementarity determining region (CDR) portion (including Chothia and Kabat CDRs) specific for Aurora Kinase B.

In other embodiments, the Aurora Kinase B inhibitor is a sense or anti-sense nucleic acid molecule which inhibits the expression of Aurora Kinase B. In various embodiments, the Aurora Kinase B inhibitor is a small interfering RNA or microRNA-based compound that inhibits the expression of Aurora Kinase B.

In various embodiments, the administration of an Aurora Kinase B inhibitor or an amount of an Aurora Kinase B inhibitor is effective to reduce the dosage of glucocorticoid by or at least by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% relative to the administration of glucocorticoid without the Aurora Kinase B inhibitor. In various embodiments, an Aurora Kinase B inhibitor or an amount of an Aurora Kinase B inhibitor is effective to reduce the dosage of glucocorticoid by between any two percentages from above relative to the administration of glucocorticoid without the Aurora Kinase B inhibitor.

In various embodiments, the demethylase inhibitor reduces or prevents demethylation of G9a or GLP.

In various embodiments, the demethylase inhibitor are inihibitors of lysine demethylases or lysine demethylase inhibitors. The lysine demethylase inhibitors of various embodiments are capable of inhibiting the function of or reducing/preventing the expression of demethylases belonging to the LSD family including KDM1 family with LSD1 (KDM1A) and LSD2 (KDM1B) or the JmjC family. The JmjC family includes demthylases containing JmjC domains with at least 24 members. Examples include, but are not limited to, the KDM2 family (KDM2A and KDM2B), KDM3 family (KDM3A, KDM3B, and JMJD1C), KDM4 family (KDM4A, KDM4B, KDM4C, and KDM4D), KDM5 family (KDM5A, KDM5B, KDM5C, and KDM5D), and KDM6 family (KDM6A, KDM6B, and UTY).

Example of demethylase inhibitors of the LSD family include OG-L002 (CAS 1357302-64-7), ORY-1001 (CAS 1431326-61-2), RG6016 (4-N-[(1R,2S)-2-phenylcyclopropyl]cyclohexane-1,4-diamine;dihydrochloride), GSK2879552 (CAS 1401966-69-5), 2-PCPA (CAS 1986-47-6), NCL-1 (N-[(2R)-4-[3-[(1S ,2R)-2-aminocyclopropyl]phenoxy]-1-(benzylamino)-1-oxobutan-2-yl]benzamide), S2101 (2-(3,5-difluoro-2-phenylmethoxyphenyl)cyclopropan-1-amine), INCB059872 (see U.S. Patent Application Publication 2015/0225379 which is incorporated in it is entirety by reference herein), IMG-7289 (see U.S. Pat. No. 9,790,195 which is incorporated in it is entirety by reference herein), CC-90011 (see U.S. Patent Application Publication 2017/01347402 which is incorporated in it is entirety by reference herein); and Tranylcypromine ((1R,2S)-2-phenylcyclopropan-l-amine; CAS 155-09-9 and 3721-26-4).

For example, the demethylase inhibitor can be a compound having Formula IV

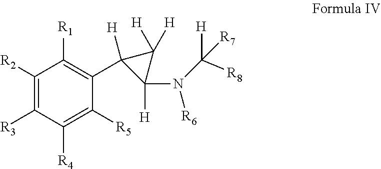

In Formula IV, each of R₁—R₅ is optionally substituted and independently chosen from —H, halo, alkyl, alkoxy, cycloalkoxy, haloalkyl, haloalkoxy, -L-aryl, -L-heteroaryl, -L-heterocyclyl, -L-carbocycle, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

R₆ is chosen from H and alkyl;

R₇ is chosen from H, alkyl, and cycloalkyl;

R₈ is chosen from H, C(═O)NRₓR_{y} and —C(═O)R_{z};

Rₓ when present is chosen from H, alkyl, alkynyl, alkenyl, -L-carbocycle, -L-aryl, -L-heterocyclyl, all of which are optionally substituted;

R_{y} when present is chosen from H, alkyl, alkynyl, alkenyl, -L-carbocycle, -L-aryl, -L-heterocyclyl, all of which are optionally substituted;

R_{z} when present is chosen from H, alkoxy, -L-carbocyclic, -L-heterocyclic, -L-aryl, wherein the aryl, heterocyclyl, or carbocycle is optionally substituted;

each L can be saturated, partially saturated, or unsaturated, and is independently chosen from H, —(CH₂)ₙ—(CH₂)—, —(CH₂)ₙC(═O)(CH₂)ₙ—, —(CH₂)C(═O)NH(CH₂)ωₙ—, —(CH₂)ₙNHC(═O)O(CH₂)ₙ—, —(CH₂)ₙNHC(═O)NH(CH₂)₁₁—, (CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8, wherein optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido.

In other examples, the demethylase inhibitor can be 3-[4-[(1R,2S)-2-aminocyclopropyl]phenyl]phenol, a compound having Formula V, or a pharmaceutically acceptable salt thereof.

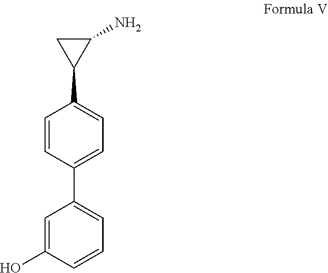 [SMILES: N[C@H]1C[C@@H]1c1ccc(-c2cccc(O)c2)cc1]

In various embodiments, the demethylase inhibitor is an inhibitor disclosed in the following patents, patent application publications, and publications that are all incorporated in their entirety by reference herein: U.S. Pat. Nos. 9,006,449; 9,676,701; U.S. Patent Application Publication No. 2014/0296255; 2014/0329833; 2016/0303095; and 2017/0209432.

In various embodiments, the demethylase inhibitor is ORY-1001 (CAS 1431326-61-2) or an inhibitor disclosed in the following patents, patent application publications, and publications that are all incorporated in their entirety by reference herein: U.S. Pat. Nos. 9,670,136 and 9,469597.

In various embodiments, the demethylase inhibitor is GSK2879552 (CAS 1401966-69-5) or an inhibitor disclosed in the following patents, patent application publications, and publications that are all incorporated in their entirety by reference herein: U.S. Pat. Nos. 8,853,490; 9,346,840; 9,795,597; and U.S. Patent Application Publication No. 2017/0183308.

Examples of inhibitors of the JmjC family include JIB04 (CAS 199596-05-9), IOX1 (CAS 5852-78-8), GSK-J1 (CAS 1373422-53-7), Daminozide (CAS 1596-84-5), or Methylstat (CAS 1310877-95-2).

For example, the demethylase inhibitor can be 5-chloro-N-[(E)-[phenyl(pyridin-2-yl)methylidene]amino]pyridin-2-amine, a compound having Formula VI, or a pharmaceutically acceptable salt thereof.

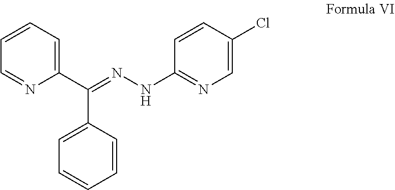 [SMILES: Clc1ccc(N/N=C(\c2ccccc2)c2ccccn2)nc1]

In various embodiments, the demethylase inhibitor is an inhibitor disclosed in the following patents, patent application publications, and publications that are all incorporated in their entirety by reference herein: U.S. Pat. No. 9,677,117 and U.S. Patent Application Publication No. 2016/0303095.

In various embodiments, the demethylase inhibitor is IOX1 (CAS 5852-78-8), 8-hydroxyquinoline-5-carboxylic acid, or an inhibitor disclosed in the following patents, patent application publications, and publications that are all incorporated in their entirety by reference herein: U.S. Pat. Nos. 4,738,796; 7,030,063; 8,871,789; 9,677,117; U.S. Patent Application Publication No. 2014/0154189; 2016/0272579; 2016/0303095; and 2017/0042842.

In various embodiments, the demethylase inhibitor is GSK-J1 (CAS 1373422-53-7), 3-[[2-pyridin-2-yl-6-(1,2,4,5-tetrahydro-3-benzazepin-3-yl)pyrimidin-4-yl]amino]propanoic acid, or an inhibitor disclosed in the following patents, patent application publications, and publications that are all incorporated in their entirety by reference herein: U.S. Patent Application Publication No. 2016/0272579; 2017/0042904; and 2016/0303095.

In various embodiments, the demethylase inhibitor is Daminozide (CAS 1596-84-5), 4-(2,2-dimethylhydrazinyl)-4-oxobutanoic acid, or an inhibitor disclosed in the following patents, patent application publications, and publications that are all incorporated in their entirety by reference herein: U.S. Pat. Nos. 9,192,608, 9,161,914, 9,072,781, U.S. Patent Application Publication No. 2017/0267629; and 2017/0128474.

In various embodiments, the demethylase inhibitor is Methylstat (CAS 1310877-95-2), (2E)-4-[Hydroxy[4-[[[4-[[[(1-naphthalenylamino)carbonyl]oxy]methyl]phenyl]methyl]amino]butyl]amino]-4-oxo-2-butenoic acid methyl ester, or an inhibitor disclosed in the following patents, patent application publications, and publications that are all incorporated in their entirety by reference herein: U.S. Pat. No. 8,735,622.

In various embodiments, demethylase inhibitor includes compounds having a half maximal inhibitory concentration (IC₅₀) or inhibitory constant (Kᵢ) for inhibiting of demethylase of less than about 1 μM. The demethylase inhibitor of various embodiments includes compounds having an IC₅₀ or Kᵢ for inhibiting of demethylase of about 0.1 nM, 0.2 nM, 0.3 nM, 0.4 nM, 0.5 nM, 0.6 nM, 0.7 nM, 0.8 nM, 0.9 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, 20 nM, 20.5 nM, 21 nM, 21.5 nM, 22 nM, 22.5 nM, 23 nM, 23.5 nM, 24 nM, 24.5 nM, 25 nM, 25.5 nM, 26 nM, 26.5 nM, 27 nM, 27.5 nM, 28 nM, 28.5 nM, 29 nM, 29.5 nM, 30 nM, 30.5 nM, 31 nM, 31.5 nM, 32 nM, 32.5 nM, 33 nM, 33.5 nM, 34 nM, 34.5 nM, 35 nM, 35.5 nM, 36 nM, 36.5 nM, 37 nM, 37.5 nM, 38 nM, 38.5 nM, 39 nM, 39.5 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, and 1000 nM. In various embodiments, the demethylase inhibitor includes compounds having an IC₅₀ or Kᵢ for inhibiting of demethylase between any two concentrations from above.

In various embodiments, the demethylase inhibitor is a plurality of demethylase inhibitors and can include various types of competitive, non-competitive, and uncompetitive inhibitors. In various embodiments, the plurality of demethylase inhibitor is 1, 2, 3, 4, 5, 6, 7, 8, 9, or 10 different demethylase inhibitors. In various embodiments, the plurality of demethylase inhibitors is a range between any number of different demethylase inhibitors listed above.

The demethylase inhibitor of various embodiments can include various compounds, antibodies, sense or anti-sense nucleic acid molecules, or combinations thereof that inhibit the function of or expression of demethylase inhibitor. In various embodiments, the demethylase inhibitor binds to at least one of demethylase inhibitor and antagonizes the activity of the demethylase inhibitor related nucleic acid or protein.

In various embodiments, the demethylase inhibitor is an isolated antibody which specifically binds to demethylase. The isolated antibody of various embodiments can have a complementarity determining region (CDR) portion (including Chothia and Kabat CDRs) specific for demethylase.

In various embodiments, the demethylase inhibitor is a small interfering RNA or microRNA-based compound that inhibits the expression of demethylase.

In various embodiments, the dosage of the demethylase inhibitor ranges from about about 1 nM to about 1000 nM. In other embodiments, the demethylase inhibitor ranges from about 10 nM to about 50 nM. In various embodiments, the dosage of demethylase inhibitor is about 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, 20 nM, 20.5 nM, 21 nM, 21.5 nM, 22 nM, 22.5 nM, 23 nM, 23.5 nM, 24 nM, 24.5 nM, 25 nM, 25.5 nM, 26 nM, 26.5 nM, 27 nM, 27.5 nM, 28 nM, 28.5 nM, 29 nM, 29.5 nM, 30 nM, 30.5 nM, 31 nM, 31.5 nM, 32 nM, 32.5 nM, 33 nM, 33.5 nM, 34 nM, 34.5 nM, 35 nM, 35.5 nM, 36 nM, 36.5 nM, 37 nM, 37.5 nM, 38 nM, 38.5 nM, 39 nM, 39.5 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, and 1000 nM. In various embodiments, the dosage of the demethylase inhibitor is between any two concentrations from above.

In various embodiments, the administration of a demethylase inhibitor or an amount of a demethylase inhibitor is effective to reduce the dosage of glucocorticoid by or at least by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% relative to the administration of glucocorticoid without the demethylase inhibitor. In various embodiments, a demethylase inhibitor or an amount of a demethylase inhibitor is effective to reduce the dosage of glucocorticoid by between any two percentages from above relative to the administration of glucocorticoid without the demethylase inhibitor.

In various embodiments, the inhibitor of any embodiment is a reversible or irreversible inhibitor.

In various embodiments, the composition of any embodiment includes a pharmaceutically acceptable excipient. Examples of pharmaceutically acceptable excipients include carriers include silicon dioxide (silica, silica gel), carbohydrates or carbohydrate polymers (polysaccharides), cyclodextrins, starches, degraded starches (starch hydrolysates), chemically or physically modified starches, modified celluloses, gum arabic, ghatti gum, tragacanth, karaya, carrageenan, guar gum, locust bean gum, alginates, pectin, inulin or xanthan gum, or hydrolysates of maltodextrins and dextrins.

In various embodiments, the composition of any embodiment includes an other anticancer agent(s). Examples of other anticancer agents include anticancer antimetabolites, anticancer antibiotics, plant-derived anticancer agents, anticancer platinum-coordinated complex compounds, anticancer camptothecin derivatives, anticancer biologics, and anticancer tyrosine kinase inhibitors.

In various embodiments are disclosed methods or systems of determining early relapse of hematologic or other malignancies in a subject including: quantifying a concentration or level of expression of Aurora Kinase B in a sample from a subject; comparing the concentration or level of expression of Aurora Kinase B in the sample to an Aurora Kinase B control; and identifying the subject as likely to have early relapse of a hematologic and other malignancy when the concentration or level of expression of Aurora Kinase B in the sample is greater than the Aurora Kinase B control.

In various embodiments are disclosed methods or systems of determining early relapse of hematologic or other malignancies in a subject and treating relapse of the hematologic or other malignancies in the subject including: quantifying a concentration or level of expression of Aurora Kinase B in a sample from a subject; comparing the concentration or level of expression of Aurora Kinase B in the sample to an Aurora Kinase B control; identifying the subject as likely to have early relapse of a hematologic and other malignancy when the concentration or level of expression of Aurora Kinase B in the sample is greater than the Aurora Kinase B control; and administering a glucocorticoid and an Aurora Kinase B inhibitor to the subject identified as likely to have early relapse of the hematologic and other malignancy when relapse of the hematologic and other malignancy occurs. In various embodiments, the administering further includes administering demethylase inhibitor to the subject identified as likely to have early relapse of the hematologic and other malignancy when relapse of the hematologic and other malignancy occurs.

In various embodiments are disclosed methods or systems of determining early relapse of hematologic or other malignancies in a subject including: quantifying a concentration or level of expression of Aurora Kinase B in a sample from a subject; comparing the concentration or level of expression of Aurora Kinase B in the sample to an Aurora Kinase B control; quantifying a concrnetration or level of expression of demethylase in the sample; comparing the concentration or level of expression of demethylase in the sample to a demethylase control; and identifying the subject as likely to have early relapse of a hematologic and other malignancy when the concentration or level of expression of Aurora Kinase B and demethylase in the sample is greater than the Aurora Kinase B and demethylase controls.

In various embodiments are disclosed methods or systems of determining early relapse of hematologic or other malignancies in a subject and treating relapse of the hematologic or other malignancies in the subject including: quantifying a concentration or level of expression of Aurora Kinase B in a sample from a subject; comparing the concentration or level of expression of Aurora Kinase B in the sample to an Aurora Kinase B control; quantifying a concentration or level of expression demethylase in the sample; comparing the concentration or level of expression of demethylase in the sample to a demethylase control; identifying the subject as likely to have early relapse of a hematologic and other malignancy when the concentration or level of expression of Aurora Kinase B and demethylase in the sample is greater than the Aurora Kinase B and demethylase controls; and administering a glucocorticoid, an Aurora Kinase B inhibitor, and a demethylase inhibitor to the subject identified as likely to have early relapse of the hematologic and other malignancy when relapse of the hematologic and other malignancy occurs.

In various embodiments are disclosed methods or systems of determining early relapse of hematologic or other malignancies in a subject including: quantifying a concentration or level of expression of demethylase in a sample from a subject; comparing the concentration or level of expression of demethylase in the sample to a demethylase control; and identifying the subject as likely to have early relapse of a hematologic and other malignancy when the concentration or level of expression of demethylase in the sample is greater than the demethylase control.

In various embodiments are disclosed methods or systems of determining early relapse of hematologic or other malignancies in a subject and treating relapse of the hematologic or other malignancies in the subject including: quantifying a concentration or level of expression of demethylase in a sample form a subject; comparing the concentration or level of expression of demethylase in the sample to a demethylase control; identifying the subject as likely to have early relapse of a hematologic and other malignancy when the concentration or level of expression of demethylase in the sample is greater than the demethylase; and administering a glucocorticoid and a demethylase inhibitor to the subject identified as likely to have early relapse of the hematologic and other malignancy when relapse of the hematologic and other malignancy occurs.

The method of various embodiments can further include isolating a sample for the subject. Examples of samples can include cell or tissues samples from the subjects such as blood samples or tumor biopsies from the subject.

In alternative embodiments, the administering step of various embodiments includes administering a glucocorticoid and at least one of an Aurora Kinase B inhibitor or demethylase inhibitor to the subject when the subject is identified as likely to have early relapse of the hematologic and other malignancy. The administering can include a plurality of administrations over a period of time (i.e. daily or monthly) to reduce the potential for relapse or prevent relapse.

In various embodiments, the administering of any embodiment is administering a composition of any embodiment to the subject.

In various embodiments, quantifying a concentration or level of expression of Aurora Kinase B or demethylase includes quantifying concentrations of protein, fragments, or portions of the protein or levels of RNA (i.e. mRNA) or complimentary DNA. Such methods of quantifying can include, for example, enzyme-linked immunosorbent assays, protein biochip arrays, microarrays including RNA and DNA microarrays, real time polymerase chain reactions, relative quantitative polymerase chain reactions, and absolute quantitative polymerase chain reactions.

In various embodiments, the Aurora Kinase B or demethylase control is a concentration of Aurora Kinase B or demethylase protein, RNA, cDNA, or portions thereof.

In various embodiments, the subject identified as likely to have early relapse of a hematologic and other malignancy has a 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, 2000%, 3000%, 4000%, 5000%, 6000%, 7000%, 8000%, 9000%, 10000%, 11000%, 12000%, 13000%, 14000%, 15000%, 16000%, 17000%, 18000%, 19000%, 20000% likelihood of relapse relative to a subject not identified as likely to have early relapse of a hematologic and other malignancy. In various embodiments, the likelihood is a range between any two percentages listed above.

In various embodiments, the subject is identified as likely to have early relapse of a hematologic and other malignancy when the amount or expression of Aurora Kinase B in the sample is greater than the control concentration of Aurora Kinase B. In various embodiments, the subject is identified as likely to have early relapse of a hematologic and other malignancy if the amount or expression of Aurora Kinase B in the sample is at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, 2000%, 3000%, 4000%, 5000%, 6000%, 7000%, 8000%, 9000%, 10000%, 11000%, 12000%, 13000%, 14000%, 15000%, 16000%, 17000%, 18000%, 19000%, 20000% more than the control concentration. In various embodiments, the subject is identified as likely to have early relapse of a hematologic and other malignancy when the amount or expression of Aurora Kinase B in the sample is at least about between any two percentages from above than the control concentration.

In various embodiments, the subject is identified as likely to have early relapse of a hematologic and other malignancy whenthe amount or expression of demethylase in the sample is greater than the control concentration of demethylase. In various embodiments, the subject is identified as likely to have early relapse of a hematologic and other malignancy when the amount or expression of demethylase in the sample is at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, 2000%, 3000%, 4000%, 5000%, 6000%, 7000%, 8000%, 9000%, 10000%, 11000%, 12000%, 13000%, 14000%, 15000%, 16000%, 17000%, 18000%, 19000%, 20000% more than the control concentration. In various embodiments, the subject is identified as likely to have early relapse of a hematologic and other malignancy when the amount or expression of demethylase in the sample is at least about between any two percentages from above than the control concentration.

The following examples illustrate the various embodiments of the present disclosure. Those skilled in the art will recognize many variations that are within the spirit of the present disclosure and scope of the claims.

### EXAMPLE 1

### Suppression of B-Cell Development Genes as Related to Glucocorticoid Efficacy in Treatment of Acute Lymphoblastic Leukemia

In various embodiments are disclosed Nextgen functional genomics identification B-cell development genes, pathways, and feedback loops that affect dexamethasone activity in B-ALL and suppression of the lymphoid-restricted PI3Kδ synergizes with dex in B-ALL by enhancing or restoring regulation of cell-death genes

Glucocorticoids (GCs), including dexamethasone (dex), are a central component of combination chemotherapy for childhood B-cell precursor acute lymphoblastic leukemia (B-ALL). GCs work by activating the glucocorticoid receptor (GR), a ligand-induced transcription factor, which in turn regulates genes that induce leukemic cell death. Which GR-regulated genes are required for GC cytotoxicity, which pathways affect their regulation, and how resistance arises are not well understood. Here we systematically integrate the transcriptional response of B-ALL to GCs with a next-generation shRNA screen to identify GC-regulated “effector” genes that contribute to cell death as well as genes that affect the sensitivity of B-ALL cells to dex. This analysis reveals a pervasive role for GCs in suppression of B-cell development genes that is linked to therapeutic response. Inhibition of PI3Kδ, a lynchpin in the pre-B-cell receptor and IL7R signaling pathways critical to B-cell development, with CAL-101 (idelalisib), interrupts a double-negative feedback loop, enhancing GC-regulated transcription to synergistically kill even highly resistant B-ALL with diverse genetic backgrounds. This work not only identifies numerous opportunities for enhanced lymphoid-specific combination chemotherapies that have the potential to overcome treatment resistance, but is also a valuable resource for understanding GC biology and the mechanistic details of GR-regulated transcription.

Although glucocorticoids (GCs or GC) have been used to treat lymphoid malignancies for over half a century^{1a}, the mechanism of their cytotoxicity is still not clear. Nonetheless, GC-based combination chemotherapy protocols are effective, particularly in children with B-cell precursor acute lymphoblastic leukemia (B-ALL). Although ˜90% of children on these protocols are cured, there are few effective treatments for the 10% who do not respond to this therapy^{1a}. Importantly, response to GCs alone is a good predictor of overall response to chemotherapy, indicating a central role for GCs in overall treatment efficacy and suggesting that the outcomes for resistant patients may be improved by enhancing GC potency^{1a}. Unfortunately, simply enhancing GC potency runs the risk of proportional increases in debilitating side effects, such as avascular necrosis and diabetes mellitus. The goal of this work in various embodiments is determining how GCs kill B-ALL and then systematically identify targets that enhance the lymphoid-specific potency of GCs in resistant patients.

GCs, such as dexamethasone (dex), induce cell death through the glucocorticoid receptor (GR), a ligand-activated transcription factor whose transcriptional activity is required for GC cytotoxicity^{1a}. GR regulates gene expression by binding DNA and nucleating the assembly of regulatory cofactors. Mutations in specific GR cofactors (CREBBP^{2a}, NCOR1, and TBL1XR1²a) diₛᵣᵤₚt GC-induced gene regulation in B-ALL and have been associated with GC-resistance. Dozens of GR-regulated genes have also been correlated with efficacy in B-ALL. Most prominently, repression of antiapoptotic BCL2 and simultaneous activation of proapoptotic BIM (BCL2L11) has been shown to tip the apoptotic balance of B-ALL toward cell death^{1a}. Regulation of these genes may be direct but also involves a feed-forward loop with KLF13, disruption of which results in high BCL2 expression and resistance^{1a}. GCs also increase expression of thioredoxin-interacting protein (TXNIP), which induces cell death by increasing reactive oxygen species and/or blocking glucose transport, effectively starving cells^{1a}. Other studies have shown that GCs may induce cell death by increasing glycolysis (via PFKFB2, PGK1, and PFKP¹)^{3a}, exhausting the depleted glycolytic reserves of lymphoid cells. Taken together, these studies suggest that dex-induced cell death is multifactorial, with faithful GR-driven gene regulation being essential for overall treatment response.

About two dozen genetic lesions have been associated with overall treatment resistance or relapse in B-ALL. In addition to mutation of GR cofactors, larger chromosomal changes such a hypodiploidy, t(9;22) (BCR-ABL), t(4;11) (MLL-AF4), and PR2Y8-CRLF2 have been associated with poor prognosis ^{1a}, but not resistance to a specific chemotherapeutic agent. Further, a growing number of resistance-associated lesions have been identified in factors that are involved in B-cell development, including CDKN2A/B, RAS, IZKF1, VREB1, and PAX5, but have not been mechanistically linked to treatment failure². Thus, how the majority of these lesions affect treatment response is not known.

### Methods

Cell lines, patient specimens, and reagents: B-ALL cell lines (697, B 1, KASUMI-2, KOPN-8, MHH-CALL4, MUTZ5, NALM-6, RCH-ACV, RS4;11, and SUP-B15) were obtained DSMZ or ATCC, who validated their genetic background, and then screened for mycoplasma contamination. The background of patient samples (HM2872, HM3101, HM3722) were tested by COG. The patient derived xenograft (ALL121) was genetically characterized previously^{1a}. B-cell lines and specimens were grown and maintained at 37° C., 5% CO₂, in RPMI medium supplemented with 10% FBS, unless otherwise noted. HEK293T cells (Clontech) were grown under the same conditions in DMEM supplemented with 10% FBS on poly-lysine-coated plates. Cells were treated with dexamethasone (Sigma, D1756) or CAL-101 (SelleckChem, S2226) dissolved in ethanol.

Gene expression microarrays: Illumina HT12 v4 microarrays were used to measure differential regulation of gene expression by dex. Cell lines and patient specimens were treated with 1 μM dex or ethanol control for 4 hours. RNA was isolated (Qiagen miRNAeasy) and run on arrays at UCLA Neurosciences Genomics Core (UNGC). At least three biological repeats were performed for each sample. Arrays were processed using the R/Bioconductor lumi package^{1a}. Batch effects were corrected using Combat from the SVA package^{2a}. Differential expression of dex-treated vs. vehicle was then calculated using ebayes from the limma package^{1a}. False discovery rate was calculated using Benjamini-Hochberg and q-value (qvalue package), each producing similar results. Data are available from the Gene Expression Omnibus (GSE94302), and code will be included with final submission.

Differential expression analysis: We used previously published xenograft data^{4a} to validate and lend power to dex-regulated genes identified in our lab (GEO No. GSE57795). We processed these arrays as described above, then combined the results with our data and filtered. A two-sided Kolmogorov-Smirnov test (KS test) was used to determine which genes were persistently up or down regulated across all samples using q-value of 10⁻⁴.

Clustering of regulated genes based on differential expression was performed using Euclidean Distance in R. Principle Component Analysis (PCA) was used on differentially regulated genes to determine the similarity of response to treatment, and Ingenuity Pathway Analysis software (Qiagen) to perform pathway and gene ontology analysis of differentially regulated genes. Additional methods: Chromatin Immunoprecipitation followed by deep sequencing (ChIP-seq)^{1a}, viral preparation^{1a}, shRNA screening^{2a}, cloning of individual shRNAs and knockdown^{5a}, quantitative polymerase chain reaction^{6a}, western blotting^{7a}, cell viability^{8a}, and patient-derived xenograft models^{9a,10a} were performed largely as previously described with additional details provided in the methods below.

Gene expression microarrays: To measure differential gene expression of cell lines and patient samples, we used Illumina HT12 v4 microarrays. Cell lines were grown in RPMI+10% FBS at 37° C., 5% CO2 to a density of ˜2 million cells/ml, then diluted in the afternoon to 1 million cells/ml in 5 ml ml per well in six-well uncoated plates. The next morning at ˜9 am, cells were then treated with 1 pM dexamethasone (dex) or ethanol control (final <0.1%) for four hours, spun down, and resuspended in 0.7 ml Qiazol/Trizol and stored at −80° C. until processing. Total RNA was isolated using Qiagen miRNAeasy kit, run on a Bioanalyzer (Agilent Technologies) to ensure quality, then sent to the UCLA Neurosciences Genomics Core (UNGC), where RNA was labelled and run on arrays. Primary tissue was obtained from the Children's Oncology Group (COG) in frozen vials. Cells were thawed, slowly resuspended in prewarmed RPMI+10% FBS, washed and plated. After one hour cells were counted and divided into aliquots in plates at a density >1 million cells/ml and at least 500,000 cells. Patient cells were treated exactly the same as cell lines.

Differential expression analysis: We used previously published xenograft data to validate and lend power to dex- regulated genes identified in our lab. Raw intensities were downloaded from the Gene Expression Omnibus (accession no. GSE57795), and processed using the same method described above for our own data. The two sets of differential expression data were then combined, filtered to obtain the single probe for each gene with the maximum mean response to dex. To determine which genes were consistently up or down across all sensitive samples, we performed a two-sided Kolmogorov-Smirnov test (KS test) using 10,000 randomly sampled probes as our null distribution. This test determines whether the distribution of differential expression values for a gene is significantly different from the null distribution, regardless of the P-Value for differential expression of the gene within a sample. We then corrected for false discovery using B-H, which produced more stable results than qvalue, and set an FDR cutoff of 0.01% based on a manual examination of the expression values for genes with higher and lower cutoffs. This combined list was also used to assess whether the response of B-ALL cell lines is similar to patient samples and xenografts. To assess this, we performed two tests. First, clustering of cell type by similarity was performed by determining the relative distance between differential expression values using a Euclidian distance measurement. Because xenografts were treated for 8 hours and our cells for 4 hours, they tended to exhibit stronger responses overall. To correct for this effect, we ranked each gene within a sample by log 2 fold change, then re-clustered. As a second check for similarity, we performed Principle Component Analysis (PCA) to determine whether the major sources of variation are similar between samples.

Chromatin Immunoprecipitation followed by deep sequencing (ChIP-seq) of GR: B1 and HM3101 cells were either grown or recovered in standard B-cell medium (RPMI+10% FBS) then diluted to 1 million cells/ml in 3m1 in 6 well plated and allowed to equilibrate for at least an hour. Cells were then treated with either 1 pM dex or ethanol control for 90 minutes. The B1 tracks represent a mixture of crosslinking conditions, all of which produced largely equivalent results, and were thus combined. B1 cells were crosslinked with 1% or 0.5% formaldehyde for 3-10 minutes, then quenched with either glycine or Tris. The best results from this experiment were used for the primary HM3101 cells: crosslinking with 1% formaldehyde for ten minutes then quenching with 750 mM Tris for 10 minutes on ice. Cells were then spun down and washed with PBS. The remaining protocol was performed using the Covaris low-SDS kit to isolate nuclei and shear chromatin. Sheared chromatin was then spun down, and the supernatant diluted 1: 1 with 2× RIPA buffer. GR was then immunoprecipitated using 6 pg antibody (n499, a generous gift from Keith Yamamoto) and magnetic protein G beads (Invitrogen). Crosslinks were reversed by addition of Proteinase K and incubation at 65° C. for at least 8 hours. DNA was then isolated, quantified by picogreen, and sequencing libraries prepared using the NuGen Ovation® Ultralow Library kit, and sequenced to depth of ˜50 million reads in 100 bp paired end mode.

Data were then processed using a standard pipeline. Reads were mapped to the hg19 version of the genome using Bowtie2 (Langmead and Salzberg, 2012) and converted to BAM files using BEDTOOLS (Quinlan and Hall, 2010). Peaks were then identified using MACS2 (Feng et al., 2012) by comparing dex to vehicle control and discarding PCR duplicates. The proximity of peaks to commonly regulated genes was measured using GRanges (R/Bioconductor), with the overrepresentation of GR binding peaks in B1 cells identified by Fisher's Exact test, which accounts for the increase in numbers of peaks in B1 cells. Figures were plotted using Gviz (R/Bioconductor).

shRNA Screen

Cell Culture: All cells were verified to be mycoplasma-negative and cultured without antibiotic for the duration of the screen. 293T cells were cultured in DMEM with 10% FBS+5 mL GlutaMAX™ (Thermo Fisher Scientific) and all B -ALL cell lines were cultured in RPMI 1640+10% FBS. All cells were cultured at 37° C.

Viral Production: We screened for genes affecting dex sensitivity using the 3rd generation (pMK1098-based) ultra- high coverage screen designed by Michael Bassik and Martin Kampmann largely as previously described (Kampmann and Bassik, 2013; Kampmann et al., 2014), but tailored for use in NALM-6 cells. To produce virus, we used low-passage Lenti-X™ 293T cells (Clontech) grown on poly-L-lysine-coated 15-cm plates to 70% confluence. We then transfected a total of 8 pg of library DNA and 8 pg of 3rd generation lentiviral packaging vectors (VSV-G, RSV, MDL, Addgene) using TransIT®-293 Transfection Reagent (Mirus Bio) and Opti-MEM according to manufacturers' instructions. For lentivirus produced with a combination of panels, the amount of library DNA contributed by each panel was proportional to the total number of genes represented in that panel to ensure even coverage. Approximately 14 hours post transfection, the medium was removed and replaced with fresh medium containing ViralBoost Reagent (ALSTEM) to increase viral titer. Approximately 60 hours post-transfection, the virus-containing supernatant was removed and spun down at 1000×g for 10 minutes, then passed through a 0.45 pM syringe filter to remove debris and any contaminant 293 cells. The virus was then used immediately for transduction. We performed the screen in two stages, first screening a panel of genes related to cancer comprising 2191 genes, then screening three additional panels (Apoptosis, Gene Expression, and Kinases) comprising 3570 genes, for a total of 5761 genes.

Transduction: NALM-6 cells were grown in T-175 flasks maintaining a cell density below 2 million cells/ml until a sufficient number of cells were obtained. In order to obtain a sufficient number of cells infected with each shRNA to measure both enrichment and depletion in response to dex selection, we sought to infect >1000 cells with each shRNA. In order to minimize double infection, we targeted an infection rate of ˜30%. For the Cancer library, we estimated that for 1000× coverage of 54,775 shRNAs at a 30% infection rate, we would have to start with ˜200 million cells. To ensure optimal infection, NALM-6 cells were plated at density of 1.5 million cells/ml in 6 well plates. Virus was added at a ratio of 1:10 viral supernatant/NALM-6 culture volume. Cells were the spinfected at 1000×g, 33° C., for 2 hours in the presence of 8 pg/ml polybrene. Cells were then washed in PBS, resuspended in fresh RPMI+10% FBS, and allowed to recover. Our actual infection rate was ˜70%, as measured by flow cytometry using the mCherry marker of the pMK1098 vector, for an approximate coverage of 2000 cells for each shRNA. Cells were then selected for infection using 0.5 pg/ml puromycin for 2 days, washed, and allowed to recover. The infected cells were then expanded and split into 5 pools: Infected (Time 0 or T0), Infected that would be untreated (TF), and three pools that would be treated with dex (R1-3). T0 and TF was composed of 100 million cells. T0 was frozen immediately, and TF was allowed to grow to a density no greater than 3 million cells/ml through the course of the experiment. Since we expected ˜80% death upon treatment, we started with 500 million cells each for R1-3.

Selection: Previous attempts at the screen had been hampered by inconsistent infection and selection with dex. We therefore decided to split TF and R1-3 into two experiments and treat with 50 nM dex, which kills ˜80% of NALM-6 cells in 3 days, and 1 pM dex, which kills >90%. TF samples were treated with equal volumes of DMSO vehicle. A separate TF sample was culture for both the 50 nM and 1 pM experiments. Cells were treated with each of these concentrations for three days, washed, allowed to recover, then treated two more times. From infection to the end of the experiment took 35 days for 50 nM dex and 46 days for 1 pM. The viabilities after each round of treatment are summarized in Table 1.

TABLE 1
Cancer Panel

| | | |
|---|---|---|
| Dexamethasone Concentration | Treatment Number | Viability |

| | | | |
|---|---|---|---|
| 50 | nM | 1 | 18% |
| 50 | nM | 2 | 72% |
| 50 | nM | 3 | 58% |
| 1 | pM | 1 | 16% |
| 1 | pM | 2 | 52% |
| 1 | pM | 3 | 67% |

Although 16% and 18% viability are slightly below the expected 20% expected viability after first treatment, the large number of infected cells likely blunted any bottlenecking effect.

Genomic DNA Library Preparation: At the completion of the experiment, 100 million cells for each sample (T0, TF, R1-3) were spun down, and genomic DNA was harvested using a QIAamp DNA Blood Maxi Kit. The DNA for each sample was digested overnight with 20 pl PvuII (NEB), then run in a single large well on a 0.8% 1×TAE agarose gel and stained with SYBR® Safe (Thermo Fisher Scientific). A slice encompassing the 1.3 kb fragment containing the integrated shRNA cassette was excised from the gel, and the DNA contained therein isolated using the Qiagen QIAquick Gel Extraction kit. Barcoded libraries were then prepared by PCR from the isolated and digested DNA and run on a 15% polyacrylamide gel. The PCR product bands (˜273 bp), which contained the barcoded libraries, were excised and extracted from the gel by electroelution. The DNA was then cleaned and concentrated using a MinElute PCR Purification Kit from Qiagen. The libraries were quantified by Bioanalyzer, mixed into one pool, and sequenced via Illumina HiSeq to a depth of >10 million 50 bp reads per sample.

AGEK Screen: Based on the results of the Cancer panel screen, we elected to screen the three other panels (Apoptosis, Gene Expression, Kinases) under 50 nM selection by dex. The screen was performed largely as described above, but our infection rate was <30% for a coverage of ˜900 cells/shRNA. Interestingly as shonw in Table 2, the viabilities in this screen remained high after each round of treatment, even though the same concentration of dex was used.

TABLE 2
Apoptosis, Gene Expression, and Kinase Panels

| Dexamethasone Concentration | Treatment Number | Viability |
|---|---|---|
| 50 nM | 1 | 52% |
| 50 nM | 2 | 66% |
| 50 nM | 3 | 65% |

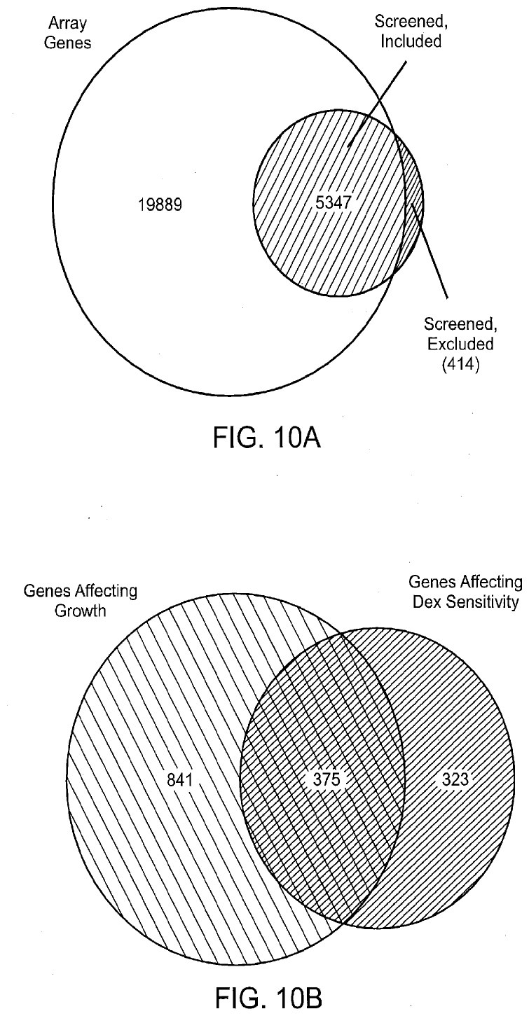

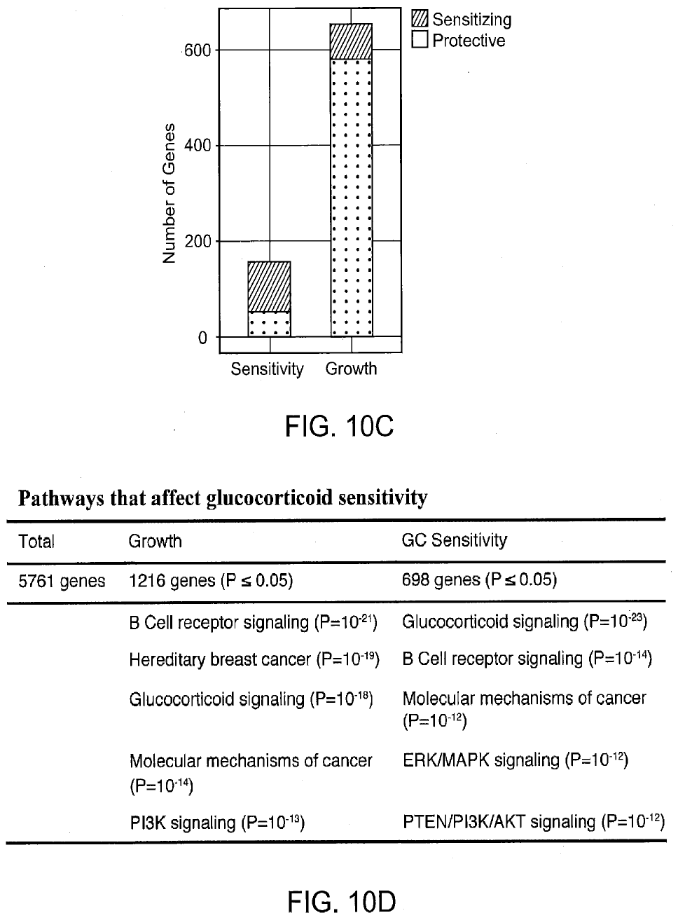

Nonetheless, as can be seen from the distribution of hits shown in FIG. 10B, this produced a better distribution of enrichment values and more significant hits, particularly for those that sensitized the cells to treatment.

Processing shRNA screen: Processing of the reads from the screen were performed essentially as described previously (5). Briefly, initial processing was performed using the latest version of Glmap (v 1.01) (http://gimap.ucsf.edu/). Raw reads were first trimmed to the essential 23 base pairs, then mapped onto their respective libraries using Bowtie. Then, the effect of each gene knockdown on growth (“gamma”), overall sensitivity (“tau”) or growth-corrected sensitivity (“rho”) was determined by comparing the initial infection (T0) sample to the untreated sample (TF), which had divided for the duration of the experiment, and then comparing these samples to each biological treatment replicates (R1—R3) using analyze_primary_screen.py. Importantly, this program determined significance by two different methods, Mann-Whitney and KS-test. A comparison graph of these was analyzed to ensure consistency. Since we have three biological replicates, we determined significant hits by averaging across the replicates using primary_avgrhos.py, which yields a table containing both the average phenotype (form ˜−1 to ˜1) and an associated P-Value.

As noted in the text, the screen was performed in two batches: The Cancer Panel and the Apoptosis, Gene Expression, and Kinase (AGEK) panels. Although the Cancer panel appeared to be under stronger selection, despite use of the same 50nM dex concentration, the results were combined after this processing step.

The data were then further processed and plotted using R/Bioconductor (See Attached Vignette). An adjusted P-Value for each gene was calculated by two methods: B-H and Q-value, which yielded similar results.

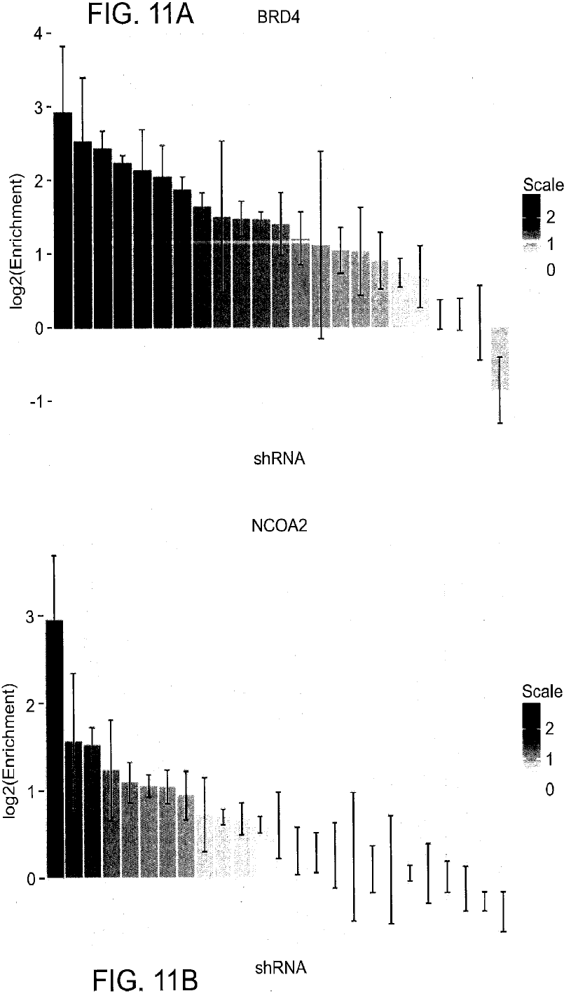

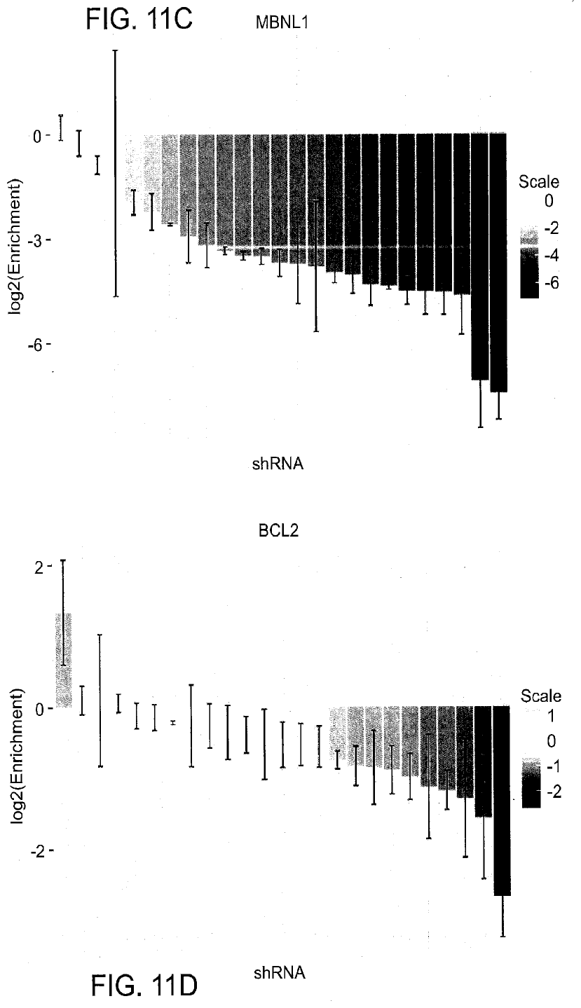

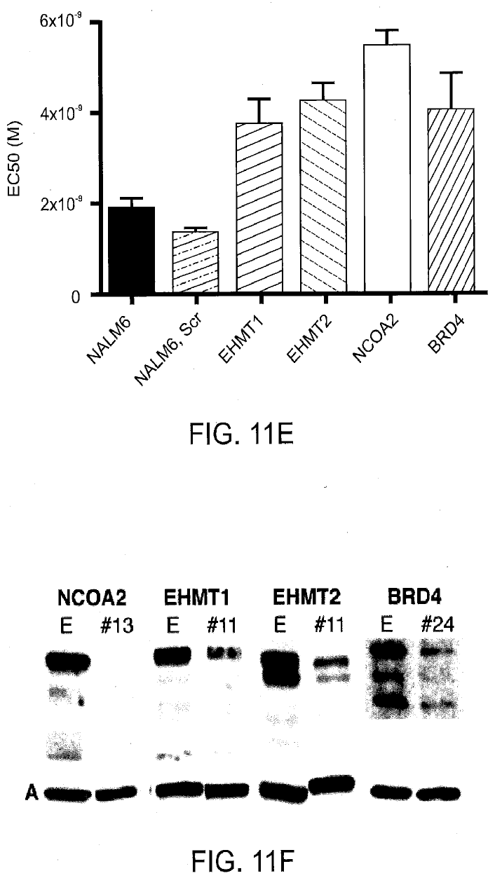

Cloning and knockdown of individual genes: In order to both validate the results of the screen and to study the effect of single gene knockdown, we recloned significantly enriched or depleted shRNAs into two lentiviral expression backbones, pMK1200 and pMK1221, which are driven by the E1A and SFFV promoters, respectively. In our hands, B-ALL cell lines were more efficiently infected by pMK1221-derived constructs, which also appeared to produce better knockdowns (data not shown). We first identified the most strongly enriched shRNAs by taking the average enrichment of each shRNA (output of analyze_primary_screen.py) in the treated vs. growth control (FIGS. 11A-11F).

Two to three were typically identified, then cloned as described (5), packaged into lentivirus, harvested, then spinfected into NALM-6 cells as described above. Knockdown was measured by western blot using antibodies against NCOA2, EHMT1, EHMT2, and BRD4 (generous gift from David Price), described below.

Cell death assays: We used PrestoBlue to measure cell viability in response to knockdown and treatment. Cells were grown to a density of 250,000 cell/ml, then dispensed into black tissue culture plates and grown overnight. In the morning, cells were treated. For both CAL-101 (Selleckchem, S2226) and dex (Sigma, D4902), viability was measured after 3 days. As PrestoBlue measures NADPH/NADH metabolites, the balance of which can be affected by GC treatment, we performed frequent cell count spot checks manually by trypan blue exclusion to ensure that fluorescence measurements reflected true viability. Both CAL-101 and dex were dissolved in ethanol, and used 1:1000 in cell culture for a final ethanol concentration of 0.1%. Combination treatments were performed in triplicate in 384 well plates with 20 dilutions of dex from 10\hat{ }M to 0 and 9 dilutions of CAL-101 from 10\hat{ }M to 0. Viabilities and EC50 were then calculated using four parameter non-least squares fitting using Prism 6. qPCR of Causative genes: Cells were grown to a density of 1 million cells/ml, then aliquoted into 6 well plates in the afternoon, then grown overnight. Cells were then treated with the drugs and times indicated, at which point cells were spun down at 400 g for 5 minutes, the medium aspirated, and the pellet resuspended in 0.7 ml Qiazol/Trizol and frozen. RNA was then isolated using the miRNAeasy protocol. cDNA was then prepared form 1\hat{ }g of RNA suing SuperScript3, and qPCR performed using BioRad iQ mix. Primers for qPCR were designed using the IDT web site, then tested for each gene. Primer pairs with efficiencies closest to 100% were used in subsequent assays (see Table 3 below). Expression levels for each gene were corrected for primer efficiency, normalized to RPL19, then compared to controls (Pfaffl, 2001).

TABLE 3
qPCR Primers

| Gene | Primer | Primer Sequence | SEQ ID NO: # |
|---|---|---|---|
| BCL2 | Forward | GTGGATGACTGAGTACCTGAAC | SEQ ID NO: 1 |
| BCL2 | Reverse | GCCAGGAGAAATCAAACAGAGG | SEQ ID NO: 2 |
| BIM | Forward | TGATTCTTCAGATGCCCTTCC | SEQ ID NO: 3 |
| BIM | Reverse | AACTTGATTTCTCCGCAACC | SEQ ID NO: 4 |
| IL7R | Forward | CTGGAGAAAGTGGCTATGCTC | SEQ ID NO: 5 |
| IL7R | Reverse | ACATCTGGGTCCTCAAAAGC | SEQ ID NO: 6 |
| Myc | Forward | GGACCCGCTTCTCTGAAAG | SEQ ID NO: 7 |
| Myc | Reverse | GTCGAGGTCATAGTTCCTGTTG | SEQ ID NO: 8 |
| PIK3CD | Forward | AGTGGAACAAGCATGAGGATG | SEQ ID NO: 9 |
| PIK3CD | Reverse | ACTTGATGGCGAAGGAGC | SEQ ID NO: 10 |
| TXNIP | Forward | GATCTGAACATCCCTGATACCC | SEQ ID NO: 11 |
| TXNIP | Reverse | CATCCATGTCATCTAGCAGAGG | SEQ ID NO: 12 |
| RPL19 | Forward | ATCGATCGCCACATGTATCA | SEQ ID NO: 13 |
| RPL19 | Reverse | GCGTCGTTCCTTGGTCTTAG | SEQ ID NO: 14 |

The optimal signal was determined by incubating cells with 1 \hat{ }M dex for 0, 4, 8, and 24 hours. Each of the genes tested reached maximum activation or repression at 24 hours (data not shown). For combination treatments, we chose the EC50 for dex for each cell line as the low concentration (NALM6: 3.9 nM, SUP-B15: 1.6 nM, RCH-ACV: 500 nM), and approximately the EC90 for dex as the high concentration (except RCH-ACV, which maintained ˜50% viability even at the highest dex concentrations) (NALM-6: 62.5 nM, SUP-B15: 4 nM, RCH-ACV: 5\hat{ }M). For CAL-101, the response of each cell line was different, and we chose concentrations that synergized with dex in all samples, one low (7.7 nM) and one higher concentration (280 nM). Cells were incubated with dex alone, CAL-101 alone, and in combination for 24 hours, then harvested and qPCR performed as described above. Each experiment was performed at least three times, and analyzed using GraphPad (Prism 6). Significant differences between drug and control and between either drug alone and combinations was determined by two sided Student's t-test (P-Value<0.05).

### Figure Descriptions

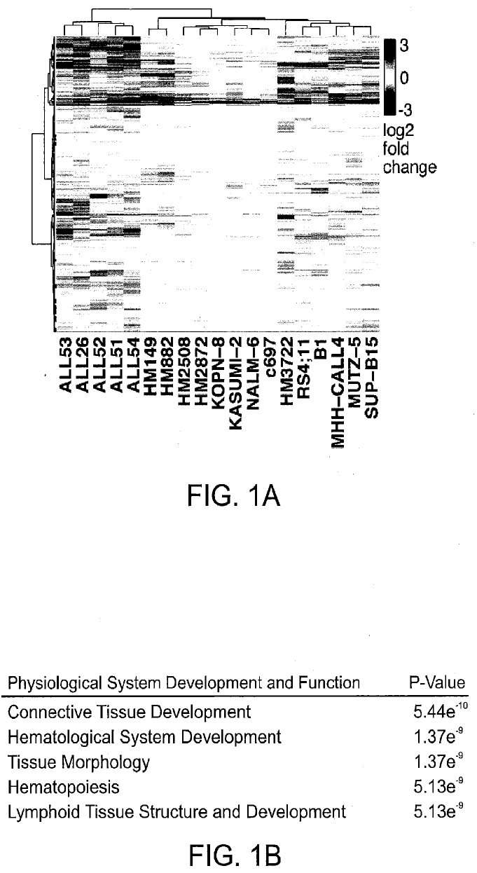

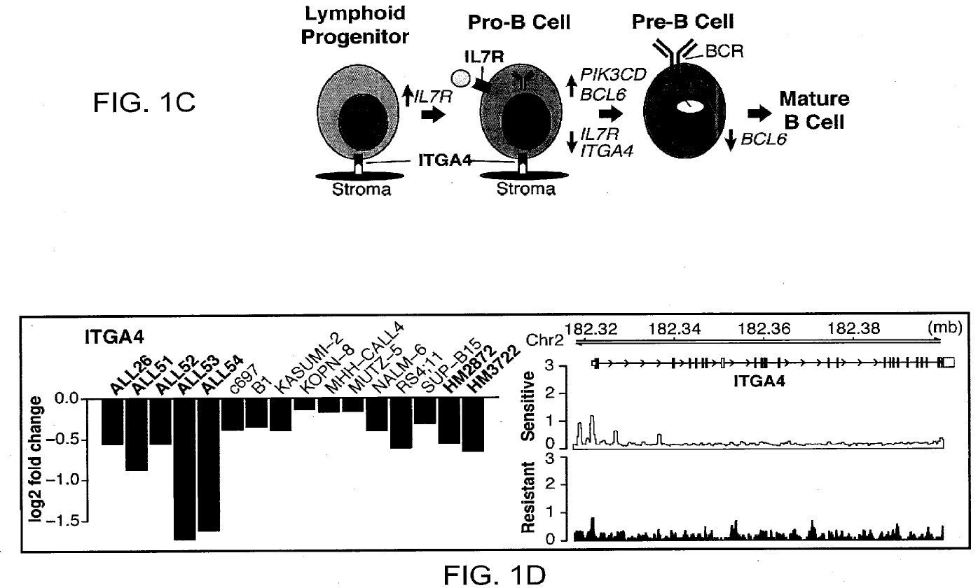

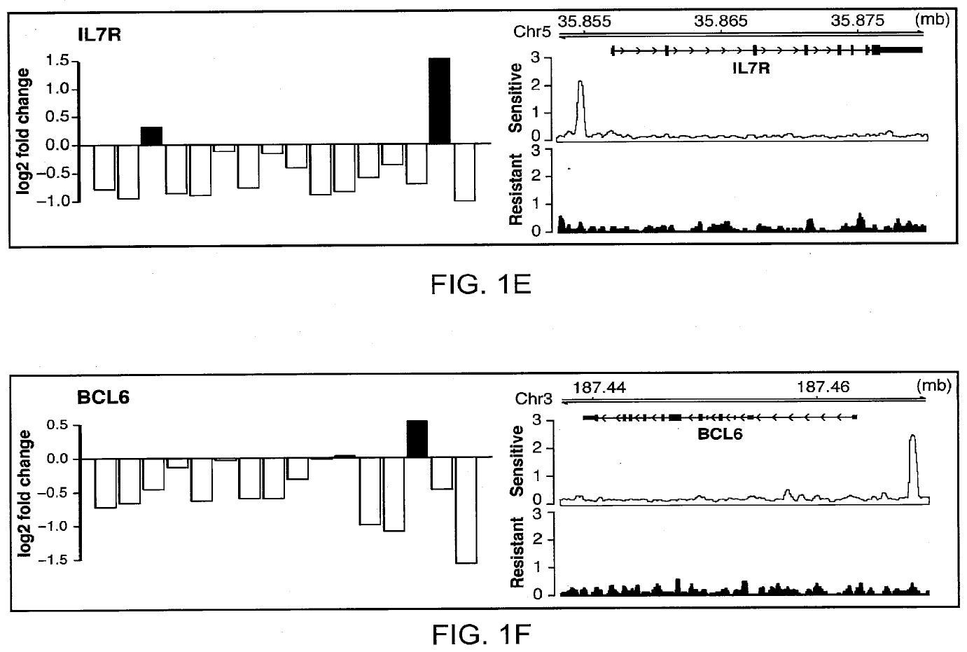

The results as shown in FIGS. 1A-1F generally highlight that Dexamethasone regulates B-cell development genes in sensitive B-ALL samples. FIG. 1A shows that Heatmap clustering genes commonly regulated (KS-Test, q-value ≤10⁻⁴) by dex across 16 samples. Primary and PDX samples are marked red (gray), cell lines black. FIG. 1B is an Ingenuity Pathway Analysis of regulated genes shows enrichment for hematological development genes. FIG. 1C shows a stop or push through model for dexamethasone in B-cell development highlighting the roles of dex-repressed ITGA4, IL7R, and BCL6. FIGS. 1D and 1F show differential gene expression values across sensitive B-ALL sample across samples measured by microarray (left) and GR occupancy in Sensitive (B1) and Resistant (HM3101) samples measured by ChIP-seq in response to dex suggest ITGA4, IL7R, and BCL6 are direct targets of GR regulation.

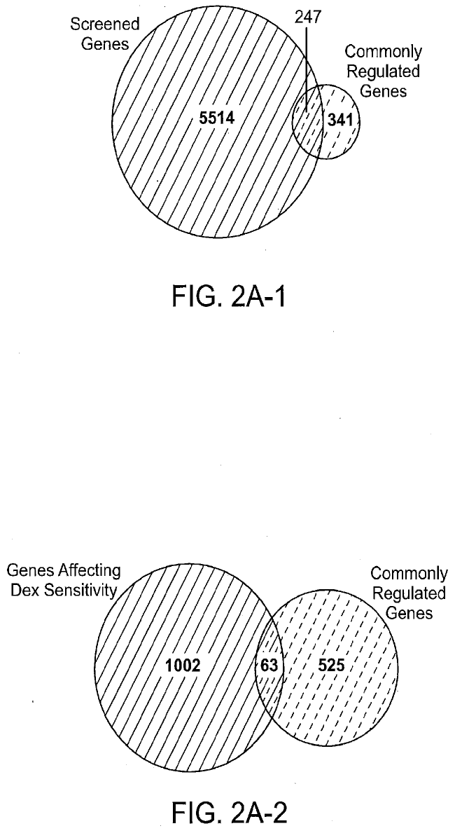

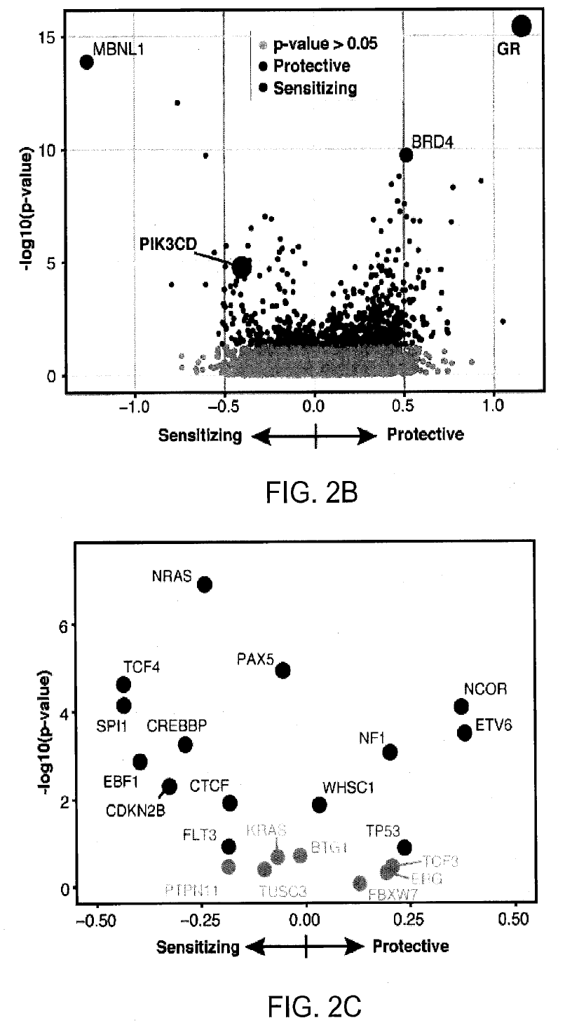

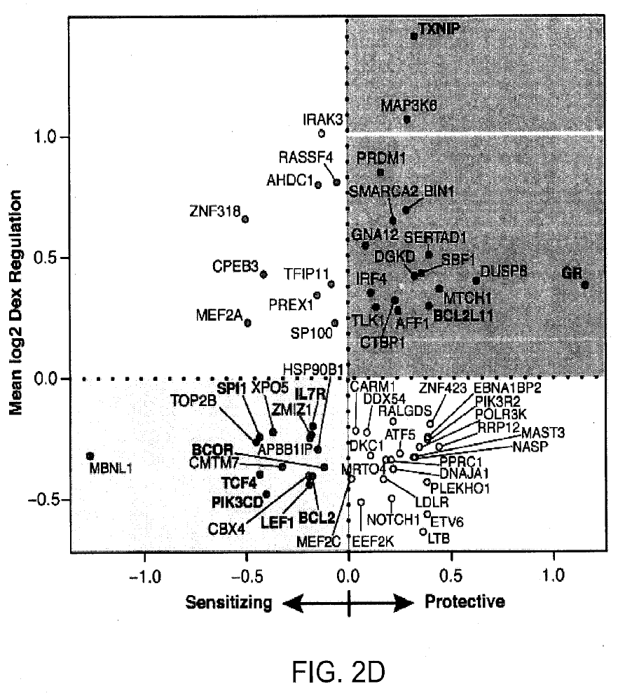

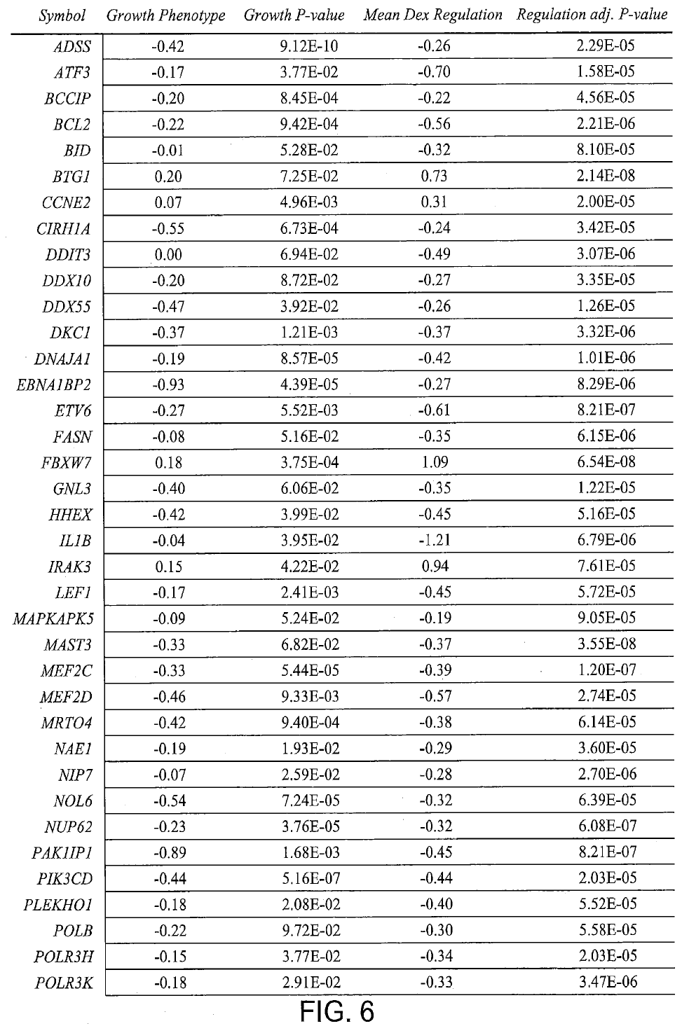

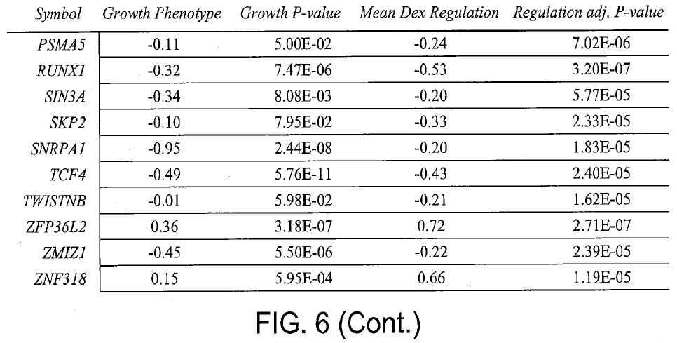

The results as shown in FIGS. 2A-2D generally highlight next generation shRNA screen identifies sources of sensitivity and resistance to dex in B-ALL. FIGS. 2A-1 and 2A-2 are Venn diagrams showing that 247 of the CRGs are covered by the screen, 63 of which affect dex-sensitivity. FIG. 2B is a Volcano plot of the effect of shRNA gene knockdown on dex-sensitivity. Each point is a gene with the log significance on the Y-axis and relative effect (phenotype) on dex-induced cell death on the X-axis. GR is the most protective when knocked down, and knockdown of PIK3CD makes NALM-6 cell more sensitive. Top hits (Mann Whitney, p-value ≤0.05) are Green: Sensitizing; Purple: Protective; Grey: p-value>0.05. FIG. 2C is a zoomed-in view of volcano plot showing genes commonly mutated in treatment resistant or relapsed patients with B-ALL have an effect on dex-sensitivity when knocked down (FIG. 6). FIG. 2D shows Identification of effector genes from among the Commonly Regulated Genes. Plot of dex-sensitivity phenotype when knocked down (X-axis) versus the average change in expression in response to dex (Y-axis) for genes that are significantly regulated by dex and are top hits in the screen. Genes validated as effectors of dex-induced cell death are either: 1) downregulated by dex and cause sensitivity when knocked down (green shaded or lighter gray) or; 2) upregulated by dex and are protective when knocked down (purple shaded or darker gray). Genes involved in B-cell development or previously identified as effectors are in bold.

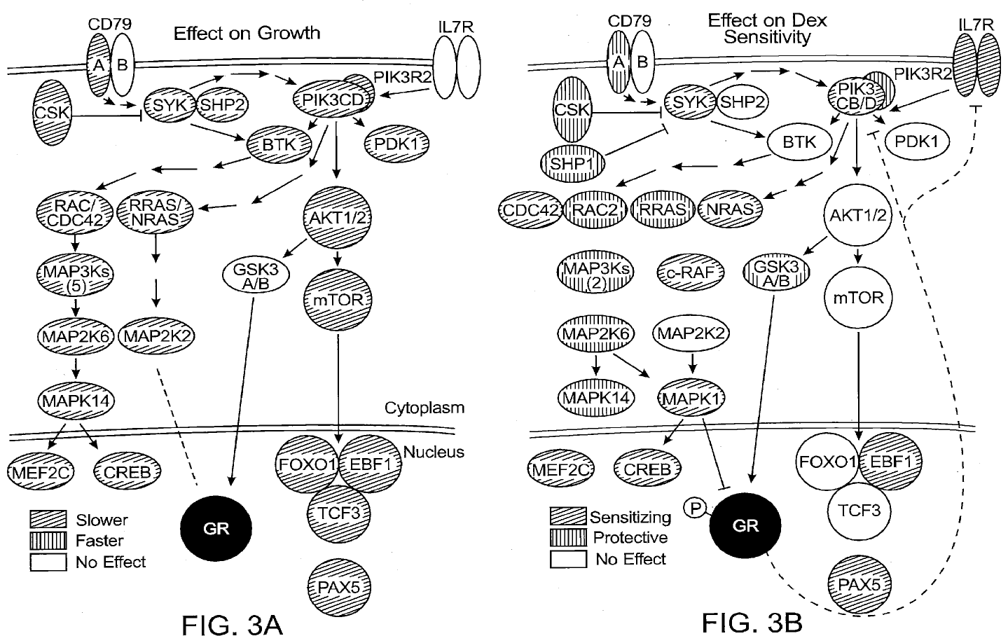

The results of FIGS. 3A-3B generally show Suppression of B-cell receptor signaling is detrimental to growth and sensitizes B-ALL to dexamethasone. The effects of gene knockdown on growth as shown in FIGS. 3A and dex-sensitivity as shown in FIG. 3B are overlaid on components of the B-cell receptor pathway. Genes are present when included in the screen, and shaded when the effect of knockdown is significant (Mann-Whitney, p-value≤0.05). Dashed lines indicate repression of PIK3CD and IL7R expression by dex. (Diagrams based on Ingenuity Pathways, and other literature^{26a,46a})

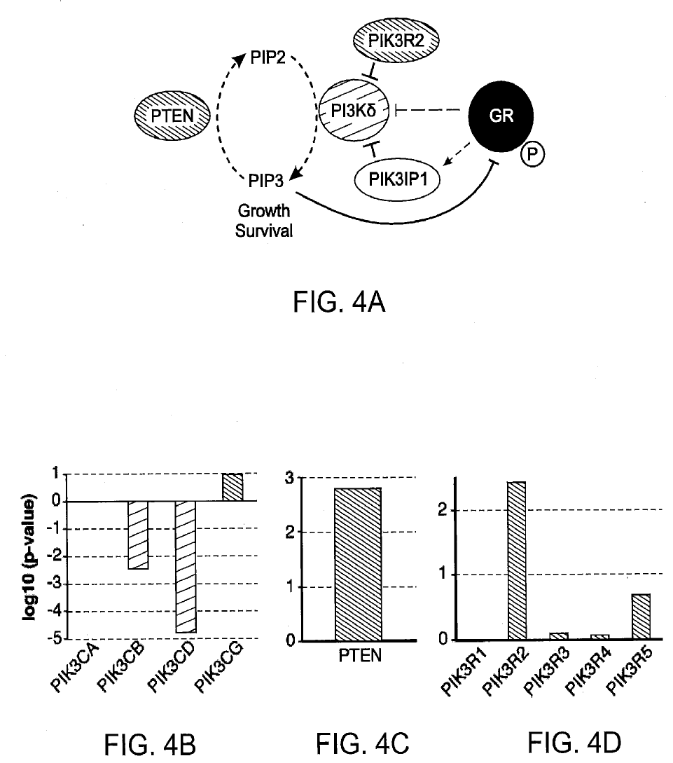

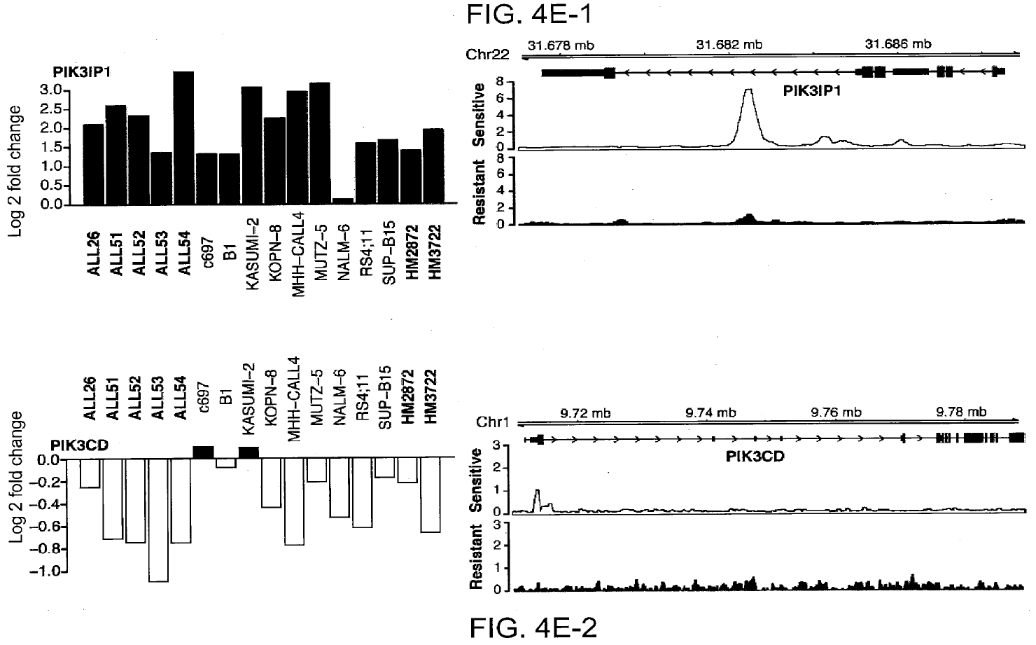

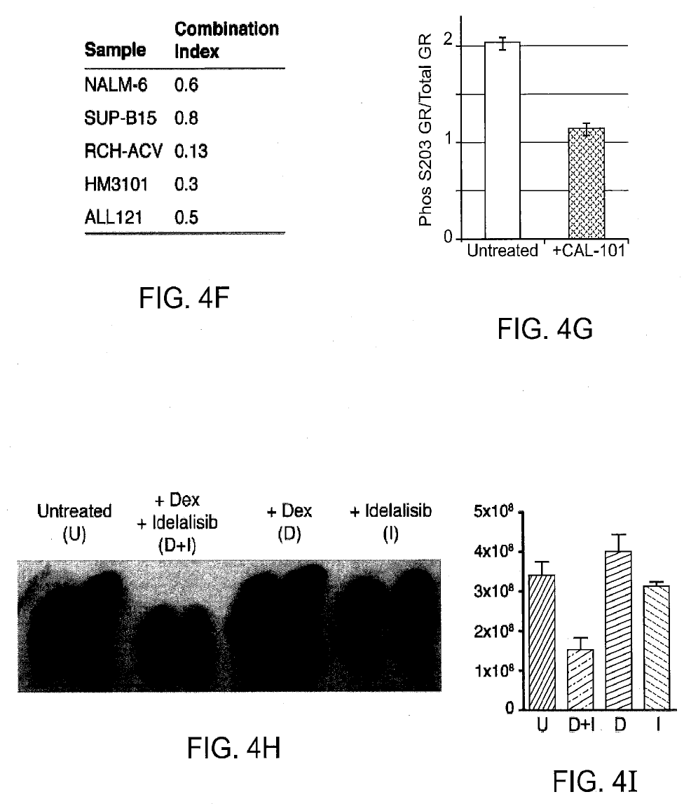

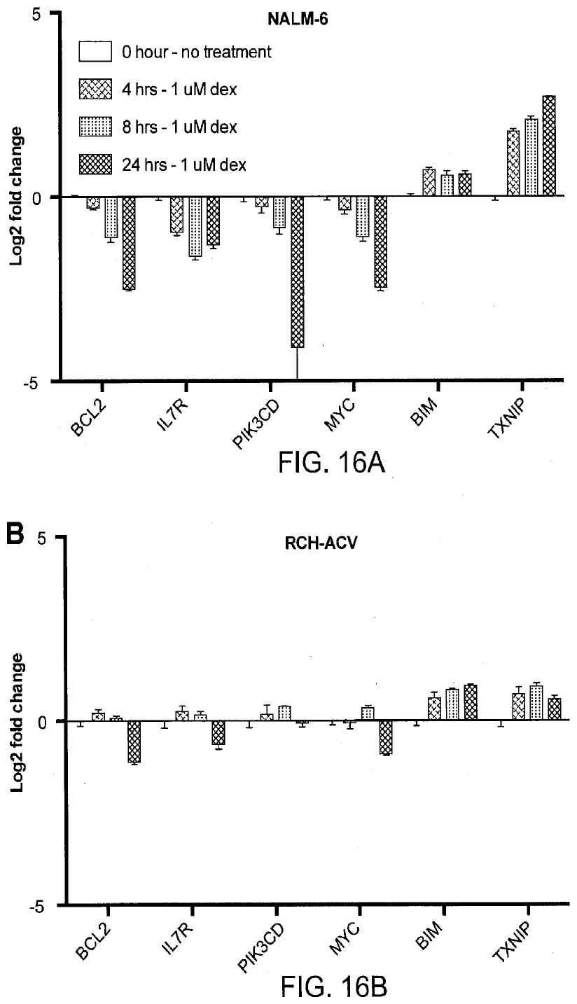

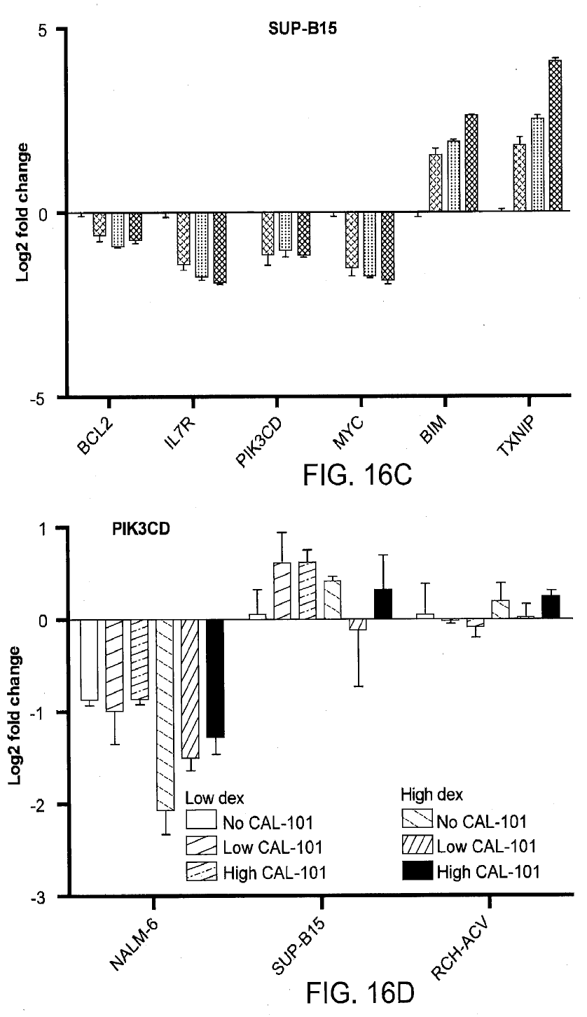

The results of FIGS. 4A-4I generally show disruption of double-negative feedback loop between PI3Kδ and GR enhances dexamethasone cytotoxicity. FIG. 4A is a schematic feedback loop based on combined data from the shRNA screen and microarray gene expression data. Dex induced repression of PIK3CD (blue blocking arrow, PI3Lδ) and activation of PIK3IP1 (red arrow or gray arrow) gene expression. shRNA knockdown of PTEN and PIK3R2 was protective (purple or darker gray), whereas knockdown specifically of PIK3CD sensitized cells to dex (green or lighter gray). Thus, interruption of PIK3δ inhibition of GR is expected to synergistically induce cell death. FIGS. 4B, 4C, and 4D highlight results of the shRNA screen. Bar graphs show the log10(p-values) of the hits from the shRNA screen. Sensitizing hits have been depicted as negative (green), protective as positive (purple). FIGS. 4E-1 and 4E-2 show the effect of dex on gene expression. Fold change of gene expression across sensitive B-ALL samples as measured by microarray (left) and GR occupancy as measured by ChIP-seq (right) post dex treatment. Primary and PDX samples are marked red, cell lines in black. ChIP-seq data are shown for Sensitive (B1) and Resistant (HM3101) samples. The presence of GR binding sites in sensitive cells for both PIK3IP1 and PIK3CD indicates potential direct regulation by dex. FIG. 4F shows the combination index of dex and CAL-101 in sensitive (NALM-6, SUP-B15) and resistant (RCH-ACV) cell lines, a resistant patient sample (HM3101), and a multiply relapsed refractory patient derived mouse xenograft (ALL121) (super additive<1, Calcusyn). Numbers reflect isobolograms depicted in FIGS. 16A-16D. FIG. 4G is a quantification of westerns against phospho-S203 of GR in the absence and presence of PI3Kδ inhibition (error bars represent SEM across 4 time points). CAL-101 treatment reduces GR 5203 phosphorylation, likely increasing GR activity. FIG. 4H shows spleens of mice (n=5 mice/cohort) engrafted with relapsed B-ALL cells (ALL121) and treated with vehicle, dexamethasone (7.5 mg/kg), idelalisib (50 mg/kg), or both for two weeks. Enlarged spleens indicate the accumulation of lymphoblasts. Treatment with either dex or Idelalisib alone failed to significantly reduce spleen size compared to untreated control; however, treatment with both dex and idelalisib significantly reduced spleen size, indicating a synergistic effect between the two drugs. FIG. 4I shows the total number of human ALL cells (y-axis) in spleens of mice in FIG. 4H as measured by quantitative flow cytometry.

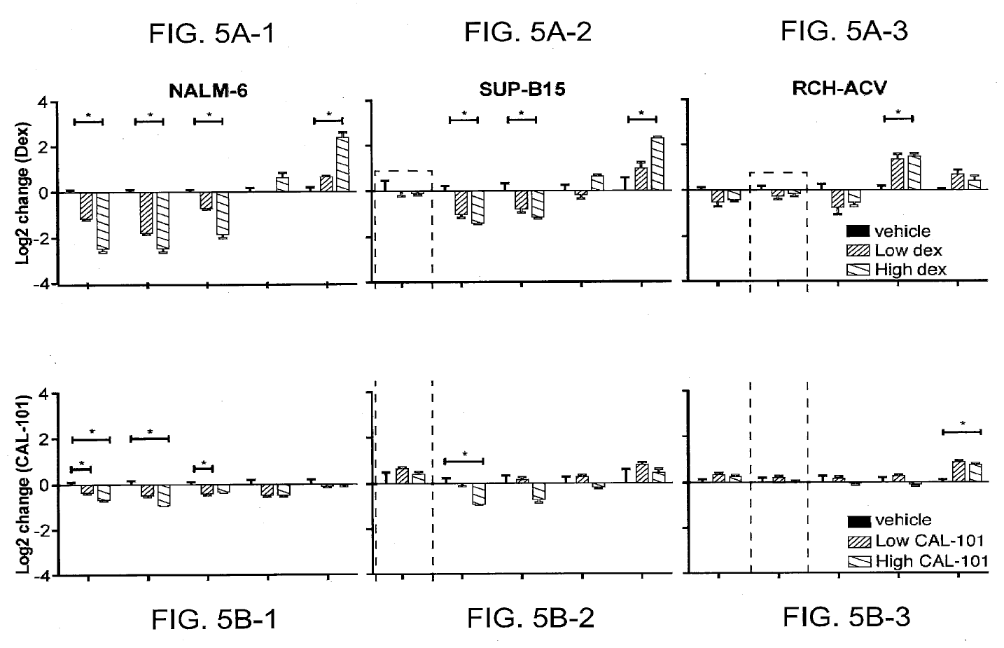

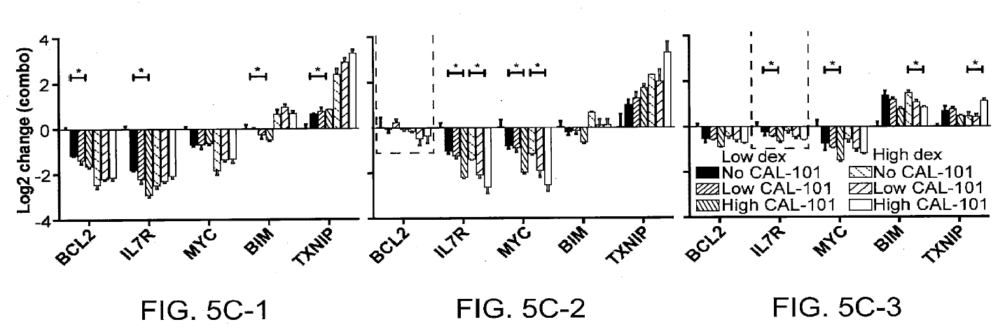

The results as shown in FIGS. 5A-5C highlight the inhibition of PI3Kδ synergizes with dex in regulating cell-death effector genes. FIGS. 5A-1, 5A-2, and 5A-3 show a change in gene expression measured by qPCR in response to two concentrations of dex at 24 hours in three cell lines. FIGS. 5B-1, 5B-2, and 5B-3 show a change in gene expression measured by qPCR in response to two concentrations of CAL-101 alone and in combination with two concentrations of dex as shown in FIGS. 5C-1, 5C-2, and 5C-3 at 24 hours in the same cell lines. Experiments represent at least 3 biological repeats, * indicates p-value≤0.05 (see Materials and Methods for details). Dashed boxes highlight genes whose regulation is restored by CAL-101 (idela).

FIG. 6 is a table listing of dexamethasone effector genes, growth.

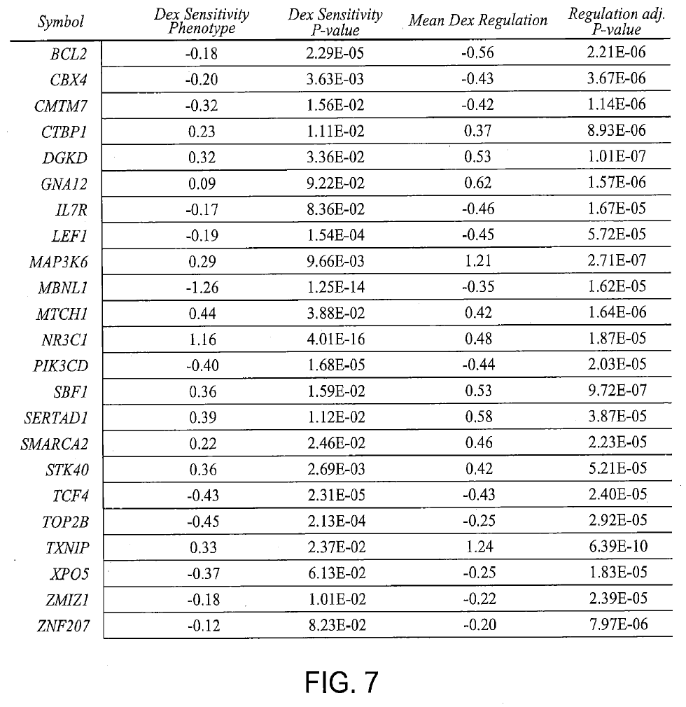

FIG. 7 is a table listing of dexamethasone effector genes, sensitivity.

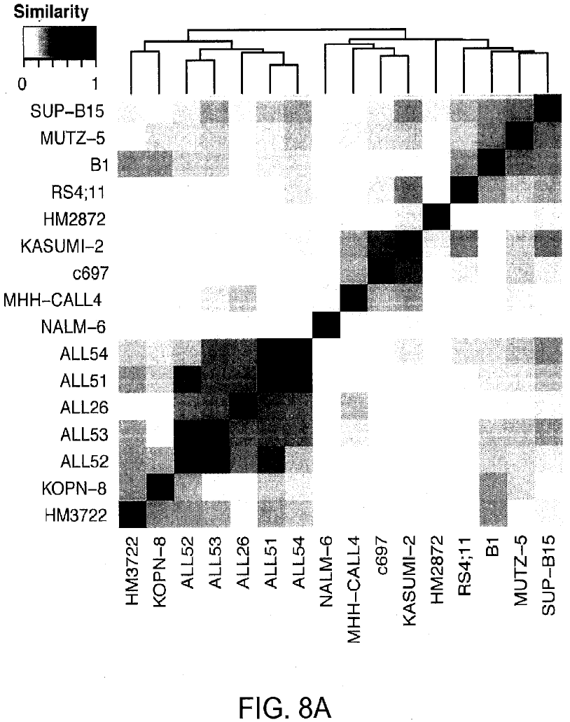

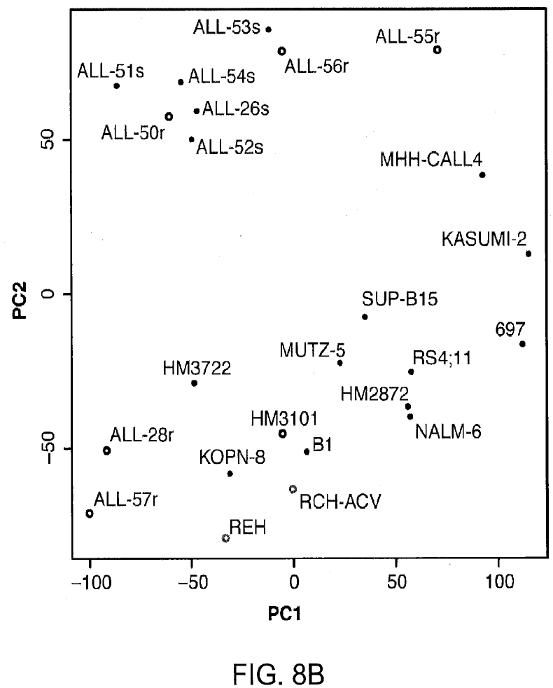

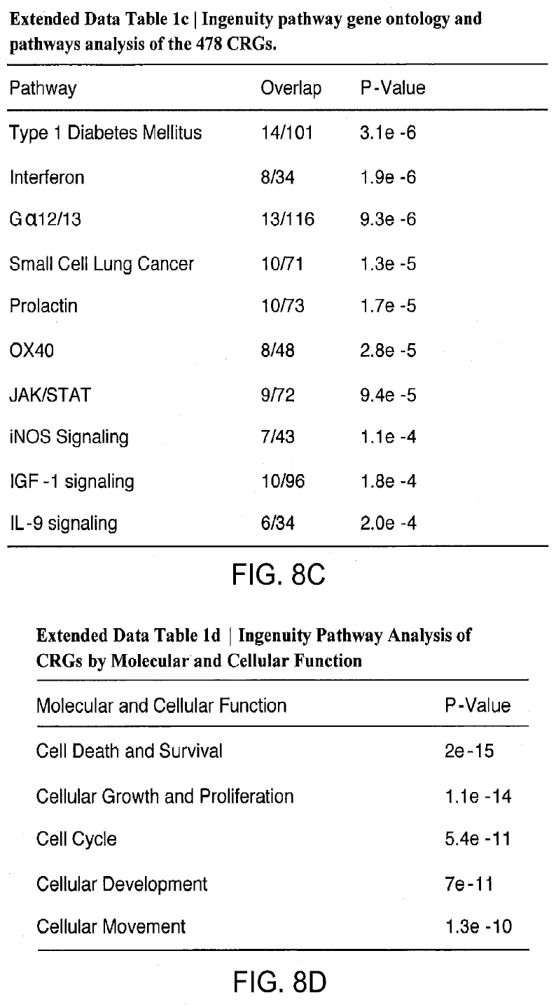

The results as shown in FIGS. 8A-8D generally show commonly regulated genes (CRGs) defined by comparison of sensitive cell lines and patient samples. Differential gene expression in response to 1 μM dex treatment for 4 hours (cell lines (Blue-SUP-15, MUTZ-5, B1, RS4;11, KASUMI-2, c697, MHH-CALL4, NALM-6, KOPN-8, RED, RCH-ACV) and patient samples (Red—any one of ALL-53s, ALL-55r, ALL-56r, ALL-54s, ALL-51s, ALL-50r, ALL-52s, HM3722, HM3101, ALL-28r, ALL-57r, HM2872, ALL54, ALL51, ALL26, ALL53, ALL52, HM3822) and 8 hours (xenografts, Red—any one of ALL-53s, ALL-55r, ALL-56r, ALL-54s, ALL-51s, ALL-50r, ALL-52s, HM3722, HM3101, ALL-28r, ALL-57r, HM2872, ALL54, ALL51, ALL26, ALL53, ALL52, HM3822) was measured using Illumina Microarrays. FIG. 8A is a Correlation heatmap comparing the similarity of transcriptional response of cells to dex (dark is most similar). Samples are grouped by unsupervised clustering showing that although the PDX samples are very similar (e.g. ALL52) a primary patient specimen (HM3722) and a cell line (KOPN-8) also respond similarly to dex. FIG. 8B is a principle component analysis shows little separation between primary samples (red—any one of ALL-53s, ALL-55r, ALL-56r, ALL-54s, ALL-51s, ALL-50r, ALL-52s, HM3722, HM3101, ALL-28r, ALL-57r, HM2872, ALL54, ALL51, ALL26, ALL53, ALL52, HM3822) and cell lines (blue-SUP-15, MUTZ-5, B1, RS4;11, KASUMI-2, c697, MHH-CALL4, NALM-6, KOPN-8, REH, RCH-ACV) in PC1, with some separation in the second component for xenograft samples. The lack of separation between dex-sensitive (closed circle—ALL-53s, ALL-54s, ALL-51s, ALL-52s, HM3722, HM2872, ALL54, ALL51, ALL26, ALL53, ALL52, HM3822, SUP-15, MUTZ-5, B1, RS4;11, KASUMI-2, c697, MHH-CALL4, NALM-6, KOPN-8) and dex-resistant (open circle—ALL56r, ALL-55r, ALL-50r, HM3101, RCH-ACV, REH, ALL-28r, ALL-57r) indicates that there is not a wholesale change in the gene regulation program of resistant samples, but more likely a change in a smaller number of key genes. These data indicate that the dex-induced gene regulation in cell lines is similar to patient samples and patient-derived xenografts making them a suitable model for studying GR function in B-ALL. FIG. 8C is an ingenuity pathway analysis for the 478 commonly regulated genes (Adj. P-Value ≤1e-4). FIG. 8D is an analysis of Molecular and Cellular Function gene sets in Ingenuity indicate a role for dex in cell survival.

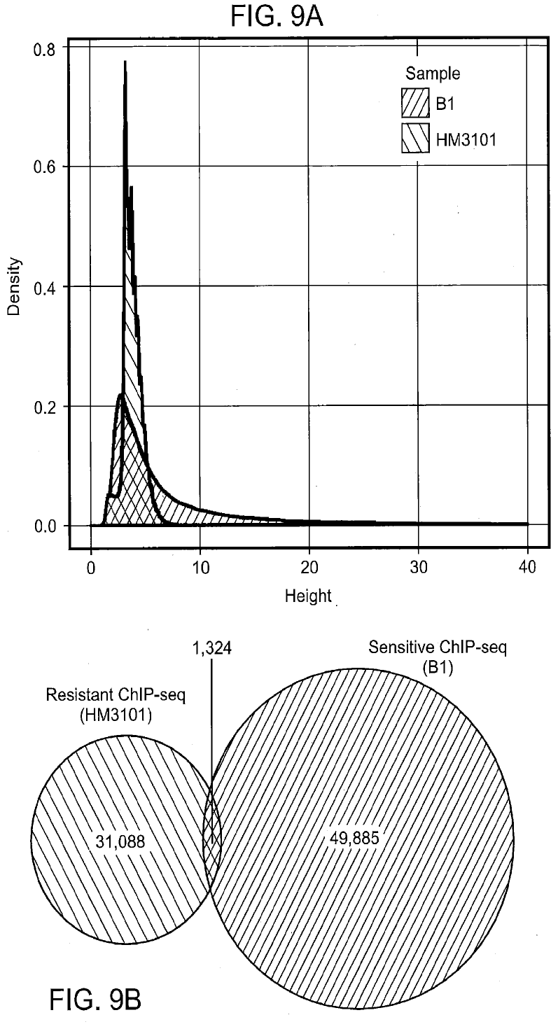

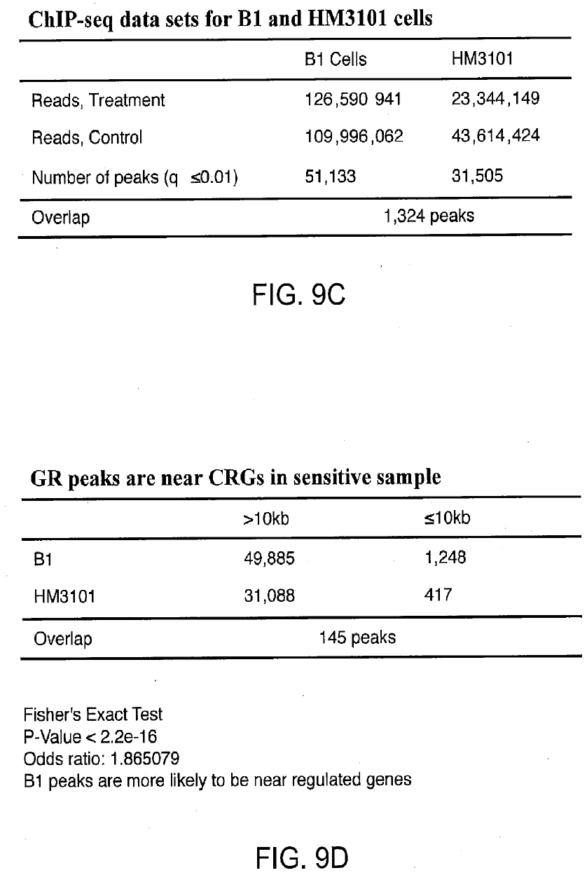

The results as shown in FIGS. 9A-9D generally highlight chromatin immunoprecipitation of the glucocorticoid receptor in glucocorticoid-sensitive (B1) and -resistant (HM3101) B-ALL samples. Samples were treated with dex or vehicle for 90 minutes before crosslinking and immunoprecipitation. Peaks were then measured against the vehicle control. FIG. 9A is a plot of the distribution of significant peak heights shows that typical enrichment of GR at binding sites in the resistant cell is low compared to the sensitive cell. FIG. 9B is a venn diagram depicting which GR binding regions are shared between the sensitive and resistant cells. Although both samples have a large number of peaks, their overlap is minimal, as shown in FIG. 9C. FIG. 9D is a table showing the distribution of binding sites with respect to the gene body of CRGs. Fisher's exact test shows that B1 binding sites are more likely to be near CRGs genes than HM3101 binding sites.

The results as shown in FIGS. 10A-10D generally highlight knockdown of cancer, apoptosis, gene expression, and kinase (CAGEK) panels. Using an ultra-complex shRNA screen, 5,761 genes were knocked down in NALM-6 cells, and their effect on growth and dex sensitivity were measured. FIG. 10A shows that of the 5,761 genes in the screen, 5,347 were measured in the gene expression arrays (left). Of these, 1,216 affect growth when knocked down and 1,065 affect dex sensitivity (PValue≤0.05) when knocked down in NALM-6 cells. FIG. 10B shows that a substantial number of genes (375) affect both growth and dex sensitivity. FIG. 10C is a bar chart depicting the number of genes that significantly (Q-Value≤0.05) affect NALM-6 Growth and Sensitivity to dex divided into protecting (or faster growth, purple or darker gray) and sensitizing (or slower growth, Green or lighter gray). FIG. 10D is an ingenuity pathway analysis reveals that disruption of genes in the B cell receptor pathway affect the growth and dex sensitivity of NALM-6 cells. Connected to this, PI3K and ERK/MAPK signaling also affect growth and sensitivity.

The results as shown in FIGS. 11A-11F generally highlight example enrichment and depletion of shRNAs across individual genes. The ultra-high content shRNA screen used to identify genes that sensitize or protect cells from dex-induced cell death contains ˜25 computationally designed shRNAs per gene. Whether a gene has a significant effect on sensitivity is based on how many of these shRNAs exhibit enrichment or depletion, the magnitude of that enrichment or depletion, and the difference between this enrichment and thousands of control shRNAs. FIGS. 11A-11D are Barplots show the log2-fold enrichment over growth controls for each shRNA in dex-treated cells. Positive enrichment as shown in FIGS. 11A-11B indicates that knockdown of the gene protects cells against GC-induced cell death (Purple or Gray- FIGS. 11A-11B), whereas negative values as shown in FIGS. 11C-11D indicate sensitization (Green or Gray- FIGS. 11C-1D). The error bars represent the standard deviation of three biological repeats of the screen. Two things are remarkable about these plots: first, the number of active shRNAs per gene is >80%; second, the consistency of the effect of each shRNA for a given gene gives remarkably high confidence that the result is significant and not due to off-target effects. FIG. 11E show that the results of the screen are robust. Knockdown of individual protective genes have the expected effect of making NALM-6 cells less sensitive to dex. FIG. 11F are western blots showing that the individual shRNAs provide substantial knockdown of their target at the protein level (A is the actin loading control band).

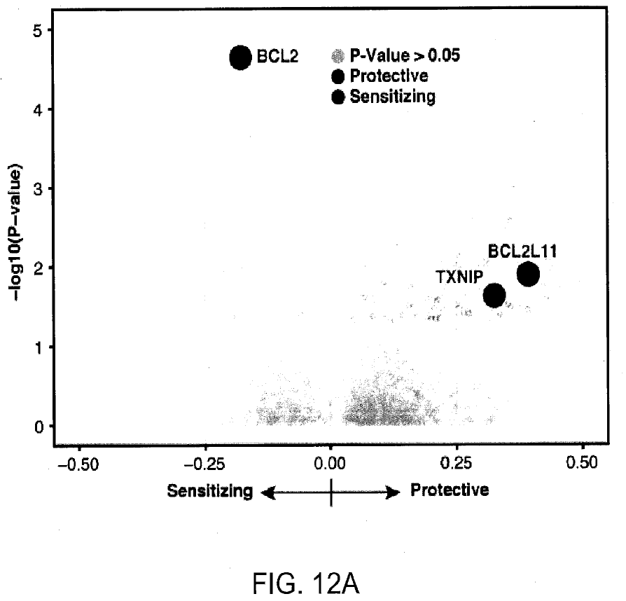

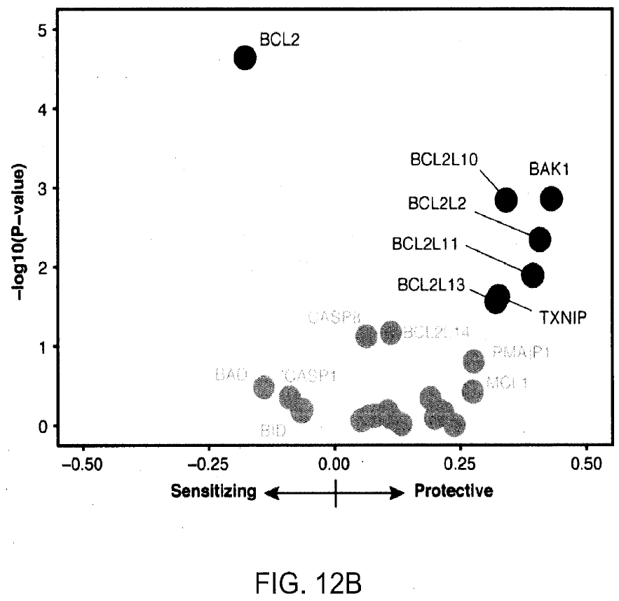

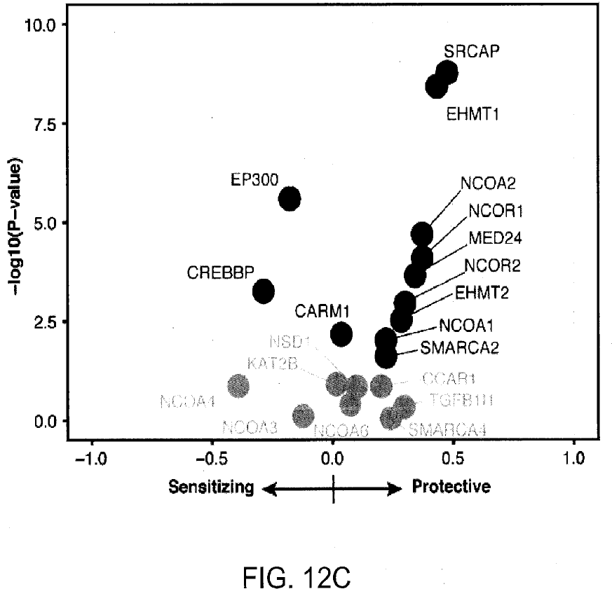

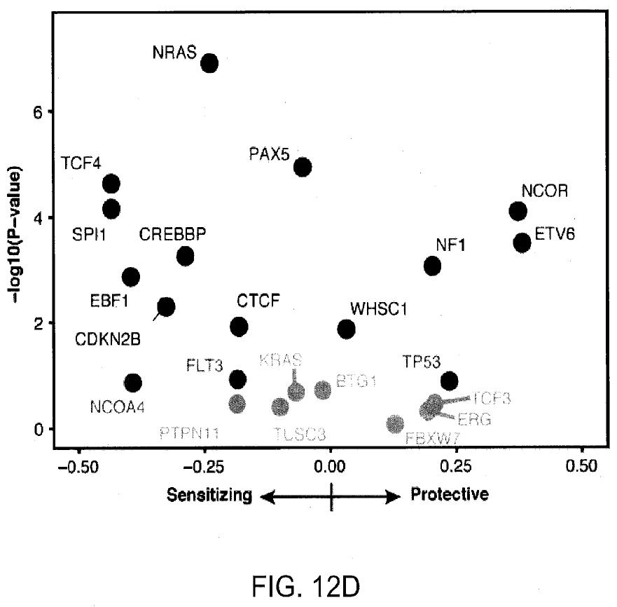

The results as shown in FIG. 12A-12D generally highlight the effect of knockdown on dex sensitivity of significant gene sets. Volcano plots of significant gene sets showing whether knockdown sensitizes (Green or lighter gray—BCL2, EP300, CREBBP, KRAS, PAX5, TCF4, SP11, CREBBP, CTCF, CDKN2B, CTCF, FLT3, NCDA4) or protects (Purple or draker gray—BCL2L11, TXNIP, BCL2L10, BCL2L13, BCL2L2, BAK1, SCRAP, EHMT1, NCOA2, NCOR1, MED24, NCOR2, EHMT2, NCOA1, SMARCA2, CARM1, NCOR, ETV6, NF2, WHSC1) NALM-6 cells form dex-induced cell death (P-Value≤0.01, or P-Value≤0.05 for light points). Note that plots are on different scales to accentuate separation between points. For apoptosis, FIG. 12A is a volcano plot for the effect of knockdown on dex sensitivity for genes having been shown previously to affect GC-induced apoptosis in BCP-ALL. FIG. 12B is a plot showing BH3-containing and other apoptosis genes. This plot validates the importance of previously identified apoptosis genes, but also identifies a substantial number of other genes that affect dex sensitivity. FIG. 12C shows the effect of known GR cofactor knockdown on dex sensitivity. Of the 20 known cofactors, 13 have a significant effect on dexsensitivity. Surprisingly, knockdown of CREBBP/P300 sensitized cells, whereas all other significant co-activators and co-repressors protected against dex-induced cell death, indicating an important function for both activation and repression of GR regulated genes. FIG. 12D is a plot of the genes most frequently mutated in refractory and relapsed ALL. Of the 22 in the screen, 14 have a significant effect on dex sensitivity, suggesting a critical role for glucocorticoid sensitivity in treatment success.

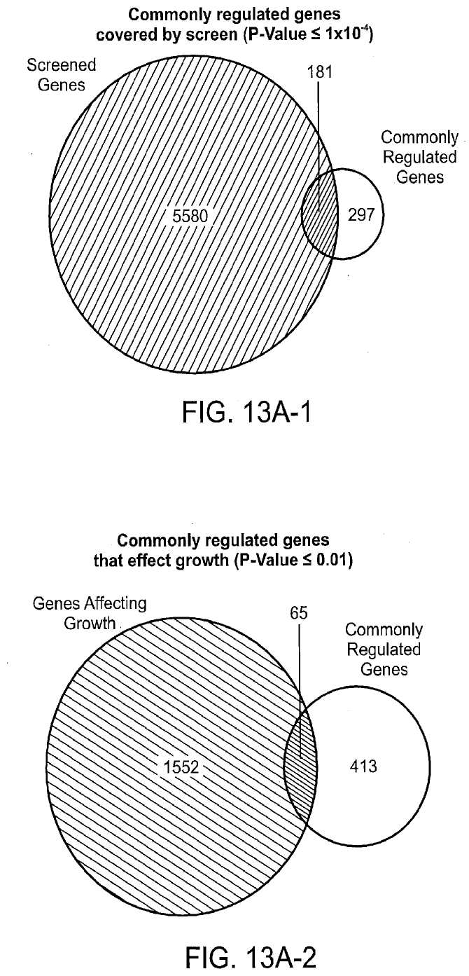

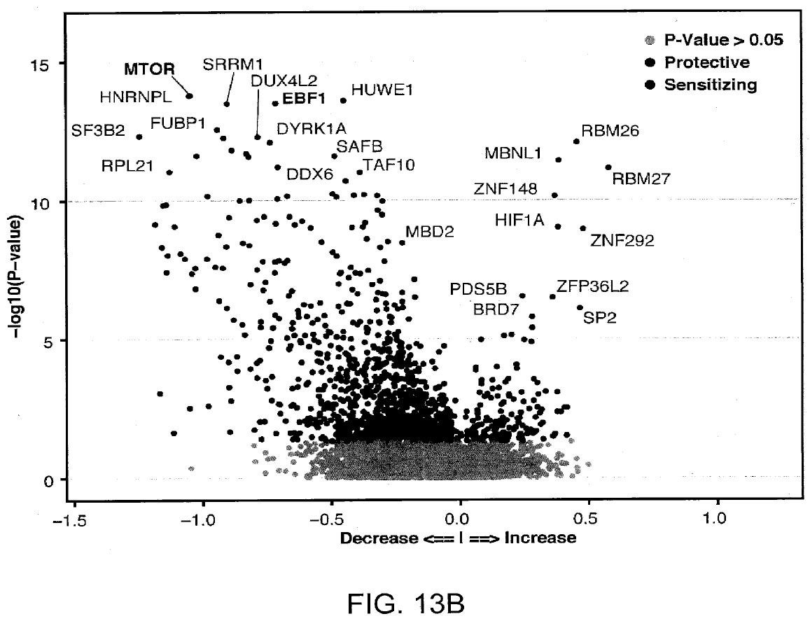

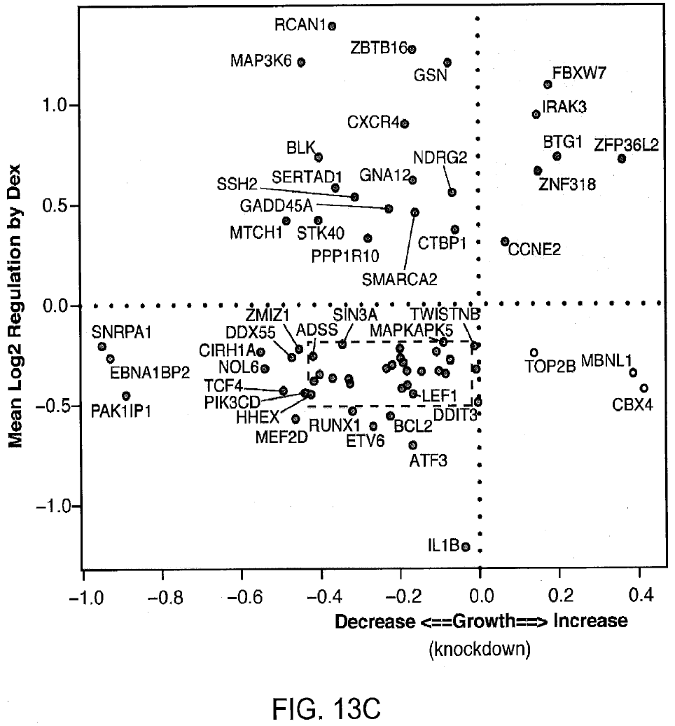

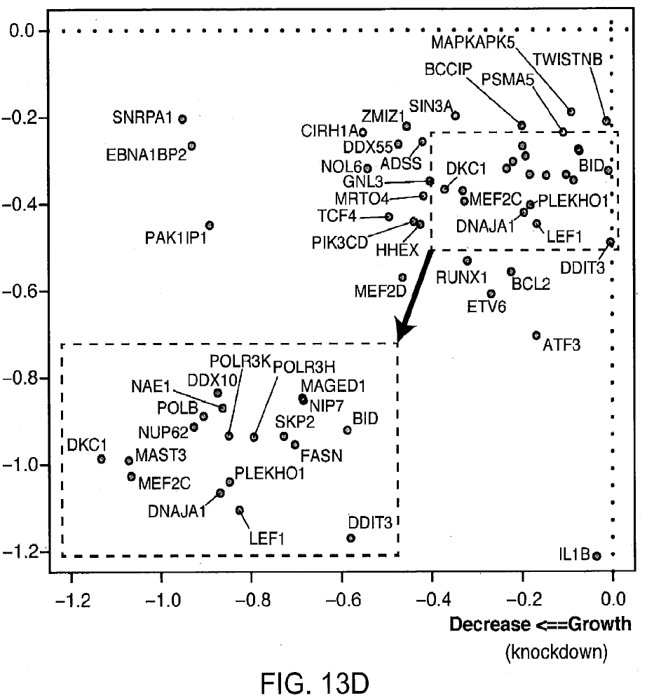

The results as shown in FIGS. 13A-13C generally highlight validation of commonly regulated genes using the effect of shRNA gene knockdown on growth. FIGS. 13A-1 and 13A-2 show that of the 478 commonly regulated genes across sensitive B-ALL samples (P-Value 1e-4), 181 are included in the next-generation shRNA screen performed (top). Of these, 65 affected the growth in NALM-6 cells when knocked down. FIG. 13B is a volcano plot depicting the effect of knocking down genes on NALM-6 cell growth. Each point represents a gene, with those that significantly slow growth in green 11 and those that increase growth in purple 10 (P-Value≤0.01). Select genes are labeled. FIG. 13C is a plot of commonly regulated genes with an effect on growth with the mean regulation across all samples on the Y-axis and the effect of knockdown on growth on the X-axis. Repression of genes that impair cell growth on knockdown (Green) 12 likely contribute to dex-induced cell death, as do activated genes that increase growth on knockdown (Purple) 13 (left). FIG. 13D is a zoomed in view of the green region 12 of FIG. 13C.

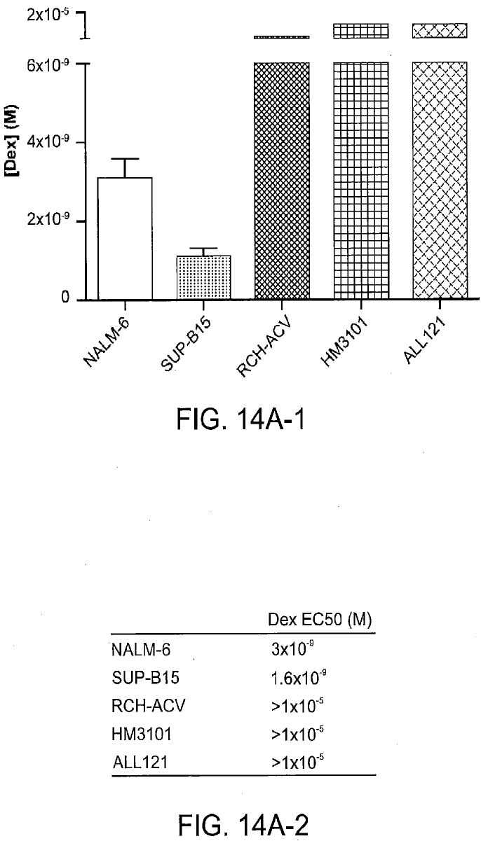

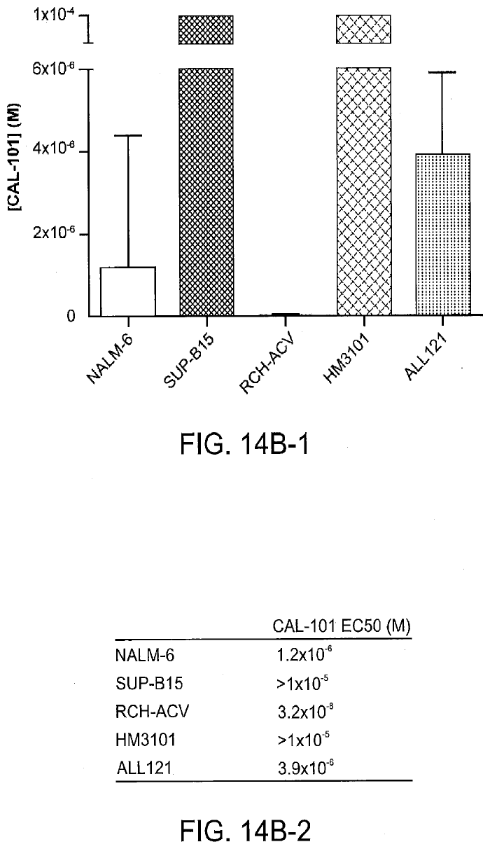

The results as shown in FIGS. 14A-14B generally highlight the EC₅₀ of BCP-ALL specimens for dexamethasone and CAL-101. Three cell lines (NALM6, SUP-B15, and RCH-ACV), one resistant patient sample (HM3101), and one relapsed Phlike xenograft (ALL121) were incubated with increasing concentrations of dex or CAL-101 for 3 days. Cell viability was measured by PrestoBlue staining. Curves, values, and errors are based on at least 3 replicates. Bar plot of the EC₅₀, calculated by four-parameter least-squares fitting (Both plotted and calculated in GraphPad Prism). As shown in FIGS. 14A-1 and 14A-2, the EC₅₀ for dex of RCH-ACV, HM3101, and ALL121 are estimates since no concentration of dex reproducibly reduced viability ≥50%. As shown in FIGS. 14B-1 and 14B-2, incubation with CAL-101 caused <50% death even at the highest concentration (10 μM) for SUP-B15 and HM3101, preventing an accurate estimation of EC50.

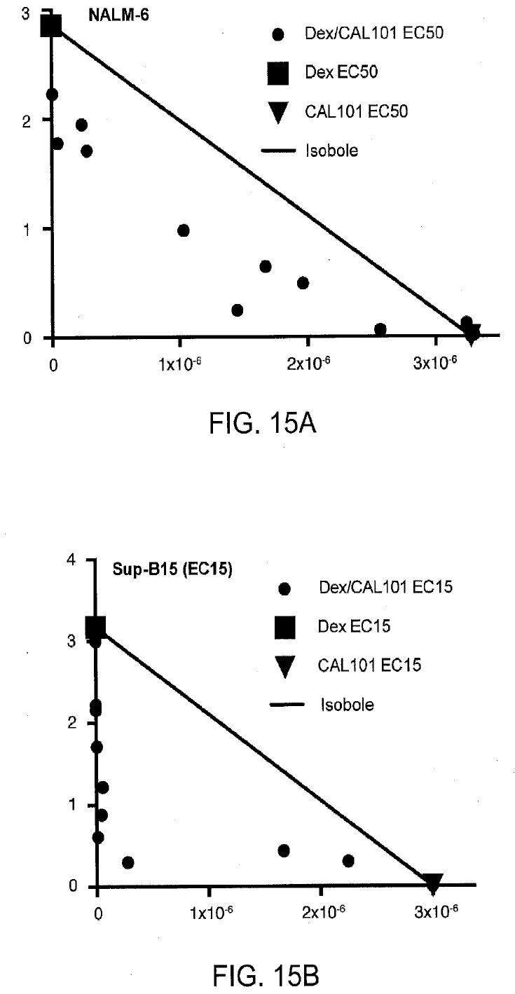

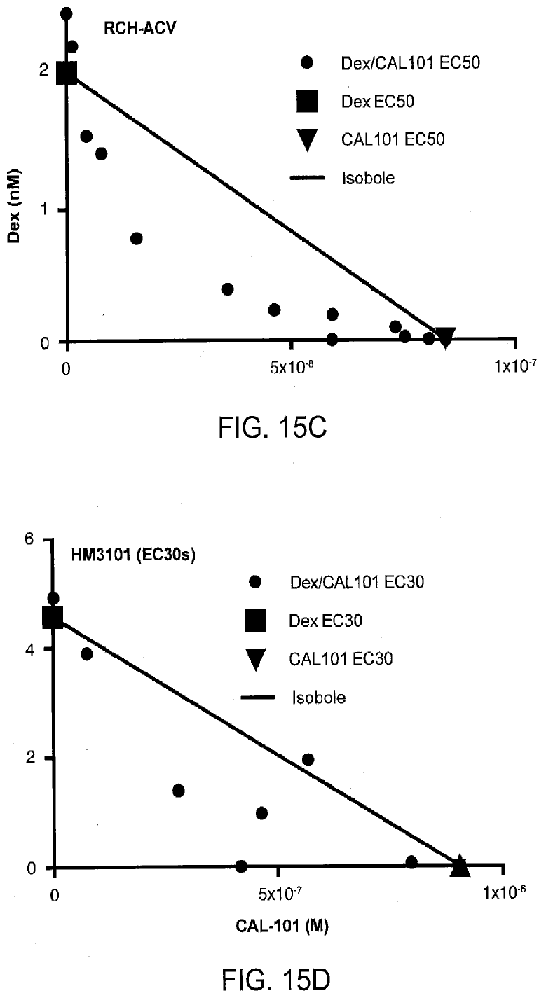

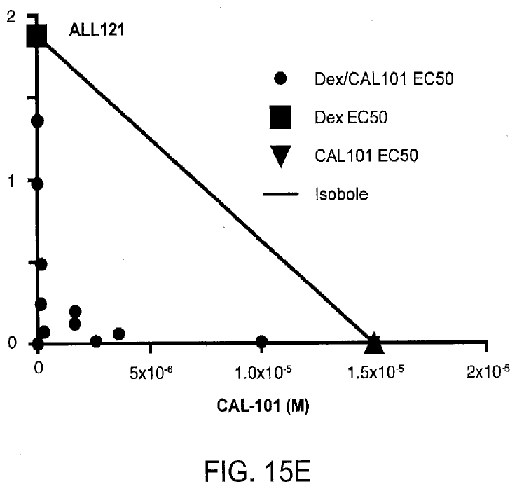

The results as shown in FIG. 15 generally highlight isoboles for the combination of dex and CAL-101. Five samples were incubated with a full grid of concentrations of dex (0 to 10 μM) and CAL-101 (0 to 10 μM) for three days. Viability was then measured, and the EC50 (or lower ECs if the samples were refractory to the drug) measured for each drug alone. The combinations of drugs that induced 50% death were then plotted with these EC50s for each sample. If the points fall below the line connecting the EC50s for each alone, the drugs are considered super-additive or synergistic. This was true for NALM-6, RCH-ACV, and ALL121. For SUP-B15 and HM3101 the response of the cells to either CAL-101 or dex did not induce 50% death, respectively. In these cases, the most stringent ECs are plotted. Nonetheless, the interaction between dex and CAL-101 is greater than additive. Combination indices were then calculated (Calcusyn, see FIG. 4F).

The results as shown in FIGS. 16A-16D highlight response of causative genes to dex over 24 hours and PIK3CD to Dex/CAL-101 combinations. Response of six causative genes (BCL2, IL7R, MYC, PIK3CD, BCL2L11(BIM), and TXNIP) in three cell lines, NALM-6 as shown in FIG. 16A, (B) SUP-B15 as shown in FIG. 16B, and RCH-ACV as shown in FIG. 16C to 1 μM dex at 4, 8, and 24 hours. Maximum response was observed in almost all cases at 24 hours, which was subsequently used as an endpoint in measuring the effect of CAL-101 on dex-induced gene regulation (FIGS. 5C-1 and 5C-2). FIG. 16D shows response of PIK3CD in three cell lines to dex and CAL-101. Cells were treated with dex at the EC50 and EC90 (NALM-6 and SUP-B15) and 0.5 and 5 μM dex (RCH-ACV) and two concentration of CAL-101 (7.7 nM and 280 nM) for 24 hours. Although dex strongly represses PIK3CD in NALM-6 cells, no significant regulation is observed in SUP-B15 or RCH-ACV cells. Further, addition of CAL-101 blunts repression in NALM-6 cells and has no significant effect in the other two cells lines.

### Results:

### Dexamethasone Regulates B-Cell Development Genes

We integrated two complementary technologies to determine how GCs induce cell death in B-ALL: dex-induced differential gene expression analysis and functional genomics by large-scale shRNA gene knockdown. By combining these methods, we identified “effector genes”: those GR-regulated genes that drive glucocorticoid-induced cell death in B-ALL. We first isolated the primary effects of GCs in sensitive B-ALL samples by measuring immediate (4-8 hour) changes in gene expression in response to high-dose dex. Using 19 human B-ALL cell lines, primary patient specimens, and existing data from patient-derived xenograft models (PDXs)^{11a}, we found that only four genes were significantly regulated (q-value≤0.05) in each sample: FKBP5, TSC22D3 (GiLZ), SMAP2, and TXNIP. Of these, only TXNIP and GiLZ have been previously linked to dex-induced cell death^{12a}. However, we identified another 588 genes that are consistently activated or repressed across samples (KS-test, Adj. p-value≤1e-4), which we term commonly regulated genes (CRGs) (FIG. 1A, diff_gene_expr.xlsx). Consistent with previous studies, CRGs include BCL2, BCL2L11, KLF13, ZBTB16, and GR itself ^{13a,14a}. Pathway and gene ontology analyses identified expected general GC functions, including diabetes and cell death and survival (FIG. 8A-8B), but also a previously unobserved enrichment for hematological and lymphoid development (FIG. 1B). Within this category, dex repressed expression of three genes, ITGA4^{15a}, IL7R^{13a}, and BCL6^{16a} that are key factors in early B-cell development (Table 4). Dex also repressed genes related to B-cell receptor (BCR) signaling (CD79B, CSK, FYN, BTK, PIK3CD, PIK3C2B, PIK3R2) and activated CXCR4, a receptor that homes B-cells to the bone marrow and germinal centers for further maturation^{17a}. These regulatory patterns suggest a pervasive role for GCs in B-cell development and provide a mechanistic explanation for observations made 30 years ago that GCs have a negative effect on early B-cell development^{18a,19a}.

To identify which CRGs are likely direct targets of GR, we performed ChIP-seq for GR in GC-sensitive and -resistant cells. In the GC-sensitive human ALL cell line B 1, we observed a greater number of stronger GR binding sites (˜50,000 sites) compared to a relapsed primary human B-ALL specimen, HM3101 (˜30,000 sites). Although fewer than 3% of the binding sites overlapped between samples, a significant fraction were within 10 kb of CRGs (FIG. 9A-9B). Importantly, binding sites were enriched near CRGs in sensitive compared to resistant cells (FIG. 9C-9D), including ITGA4, IL7R, and BCL6, suggesting that they are direct targets of GR (FIG. 1D-F). The striking shift in binding pattern suggests a difference in the accessibility of binding sites (as in^{20a}), the viability of transcriptional cofactors that direct GR binding (as in^{21a}), or a previously unrecognized signaling pathway that changes GR function. Most importantly, this demonstrates that GR can be active in resistant B-ALL, but also fail to bind and regulate effector genes.

Next-Generation shRNA Screen Identifies Effectors of GC-Induced Cell Death

Next, we conducted a large-scale next-generation RNA interference screen to determine which GR-regulated genes contribute to cell death and to pinpoint pathways that modulate GC potencyl^{10a,22a}. We identified an appropriate cell line model for screening by comparing the dex-induced transcriptional response of cell lines to primary specimens. Although the mRNA levels in unstimulated specimens were different (data not shown), the dex-induced changes in cell lines were similar to the patient specimens and PDX samples (FIG. 1A-1F, 8C-8D), indicating that cell lines are a reasonable model for dex response in B-ALL. We chose NALM-6 cells, which have intermediate dex-sensitivity and relatively rapid growth.

The next-generation screen is composed of an ultra-complex shRNA library^{10a,22a,23a} that has four advantages over other screens: 1) thousands of negative control shRNAs are included to increase statistical confidence and identification of true hits; 2) the large number (25) of shRNAs per gene decreases both the false-positive and false-negative rates; 3) the shRNAs are more active^{24a}, allowing a quantitative analysis of gene knockdown; and 4) both synthetic interactions (the effect of knockdown on dex sensitivity) and the effect of knockdown on NALM-6 growth can be calculated from the data. We adapted how the screen was performed previously^{10a} (See Materials and Methods) using an intermediate dose of dex (35 nM) to enable identification of sensitizing and protective hits. The four most relevant of 11 total panels were screened (Cancer, Apoptosis, Gene Expression, Kinases (CAGEK)), comprising ˜5,800 genes and >140,000 shRNAs, covering about 40% of the CRGs (FIGS. 2A-1, 2A-2, 10A).

The data from the screen were robust, sensitive, and consistent with the known features of dex-induced B-ALL death. Good agreement was observed among three biological replicates, and the shRNAs show remarkably consistent activities within any given gene (FIG. 11A-11D). Importantly, genes that, when knocked down, either significantly protect (resulting in shRNA enrichment) or sensitize (resulting in shRNA depletion) NALM-6 cells to dex are evident (FIG. 2B, shRNA.xlsx). Overall, knockdown of 156 out of 5761 CAGEK genes had highly significant effects (FDR≤5%) on NALM-6 dex sensitivity, and those we tested individually validated well (FIGS. 11E, 11F). About 10% of the genes screened (653) had an effect on NALM-6 growth. In addition to these highly significant genes, a number of other genes (p-value≤0.05), which we term top hits, likely also affect GC-sensitivity (FIGS. 2B, 12B).

It should be noted that although knockdown of GR resulted in complete resistance, protective hits from the screen generally decreased sensitivity only 2- to 3-fold (FIG. 11E). This could be the result of incomplete knockdown (FIG. 11F) or compensation by other factors. However, an alternative model is that GC-induced cell death is multifactorial; having multiple downstream effectors of cytotoxicity, and a network of signals upstream that collaborate with GR to efficiently induce cell death. This model is supported by the identification of many unexpected modulators of dex-sensitivity (FIGS. 2B, 13A, 13B). For example, the screen not only confirmed the importance of BCL2, which was sensitizing upon knockdown, and BCL2L11 and TXNIP, which were protective, but also identified four other key BH3-containing factors that affected dex-induced cell death (FIG. 13C). This demonstrates that no apoptosis gene is absolutely required. Further, the partial effects of these genes reinforce the idea that multiple factors contribute to GC cytotoxicity.

The screen also revealed important new insights into the cellular factors that affect GC cytotoxicity and sensitivity. Among the genes screened are 21 genes that are frequently mutated in refractory/relapsed B-ALL, 16 (˜70%) of which are among our top hits (FIGS. 2C, 6)^{1a,25a}. Most of these genes are sensitizing when knocked down, suggesting that rare gain-of-function mutations conferring resistance to GCs are selected for during treatment. These data demonstrate that perturbing GC function is perhaps the most prevalent source of overall treatment resistance.

Most strikingly, pathway analysis of hits affecting GC sensitivity revealed a role for B-cell development in GC cytotoxicity. The B-cell receptor (BCR) pathway (FIG. 10D) has the most significant effect on both the growth and sensitivity of NALM-6 cells to dex. The BCR pathway is a potent growth and survival signal that works in part through stimulation of the PI3K and ERK/MAPK pathways^{26a}, knockdown of which also exhibited significant effects on growth and sensitivity (mapped in FIG. 3). The mechanism of how BCR/PI3K signaling affects GC cytotoxicity is revealed by integrating the functional genomic and gene expression data.

One challenge in interpreting differential gene expression data sets is identifying which regulated genes cause the phenotype, in this case cell death. We used the screen to identify these “effector genes” from among the CRGs en masse. Dex-activated effector genes (those that induce cell death when activated) increase growth or protect cells from dex-induced cell death when knocked down. Conversely, knockdown of dex-repressed effector genes (those whose repression induces cell death) decreases growth or increases sensitivity to dex. Of the CRGs included in the screen (247 out of 588), 85 of those knocked down caused a growth phenotype, including 56 that match the effector phenotype (FIGS. 12A, 12C, 14A-1, 14A-2). Several of the activated effector genes are transcriptional cofactors, including BTG1, which is required for the GR autoinduction: a consistent feature of dex-sensitive B-ALL^{27a}. A larger number of repressed genes exhibited an effector phenotype, including key regulators of lymphoid and B-cell development (MEF2C/D, LEF1, RUNX1, ETV6, BCL2, and TCF4; FIGS. 14A-1, 14A-2), supporting our model that GC regulation of B-cell development genes contributes to its cytotoxicity.

The importance of B-cell development pathways in GC cytotoxicity is even more evident from analysis of effector genes identified by dex-sensitivity in the screen. Of the 63 GC-regulated genes whose knockdown affects dex-sensitivity, about half (32) exhibited an effector phenotype (FIGS. 2A, 2D, FIG. 14B). Genes that are activated and protective are overrepresented for transcription-related factors (GR, SERTAD1, SMARCA2, CTBP1, SBF1, STK40), which, like BTG1, may be required for GR gene regulation or to enhance downstream transcriptional programs. A striking number of repressed and sensitizing effector genes are involved in lymphoid and B-cell development, including BCL2, LEF1, IL7R, CBX4, CMTM7, ZMIZ1, TCF4, and PIK3CD. This not only supports the link between development and GC efficacy, but suggest that these synthetic interactions can be exploited with inhibitors to synergize with GCs.

### PI3Kδ and the BCR Pathway are Tightly Regulated by GCs

An intimate connection between GCs and B-cell development is evident in the BCR pathway. Tonic signaling through the pre-BCR is present in ˜15% of B-ALL, including NALM-6 cells, and is essential for B-cell development and survival^{28a}. Indeed, our screen data indicate that knockdown of almost any BCR component is detrimental to growth, confirming the importance of the pathway (FIG. 3A). In contrast, only the PI3K/RAS/MAPK branch of the BCR pathway sensitizes cells to dex (FIG. 3B). Thus, pre-BCR signaling not only drives of proliferation B-ALL cells, it also specifically opposes dex-induced cell death.

The importance of BCR signaling in treatment sensitivity of B-ALL has been shown previously through inhibition of the mTOR/AKT branch with rapamycin^{29a}. Although we also observe an effect of mTOR/AKT knockdown on growth, it does not sensitize B-ALL to GCs. This is in contrast to T-cell ALL, where AKT inhibition does synergize with dex^{30a}. Instead, our data highlight the PI3K/MAPK branch, which can be activated from the BCR proximal SYK or from IL7R, which converge specifically on the lymphoid-restricted PI3Kδ (PIK3CD), through NRAS, eventually inhibiting GR function through phosphorylation by Erk2 (MAPK1) (FIG. 3B).

Tight control of specific PI3 kinase signaling components by GCs is apparent from the gene expression and shRNA screen data (FIG. 4A). Of the p110 PI3K subunits, knockdown of only PIK3CD both inhibited cell growth and sensitized cells to dex (FIG. 4B), whereas knockdown of PTEN protected cells from dex (FIG. 4C). A specific regulatory mechanism is evident, as knockdown of only one p85 regulatory subunit, PIK3R2, protected cells from dex-induced cell death (FIG. 4C), consistent with its role in restraining production of PIP3^{31a}. Not only do these specific PI3K components affect GC sensitivity, but their expression is regulated by dex. Expression of PI3Kδ is strongly repressed by dex, and PIK3IP1, a negative regulator of PI3 kinases³²a, is strongly activated (FIGS. 4E-1, 4E-2, left). The presence of GR binding sites in sensitive cells for both of these genes indicate direct regulation by dex (FIGS. 4E-1, 4E-2, right). Together, these data map a double-negative feedback loop between PI3Kδ and GR: Addition of GCs suppresses PI3K activity, which in turn sensitizes cells to GCs (FIG. 4A).

To test this hypothesis, we inhibited PI3Kδ using CAL-101 (idelalisib or idela), an FDA-approved drug used in monotherapy treatment of chronic lymphocytic leukemia and indolent Non-Hodgkin's Lymphoma^{33a}. Although CAL-101/idela monotherapy shows an effect in patient-derived xenograft models of treatment-refractory paediatric B-ALL, it is does not clear the disease^{34a}. We tested the combination of CAL-101 and dex in five B-ALL samples: two sensitive cell lines (NALM-6, SUP-B15) and three resistant samples (cell line RCH-ACV, relapsed patient sample HM3101, and relapsed PDX, ALL121). The response of cells to CAL-101 was different than to dex, with SUP-B15 being the least sensitive and RCH-ACV being the most sensitive (FIG. 15C). Graphing the isoboles^{35a} revealed that dex and CAL-101 are superadditive in all samples, including the most refractory B-ALL patient samples (FIG. 16A-16D). With a combination index as low as 0.13 (FIG. 4F), the superadditivity in all backgrounds indicates that addition of CAL-101 sensitizes B-ALL to dex and may be effective in overcoming resistance. This synergy, surprisingly, is independent of pre-BCR status^{36a} suggesting that PI3Kδ may be activated by IL7R or other pathways in the absence of tonic pre-BCR signaling. Thus, although synergy had also been observed with a pan-PI3K inhibitor^{37a}, the resolution of our data allow targeting of the lymphoid-restricted PI3Kδ, which is likely to have fewer side effects.

As proof of concept, we tested this combination in a PDX model of ALL. NOD scid gamma-deficient (NSG) mice engrafted with leukemia cells from a child with multiply-relapsed B-ALL (ALL121, Ph-like, pre-BCR⁻) were treated with vehicle, dex (7.5 mg/kg daily), idela (50 mg/kg daily), or both for two weeks. Harvested spleens from treated animals demonstrated no effect from dex or idela monotherapy, but markedly reduced spleen size and decreased human leukemia burden using the dex/idela combination (FIGS. 4H, 4I). The combination shows a more pronounced synergy in this model than predicted from in vitro cultures. Although more preclinical work is needed to determine how prevalent the efficacy of this combination is, the PDX model shows the utility of this integrated functional genomic approach in identifying promising combination chemotherapeutics.

### PI3Kδ Inhibition Potentiates Regulation of Effector Genes

The synergy of dex and CAL-101/idela is due, at least in part, to enhanced GC-regulation of effector genes. Using combinations of dex and CAL-101, we monitored four repressed (BCL2, IL7R, MYC, PIK3CD) and two activated (BCL2L11 and TXNIP) effector genes in three cell lines: NALM-6 (sensitive to both drugs), SUP-B15 (sensitive to dex but resistant to CAL-101), and RCH-ACV (resistant to dex and sensitive to CAL-101) (FIGS. 5A-1, 5A-2, 5A-3, 5B-1, 5B-2, 5B-3, 5C-1, 5C-2). Inhibition of PI3Kδ significantly enhanced dex-induced repression of BCL2 and IL7R (at low doses of dex), and activation of TXNIP in all cells tested (FIGS. 5A-1, 5A-2, 5A-3, 5B-1, 5B-2, 5B-3, 5C-1, 5C-2). Surprisingly, inhibition of PI3Kδ had little, or even an opposing, effect on dex-repression of PIK3CD, indicating that inhibition of PI3Kδ does not feed back on itself through GR. For other effector genes, the combined regulation is cell-type specific, exemplified by the effect of PI3Kδ inhibition on MYC repression, which is enhanced in SUP-B15 and RCH-ACV cells, but not in NALM-6. In addition, dex-induced activation of BIM, thought to be a crucial component of dex-induced B-ALL cell death, is blunted by PI3Kδ inhibition, again suggesting that other BH3 family members may be important in driving apoptosis. This potentiation can work directly through GR at genes such as TXNIP in NALM-6 cells, where CAL-101 alone has no effect on regulation yet enhances dex-induced activation. Potentiation can also be combinatorial for some genes, as is the case with MYC in RCH-ACV cells: CAL-101 and dex both regulate the gene in the same direction, but they regulate more strongly together. These data indicate that inhibition of PI3Kδ synergizes with dex in a cell-type specific manner by selectively potentiating regulation of different sets of effector genes.

CAL-101/idela administration can also restore GC-induced regulation of quiescent genes. BCL2 in SUP-B 15 cells and IL7R in RCH-ACV cells do not respond to dex alone, but when treated with a combination of CAL-101 and dex, they are repressed (FIGS. 5A-1, 5A-2, 5A-3, 5B-1, 5B-2, 5B-3, 5C-1, 5C-2, dashed boxes). This result supports the model that resistance to GCs can be due to a failure of GR to regulate key genes, and, further, that a latent GC-regulated cell death program can be re-established by manipulation of key pathways that converge on GR. To test convergence, we inhibited PI3Kδ and probed three phosphorylation sites on GR known to modulate its function, S203, S211, and S226. Administration of CAL-101 specifically reduces S203 phosphorylation levels (FIG. 4G), which has been shown to inhibit GR function^{38a,39a}. CDK2A and can phosphorylate S203^{40a,41a}, but our screen indicates that only MAPK1 has an effect on GC sensitivity, indicating that the PI3K/MAPK pathway inhibits specific GR functions.

### Discussion

The link between B-cell development and cytotoxicity suggests that GCs perform a function in normal B-cell development as one of many signals that influence B-cell selection. This connection echoes the positive and negative effect of GCs in early T-cell development^{42a}, and is reminiscent of the therapeutic effect of all-trans retinoic acid on another nuclear hormone receptor, the retinoic acid receptor, whose cytotoxic effect on acute promyelocytic leukemia results from pushing through a developmental block^{43a}. The ability of GCs to suppress B-cell checkpoint genes that are required across multiple developmental stages helps explain their efficacy in treating lymphoid malignancies that are blocked at these different stages. Indeed, in NALM-6 cells, knockdown of the BCR pathway and IL7R enhances dex-induced death, suggesting arrest at the pre-B stage, which is consistent with the genetic and cytological features of these cells^{44a}. We therefore propose the model that GCs can either stop or push B-cells through development (FIG. 1C). In this model, supraphysiological levels of GCs can either push immature cells to the next stage of development (through BCL6 or CXCR4, for example), which may trigger apoptotic programs, or they may arrest cells by removing a positive growth signal (such as IL7R, PIK3CD, or ITGA4). In the case of IL7R and PIK3CD, gene repression may further accentuate GR function, forming a positive feedback loop that drives cells toward death. Further study is needed to validate this model, but it provides a long-sought mechanism for how and why GCs induce lymphoid cell death.

The resolution of next-generation shRNA screening establishes it as an essential tool for rational identification of combination chemotherapeutics^{45a}. Hits can be filtered for potential synergy, tissue restriction, and for the availability of existing drugs. Using these criteria, we demonstrate a pipeline to rapidly identify potent combination therapies that are likely to have fewer side effects and accelerated time to pre-clinical and clinical testing.

The following publication is incorporated in its entirety by reference herein: Kruth, Karina A. et al “Suppression of B-cell development genes is key to glucocorticoid efficacy in treatment of acute lymphoblastic leukemia.” Blood 129.22 (2017): 3000-3008.

### EXAMPLE 2

### A Post-Translational Modification Switch Controling Coactivator Function of Histone Methyltransferases G9a and GLP

Like many transcription regulators, histone methyltransferases G9a and G9a-like protein (GLP) can act gene-specifically as coactivator or corepressor, but mechanisms controlling such dichotomies are mostly unknown. We show that adjacent post-translational methylation and phosphorylation regulate binding of G9a and GLP to heterochromatin protein 1 gamma (HP1γ), formation of a ternary complex with the glucocorticoid receptor (GR) on chromatin, and function of G9a and GLP as coactivators for a subset of GR target genes. HP1γ is recruited by G9a and GLP to GR binding sites associated with genes that require G9a, GLP and HP1γ for glucocorticoid-stimulated transcription. At the physiological level, G9a and GLP coactivator function is required for glucocorticoid activation of genes that repress cell migration in A549 lung cancer cells. Thus regulated methylation and phosphorylation serve as a switch controlling G9a and GLP coactivator function, suggesting that this mechanism may be a general paradigm for directing specific transcription factor and coregulator actions on different genes.

DNA-binding transcription factors activate and repress transcription of their target genes by recruiting coregulator proteins to the promoter/enhancer regions of their target genes. Coregulators remodel chromatin structure and promote or inhibit the assembly of an active transcription complex. Most of the known coregulators were discovered either for their roles in transcriptional activation or repression. However, many coregulators, including the lysine methyltransferases G9a and G9a-like protein (GLP), function in both activation and repression of transcription, depending on the specific gene and cellular environment [1b-5b]. The factors that determine whether transcription factors and coregulators positively or negatively regulate a specific target gene are mostly unknown.

Many coregulators regulate local chromatin structure by adding post-translational modifications (PTM) to histones. While methylation of histone H3 at lysine 9 (H3K9) is an extremely abundant repressive histone mark in heterochromatin made by several different coregulators, it is also found in euchromatin at repressed promoter/enhancer regions and in the gene bodies of actively transcribed genes [6b]. Histone methyltransferases G9a (also known as EHMT2 or KMT1C) and G9a-like protein (GLP, also known as EHMT1 or KMT1D) are the major H3K9 methyltransferases in euchromatin and are responsible for the majority of mono- and dimethylation of H3K9 in most if not all mammalian cell types [7b]. G9a and GLP repress many genes involved in a variety of cellular processes in embryonic development and adult tissues [8b, 9b], and are overexpressed in a variety of human cancers, where they repress important tumor suppressor genes [10b]. However, G9a functions also as a coactivator for several transcription factors, including steroid hormone receptors (SR) [4b, 11b, 12b], RUNX2 [13b] and hematopoietic activator NF-E2 [14b]. G9a coactivator function has been implicated in physiological processes, such as adult erythroid cell differentiation [14b] and T helper cell differentiation and function [15b].

Whether transcription factors and coregulators act positively or negatively on a specific gene target presumably depends upon signals, such as protein-protein interactions and post-translational modifications (PTM), arising from the unique local regulatory environment of each target gene. Here we investigate the role of PTM in controlling whether G9a and GLP act as coactivators, using as our model system genes regulated by the glucocorticoid receptor (GR, also known as NR3C1), a steroid hormone activated transcription factor, in A549 lung cancer cells. In addition to histones, G9a also methylates some non-histone proteins involved in transcriptional regulation [10b], including itself. G9a is auto-methylated on lysine 185 (K185) and phosphorylated, at least in vitro, by Aurora kinase B on threonine 186 (T186) in the N terminal domain of the protein [16b, 17b]. Heterochromatin protein 1 gamma (HP1γ, also known as CBX3) specifically binds the K185-methylated form of G9a, and this binding is inhibited by T186 phosphorylation [17b], but the biological function of these two PTMs and of the G9a interaction with HP1γ is unknown.

G9a forms heterodimers with its paralogous partner GLP in cells. As they share a similar sequence in their N-terminal domain, we tested whether methylation and phosphorylation occur at the homologous sites on GLP. Moreover, in these cells, G9a potentiates gene activation and gene repression on distinct subsets of GR target genes and is selectively recruited to GR binding regions (GBR) associated with GR target genes that require G9a as a coregulator, indicating that G9a acts directly on these target genes [4b].

As we previously showed that the N-terminal domain of G9a, which includes these two PTM sites, is required for the coactivator function of G9a in the context of steroid hormone receptors (SR) [12b], and since HP1γ has previously been shown to act as a coactivator as well as a corepressor [18b], we hypothesized that these PTMs and HP1γ could be involved in the regulation of the coactivator function of G9a and GLP. Here we report the effects of point mutations at the PTM sites and of inhibitors of methylation and phosphorylation on the ability of G9a and GLP to form ternary complexes with GR and HP1γ and to cooperate with HP1γ as coactivators for glucocorticoid regulation of transient reporter genes and a subset of endogenous GR target genes that require both G9a and GLP as coactivators. Additional endogenous genes that are activated by GR but do not require G9a or GLP for this activation serve as important internal controls to demonstrate the gene-specific mechanisms of the coactivator functions and gene-specific requirements for G9a, GLP and HP1γ. The results support an important role for these G9a and GLP PTMs and HP1γ in G9a and GLP coactivator function and thus provide key insights into the mechanisms that control whether G9a exerts positive regulation on specific target genes. At the physiological level, we also explore the involvement of G9a and GLP as coactivators for GR regulation of genes that control cell migration and other cellular functions.

### Materials and Methods

### Plasmids

The following plasmids were described previously: luciferase reporter plasmid MMTV-LUC (which contains glucocorticoid responsive elements), along with mammalian protein expression vectors for hG9a and hG9a fragments, hGLP, hGR, and mGrip1 [11b, 12b, 44b]; and bacterial expression vector for GST-hG9a N (1-280) [4b]. PCR-amplified DNA fragments encoding hG9a ΔN (735-1210), hGLP ΔN (814-1279) and hGLP N (31-357) were cloned into the EcoRI-BamHI, BamHI or EcoRI-XhoI sites,respectively, of the vector pgex-4t1. PCR-amplified cDNA fragment encoding hG9a and hGLP were cloned into the EcoRI site of the lentiviral vector of FUW.FTRT.GFP provided by Dr. Wange Lu (USC). For lentiviral production, the packaging vector psPAX2 and the envelope plasmid pMD2.G were used. G9a and GLP point mutants were generated with the QuikChange site-directed mutagenesis kit (Stratagene) using pSG5.HA-hG9a, pSG5.HA-hGLP, FUW.FTRT.GFP-hG9a, pGEX.4T1-hG9a (1-280) or pGEX.4T1-hGLP (31-357) as templates. The pcDNA-FLAG-Aurora-B-WT plasmid encoding human Aurora kinase B was provided by Dr. Masaaki Tatsuka (University of Hiroshima).

### Cell Culture

Cos-7, CV-1, MCF-7 and A549 cells were purchased from American Type Culture Collection (ATCC) and maintained in Dulbecco's modified Eagle's medium (DMEM) supplemented with 10% fetal bovine serum (FBS) at 37° C. and 5% CO2. For ZM447439 (Tocris) aurora kinase B inhibitor, UNC0638, UNC0642 and UNC0646 (Sigma) G9a/GLP catalytic activity inhibitors, cells were treated with the indicated concentration of the compound or with the equivalent volume of DMSO for the indicated amount of time.

For lentivirus particle production, 293T cells were plated in 100-mm dishes and transiently transfected by lypofectamine 3000 (Invitrogen) according to the manufacturer's protocol with the transducing vector (FUW.FTRT.GFP-HA-G9a wild type or K185R mutant, or FUW.FTRT.GFP-HA-GLP wild type or K205R mutant), the packaging vector psPAX2 and the envelope plasmid pMD2.G. The medium was changed the next day, and viruses were harvested by collecting the medium at 48 and 72 h post-transfection. Virus-containing medium from 2 harvests was pooled, passed through a 0.45 μm filter, and stored at −80° C. For lentiviral transduction, A549 cells were seeded a day before to reach 80% of confluency at the day of infection. Medium containing virus was added to cells along with Polybrene (Millipore) at the final concentration of 6 μg/ml. 24 h after infection, virus-containing medium was replaced with culture medium containing puromycin (1 μg/ml) for selection of infected cells. The resistant cell populations were used for the indicated experiments.

Protein Depletion by siRNA

SMARTpool siRNAs used for depletion of G9a, GLP, HP1γ, HP1α, HP1β, CDH1 and aurora kinase B and ON-TARGETplus Non-targeting siRNA#2 used as control non-specific siRNA (siNS) (Dharmacon), were transfected into A549 cells using lipofectamine siRNAi max (Invitrogen) according to the manufacturer's protocol. In various embodiments, we used a pool of siRNAs against Aurora kinase B that was purchased from Dharmacon, catalogue #L-003326-00-0020—Smart pool On-targetplus human AurKB siRNA.

Lentivirus Production and Delivery of Anti-GLP shRNA followed by Microarray Analysis

All the procedures were performed in parallel with the anti-G9a shRNA analysis, using four biological replicates from four independent experiments performed on different days, as previously described for G9a [4b]. Global gene expression analysis was performed with Illumina Human-Ref8v3 microarrays, using total RNA samples from four biological replicates from independent experiments performed on different days. Each experiment included noninfected A549 cells or A549 cells infected with lentivirus encoding shNS or shGLP, treated with 100 nM dex or equivalent volume of vehicle ethanol for 24 h. Data analysis methods were described previously [4b]. To define hormone-regulated genes, the untreated control gene set (pooled data of uninfected cells and cells infected with the virus encoding shNS) was compared with the control gene set that was hormone-treated; a q value cutoff of 0.01 was applied along with a hormonal regulation fold change cutoff of 1.5 to facilitate subsequent experimental target gene validation and reduce the number of potential false positives. To define GLP-regulated genes, the control gene set that was hormone treated was compared with the shGLP gene set that was hormone treated, and a q value cutoff of 0.05 with no fold change cutoff was applied. Forward primer used for PCR to create shGLP is as follows: 5′-CTTGTGGAAAGGACGAAACACCGAAGTTCGAGGAGCTAGAAATCATATTCAAGAGATA TGATCTCTAGCTTCTCGAACTTCTTTTTCTGCAG-3′ (SEQ ID NO: 15; bold indicates shRNA targeting sequence). The complete microarray data has been deposited in GEO with accession number GSE94646.

### Immunoprecipitation and Immunoblot

Cos-7 or A549 cells were seeded on 10-cm dishes the day before transfection. Cells were transiently transfected (where indicated) using Lipofectamine 2000 (Invitrogen) with 5 μg each of the indicated plasmids according to the manufacturer's protocol. At 48 h after transfection, cells were treated (or not) with dex for the indicated time period, and cell extracts were prepared in RIPA buffer (50 mM Tris-HCl, pH 8, 150 mM NaCl, 1 mM EDTA, 1% NP-40 and 0.25% deoxycholate) supplemented with protease inhibitor tablets (Roche Molecular Biochemicals) and phosphatase inhibitors (1 mM NaF, 1 mM Na₃VO₄ and 1 mM β-glycerophosphate). Protein extracts were incubated with 1 μg of the indicated primary antibodies overnight at 4° C. with shaking. Protein A/G Plus Agarose (Santa Cruz sc-2003) beads were added and the mixture was incubated 2 hr at 4° C. The immunoprecipitates were separated on SDS-PAGE. Immunoblotting was conducted with primary antibodies against HA epitope (3F10 Roche Applied Science); G9a (G6919), FLAG (F1804), (3-actin (A5441), or GAPDH (G9545) from Sigma; aurora kinase B (ab2254), HP1γ (ab10480), HP1γ (ab56978), phospho-593-HP1γ (ab45270) or pan methyllysine (ab23366) from Abcam; GR (sc-8992) from Santa Cruz; GLP (09-078), or pan phospho-threonine (AB1607) from Millipore; phospho-S211-GR (#4161), H3K9me3 (#13969), H3S10ph (#53348), Histone H3 (#4499) from Cell signaling; or E-cadherin (610182) from BD transduction laboratories. Secondary antibodies from Santa Cruz Biotechnology (anti-rat) and Promega (anti-rabbit and anti-mouse) were used for chemiluminescence detection using Super Signal West Dura (Thermo Scientific) for proteins with low expression levels and ECL prime detection reagent (Amersham) for all other proteins according to the manufacturers' instructions. In immunoprecipitation experiments 3% of the input of each sample was analyzed by immunoblot using the antibodies listed.

### Methyltransferase Assays

Bacterially produced GST fusion proteins (2 μg) of N-terminal fragments of G9a or GLP (GST-hG9a N or GST-hGLP N), mutant (GST-hG9a N K185R or GST-hGLP N K205R or GST-hG9a N T186A) or GST alone were incubated 90 min at 30° C. with GST-hG9a ΔN or GST-hGLP ΔN in the presence (or not) of 1 mM of unradiolabeled SAM (New England Biolabs, B9003S). Methylated products were analyzed by standard SDS gel electrophoresis followed by immunoblot. The radioactive methylation assay were performed in the same experimental conditions in the presence of 1 μCi/ml of 5-adenosyl-L[methyl-3H]methionine (55-85 Ci/mmol; Perkin Elmer; NET155H250UC). Methylation reactions were separated on SDS-PAGE. Following electrophoresis, gels were incubated in Amplify fluorographic reagent (Amersham Biosciences) according to the manufacturer's instructions and visualized by fluorography.

### Luciferase Assays

CV-1 cells were plated in hormone-free medium with 5% charcoal-stripped serum in 24-well plates the day before transfection. Cells were transfected using Lipofectamine 2000 (Invitrogen) with the indicated plasmids according to the manufacturer's protocol. After transfection, the cells were grown in hormone-free medium for 48 h in the presence or absence of 100 nM dex. Cell lysis and luciferase assays on cell extracts were performed with Promega luciferase assay kit. An aliquot of the cell lysate was reserved for immunoblot analysis of input samples. The results were normalized as indicated and presented as the mean±SEM of at least four independent experiments.

### Chromatin Immunoprecipitation

ChIP experiments were performed according to previously described protocols [4b] with antibodies against GR (Santa Cruz sc-8992X), HP1γ (Abcam ab10480), HP1α (Cell signaling #2616), HP1β (Cell signaling #8676), Phospho-Rpb1 CTD (Ser5) (Cell signaling #13523), H3K9me3 (Cell signaling #13969), H3S10ph (Cell signaling #53348) and HA epitope (3F10 Roche Applied Science). Results are expressed relative to the signal obtained from input chromatin. Primer sequences are indicated below in Tables 4 and 5.

TABLE 4

| Primer Name | Sense | SEQ ID NO: # |
|---|---|---|
| ENaCα -2.5 kb | 5′ AAACTCCAGTCTCCCTTGAGC 3′ | SEQ ID NO: 16 |
| ENaCα GBR (-1.3 kb) | 5′ CACCTTCAGTGCCTGCTTTC 3′ | SEQ ID NO: 17 |
| ENaCα TSS | 5′ TCAACTGGAAAGGAACCAGTC 3′ | SEQ ID NO: 18 |
| ENaCα +2.1 kb | 5′ CAACGAAATGACCTGGCTTT 3′ | SEQ ID NO: 19 |
| ENaCα +5.7 kb | 5′ GACCTTTTGGGAGAGTGAAGG 3′ | SEQ ID NO: 20 |
| ENaCα +11 kb | 5′ CCGGAAATTAAAGAGGAGCTG 3′ | SEQ ID NO: 21 |
| CDH16 -1.5 kb | 5′ GCCAAGGTCCATACATTCCTT 3′ | SEQ ID NO: 22 |
| CDH16 GBR (-0.36 kb) | 5′ TTGAGCTGAGCACTGAAGCATG 3′ | SEQ ID NO: 23 |
| CDH16 TSS | 5′ TGGCTTTCCAAAGTCAATGAG 3′ | SEQ ID NO: 24 |
| CDH16 +2.5 kb | 5′ ATCTCCGGAGTCCTGATGTG 3′ | SEQ ID NO: 25 |
| CDH16 +5 kb | 5′ AGTGGGTGGGGTAAGGTCTC 3′ | SEQ ID NO: 26 |
| CDH1 GBR (+21 kb) | 5′ CCTGCTCATCTTCTCCCAGA 3′ | SEQ ID NO: 27 |
| HSD11B2 GBR (-7.5 kb) | 5′ TGTAACTGGTGCGACTTGGAA 3′ | SEQ ID NO: 28 |
| HSD11B2 TSS | 5′ GGGACTGGACACTCAACAGG 3′ | SEQ ID NO: 29 |
| PPL GBR (-7.7 kb) | 5′ CAGCTTCACCCCTGTTTTGTA 3′ | SEQ ID NO: 30 |
| FKBP5 GBR (+86 kb) | 5′ TGTGCCAGCCACATTCAGAACA 3′ | SEQ ID NO: 31 |
| FKBP5 TSS | 5′ TCCCATCTAGCTCTGGTCTCA 3′ | SEQ ID NO: 32 |
| CITED2 GBR (-0.93 kb) | 5′ AGTTTGCGTTTGCAGCTCTT 3′ | SEQ ID NO: 33 |
| FOXO1 GBR (-0.2 kb) | 5′ AGATTTGGGGGAACGAAGCC 3′ | SEQ ID NO: 34 |
| H3K9me3 positive region | TCTTGGAGCTTGCCTTTCAT | SEQ ID NO: 35 |
| H3K9me3 negative region | CAGCTAATCAGCCTCCTTGG | SEQ ID NO: 36 |

TABLE 5

| Primer Name | Antisense | SEQ ID NO: # |
|---|---|---|
| ENaCα -2.5 kb | 5′ CCATGCTGCCTTAAGCTAGTG 3′ | SEQ ID NO: 37 |
| ENaCα GBR (-1.3 kb) | 5′ AGGCCAGGAATGTGTAATCG 3′ | SEQ ID NO: 38 |
| ENaCα TSS | 5′ CTCGAGCTGTGTCCTGATTCT 3′ | SEQ ID NO: 39 |
| ENaCα +2.1 kb | 5′ GGCCCCTTCGTATATTCCAT 3′ | SEQ ID NO: 40 |
| ENaCα +5.7 kb | 5′ CCACACACACAAACCTGTGAC 3′ | SEQ ID NO: 41 |
| ENaCα +11 kb | 5′ TACAGGTCAAAGAGCGTCTGC 3′ | SEQ ID NO: 42 |
| CDH16 -1.5 kb | 5′ CTCCTGCCATTCAATAAGCTG 3′ | SEQ ID NO: 43 |
| CDH16 GBR (-0.36 kb) | 5′ TGCAGCCACACCTTTTCACAC 3′ | SEQ ID NO: 44 |
| CDH16 TSS | 5′ GGCACTTGAGCAGGTAGGAG 3′ | SEQ ID NO: 45 |
| CDH16 +2.5 kb | 5′ TGAAGCCTCAAGGAAGAGGA 3′ | SEQ ID NO: 46 |
| CDH16 +5 kb | 5′ CAGGGCTCAGGAGCTGATAC 3′ | SEQ ID NO: 47 |
| CDH1 GBR (+21 kb) | 5′ TGCACCAAGAACGCTTTATG 3′ | SEQ ID NO: 48 |
| HSD11B2 GBR (-7.5 kb) | 5′ TTCCAAACACCTTGTCCCCAA 3′ | SEQ ID NO: 49 |
| HSD11B2 TSS | 5′ GGTGGAGAACTCTCCCACTCT 3′ | SEQ ID NO: 50 |
| PPL GBR (-7.7 kb) | 5′ GGCCAGCACAATTTTCCACT 3′ | SEQ ID NO: 51 |
| FKBP5 GBR (+86 kb) | 5′ GTAACCACATCAAGCGAGCTG 3′ | SEQ ID NO: 52 |
| FKBP5 TSS | 3′ GGGACTGCTTCTCACCATGT 3′ | SEQ ID NO: 53 |
| CITED2 GBR (-0.93 kb) | 5′ AAGGTGGATCTGGGGACGAG 3′ | SEQ ID NO: 54 |
| FOXO1 GBR (-0.2 kb) | 5′ GATGGCCCCGCGAAGTTAAG 3′ | SEQ ID NO: 55 |
| H3K9me3 positive region | TTCAATGACCTCAGCAGCAG | SEQ ID NO: 56 |
| H3K9me3 negative region | GCCTCAAGAAGCTGGACATC | SEQ ID NO: 57 |

### Real-Time RT-qPCR

RNA was isolated using Trizol (Invitrogen) according to the manufacturer's instructions. Reverse transcription reaction was performed using iScript (Biorad) according to specifications with 0.8 μg of total RNA as template. Quantitative PCR amplification of the resulting cDNA was performed on a Roche LightCycler 480 using SYBR green I master mix (Roche). mRNA levels were normalized to the level of β-actin mRNA. Primer sequences are specified below in Tables 6 and 7.

TABLE 6

| Gene Name | Sense | SEQ ID NO: # |
|---|---|---|
| CDH16 | 5′ TCGGCAGTGGGCATCCTTGTA 3′ | SEQ ID NO: 58 |
| ENaCα | 5′ AACGGTCTGTCCCTGATGCT 3′ | SEQ ID NO: 59 |
| HSD11B2 | 5′ GACCTGACCAAACCAGGAGA 3′ | SEQ ID NO: 60 |
| PPL | 5′ CAGGAGATCCTCCAATTCCA 3′ | SEQ ID NO: 61 |
| CDH1 | 5′ TTCCCAACTCCTCTCCTG 3′ | SEQ ID NO: 62 |
| FKBP5 | 5′ AGGCTGCAAGACTGCAGATC 3′ | SEQ ID NO: 63 |
| CITED2 | 5′ GCCAGGTTTAACAACTCCCA 3′ | SEQ ID NO: 64 |
| FOXO1 | 5′ ACAGTTTTCCAAATGGCCTG 3′ | SEQ ID NO: 65 |
| β-actin | 5′ CCACACTGTGCCCATCTACG 3′ | SEQ ID NO: 66 |
| HP1α | 5′ GATGTCATCGGCACTGTTTG 3′ | SEQ ID NO: 67 |
| HP1β | 5′ TTTGGTTTGCTCTCCTCTCC 3′ | SEQ ID NO: 68 |
| HP1γ | 5′ AAGAGGCAGAGCCTGAAGAA 3′ | SEQ ID NO: 69 |
| G9a | 5′ ATGGGTGAAGCCGTCTCGGA 3′ | SEQ ID NO: 70 |
| GLP | 5′ GATAGCGGAAAATGGGGTTT 3′ | SEQ ID NO: 71 |

TABLE 7

| Gene Name | Antisense | SEQ ID NO: # |
|---|---|---|
| CDH16 | 5′ GCACGCTGTCTGCTGGTTGAT 3′ | SEQ ID NO: 72 |
| ENaCα | 5′ TTGGTGCAGTCGCCATAATC 3′ | SEQ ID NO: 73 |
| HSD11B2 | 5′ CCGCATCAGCAACTACTTCA 3′ | SEQ ID NO: 74 |
| PPL | 5′ CTGGGAAGCTCTTTCCCTCT 3′ | SEQ ID NO: 75 |
| CDH1 | 5′ AAACCTTGCCTTCTTTGTC 3′ | SEQ ID NO: 76 |
| FKBP5 | 5′ CTTGCCCATTGCTTTATTGG 3′ | SEQ ID NO: 77 |
| CITED2 | 5′ CTGGTTTGTCCCGTTCATCT 3′ | SEQ ID NO: 78 |
| FOXO1 | 5′ CATCCCCTTCTCCAAGATCA 3′ | SEQ ID NO: 79 |
| β-actin | 5′ AGGATCTTCATGAGGTAGTCAGTCAG 3′ | SEQ ID NO: 80 |
| HP1α | 5′ GCACAATACTTGGGAACCTGA 3′ | SEQ ID NO: 81 |
| HP1β | 5′ AACACATGGGAGCCAGAAGA 3′ | SEQ ID NO: 82 |
| HP17 | 5′ TCTGTAAATCCCTTCCACTTCA 3′ | SEQ ID NO: 83 |
| G9a | 5′ ATCTTGGGTGCCTCCATGCG 3′ | SEQ ID NO: 84 |
| GLP | 5′ GTAGTCCTCAAGGGCTGTGC 3′ | SEQ ID NO: 85 |

### Proximity Ligation Assay

The experiments were performed following the manufacturer's instructions as previously described [21, 45]. Cells were grown on coverslips in 12-well plates, fixed in methanol for 2 min, and then washed twice in PBS. Firstly, the samples were saturated using the blocking solution, then different pairs of primary antibodies (HP1γ (Abcam ab10480) and GR (Santa Cruz sc-393232) in order to analyze HP1γ-GR, G9a (Sigma G6919) and HP1γ (Abcam ab56978) in order to analyze HP1γ-G9a interaction and HA-Tag (6E2) (Cell signaling #2367) and HP1γ (Abcam ab10480) in order to analyze HA- HP1γ interaction) were incubated with the fixed cells for 1 h at 37° C. After washes, the PLA minus and plus probes (containing the secondary antibodies conjugated with complementary oligonucleotides) were added and incubated 1 h at 37° C. After the ligation of oligonucleotides into a circular template, the addition of nucleotides and DNA polymerase allows a rolling-circle amplification reaction during an incubation of 100 min at 37° C. The amplification solution also contains fluorescently labeled oligonucleotides that hybridize to the amplification product. Afterwards, the samples were mounted with Duolink II Mounting Medium containing Dapi in order to counterstain nuclei, and then analyzed on Zeiss Imager.Z1 fluorescence microscope. For each sample interactions were counted for 1000 cells using Image J software [46b].

### Immunofluorescence

Cells were grown on coverslips in 12-well plates. Cells were then fixed in cold methanol for 2 minutes, washed twice in PBS and incubated in PBS1X gelatin for 30 minutes. Then, the cells were incubated with E-cadherin antibody (610182) from BD Transduction Laboratories in Dako diluent (S0809) for 1 hour at 37° C. After PBS washes, the cells were incubated for 1 hour at 37° C. with the mouse secondary antibodies coupled with Alexa Fluor 488 from Invitrogen (1:3000) in Dako antibody diluent, then washed in PBS and mounted on glass slides in mounting solution (Dako). Slides were analyzed on Zeiss Imager.Z1 fluorescence microscope.

### Cell Migration

A549 cells suspended in serum free medium were plated in the upper part of a 24-well, 8-μm pore, cell Transwell migration chamber (Cell biolabs inc, San Diego, Calif., USA) according to the manufacturer's protocol. Medium with 10% FBS was placed in the lower wells. 0.4×10⁶ cells were incubated for migration at 37° C. with 5% CO₂ for 24 hours. Then cells were fixed and stained with Cell Staining Solution (Cell Biolabs). After washing, images of migrated cells on the opposite side of the membrane were captured with an inverted microscope. Migratory cells were dissociated from the membrane using Extraction Solution (Cell Biolabs). Optical density of the dye was measured at 560 nm in a 96-well microtiter plate.

### Proliferation

Twenty-four hours after transfection with the appropriate siRNA, MCF-7 cells were plated in triplicate in 96-well plates at a density of 2500 cells per well. One plate was harvested and analyzed each day of the time course. At each time point, cells were treated with MTS (Promega G3581) and incubated 1 h at 37° C. Absorbance was monitored at 490 nm with a 96-well plate reader.

### Figure Descriptions

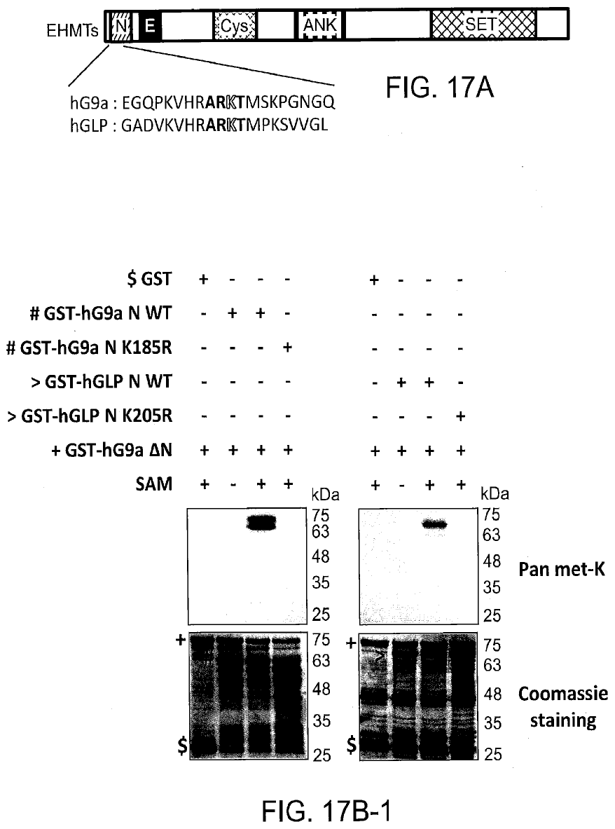

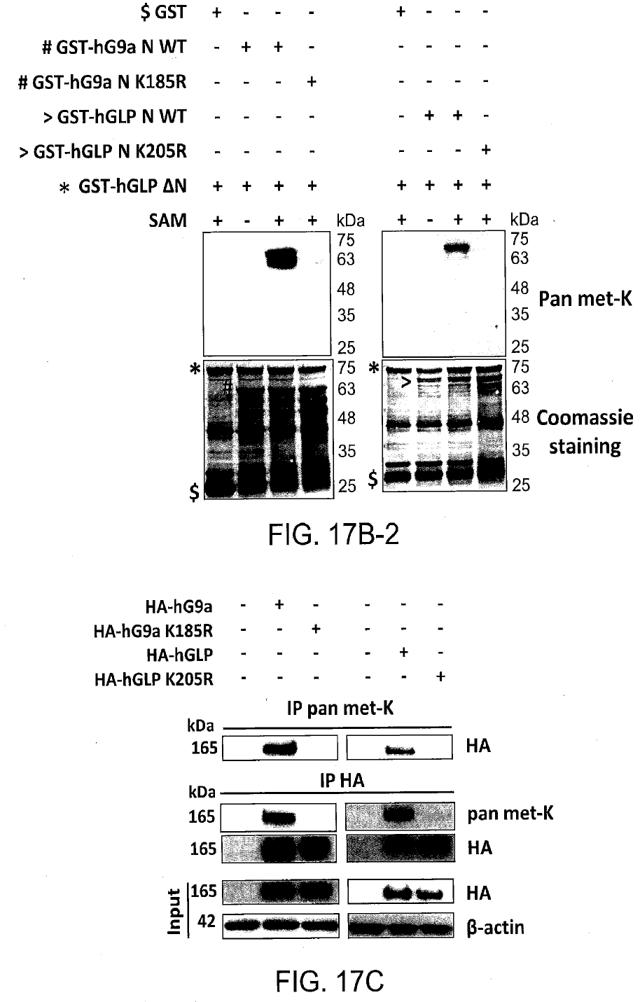

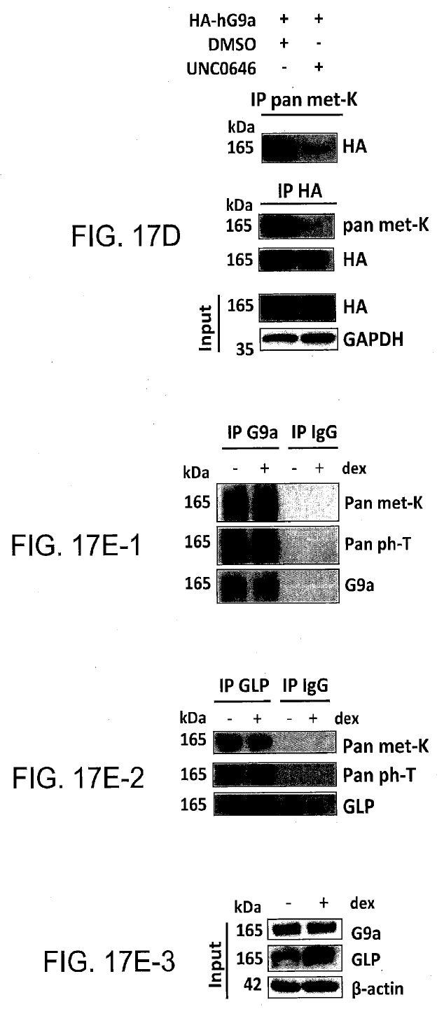

The results as shown in FIGS. 17A-17E generally highlight that G9a and GLP are methylated on their N-terminal domain in cells. FIG. 17A is a schematic representation of the related proteins GLP (EHMT1) and G9a (EHMT2). N: N-terminal coactivator domain, E: Polyglutamate domain, Cys: Cysteine-rich region, ANK: Six ankyrin repeats, SET: SET-domain containing methyltransferase activity. Partial protein sequence of hG9a and hGLP homologs shows the hypothetical methylated lysine residues (K) in red. For FIGS. 17B-1 and 17B-2, after protein methylation reactions in vitro methylated proteins were detected by immunoblot with pan methyllysine antibody (pan met-K). The corresponding Coomassie-stained gels are shown as loading controls. SAM, S-adenosylmethionine. For FIG. 17C, Cos-7 cells were transfected with plasmids encoding full length HA-hG9a wild type or K185R mutant, or full length HA-hGLP wild type or K205R mutant. Lysates were immunoprecipitated (IP) with pan met-K antibody and immunoblotted with HA antibody (top), or the usage of the two antibodies was reversed (bottom). Expression of HA-tagged proteins and β-actin (loading control) in the unfractionated extracts is shown at the bottom (Input). For FIG. 17D, Cos-7 cells were transfected with a plasmid encoding full length HA-hG9a and treated with 2 μM UNC0646 or vehicle DMSO for 24 h. Lysates were immunoprecipitated with pan met-K antibody and immunoblotted with HA antibody (top), or the usage of the two antibodies was reversed (bottom). For FIGS. 17E-1, 17E-2, and 17E-3, methylation and phosphorylation of endogenous G9a and GLP in A549 cells treated with 100 nM dex for 4 h was analyzed by immunoprecipitation with control IgG antibody, anti-G9a (top) or anti-GLP (bottom), followed by immunoblot with antibodies listed. Expression of G9a, GLP and β-actin (loading control) in the unfractionated extracts is shown at the right (Input).

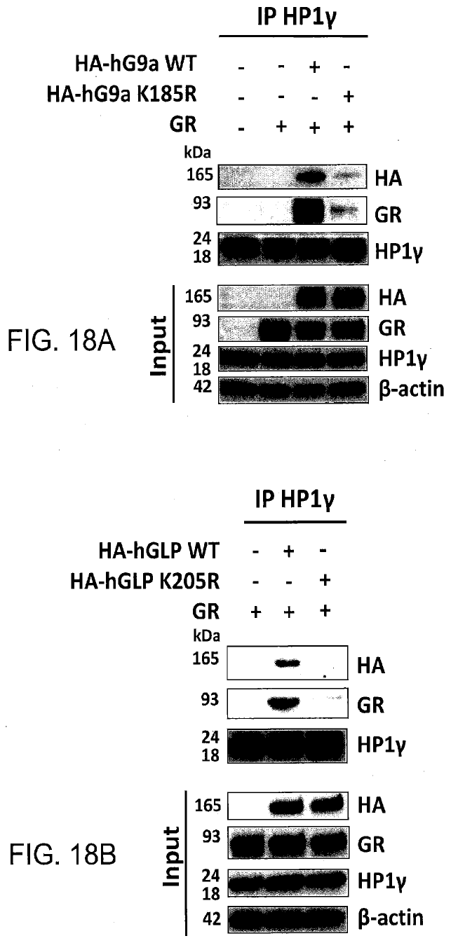

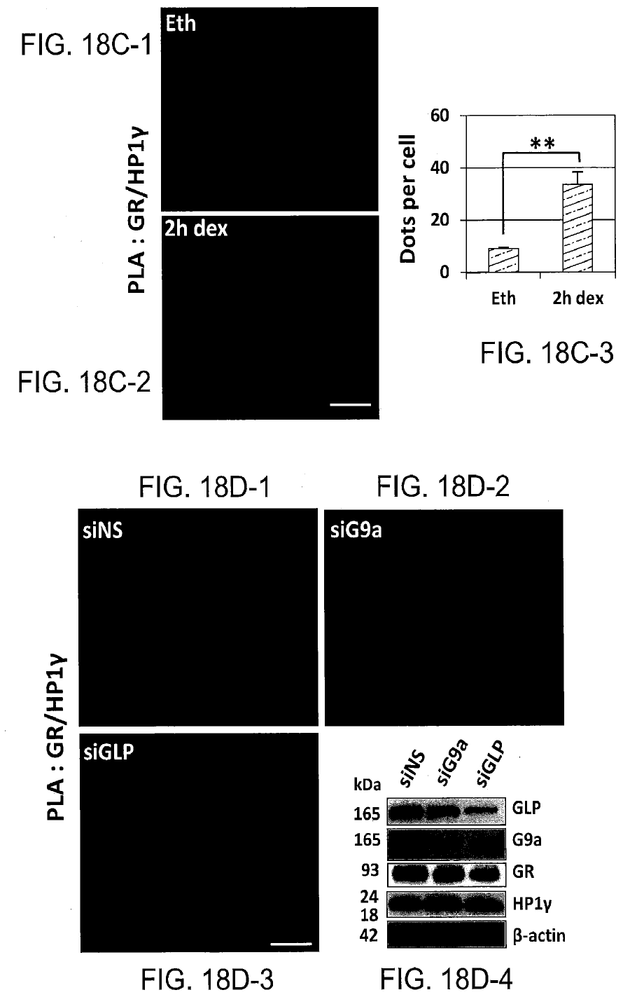

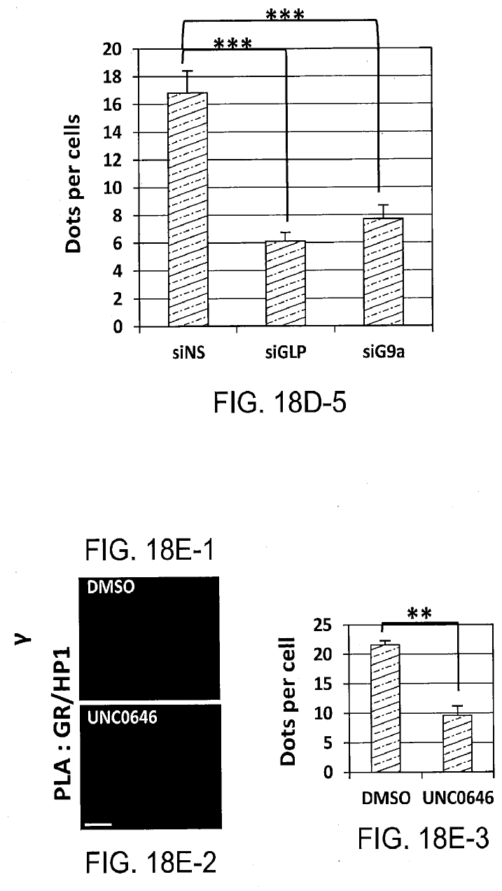

The results as shown in FIGS. 18A-18E generally highlight that G9a and GLP methylation is required for HP1γ-G9a/GLP-GR ternary complex formation. For FIG. 18A, Cos-7 cells were transfected with plasmids encoding hGR and full length HA-hG9a wild type or the K185R mutant. Lysates supplemented with 15 U/ml of DNAse I were immunoprecipitated with HP1γ antibody and immunoblotted using antibodies listed. For FIG. 18B, Cos-7 cells were transfected with plasmids encoding hGR and full length HA-hGLP wild type or the K205R mutant and were treated and analyzed as in A. For FIGS. 18C-1, 18C-2, and 18C-3, to analyze interaction of endogenous GR and HP1γ by PLA, A549 cells were treated with 100 nM dex or the equivalent volume of vehicle ethanol (Eth) for 2 h as well as analysis of the imaging. After cell fixation, PLA with antibodies against GR and HP1γ was performed. The detected interactions are indicated by red dots. The nuclei were counterstained with DAPI (blue). The number of interactions detected by Image J analysis is shown as the mean±SEM of three independent experiments. p-value was determined using a paired t-test. **p≤0.01. Scale bar represents 10 μm. For FIGS. 18D-1, 18D-2, 18D-3, 18D-4, and 18D-5, PLA was conducted as in FIGS. 18C-1, 18C-2, and 18C-3 after transfection of A549 cells with siRNA for G9a (siG9a), GLP (siGLP) or non-specific siRNA (siNS) and treatment of cells with 100 nM dex for 2 h. Detected interactions are shown as the mean±SEM of three independent experiments. p-value was determined using a paired t-test. ***p≤0.001. Scale bar represents 10 μm. Whole-cell extracts were analyzed for G9a, GLP, GR, HP1γ and β-actin expression by immunoblot. For FIGS. 18E-1, 18E-2, and 18E-3, PLA was conducted as in FIGS. 18C-1, 18C-2, and 18C-3, after treatment of cells with 2 μM UNC0646 or equivalent volume of vehicle DMSO for 24 h and with 100 nM dex for the final 2 h. Detected interactions are shown as the mean±SEM of three independent experiments. p-value was determined using a paired t-test. **p≤0.01. Scale bar represents 10 μm.

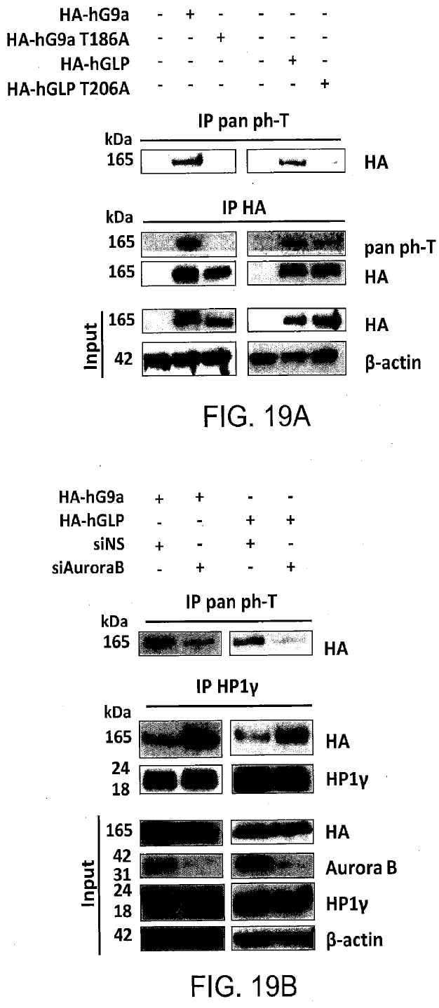

The results as shown in FIGS. 19A and 19B generally highlight that G9a and GLP phosphorylation in cells by aurora kinase B antagonizes HP1γ recognition. For FIG. 19A, Cos-7 cells were transfected with plasmids encoding full length HA-hG9a wild type or T186A mutant, or full length HA-hGLP wild type or T206A mutant. Lysates were immunoprecipitated with pan phospho-threonine antibody (IP pan ph-T) and immunoblotted with HA antibody (top), or the usage of the two antibodies was reversed (bottom). For FIG. 19B, Cos-7 cells were transfected with a plasmid encoding HA-hG9a or HA-hGLP and siRNA against Aurora kinase B (siAuroraB) or non-specific siRNA (siNS). Lysates were immunoprecipitated with pan ph-T antibody and immunoblotted with HA antibody (top). Then, lysates were immunoprecipitated with HP1γ antibody and immunoblotted with indicated antibodies (bottom).

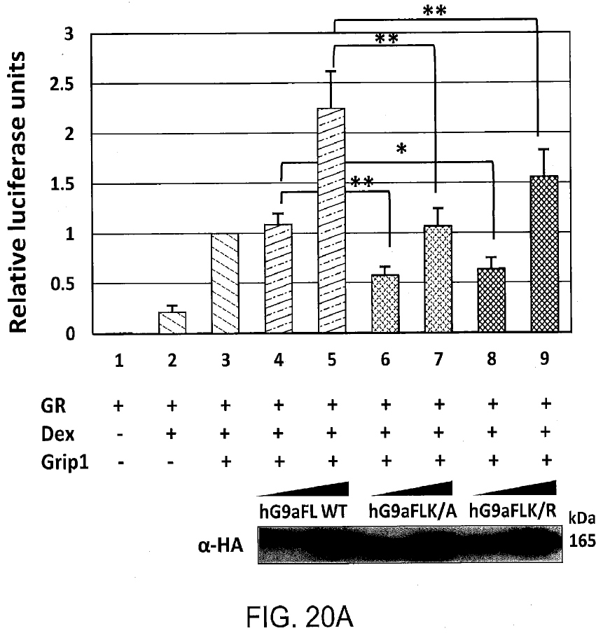

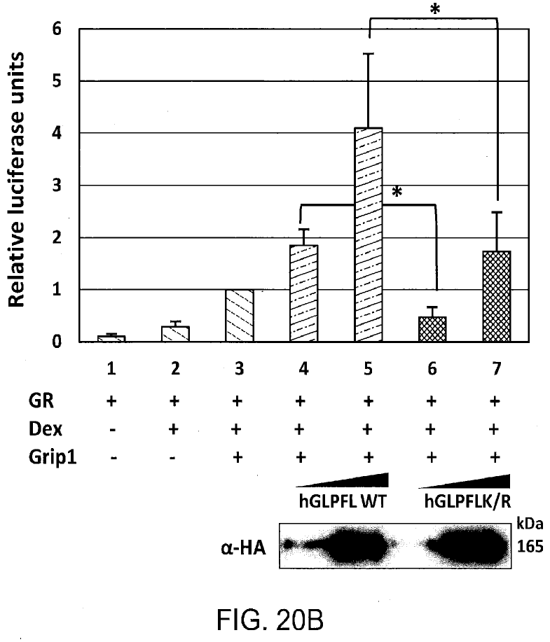

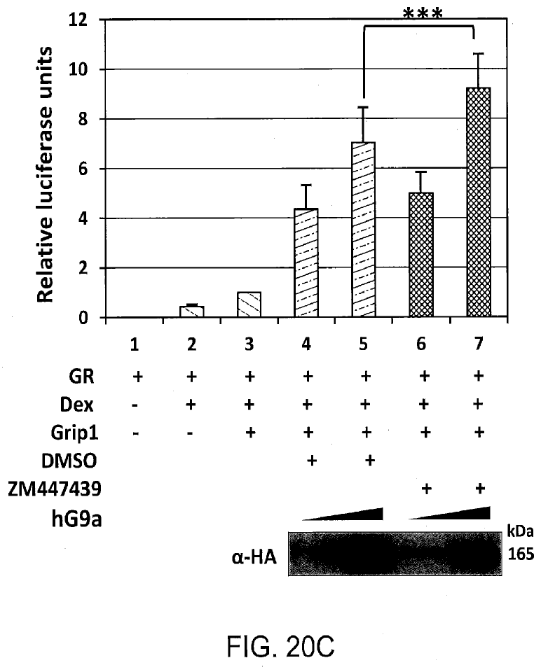

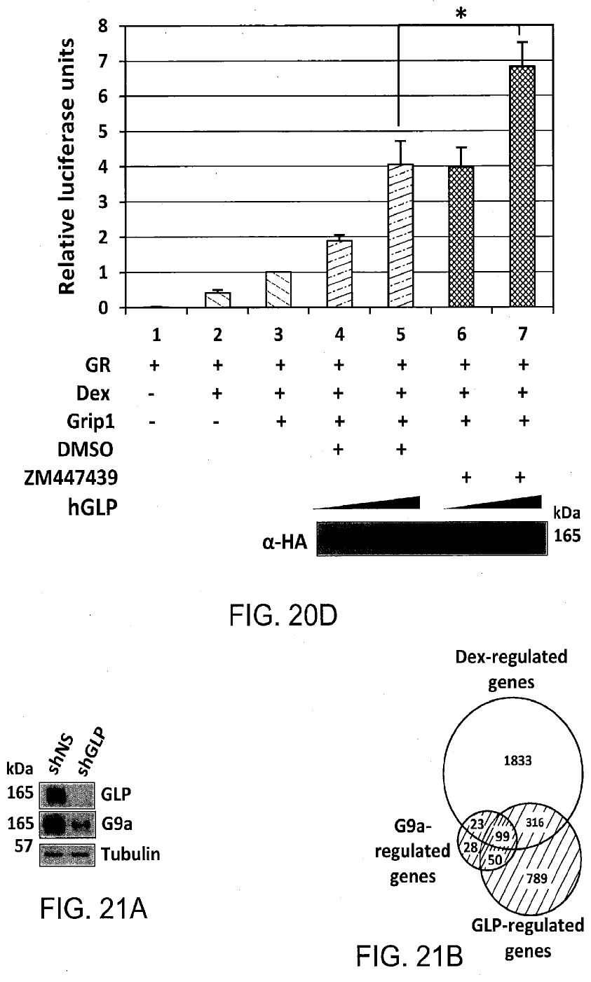

The results as shown in FIGS. 20A-20D generally highlight that G9a and GLP PTMs regulate their coactivator function. For FIG. 20A, CV-1 cells were transfected with MMTV-LUC reporter plasmid (200 ng) and plasmids encoding GR (1 ng), Grip1 (100 ng) and HA-labeled full length (FL) hG9a wild type or K185A or K185R mutants (150 or 400 ng) as indicated. Cells were grown with 100 nM dex or the equivalent amount of ethanol for 48 h and assayed for luciferase activity. Relative luciferase units are normalized to sample 3 and represent mean±SEM for eight independent experiments. p-value was calculated using a paired t-test. *p≤0.05, **p≤0.01. Whole-cell extracts were analyzed for G9a expression by immunoblot with anti-HA antibody. For FIG. 20B, transient reporter gene assays were performed as in A with HA-labeled hGLP WT or hGLP K205A (150 or 400 ng) as indicated. Relative luciferase units are normalized to sample 3 and represent mean±SEM for six independent experiments. p-value was calculated using a paired t-test. *p≤0.05. For FIG. 20C, transient reporter gene assays were performed as in A after transfected cells were treated or not with 100 nM dex and 2 μM ZM447439 (ZM) or equivalent volume of DMSO for 48 h as indicated. Relative luciferase units are normalized to sample 3 and represent mean±SEM for four independent experiments. p-value was calculated using a paired t-test. * * *p≤0.001. For FIG. 20D, Transient reporter gene assays were performed as in C, except with hGLP instead of hG9a. Relative luciferase units are normalized to sample 3 and represent mean±SEM for four independent experiments. p-value was calculated using a paired t-test. *p≤0.05.

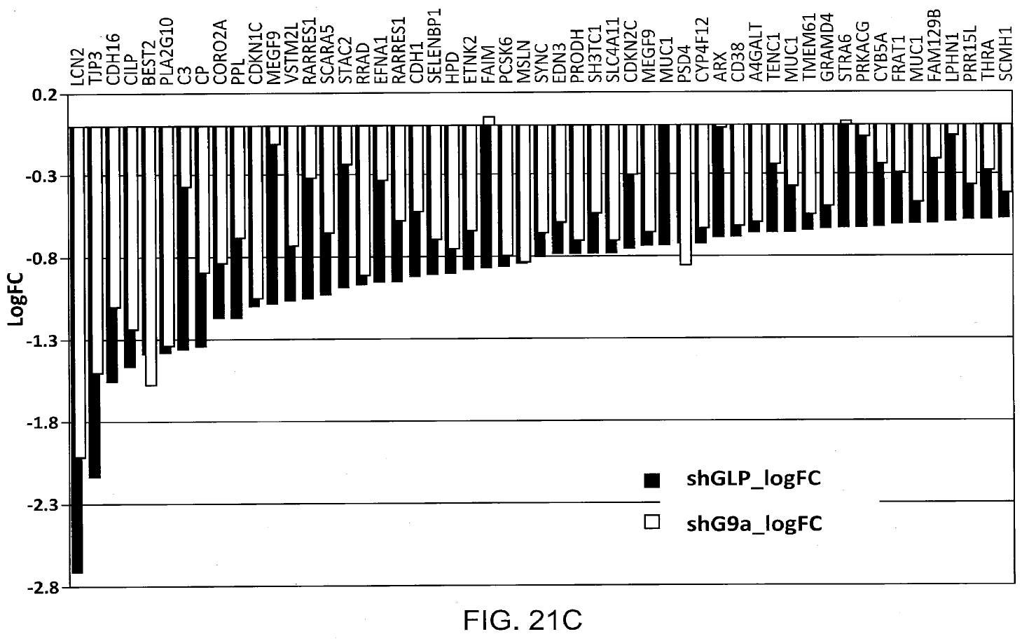

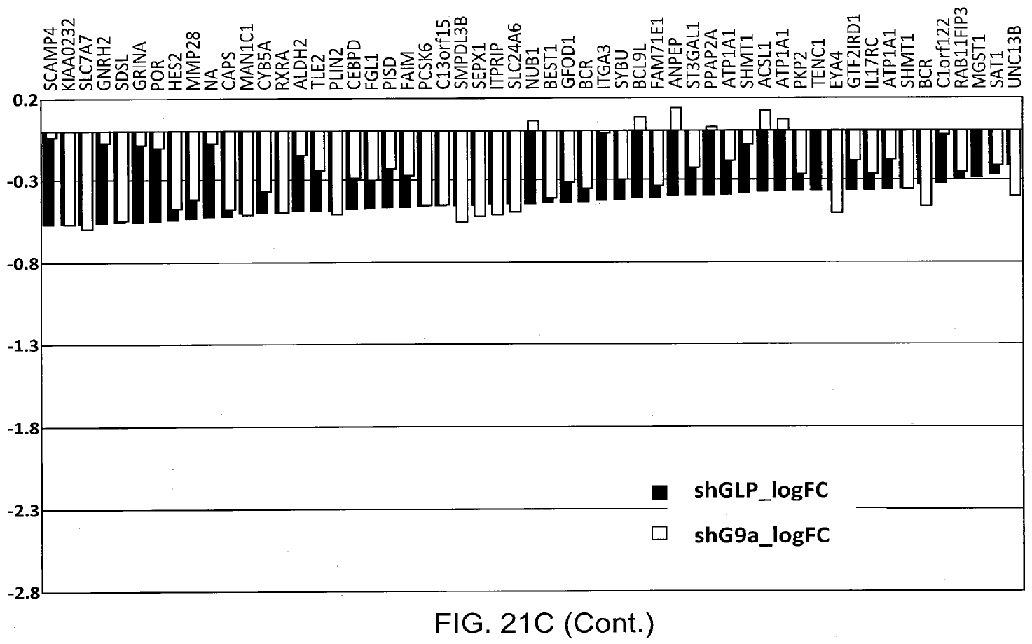

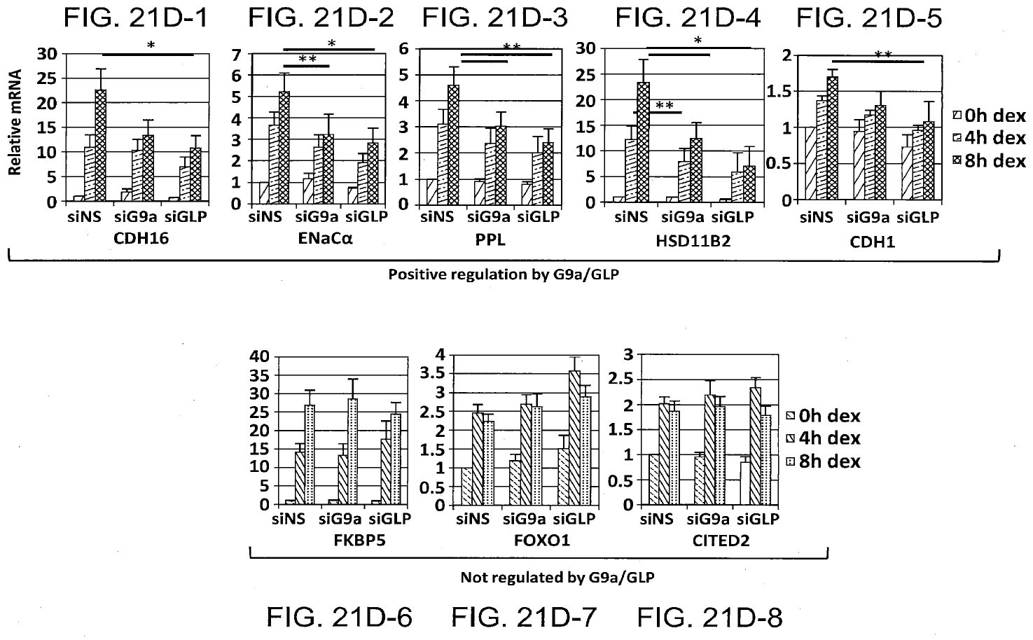

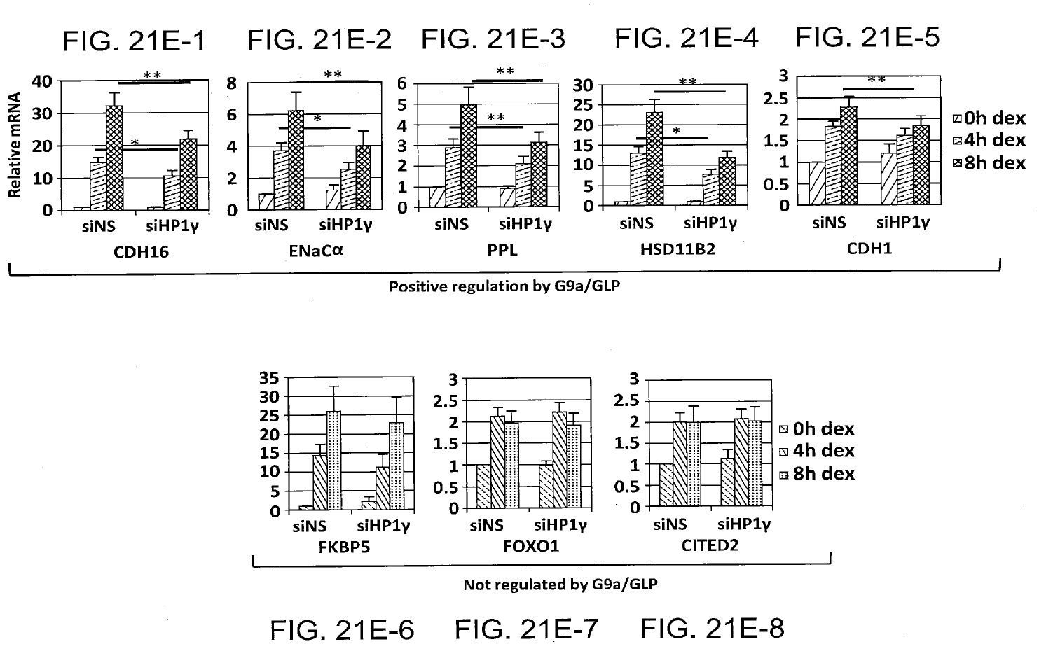

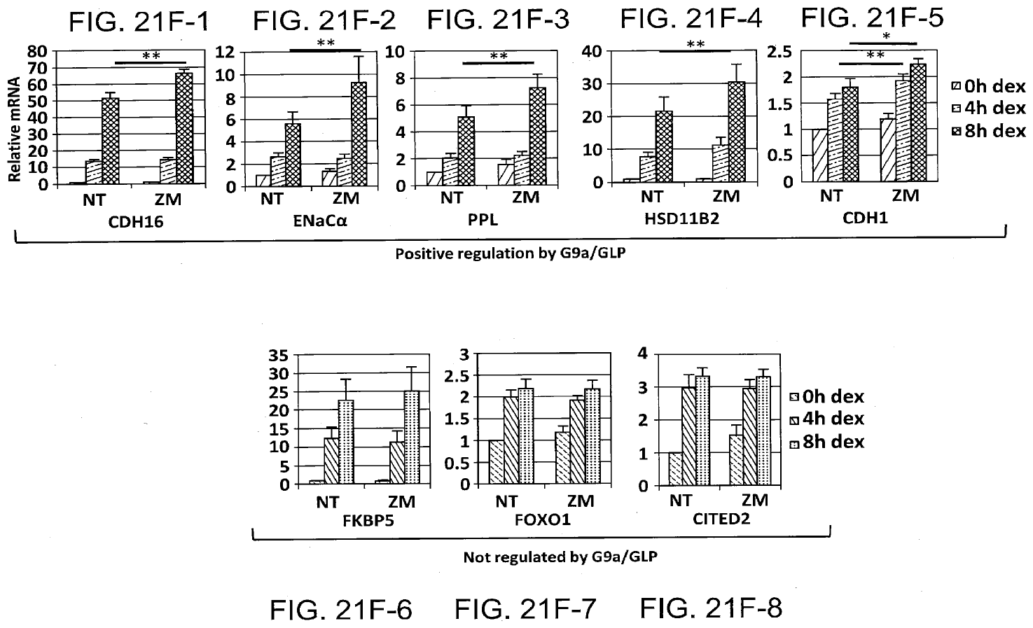

The results as shown in FIGS. 21A-21E generally highlight that G9a and GLP act as coactivators for a subset of endogenous GR target genes. FIG. 21A is an immunoblot showing GLP, G9a and tubulin protein levels in whole cell extracts from A549 cells that were transduced with a control lentivirus encoding a non-specific shRNA (shNS) or lentivirus encoding an shRNA targeting GLP (shGLP). For FIG. 21B, the large black Venn diagram 14 represents the total number of dex-regulated genes from the microarray analysis (q-value≤0.01 and at least 1.5-fold increase or decrease) for cells transfected with siNS and treated with 100 nM dex for 24 h compared with ethanol. Blue blue Venn diagram 15 represents the number of GLP-regulated genes with significantly different expression (q-value≤0.05) in dex-treated cells expressing shGLP versus dex-treated cells expressing siNS. Small purple Venn diagram 16 represents the number of G9a-regulated genes with significantly different expression (q-value≤0.05) in dex-treated cells expressing shG9a versus dex-treated cells expressing siNS [4b]. Overlap areas indicates the number of genes shared among sets. As shown in FIG. 21C, for all 108 dex-induced genes that require GLP as a coactivator according to microarray analysis (x-axis), the loge fold change due to GLP depletion for the 24 h-dex-induced mRNA levels is shown by blue or darker gray bars (y-axis). The log₂ fold change for the same genes caused by G9a depletion [4b] is shown by superimposed purple bars. For FIGS. 21D-1, 21D-2, 21D-3, 21D-4, 21D-5, 21D-6, 21D-7, and 21D-8, A549 cells transfected with non-specific siRNA (siNS) or with SMART-pool siRNA targeting G9a (siG9a) or GLP (siGLP) were treated with 100 nM dex for the indicated times (0 h dex indicates ethanol treatment for 8 hours). mRNA levels for the indicated GR target genes were measured by reverse transcriptase followed by qPCR and normalized to (3-actin mRNA levels. Results shown are mean±SEM for four independent experiments. β-value was calculated using a paired t-test. p≤0.05, **p≤0.01. For FIGS. 21E-1, 21E-2, 21E-3, 21E-4, 21E-5, 21E-6, 21E-7, and 21E-8, mRNA levels for the indicated GR target genes were determined as in FIGS. 21D-1, 21D-2, 21D-3, 21D-4, 21D-5, 21D-6, 21D-7, and 21D-8, using A549 cells transfected with non-specific siRNA (siNS) or with SMART-pool siRNA targeting HP1Δ (siHP1γ). Results shown are mean±SEM for five independent experiments. p-value was calculated using a paired t-test. *p≤0.05, **p≤0.01. For FIGS. 21F-1, 21F-2, 21F-3, 21F-4, 21F-5, 21F-6, 21F-7, and 21F-8, mRNA levels for the indicated GR target genes were determined as in FIGS. 21D-1, 21D-2, 21D-3, 21D-4, 21D-5, 21D-6, 21D-7, and 21D-8, using A549 cells which were not transfected with siRNA. 1 h prior to hormone or ethanol treatment, 2 μM ZM447439 or equivalent volume of DMSO was added. Results shown are mean±SEM for at least four independent experiments. p-value was calculated using a paired t-test. *p≤0.05, **p≤0.01.

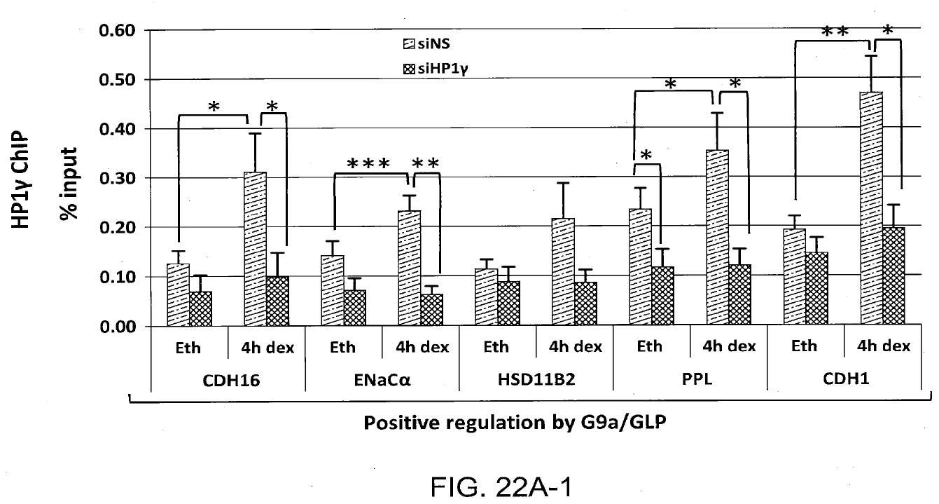

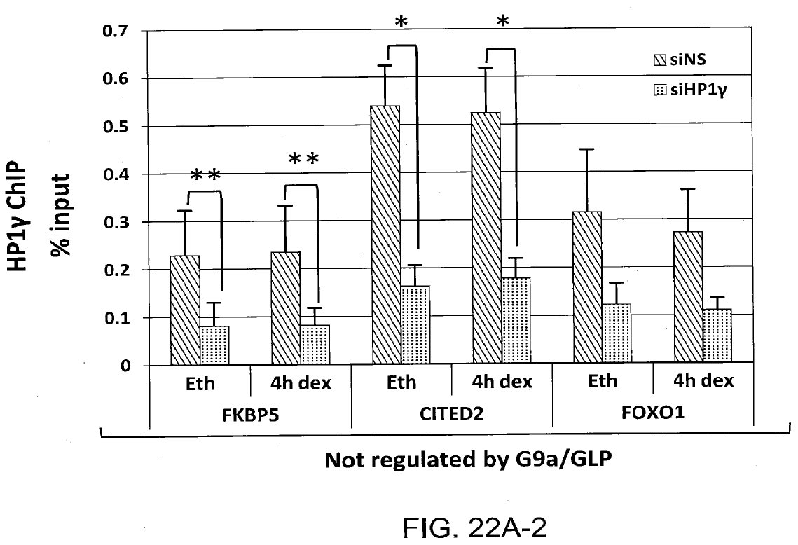

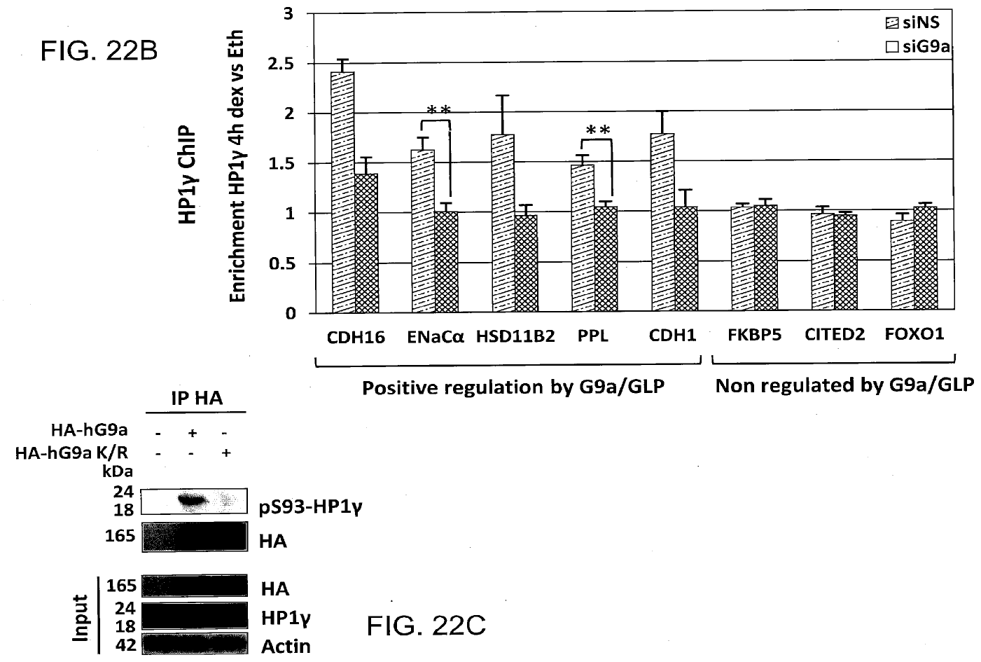

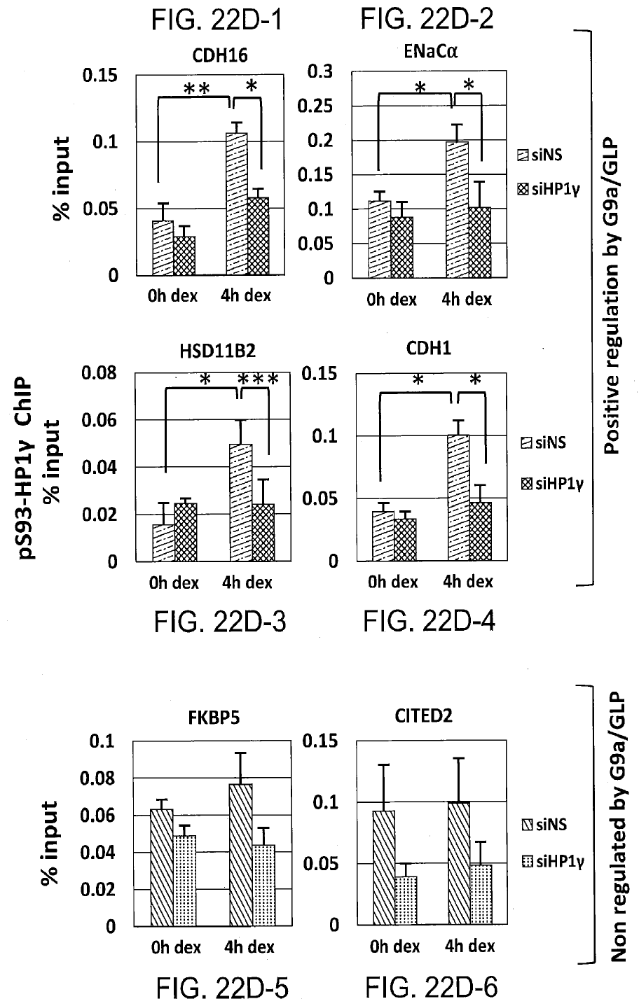

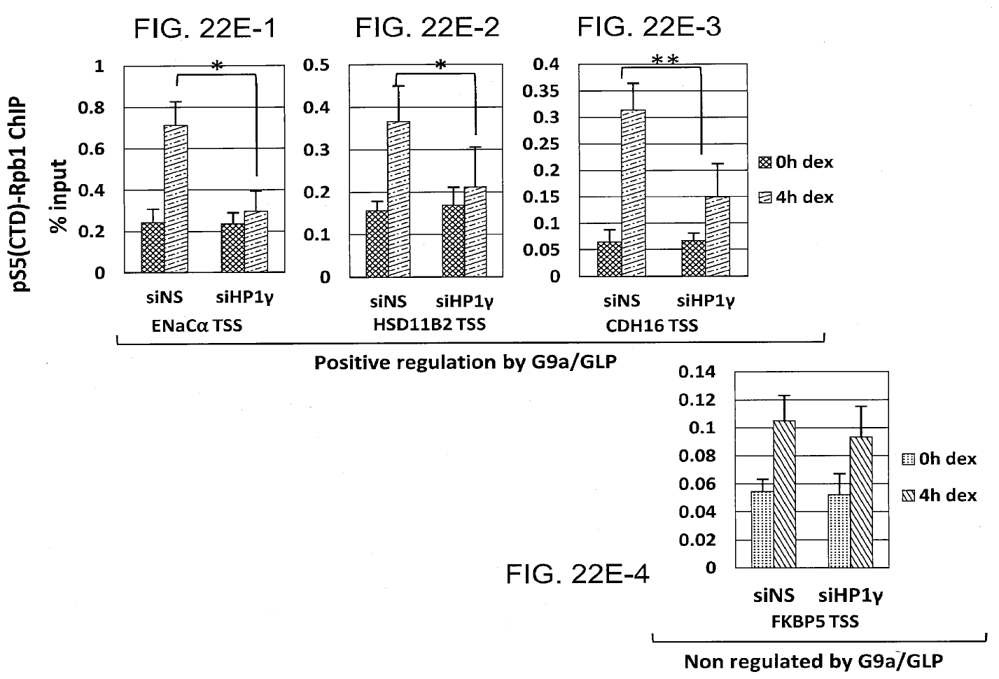

The results as shown in FIGS. 22A-22D generally highlight occupancy of HP1γ on GR binding regions (GBR) of GR target genes. For FIGS. 22A-1 and 22A-2, A549 cells were transfected with non-specific siRNA (siNS, dark blue or gray bars) or with SMART-pool siRNA targeting HP1γ (siHP1γ, light blue or gray bars) and treated with 100 nM dex or ethanol for 4 h. Immunoprecipitated DNA was analyzed by qPCR using primers that amplify the GBRs associated with the indicated GR target genes. Results are normalized to input chromatin and shown as mean±SEM for four independent experiments. p-value was calculated using a paired t-test. *p≤0.05, **p≤0.01, ***p≤0.001. For FIG. 22B, A549 cells transfected with non-specific siRNA (siNS, dark blue bars) or with SMART-pool siRNA targeting G9a (siG9a, light blue bars) were treated with 100 nM dex or ethanol for 4 h. ChIP was performed with HP1γ antibody and immunoprecipitated DNA was analyzed by qPCR using primers specific for the GBRs associated with the indicated genes. Results are normalized to input chromatin, and the mean±SEM of the ratio between 4 h dex or ethanol treatment for three independent experiments is shown. p-value was calculated using a paired t-test. **p≤0.01. For FIG. 22C, Cos-7 cells were transfected with plasmids encoding full length HA-hG9a wild type or K185R mutant. Lysates were immunoprecipitated (IP) with HA antibody and immunoblotted with phospho-S93-HP1γ (pS93-HP1γ) or HA antibodies. Expression of HA-tagged G9a, HP1γ and β-actin (loading control) in the unfractionated extracts is shown at the bottom (Input). For FIGS. 22D-1, 22D-2, 22D-3, 22D-4, 22D-5, and 22D-6, A549 cells transfected with non-specific siRNA (siNS, dark blue bars) or with SMART-pool siRNA targeting HP1γ (siHP1γ, light blue bars) were treated with 100 nM dex or ethanol for 4 h. ChIP was performed with phospho-S93-HP1γ antibody, and immunoprecipitated DNA was analyzed by qPCR using primers that amplify the GBRs associated with the indicated GR target genes. Results are normalized to input chromatin and shown as mean±SEM for three independent experiments. p-value was calculated using a paired t-test. *p≤0.05, **p≤0.01, * * *p≤0.001. For FIGS. 22E-1, 22E-2, 22E-3, and 22E-4, A549 cells were treated as in FIGS. 22D-1, 22D-2, 22D-3, 22D-4, 22D-5, and 22D-6. ChIP was performed with antibodies against RNA polymerase II phosphorylated on S5 of the C-terminal domain repeats (pS5(CTD)-Rpb1), and immunoprecipitated DNA was analyzed by qPCR using primers that amplify the TSS associated with the indicated GR target genes. Results are normalized to input chromatin and shown as mean±SEM for three independent experiments. p-value was calculated using a paired t-test. *p≤0.05, **p≤0.01.

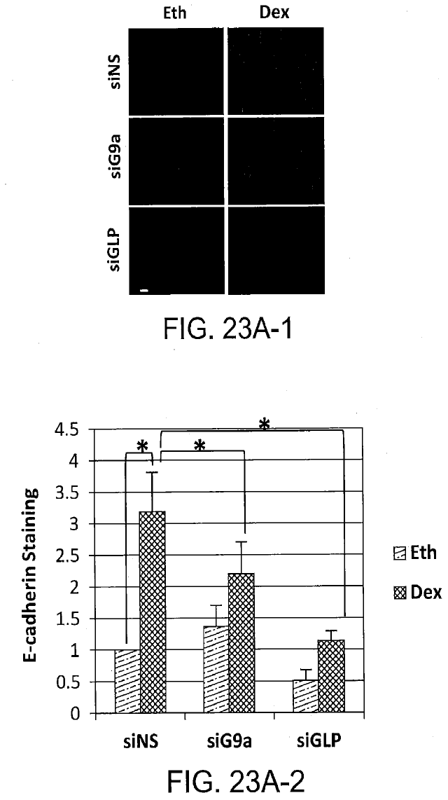

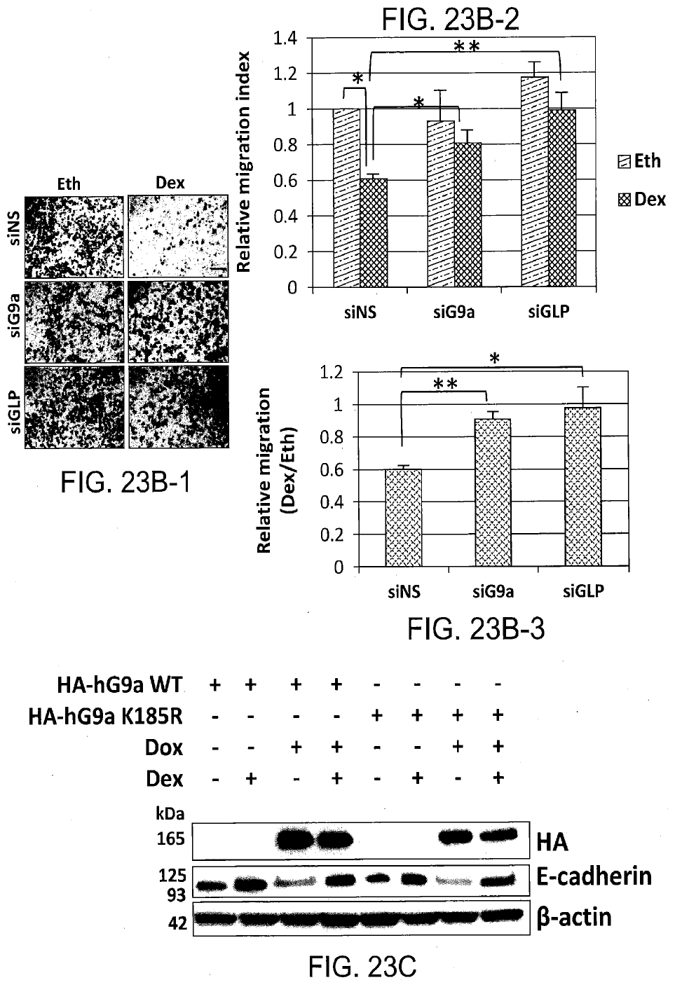

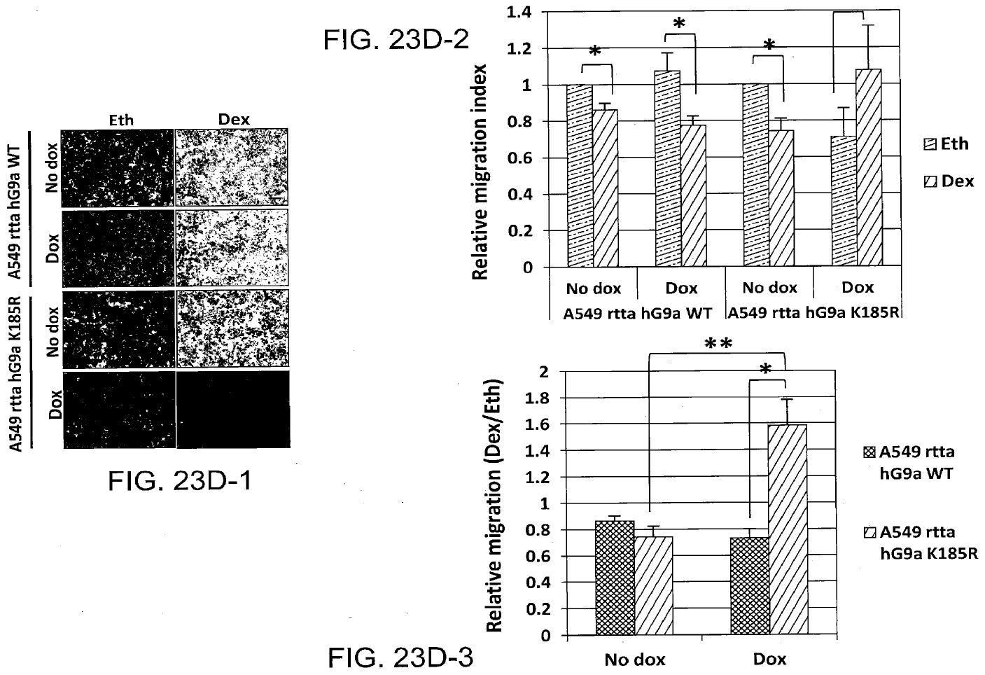

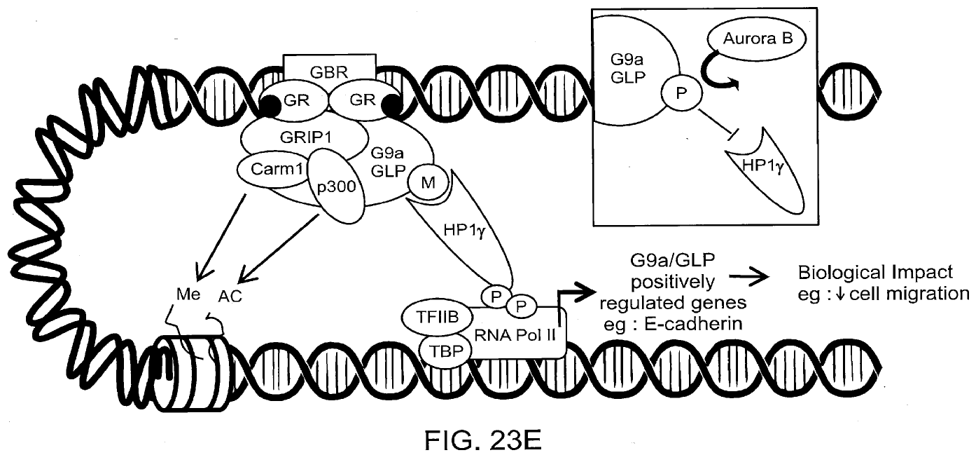

The results as shown in FIGS. 23A-23E generally highlight that G9a and GLP mediate glucocorticoid repression of cell migration. For FIGS. 23A-1 and 23A-2, E-cadherin expression was analyzed by immunofluorescence. A549 cells transfected with non-specific siRNA (siNS) or SMART-pool siRNA targeting G9a (siG9a) or GLP (siGLP) were treated with 100 nM dex or ethanol for 24 h. The nuclei were counterstained with DAPI (blue 17). Representative images are shown. E-cadherin expression (green 18) per cell quantified by image analysis for at least 1500 cells per experiments is shown as the mean±SEM of four independent experiments. p-value was determined using a paired t-test. *p≤0.05. Scale bar represents 10 μm. For FIGS. 23B-1, 23B-2, and 23B-3, A549 cell migration was analyzed using Transwell migration assays for the same cells as described in A. Migratory cells on the bottom of the polycarbonate membrane were stained. Representative images are shown (left panel). Then, dye extracted from the cells was quantified at OD 560 nm. Relative migration index is shown as the mean±SEM of four independent experiments (right top panel). The ratio of migration for cells treated with dex versus ethanol (Eth) from these four experiments is shown on the right bottom panel. p-value was determined using a paired t-test. *p≤0.05, **p≤0.01. Scale bar represents 100 μm. For FIG. 23C, A549 rtta cell lines containing a stably-integrated doxycyline-regulated G9a WT or K185R transgene were treated or not with 10 ng/ml of doxycycline for 24 h prior to and during 24 h of dex treatment. A fraction of the cells was analyzed by immunoblot using indicated antibody. For FIGS. 23D-1, 23D-2, and 23D-3, using the same cells described in C, cell migration was assessed using Transwell migration assays as described in B. Analyses are shown as the mean±SEM of four independent experiments. p-value was determined using a paired t-test. *p≤0.05, **p≤0.01. Scale bar represents 100 μm. FIG. 23E shows a model for transcriptional regulation of G9a/GLP-dependent GR target genes by G9a and GLP PTMs. After stimulation with hormone (filled black circle), GR binds to GR binding regions (GBR) on DNA and recruits G9a and GLP. G9a facilitates recruitment of p300 and Carm1 coactivators, which acetylate histones H3 and H4 (Ac) and methylate histone H3 at R17 (Me) respectively. If G9a and GLP are methylated they recruit phospho-S93-HP1γ, which facilitates recruitment of RNA polymerase II (PolII), which is phosphorylated (P) on S5 of the C-terminal domain repeats to activate G9a/GLP-dependent GR target genes. Dex-induced, G9a/GLP-dependent GR target genes include CDH1 (encoding E-cadherin) which is important for the decreased cell migration caused by dex. However, if G9a or GLP are phosphorylated by Aurora kinase B, HP1γ recruitment by G9a or GLP is prevented, thereby inhibiting dex-induced expression of the G9a/GLP-dependent GR target genes. Dex-induced, G9a/GLP-dependent GR target genes include CDH1 (encoding E-cadherin) which is important for the decreased cell migration caused by dex.

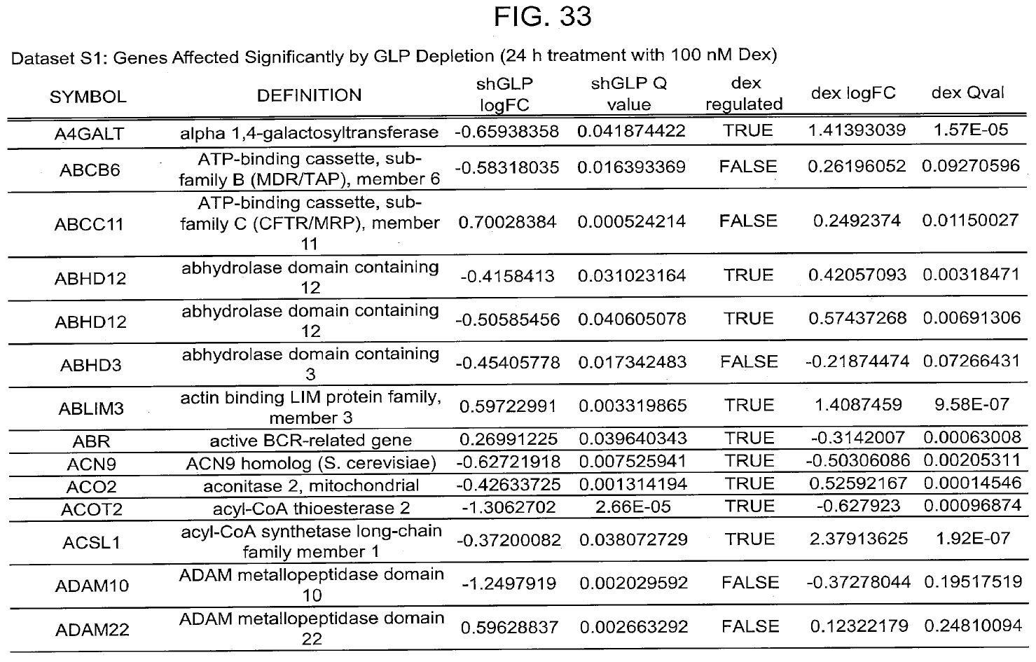

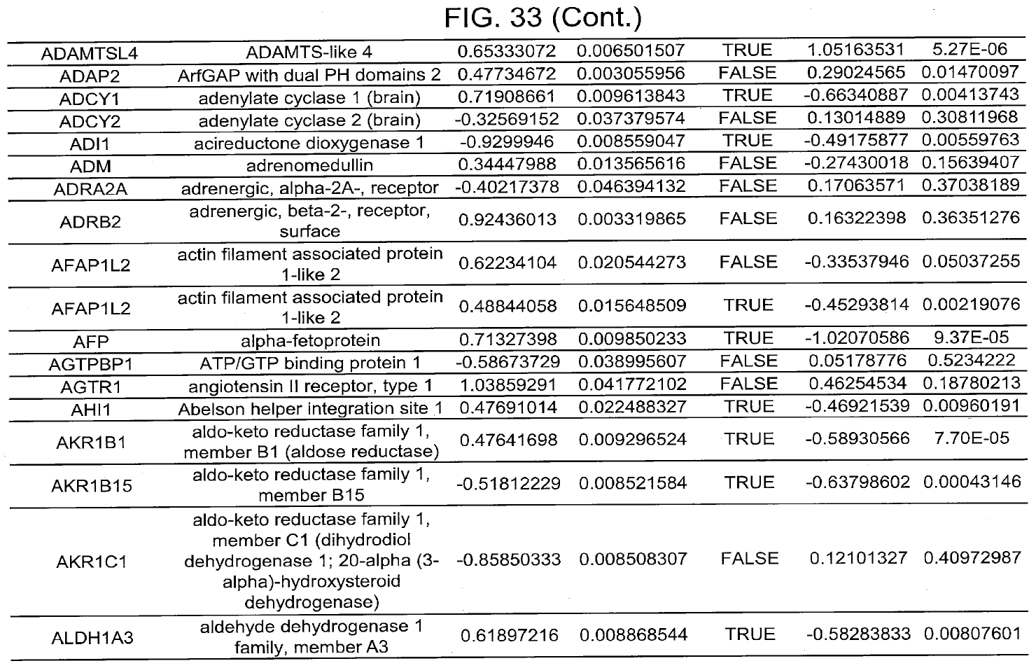

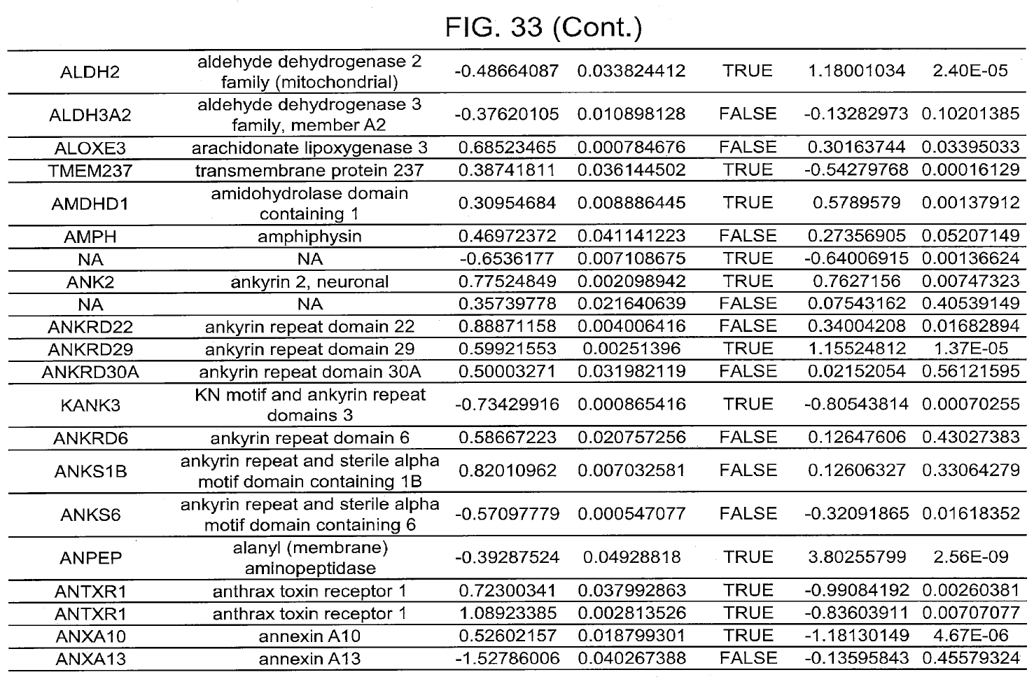

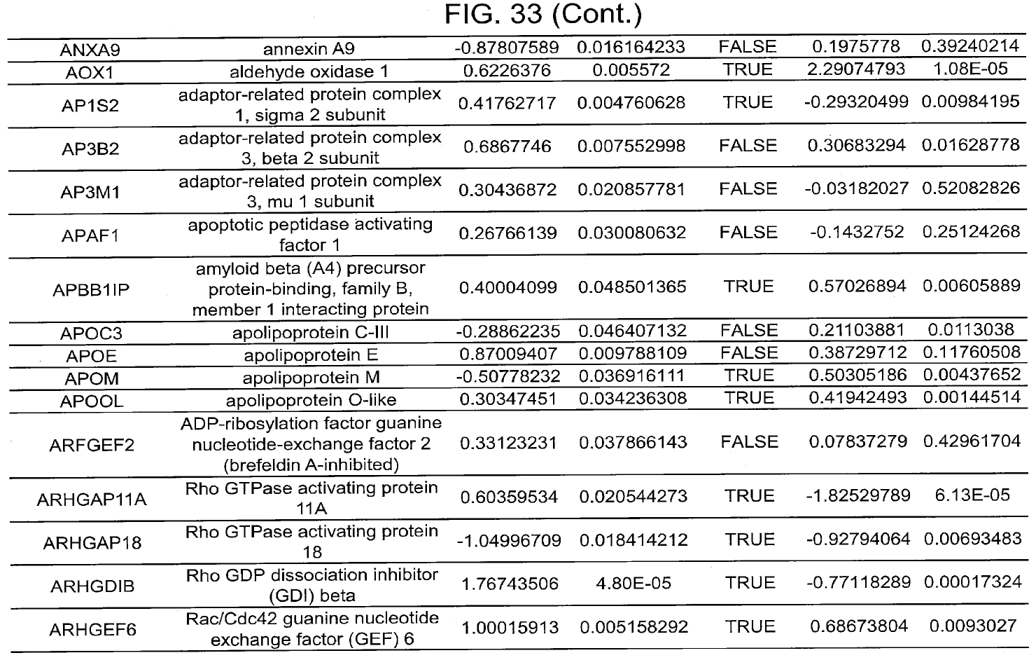

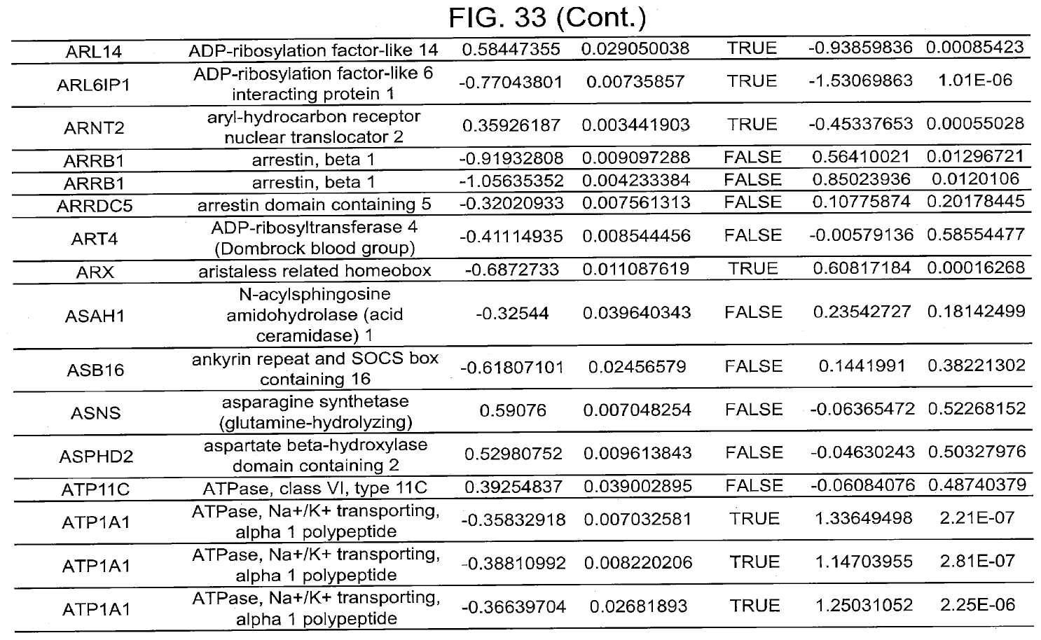

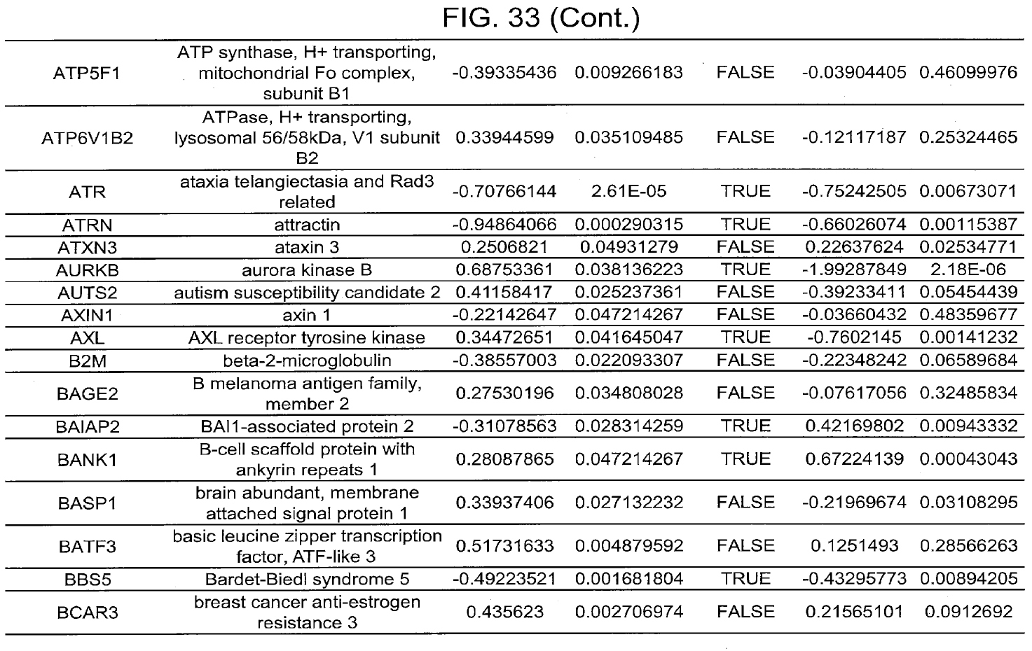

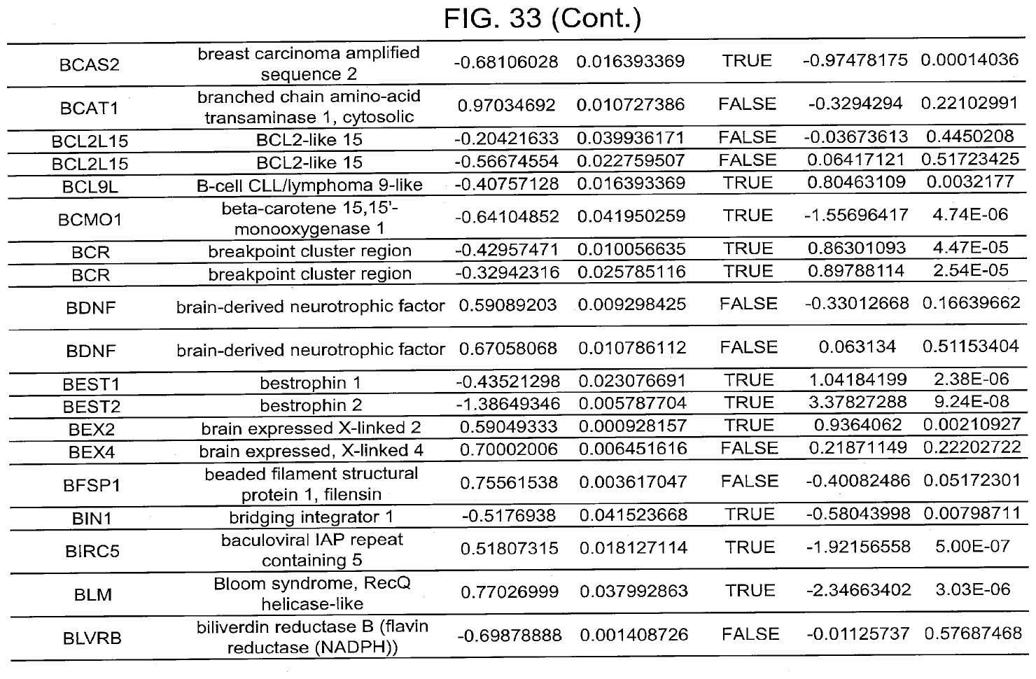

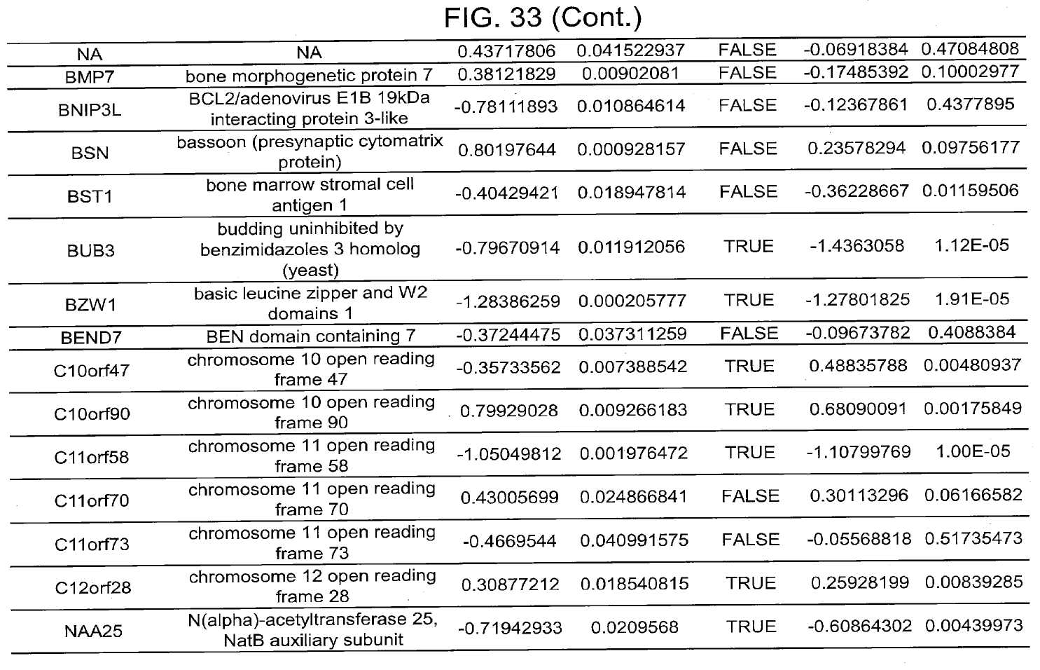

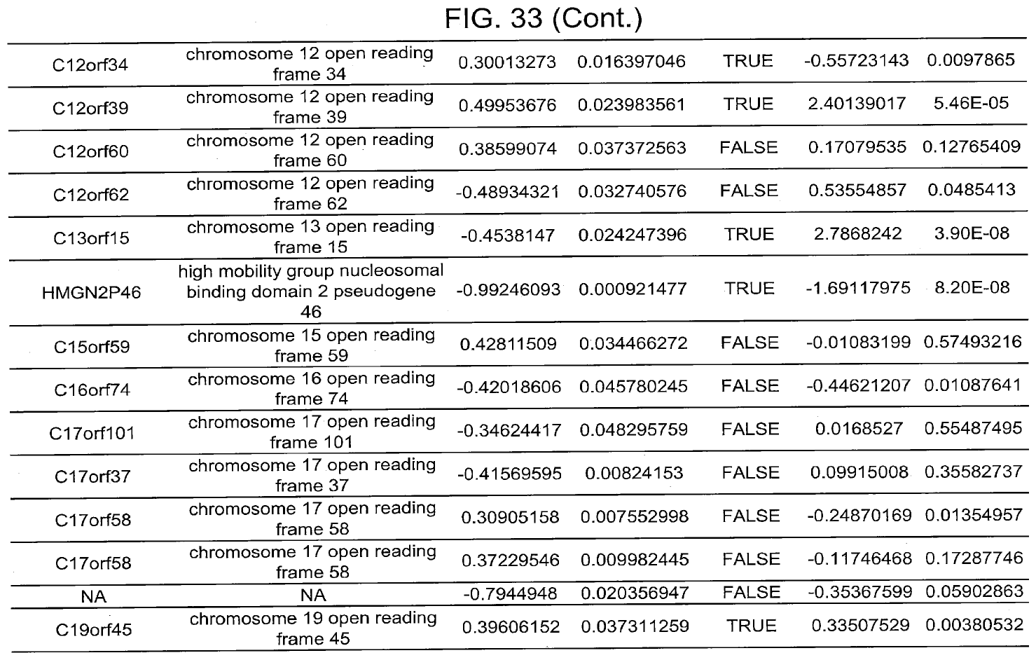

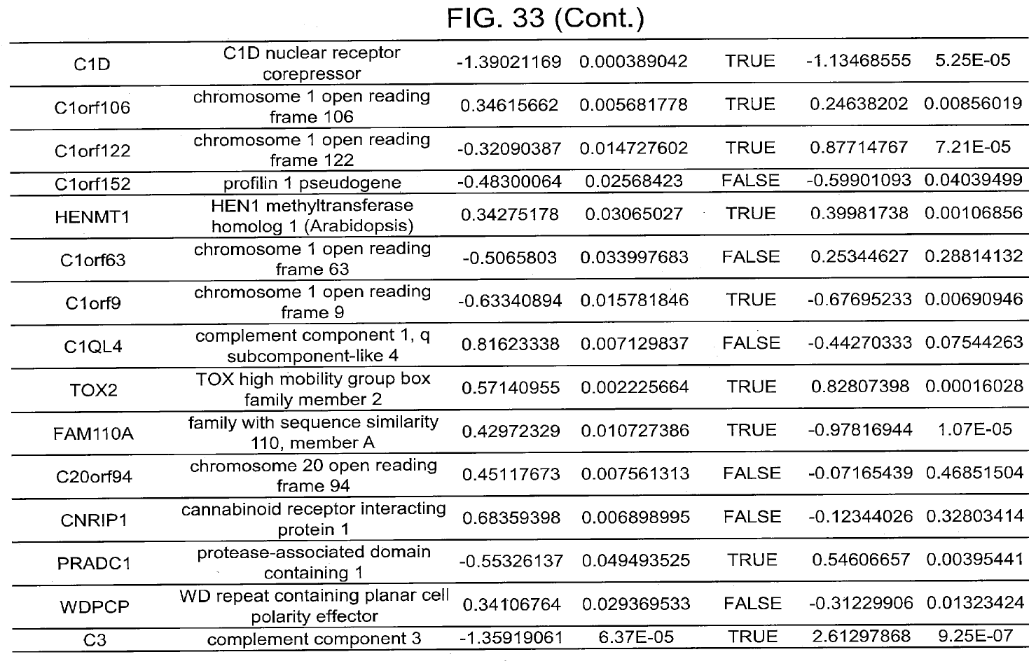

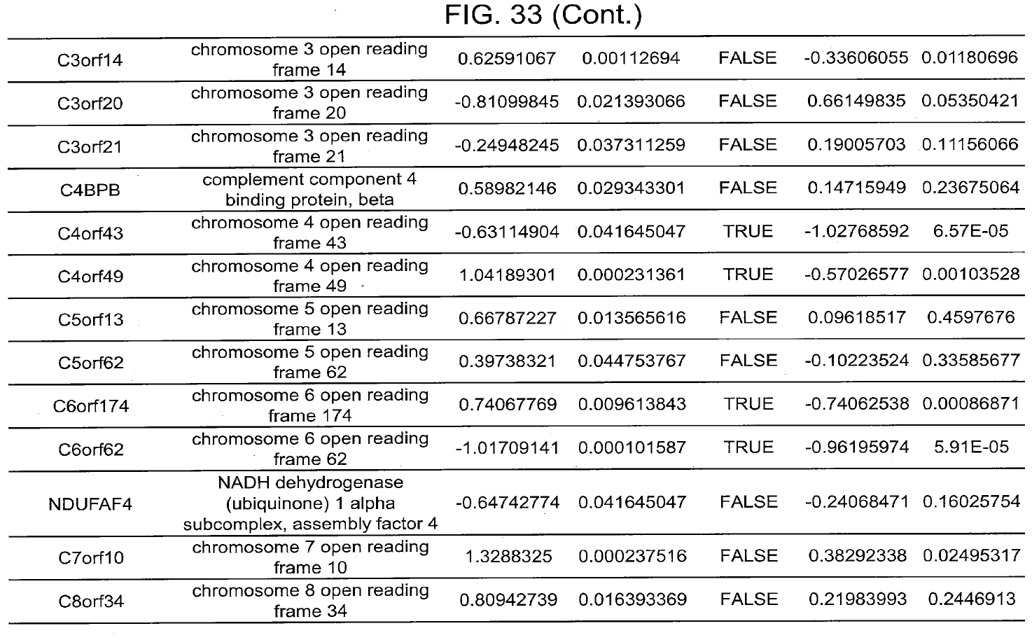

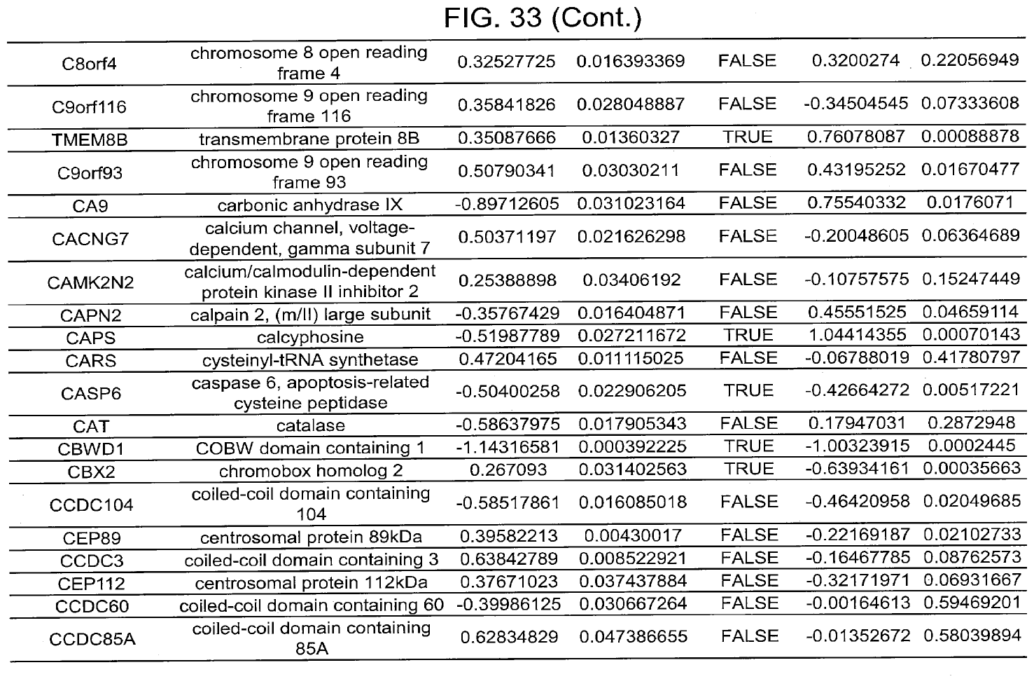

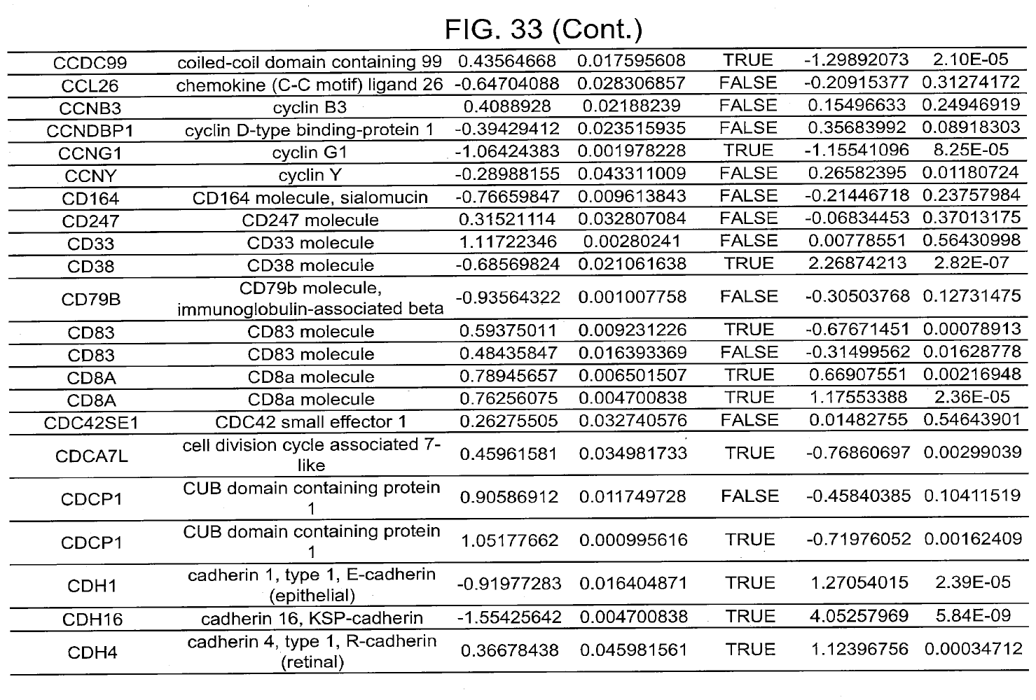

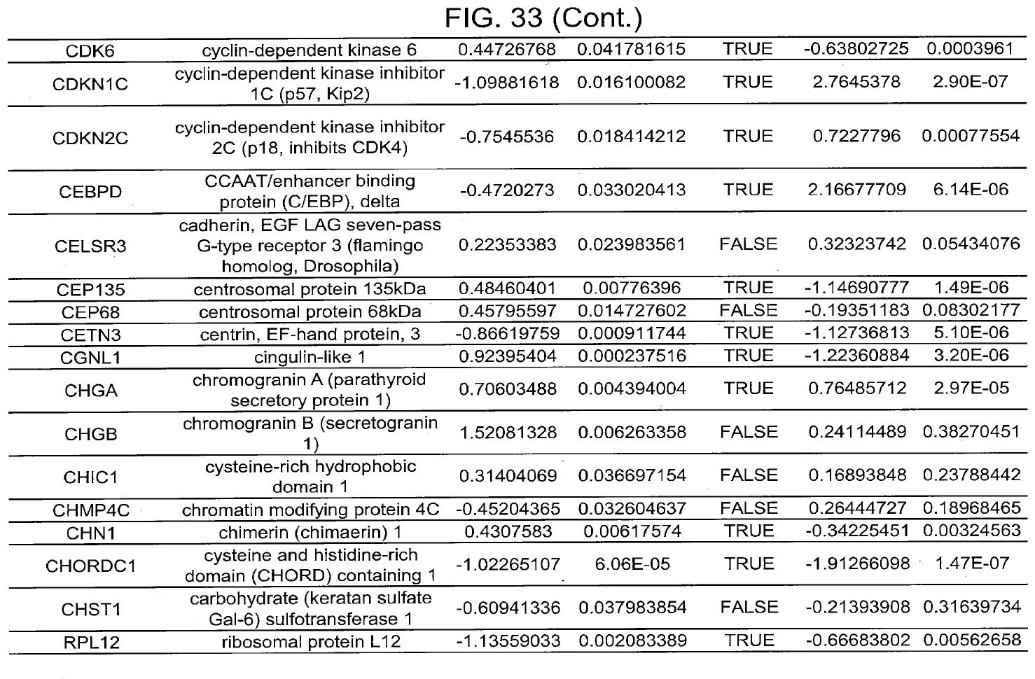

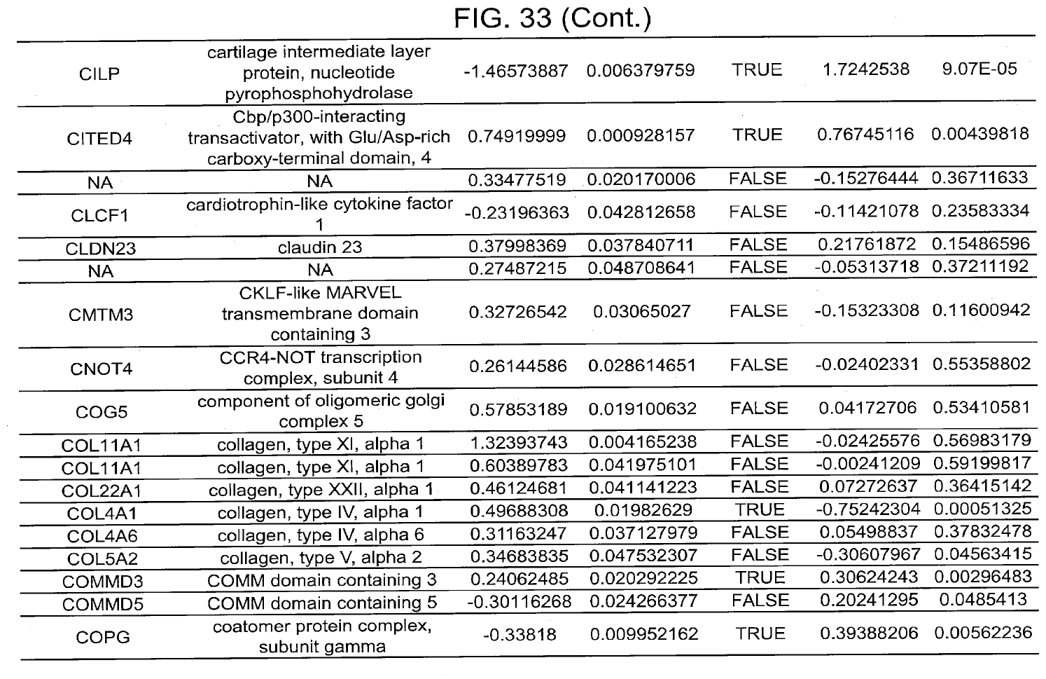

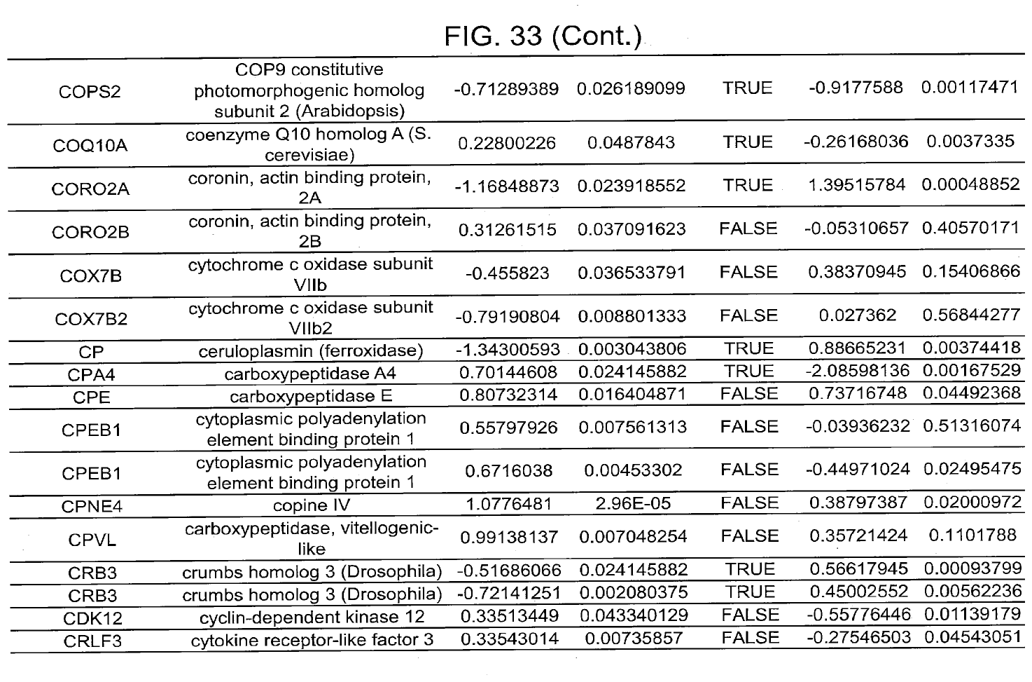

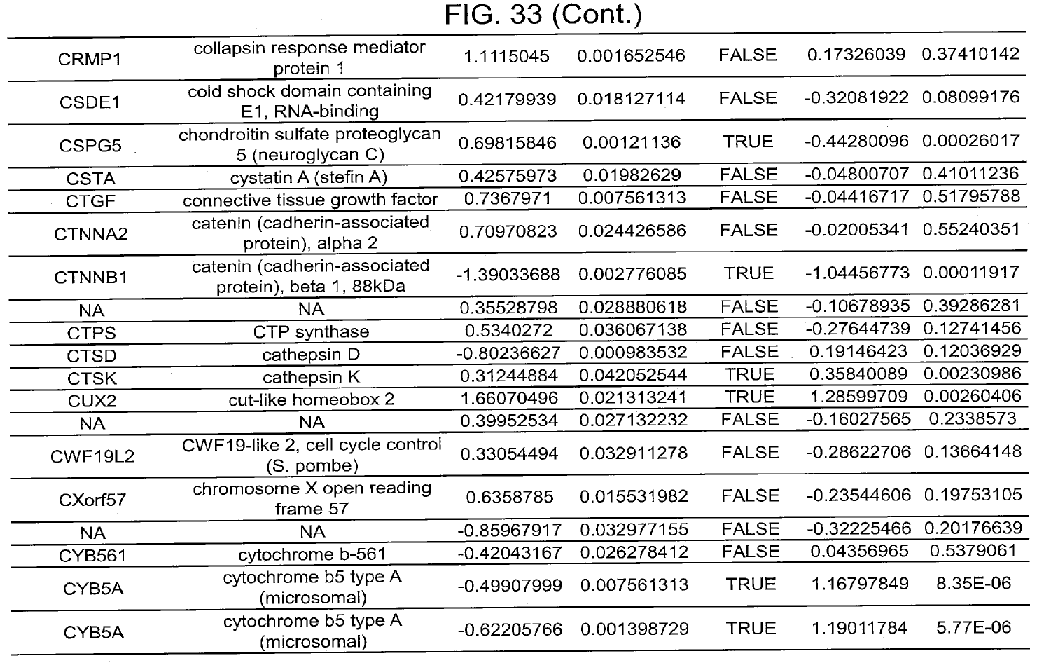

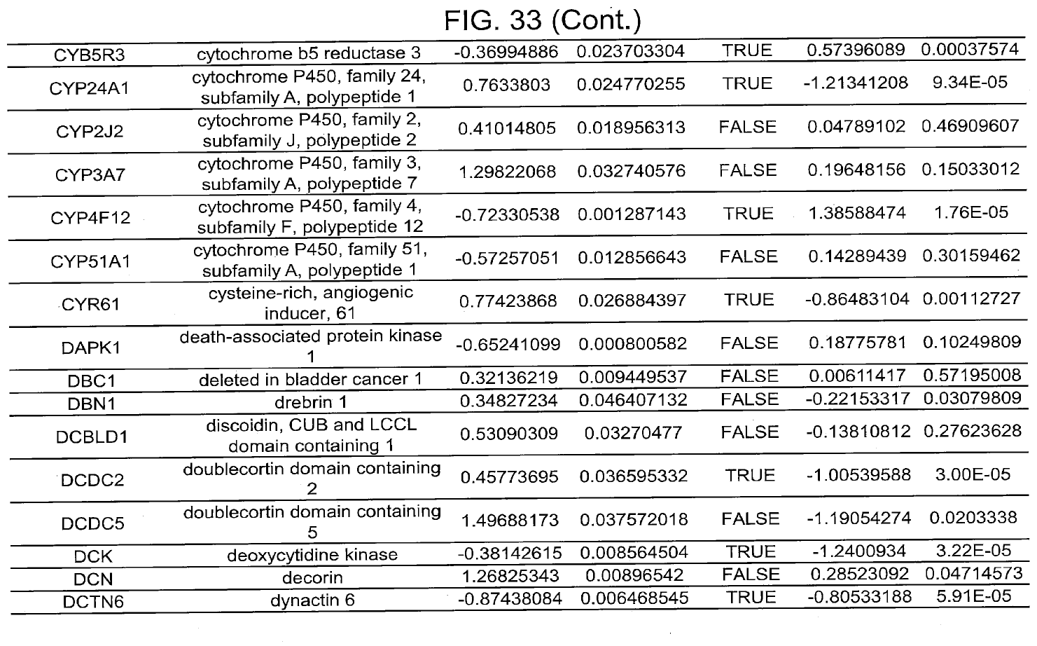

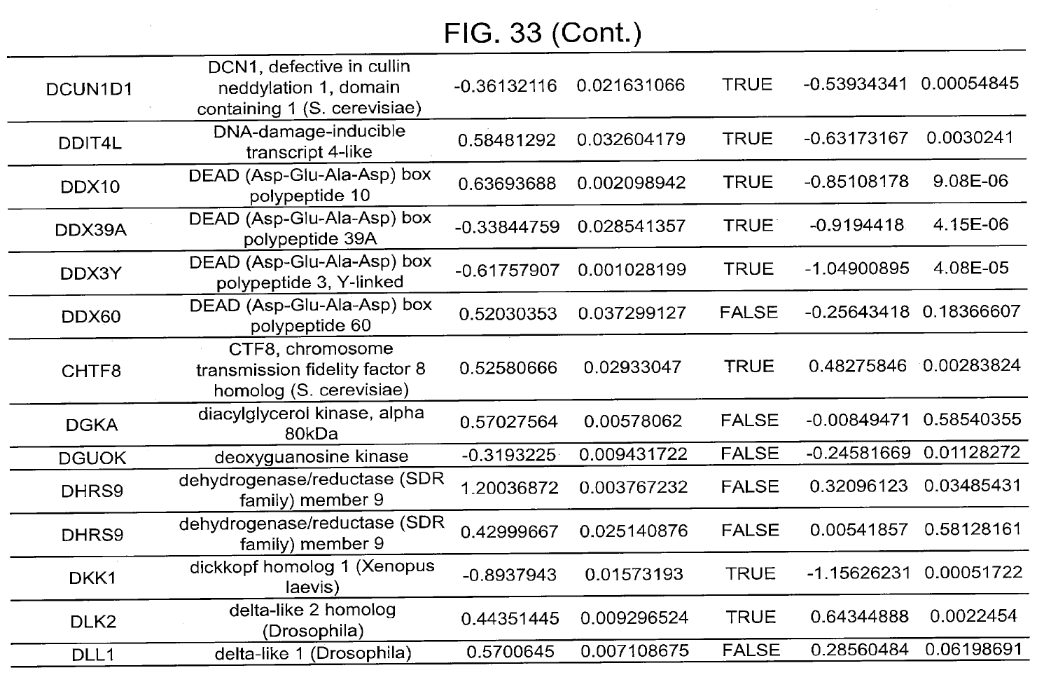

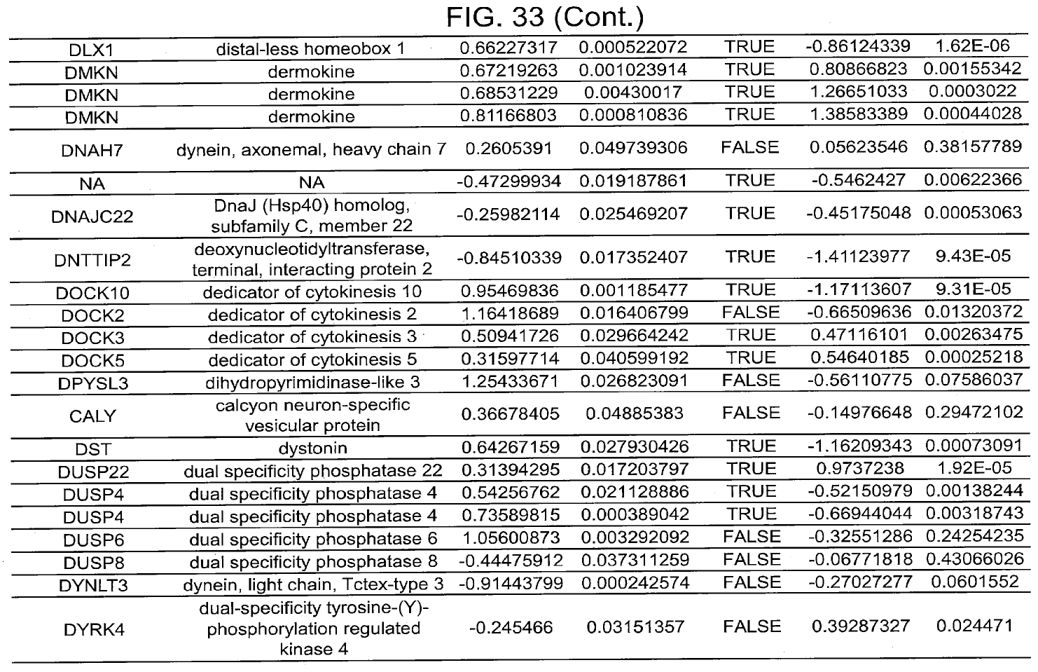

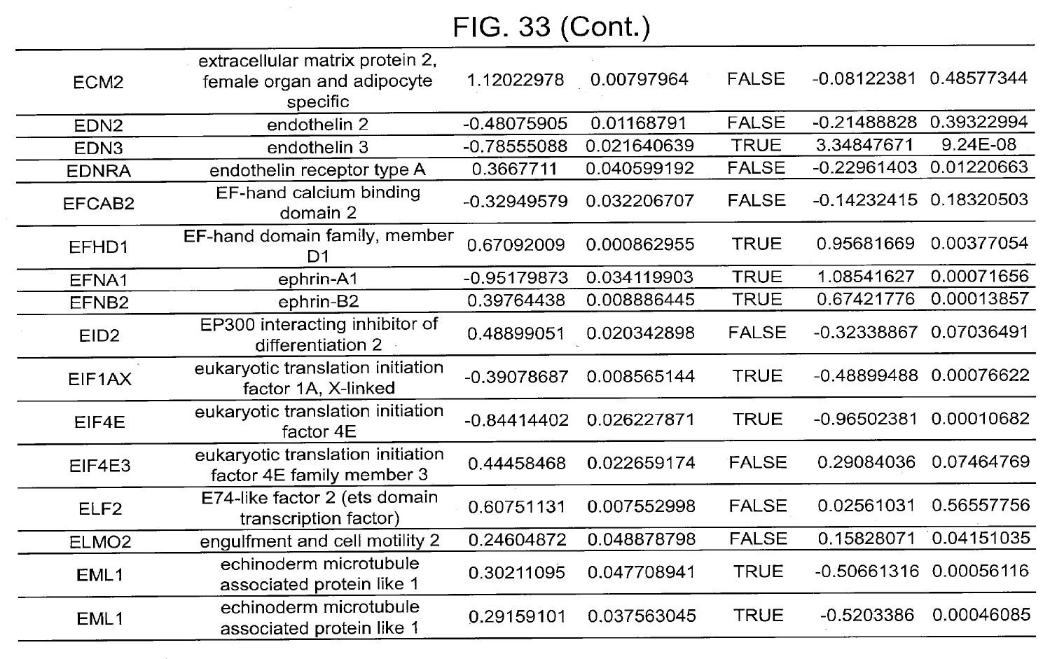

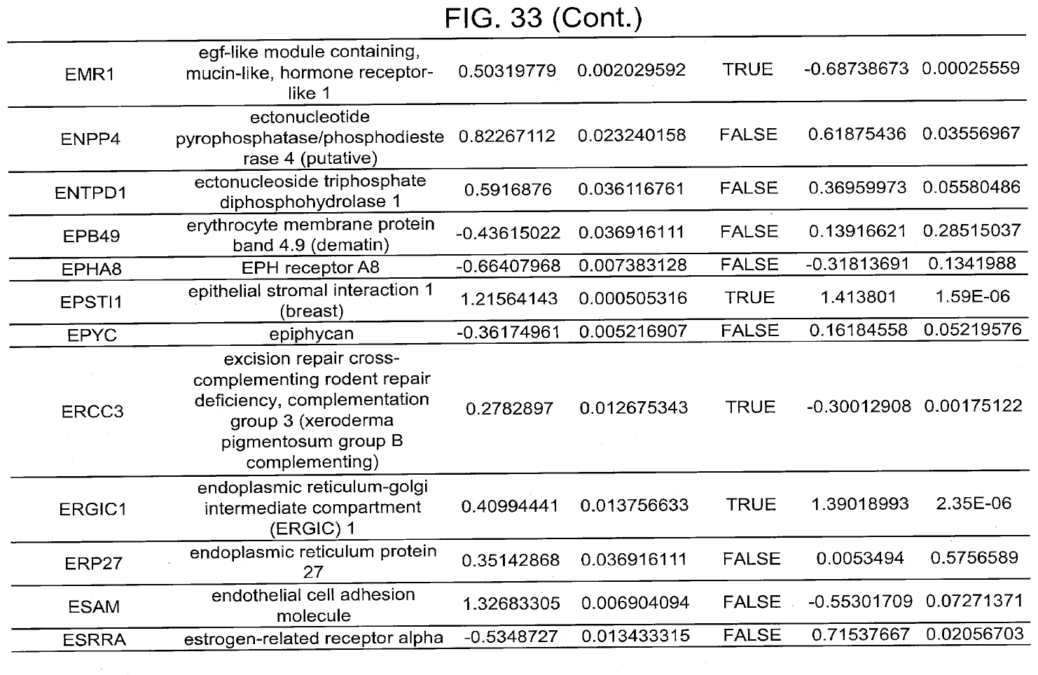

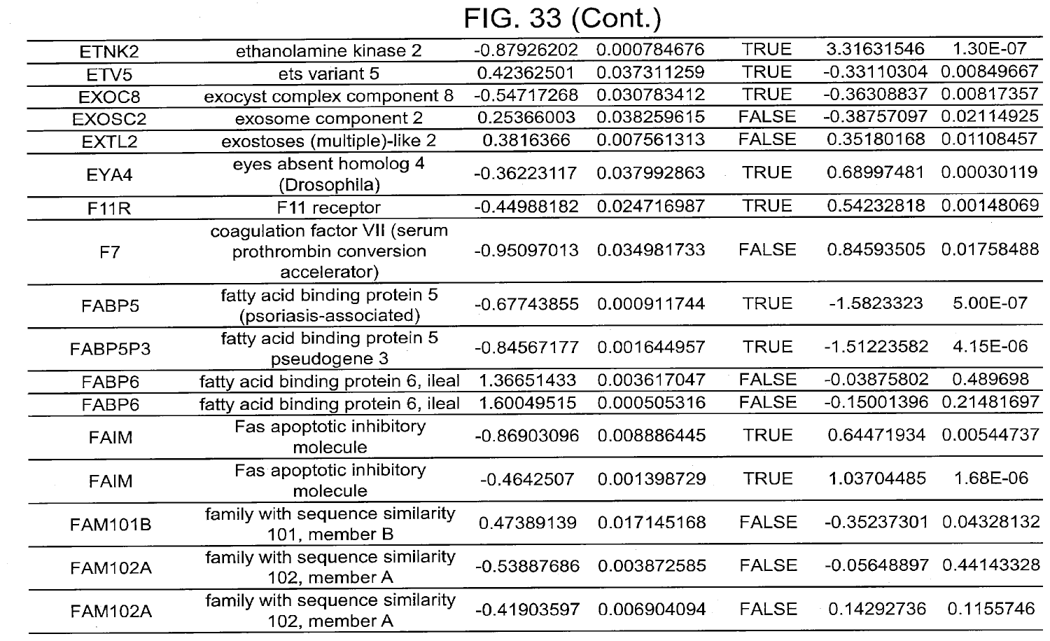

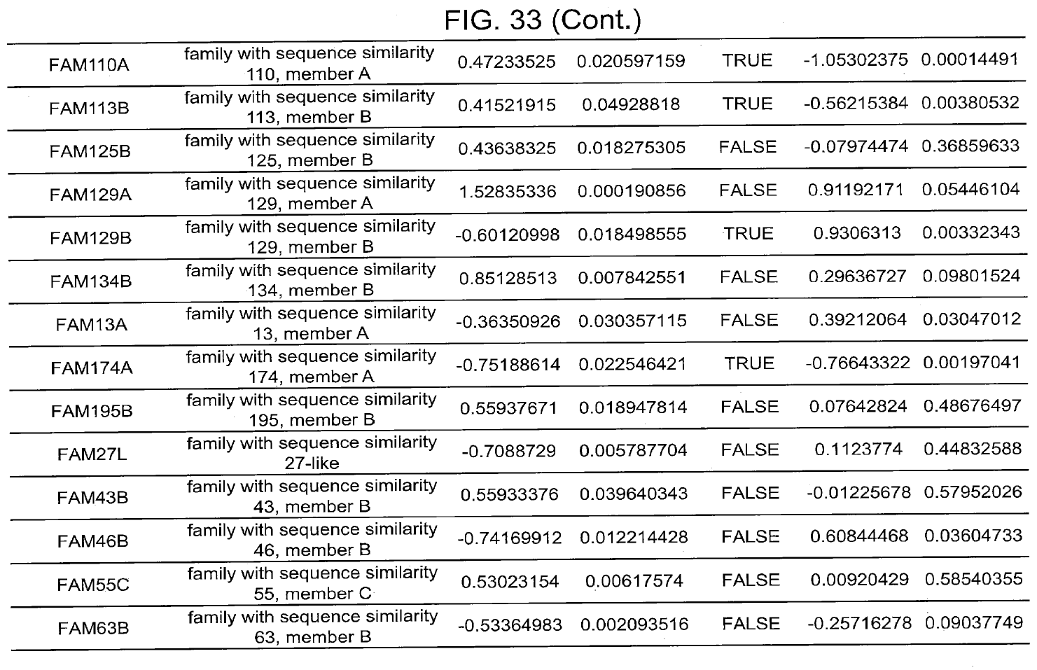

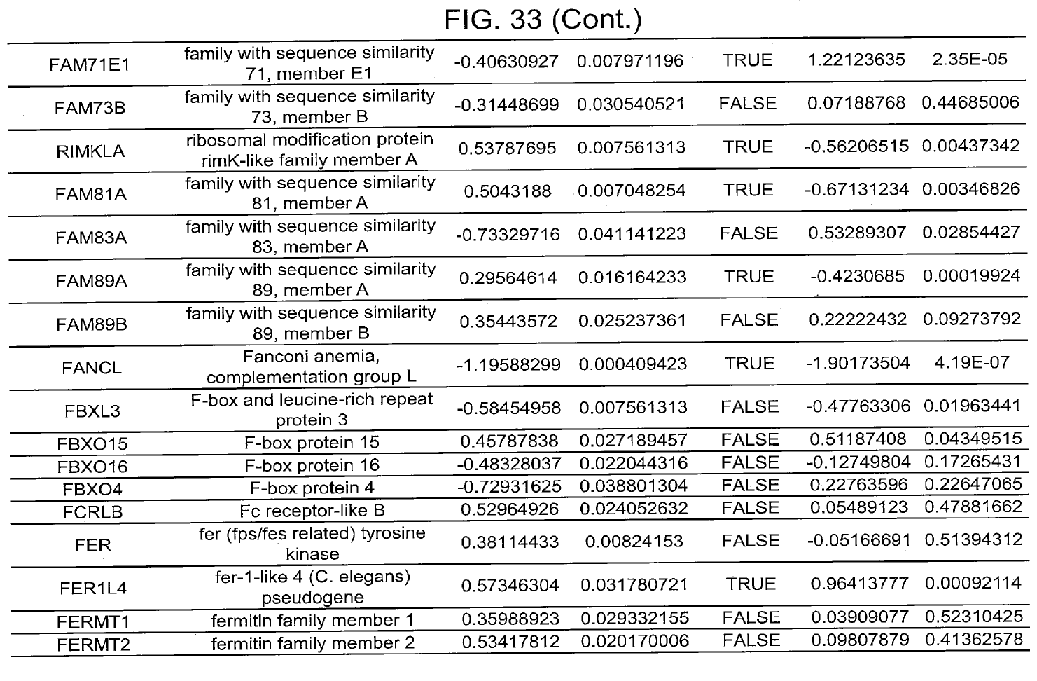

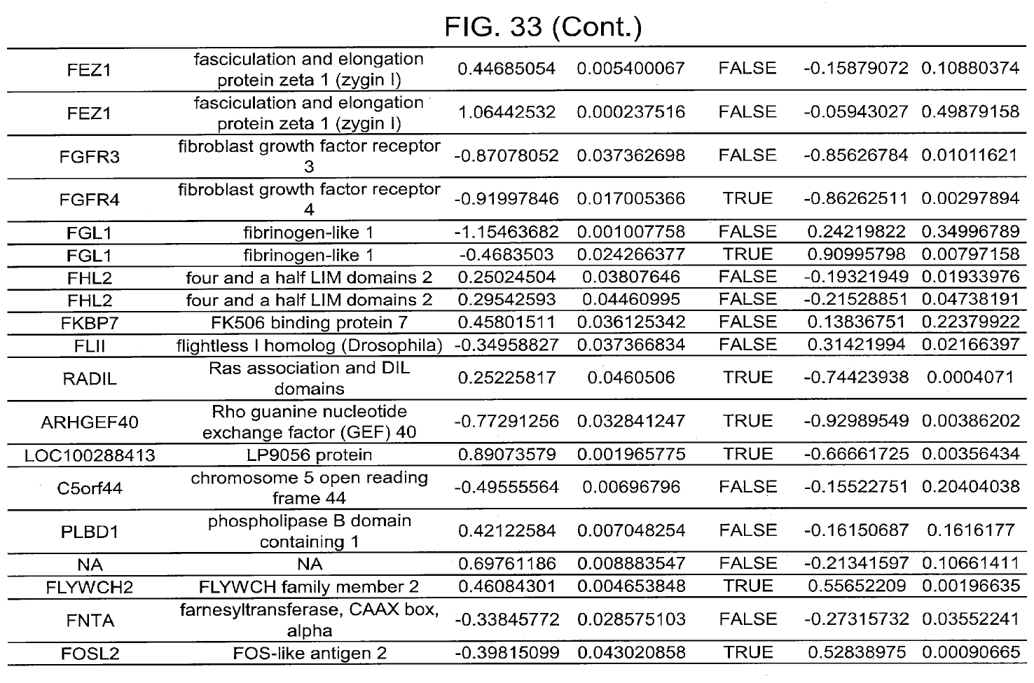

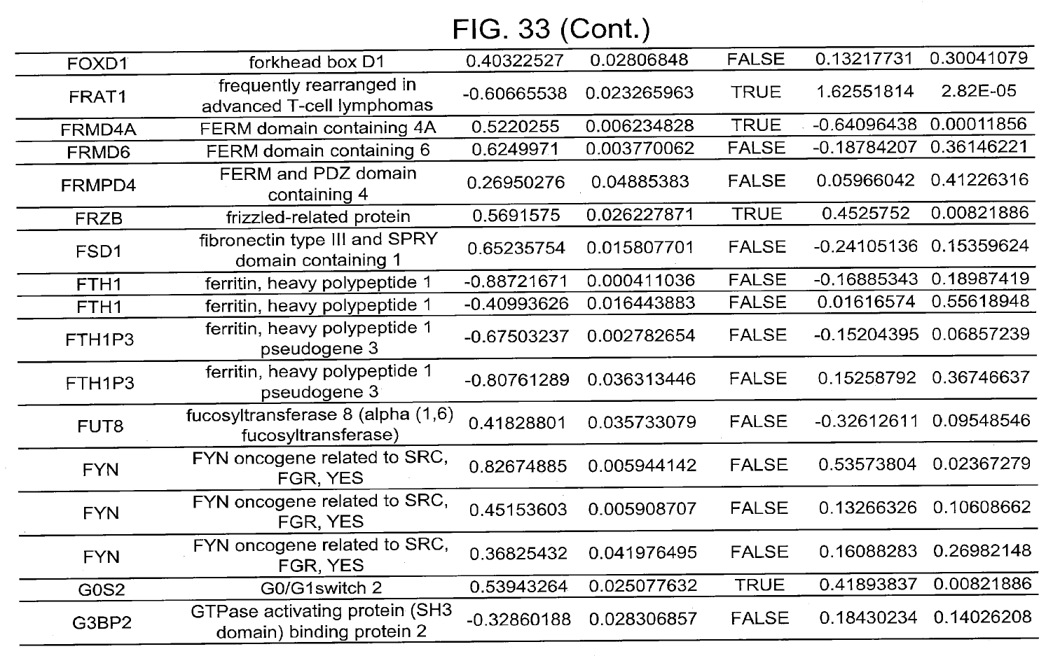

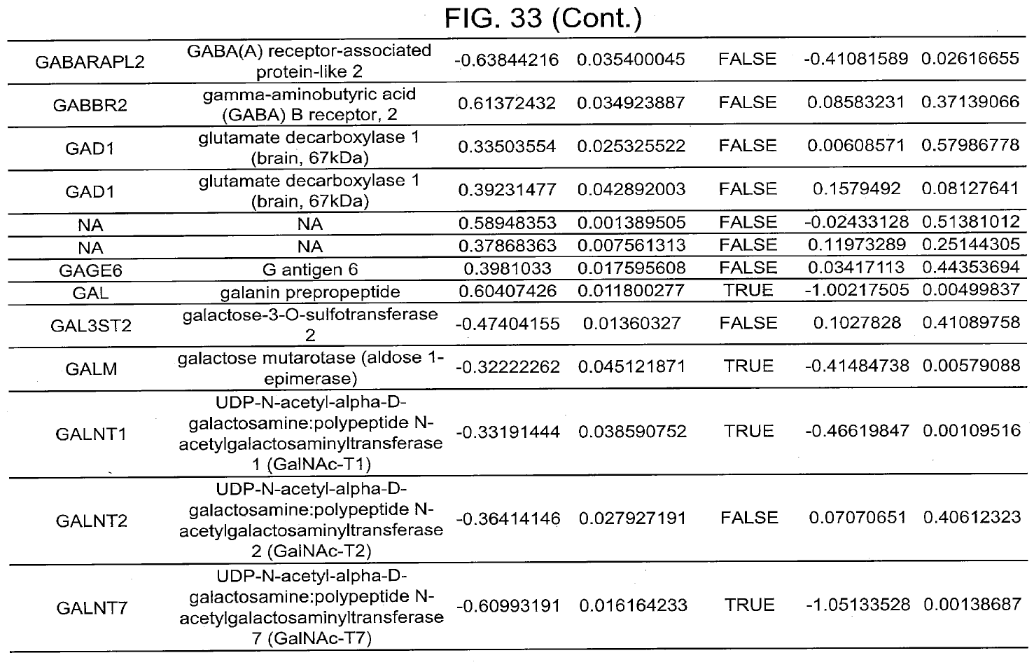

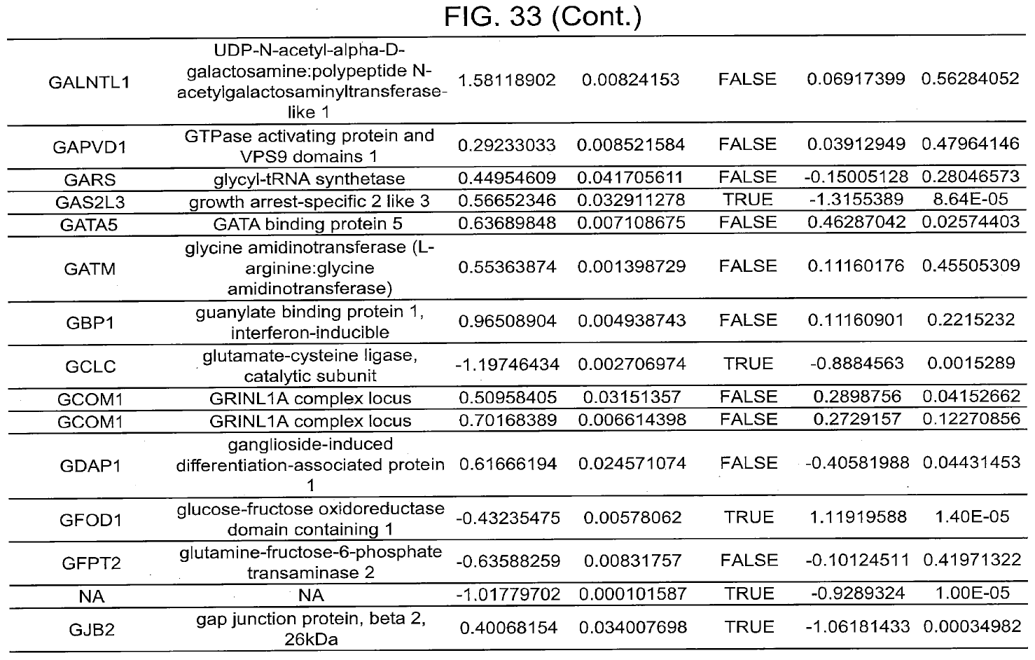

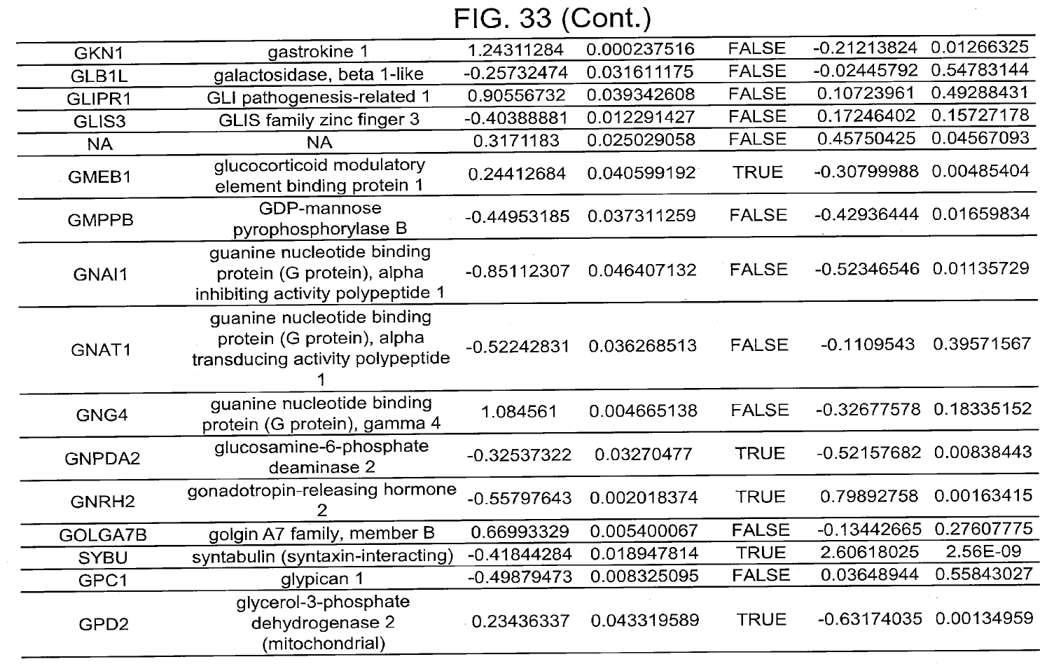

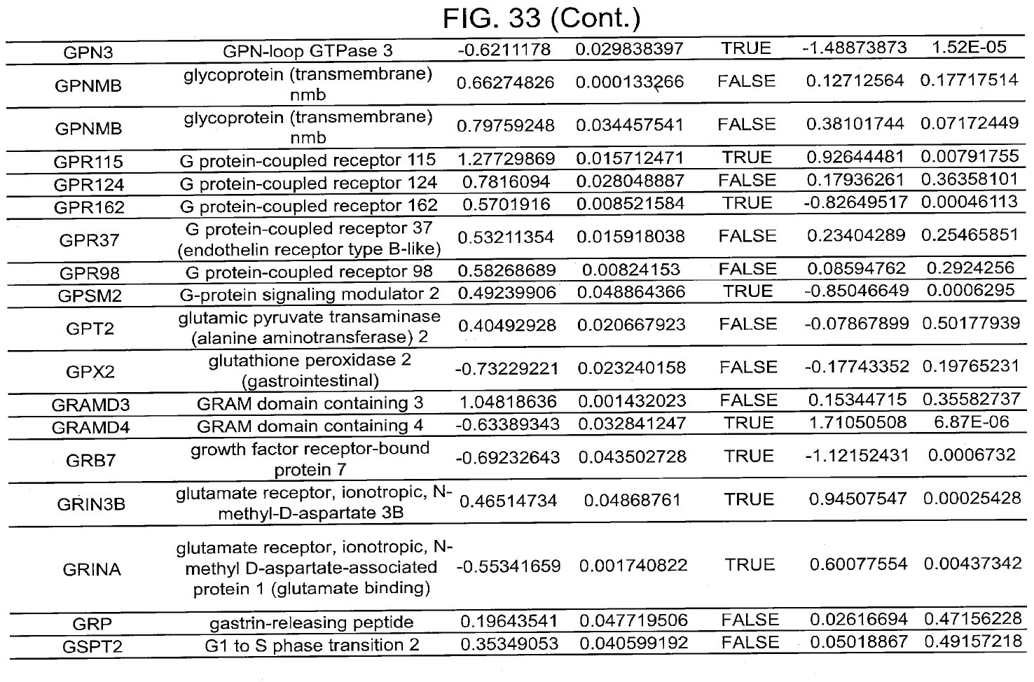

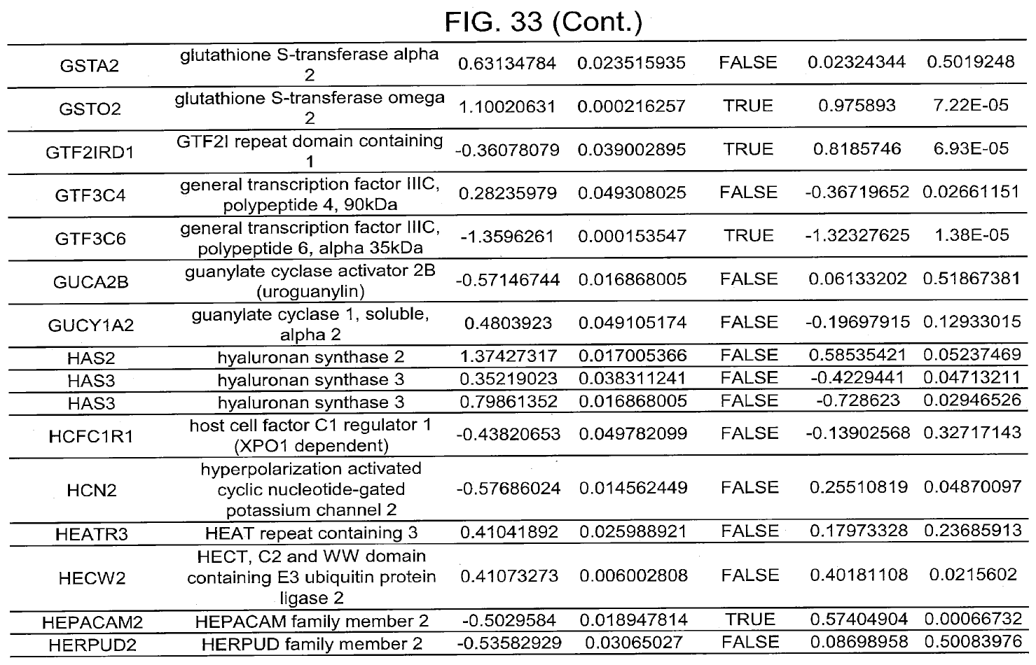

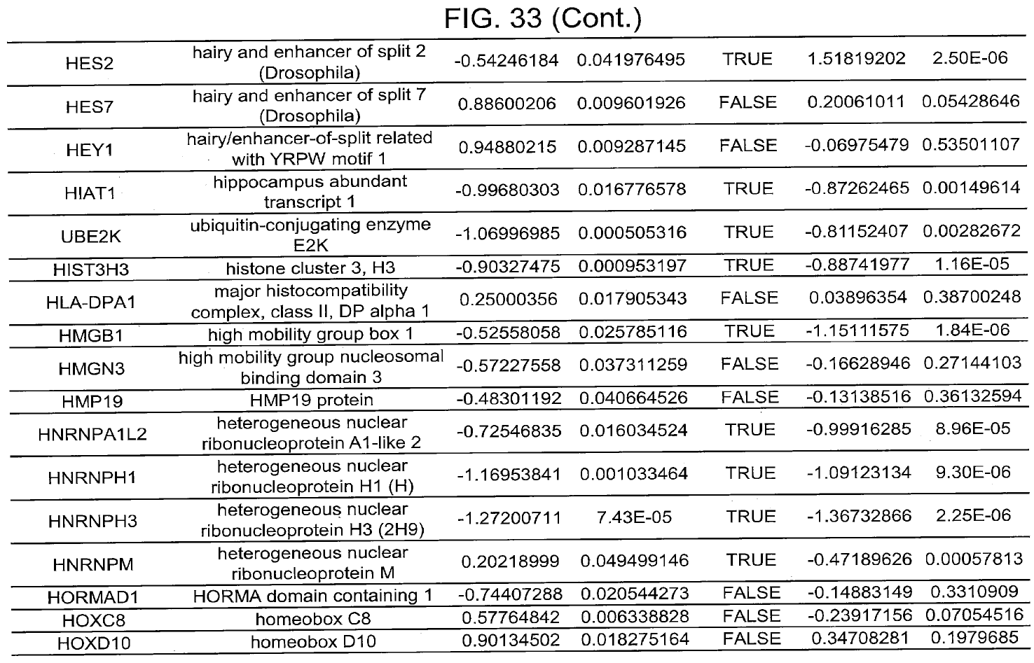

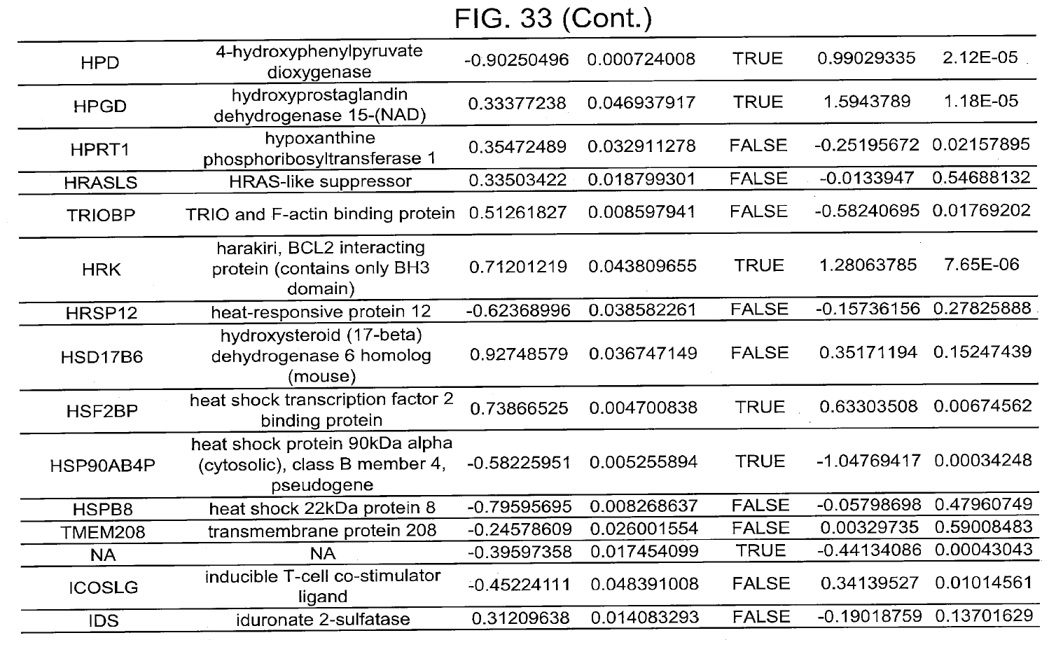

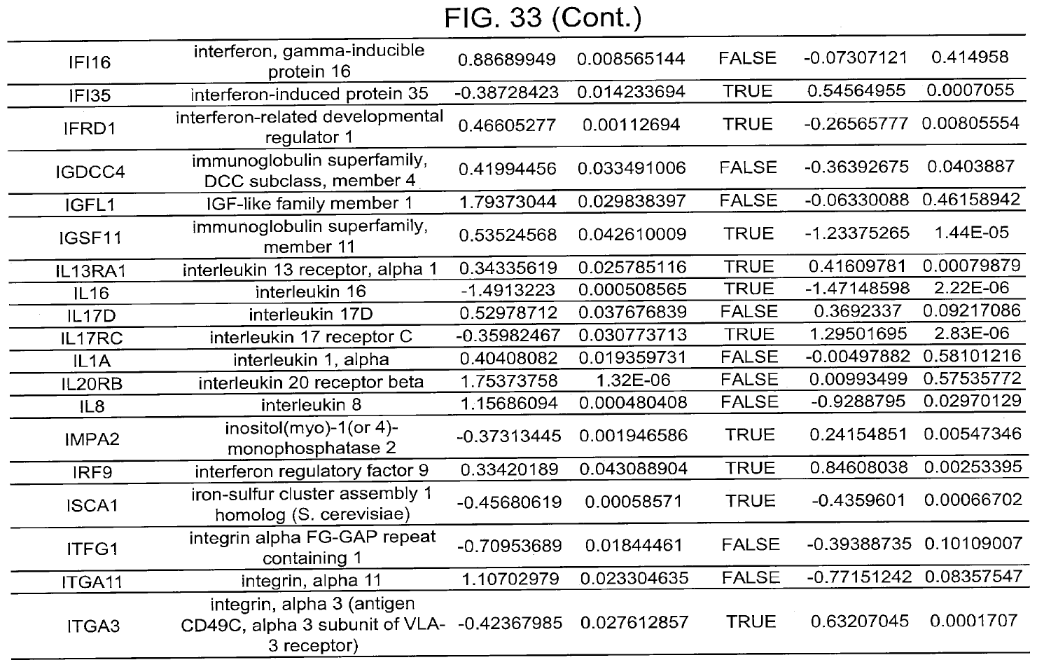

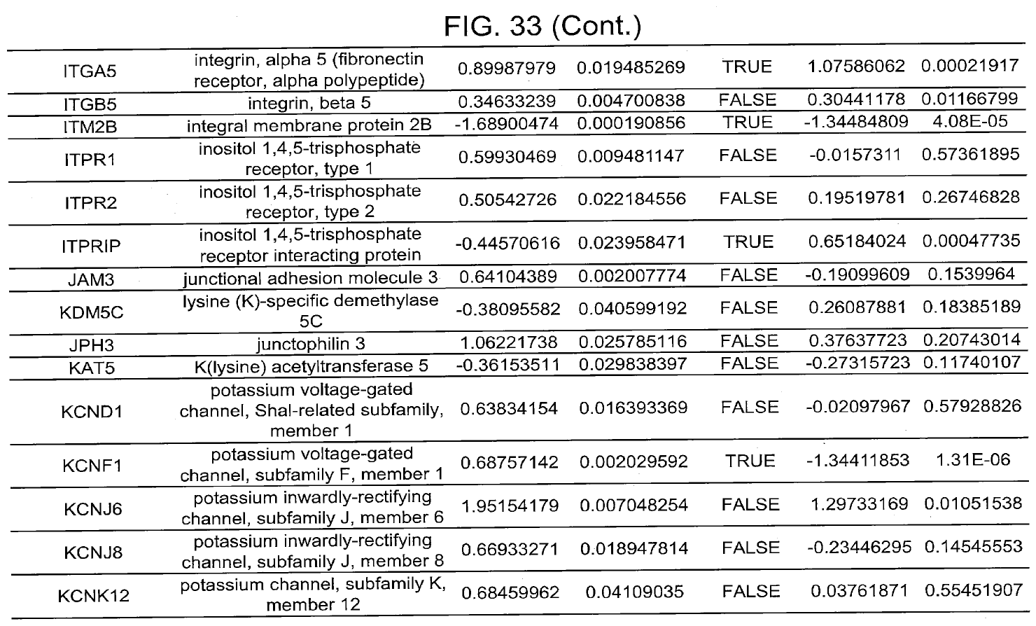

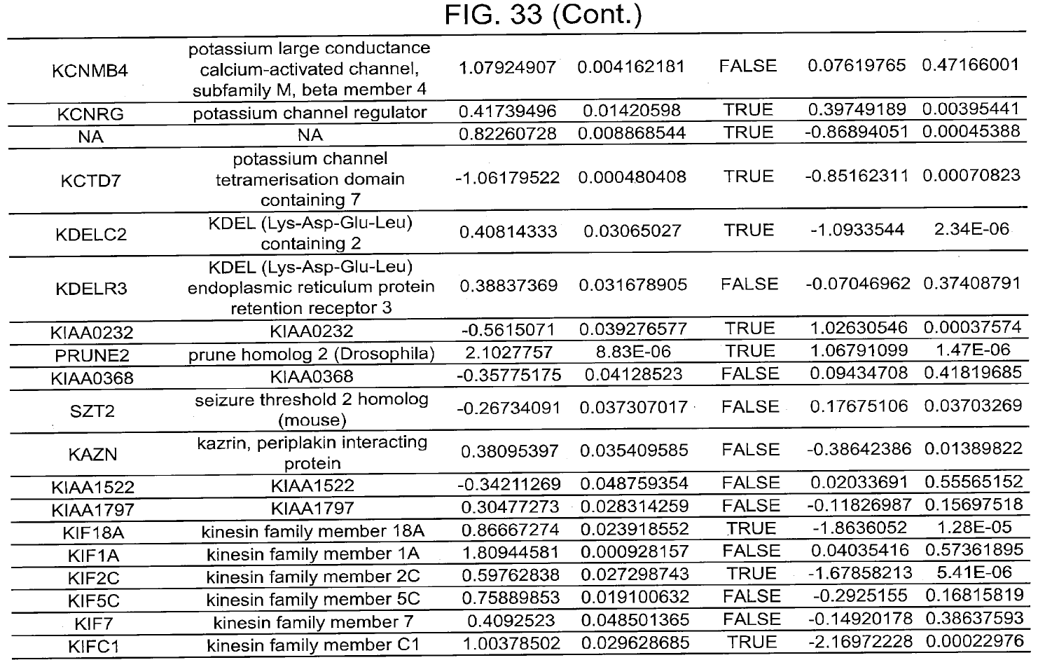

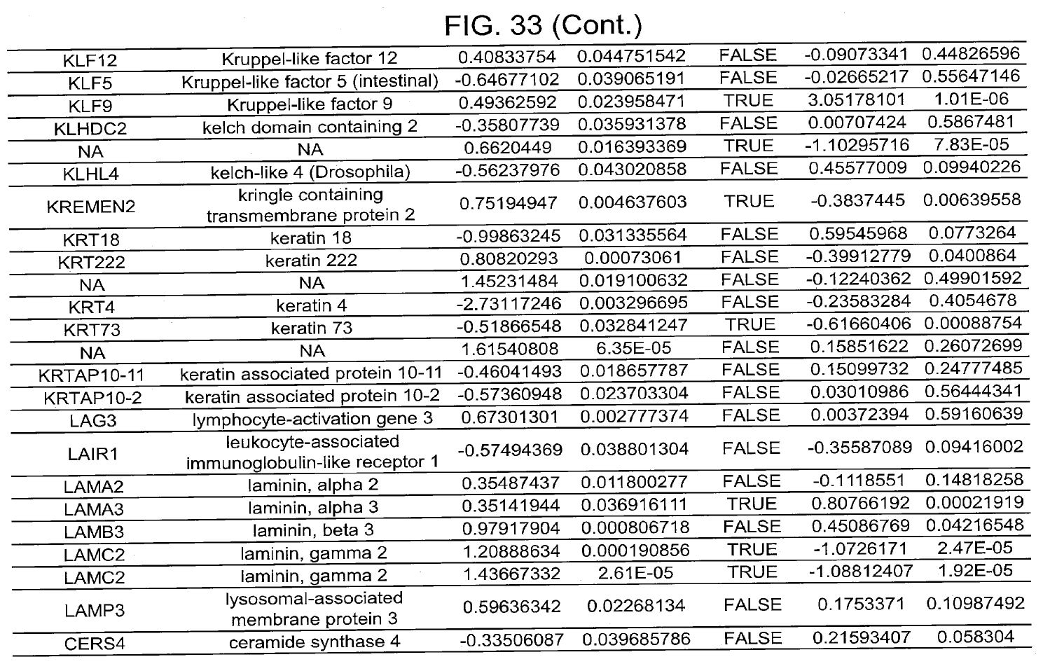

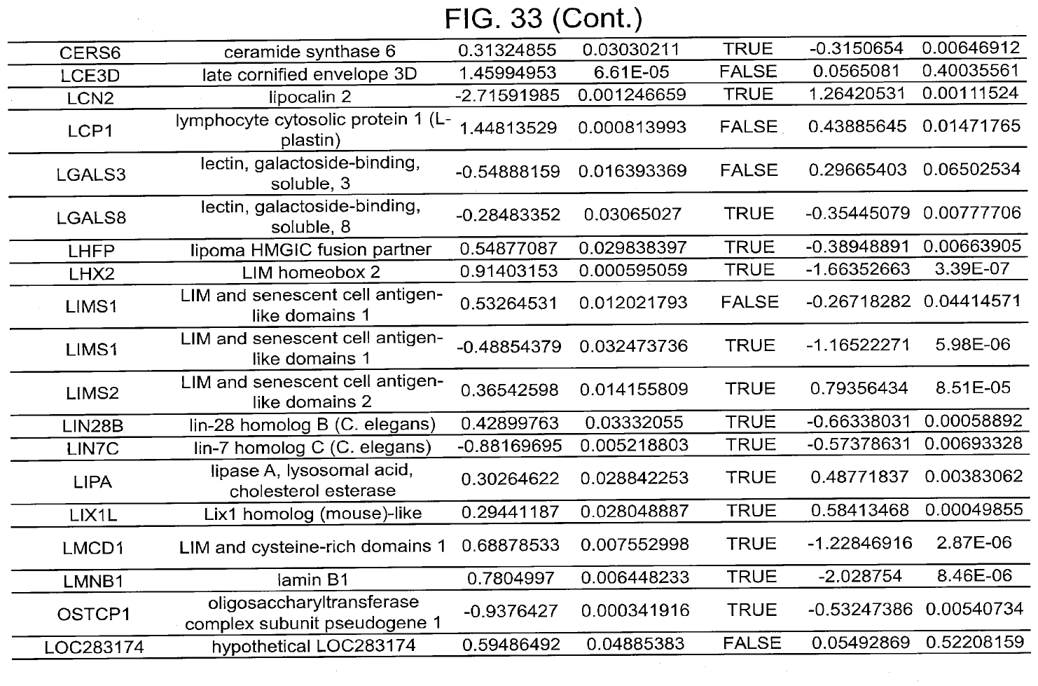

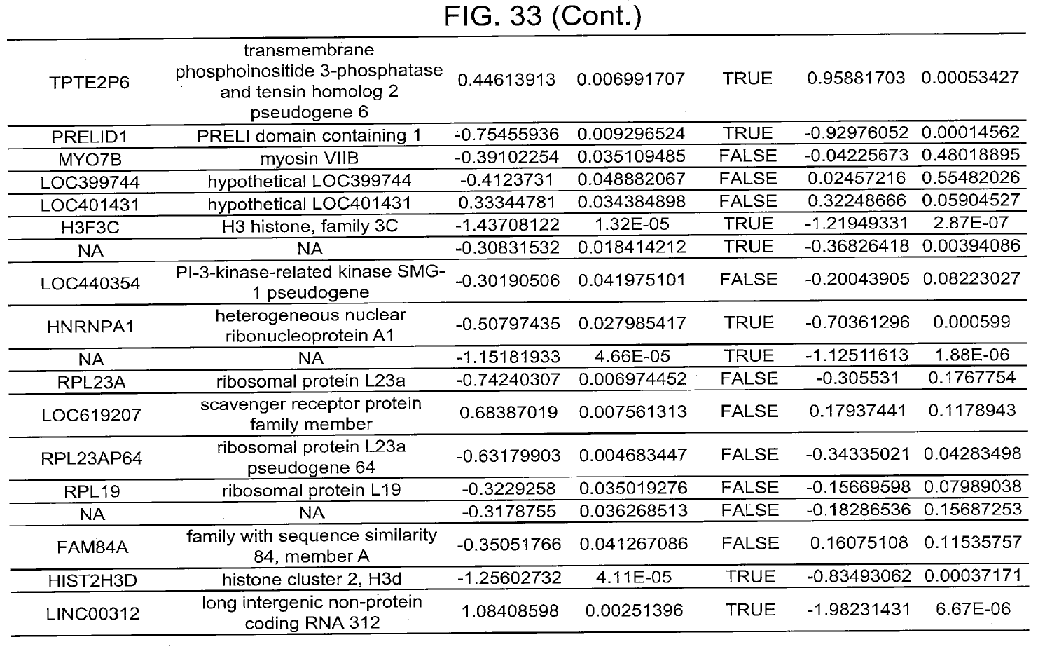

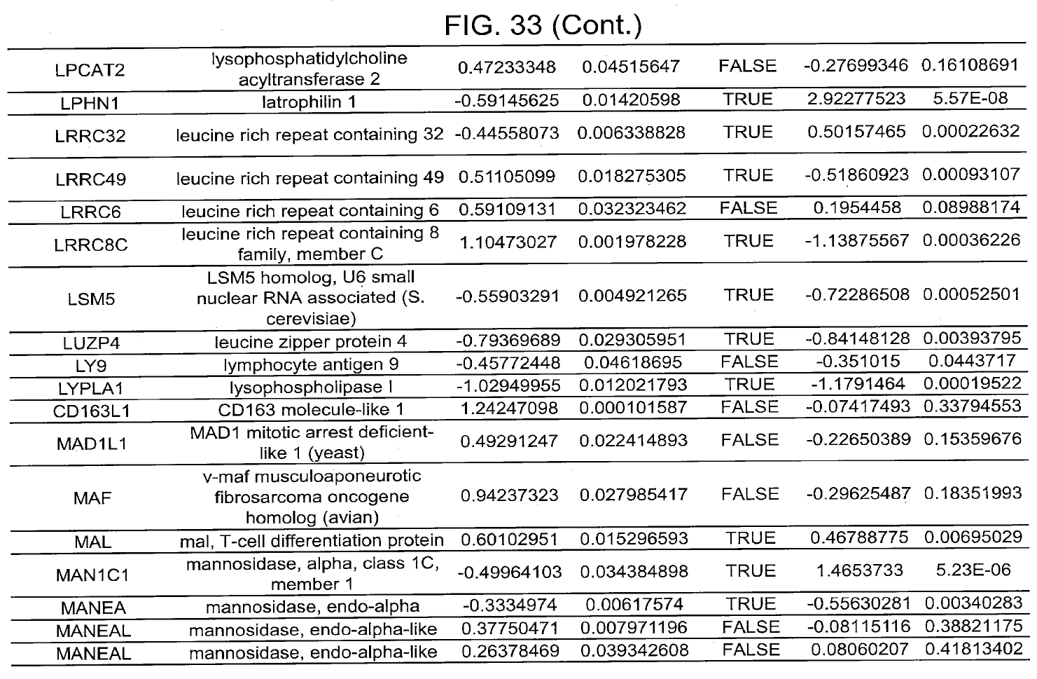

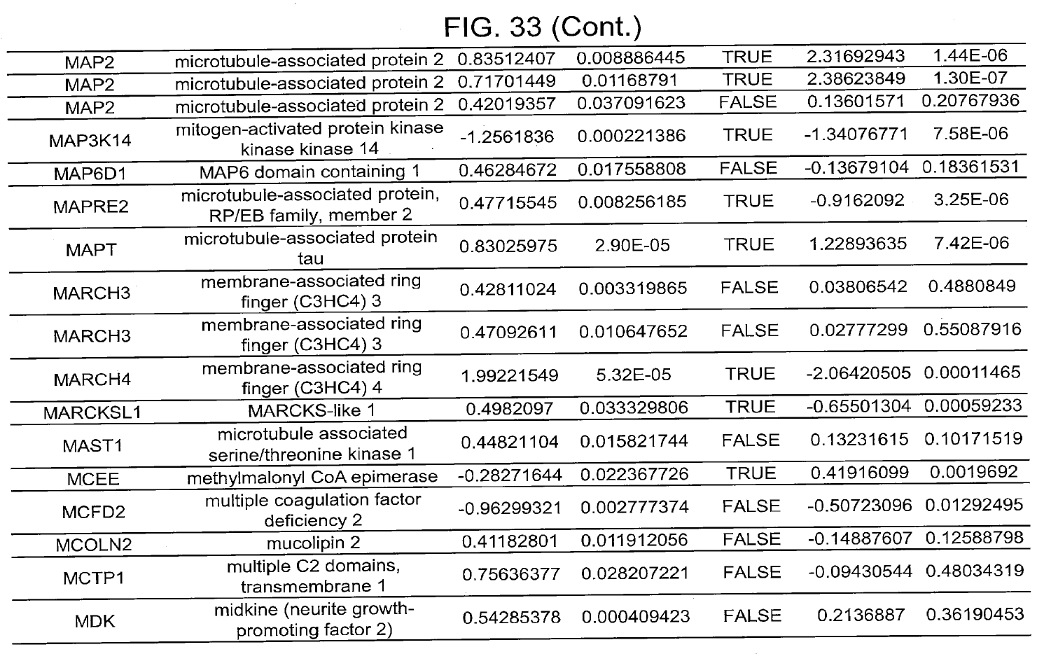

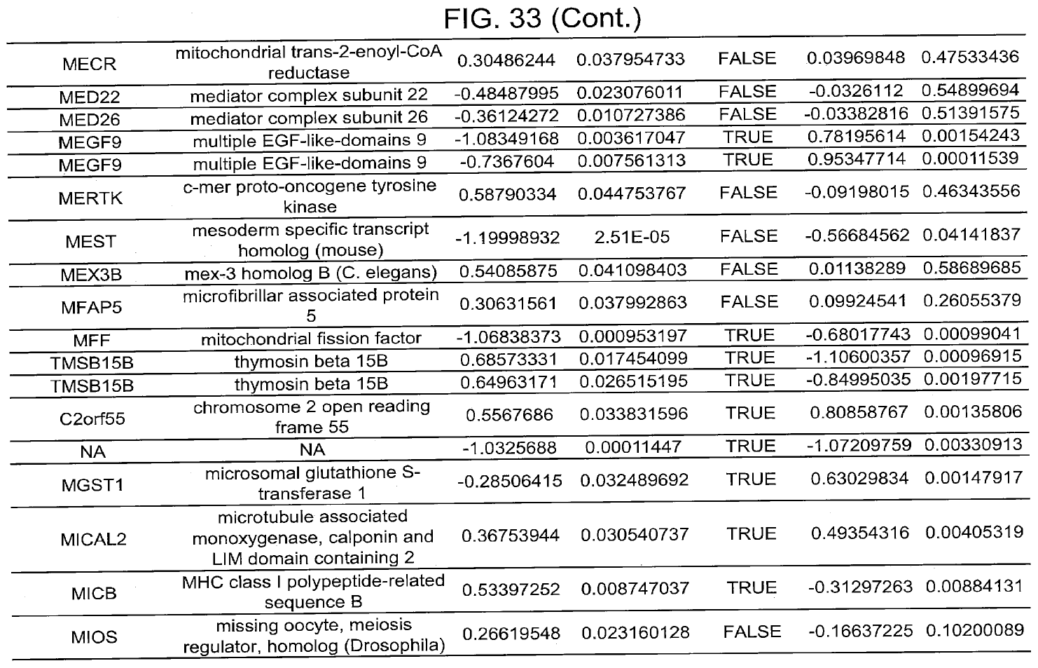

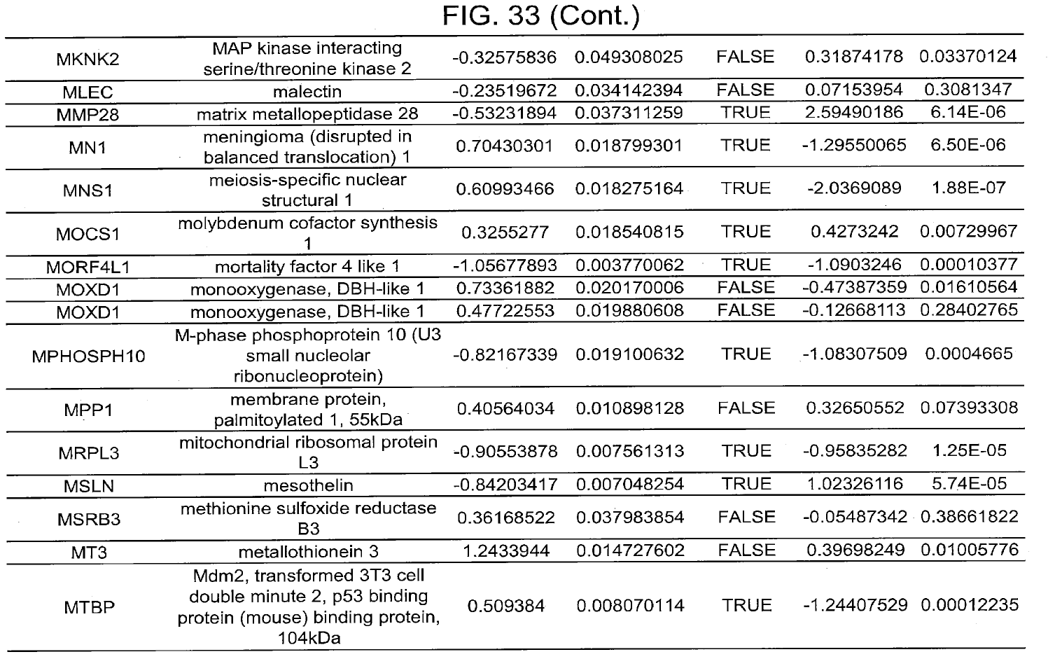

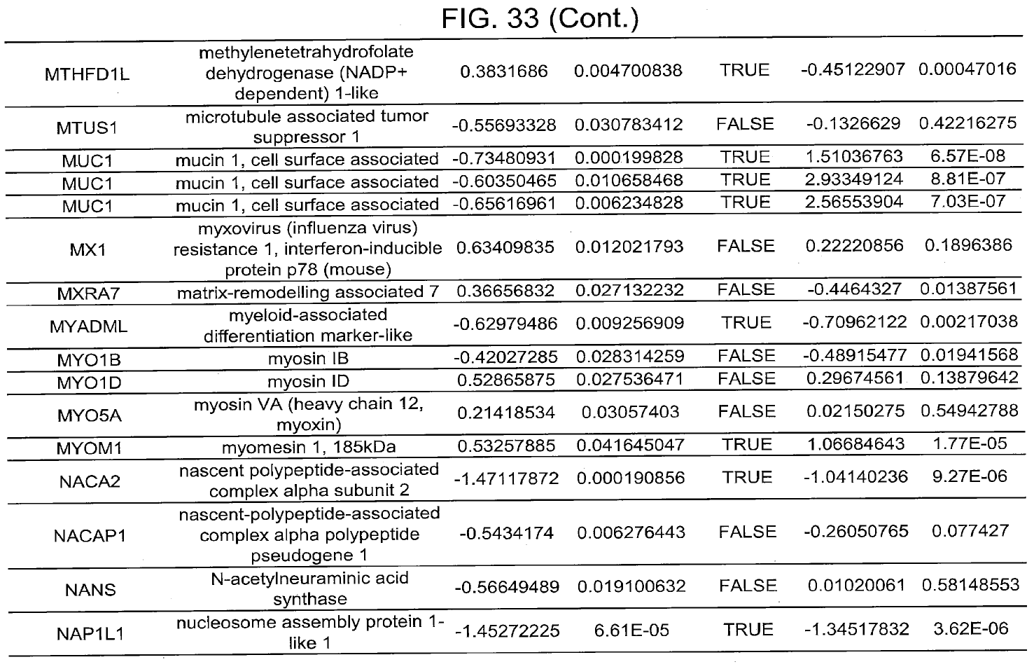

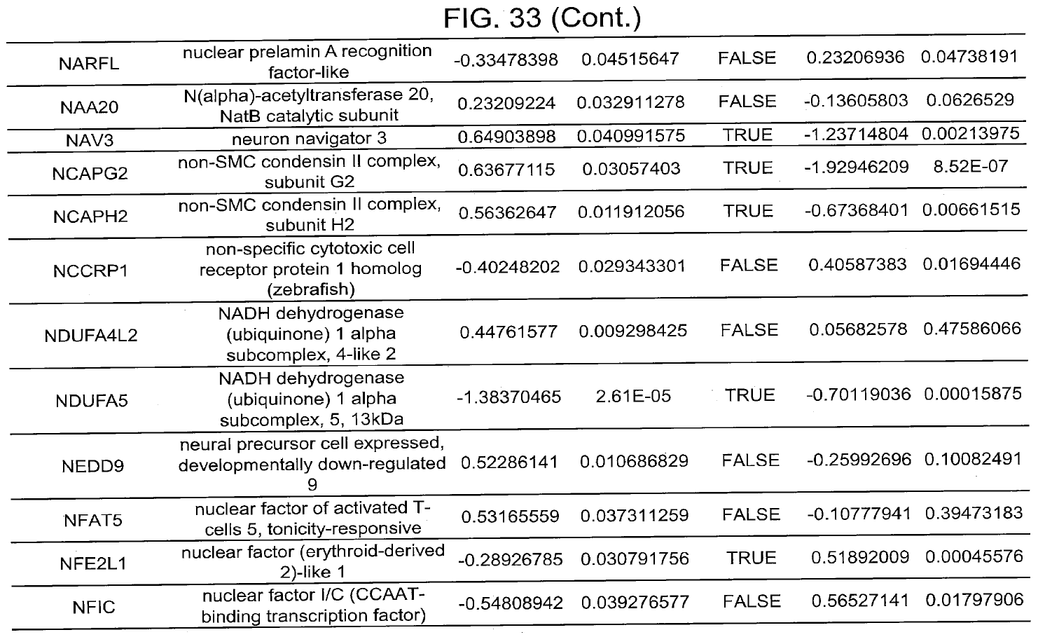

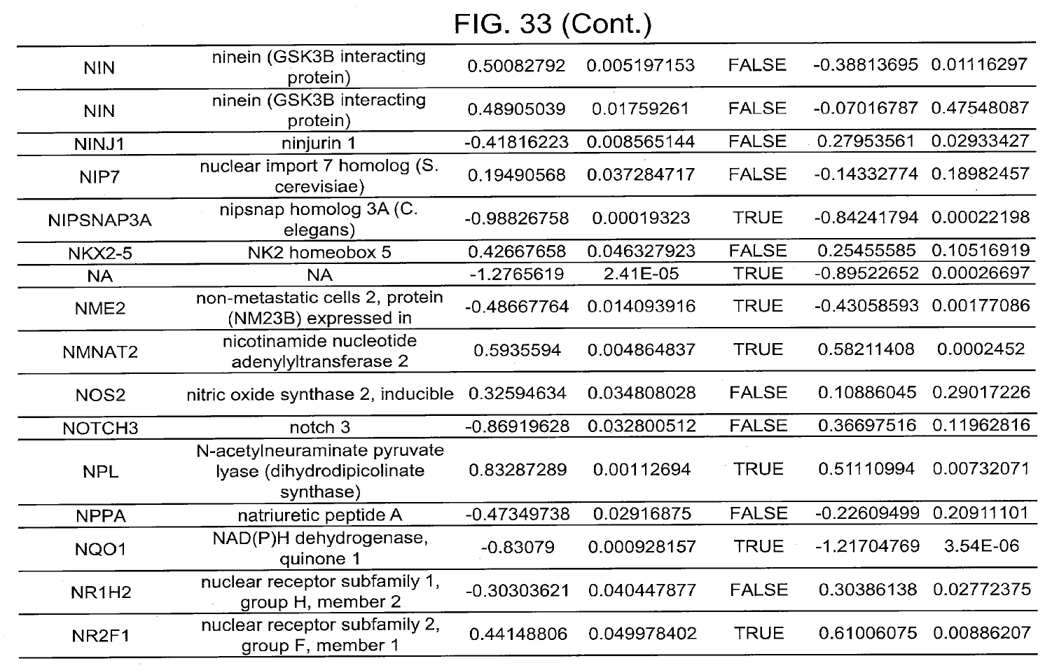

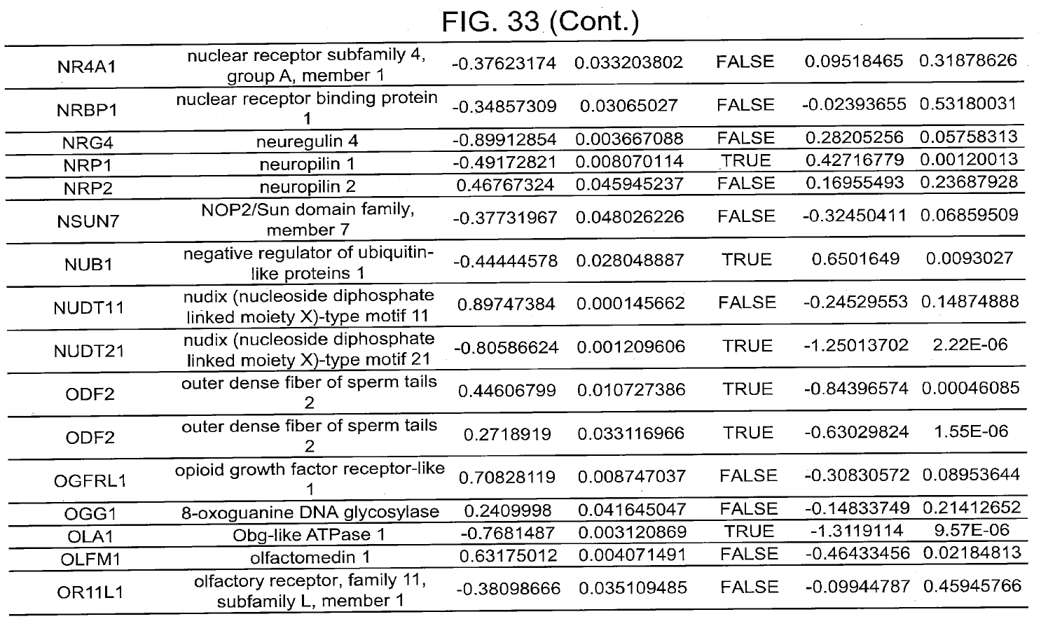

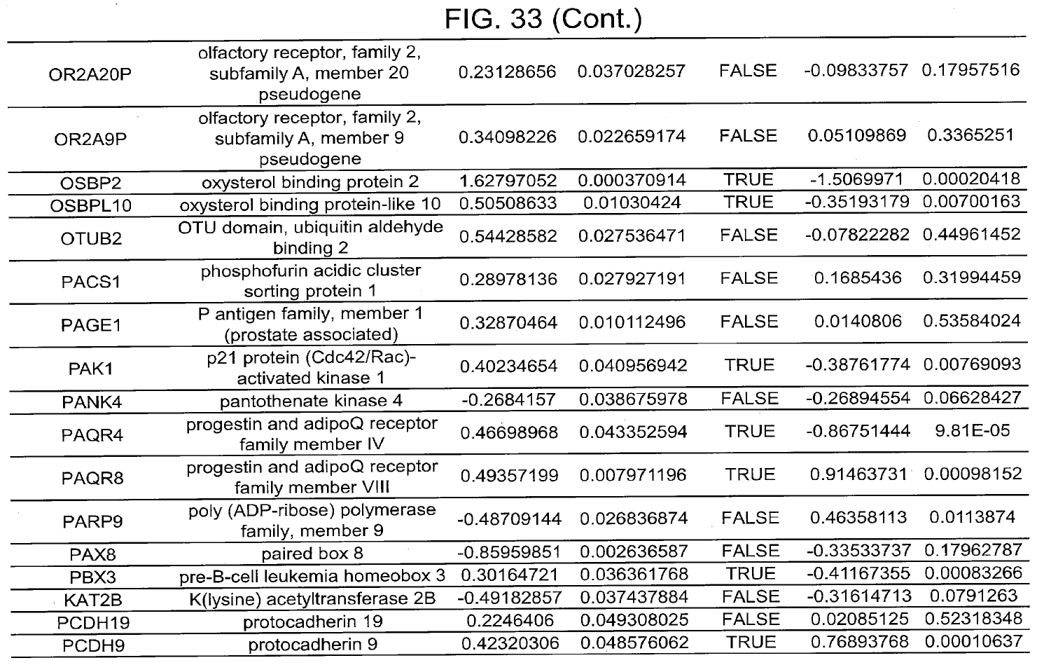

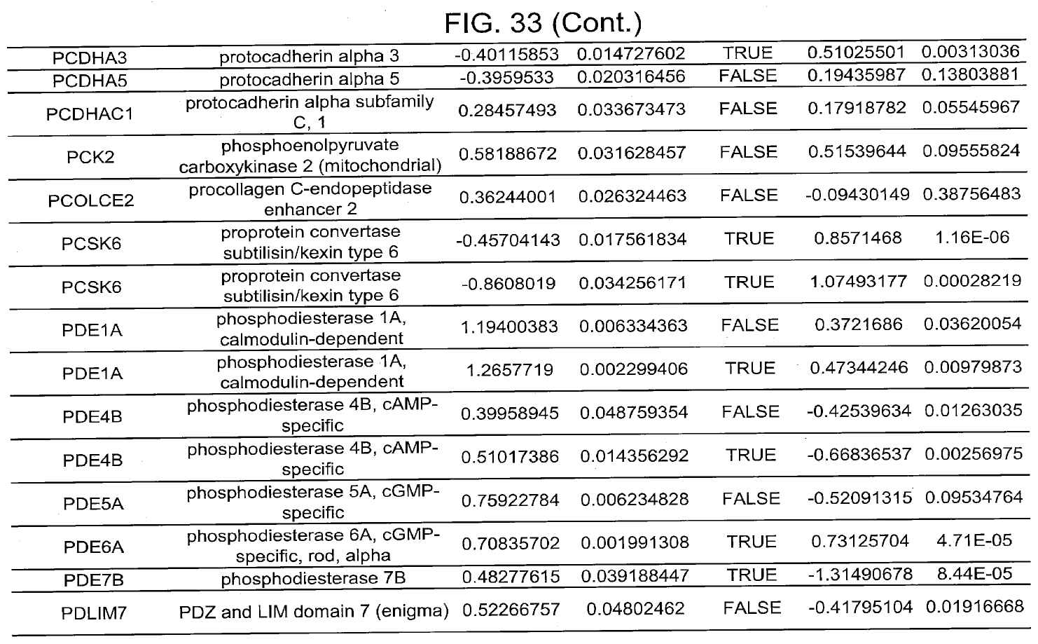

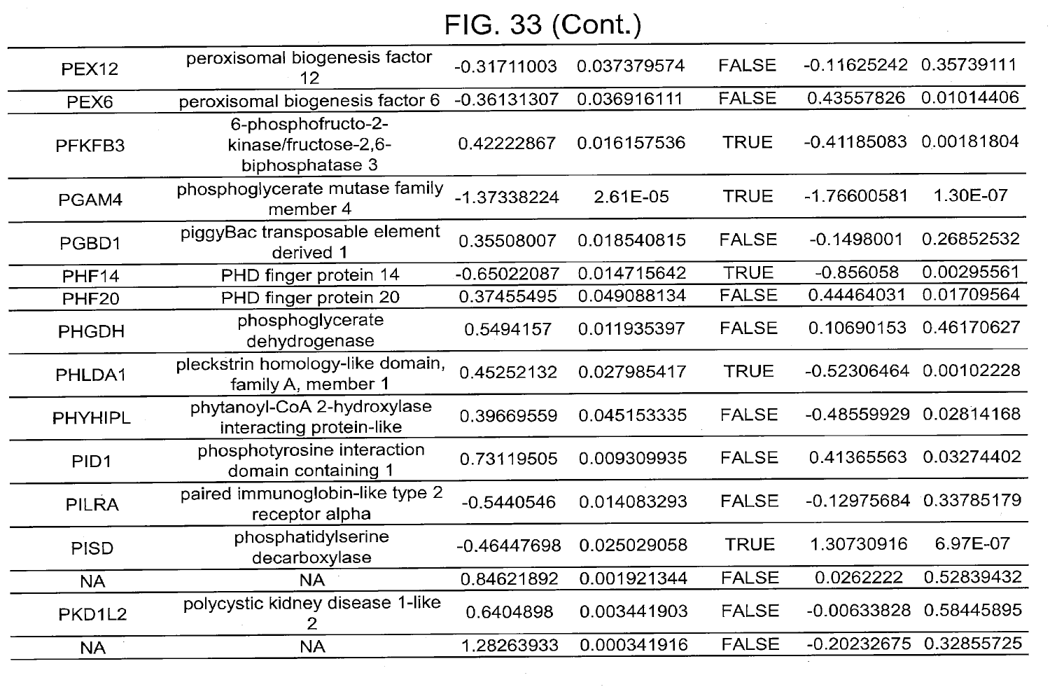

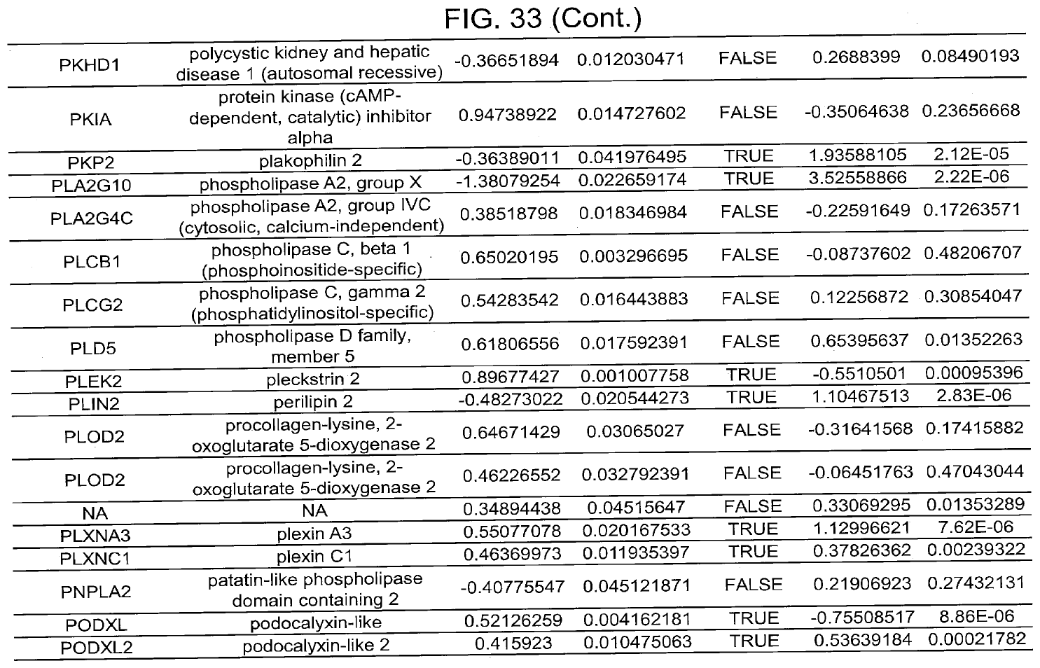

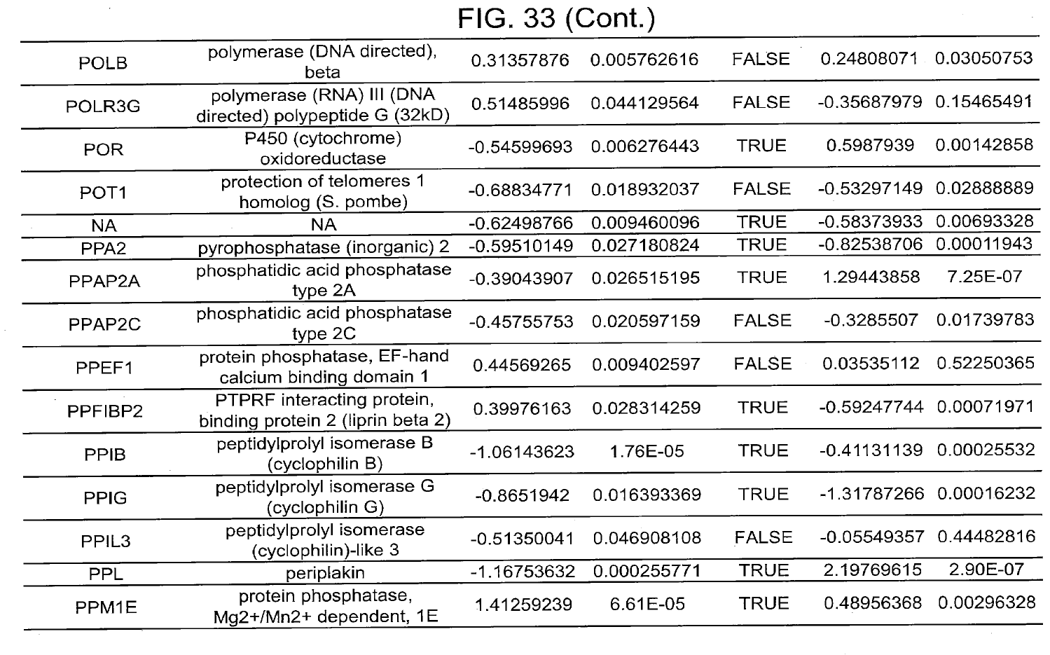

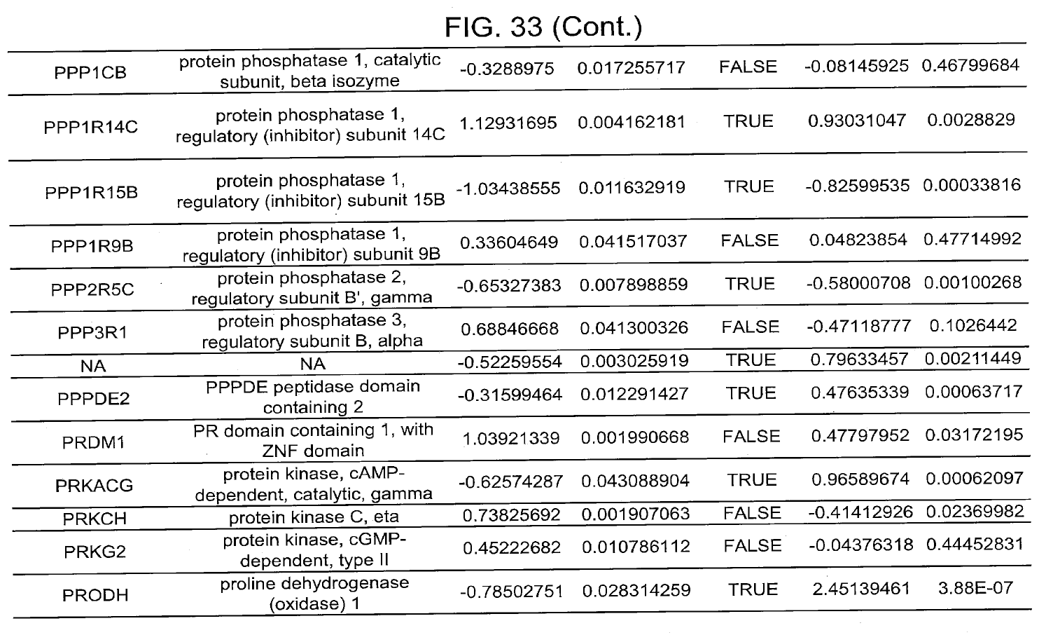

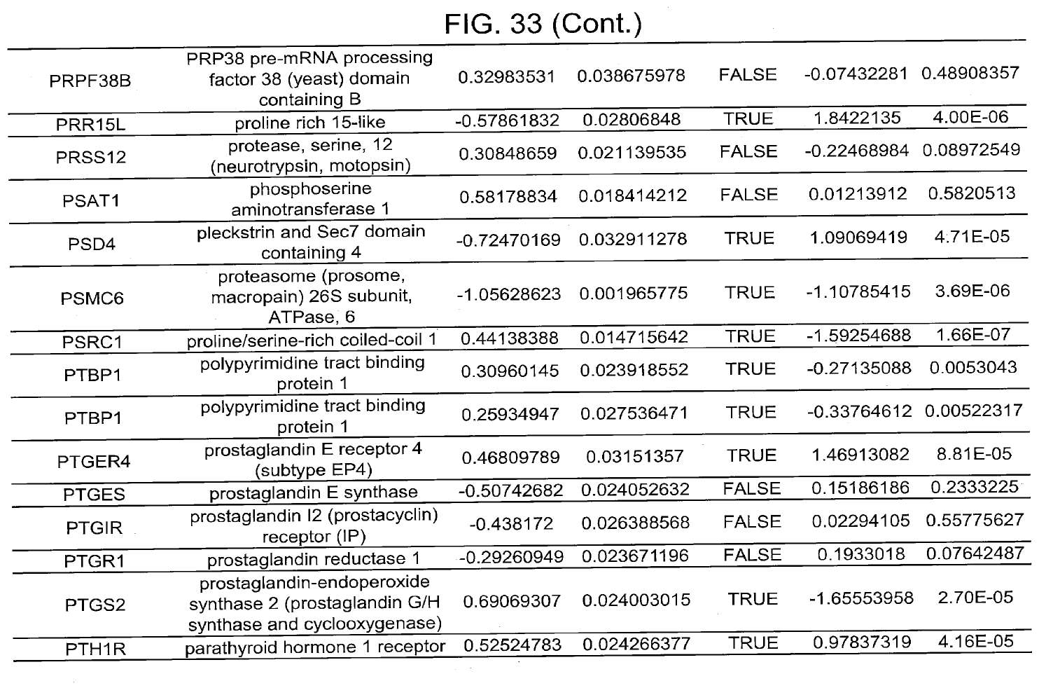

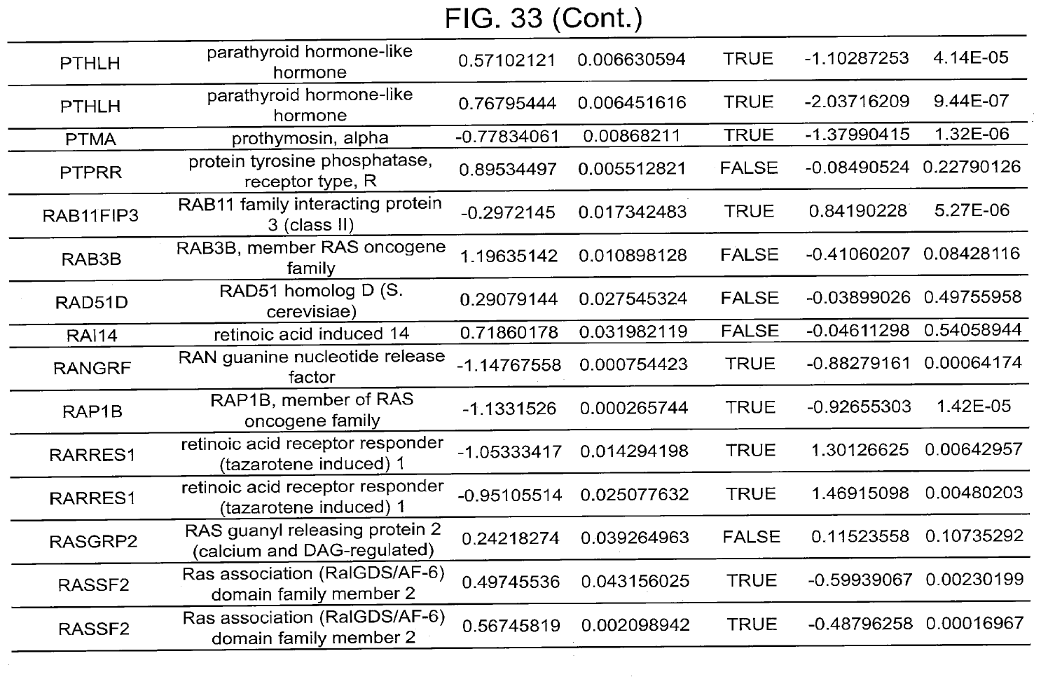

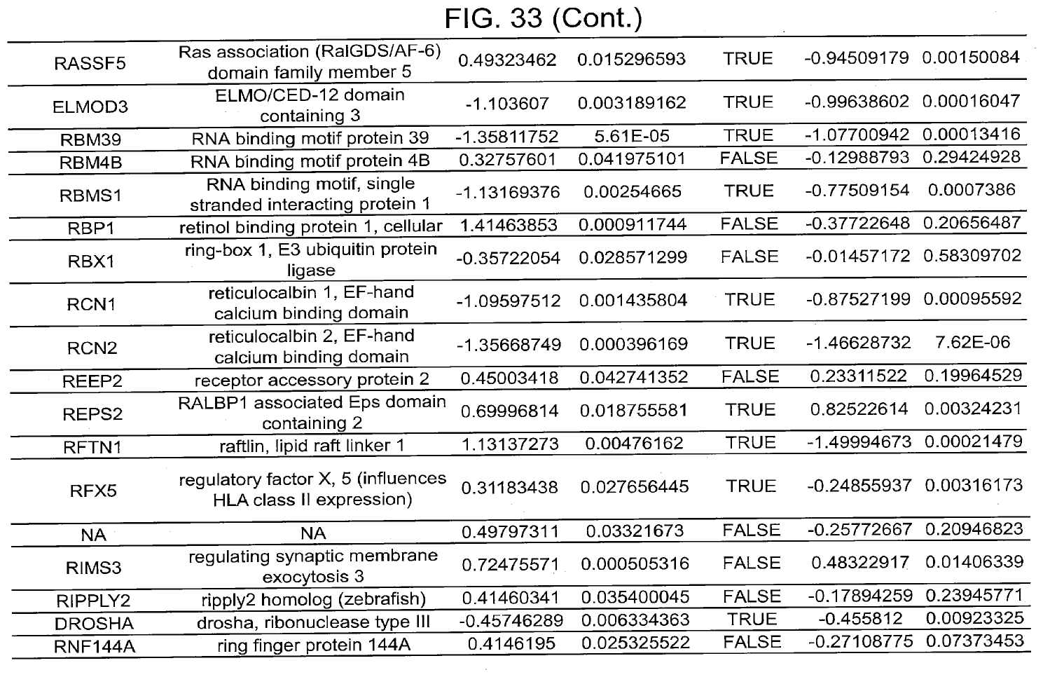

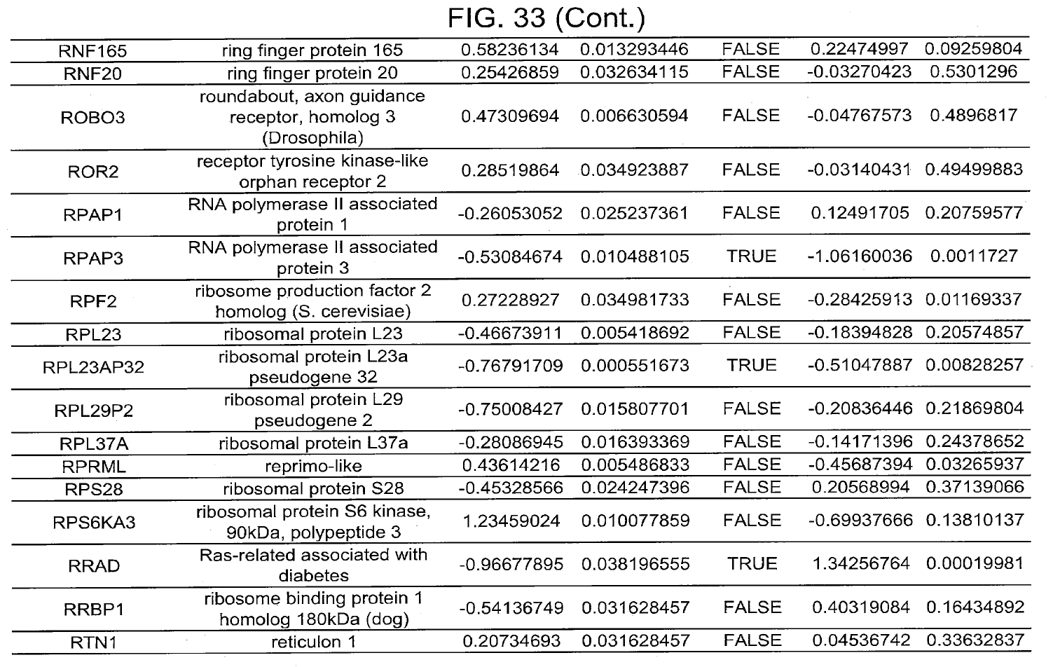

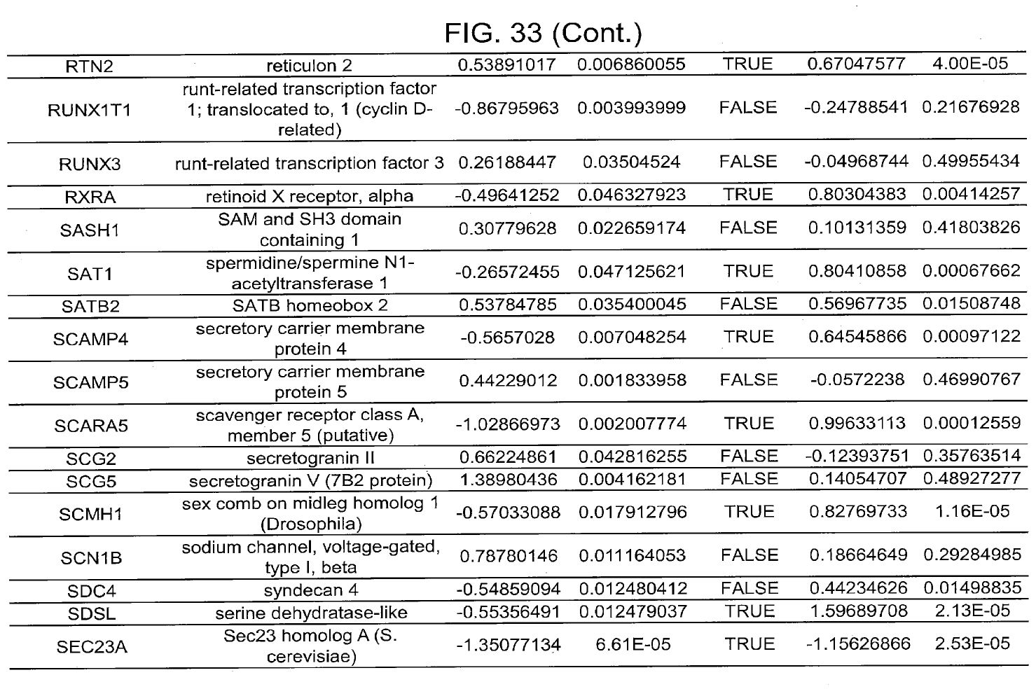

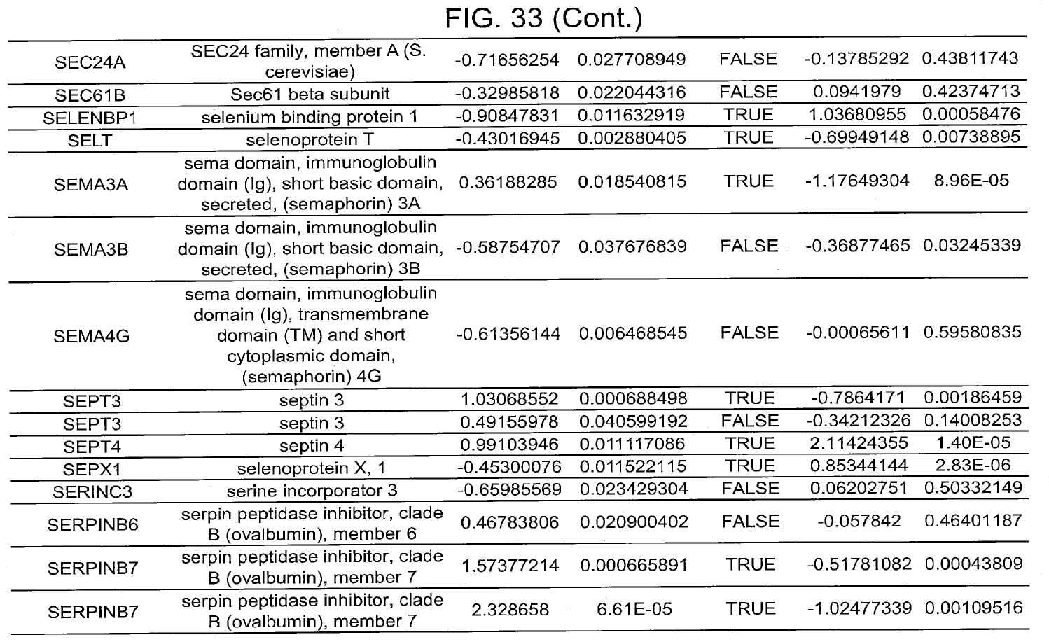

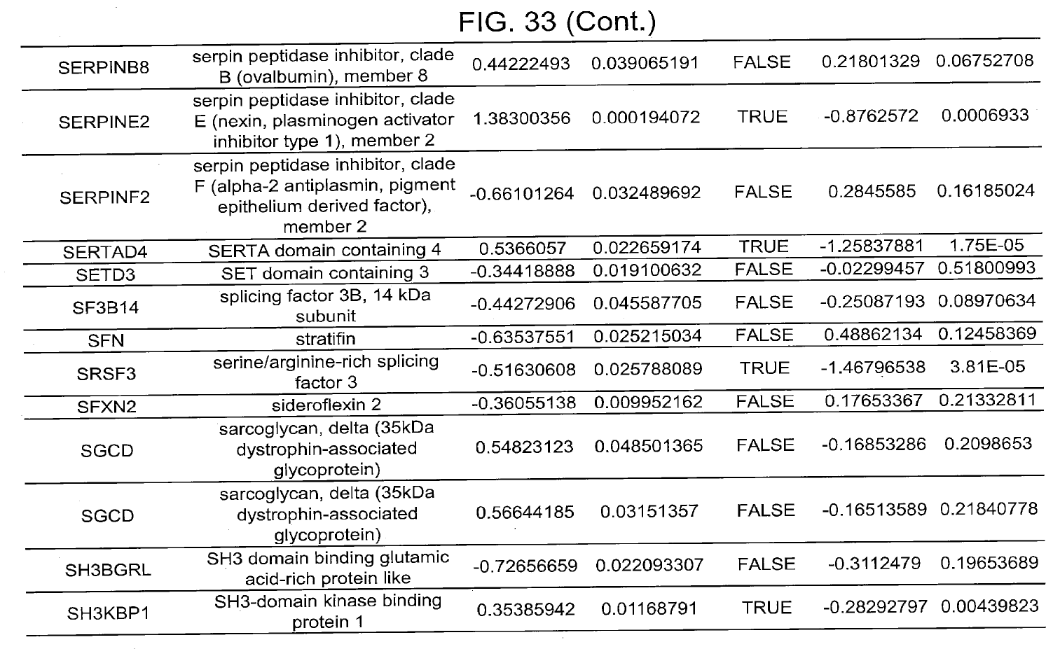

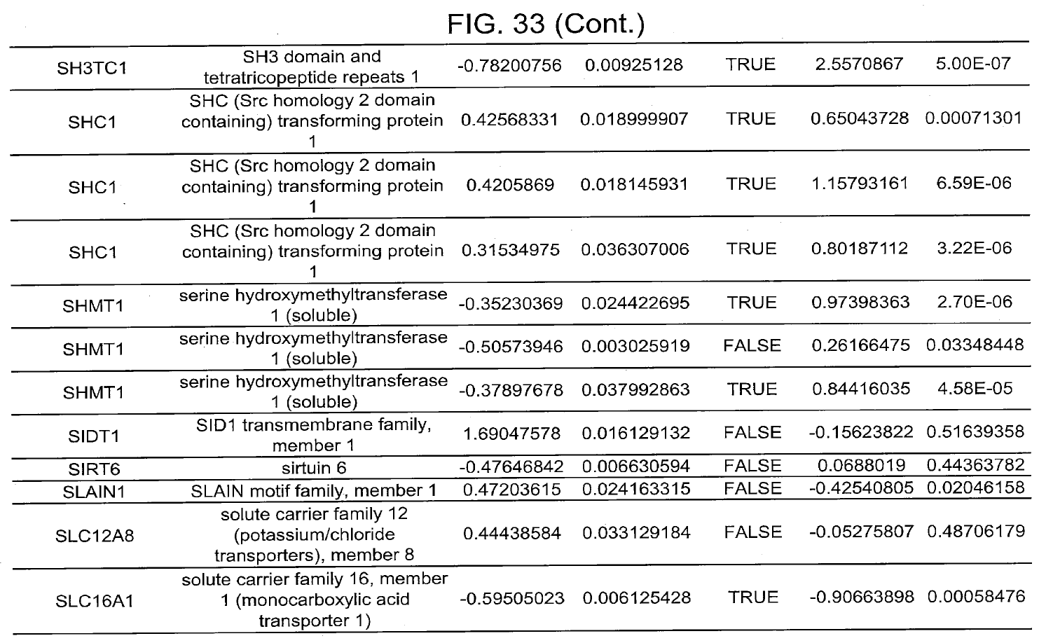

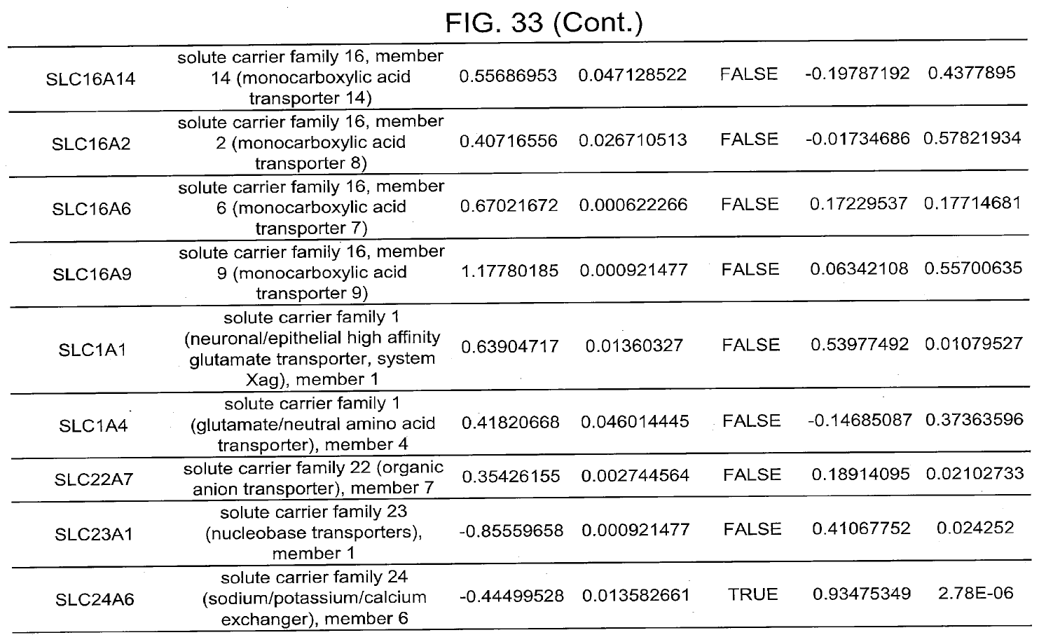

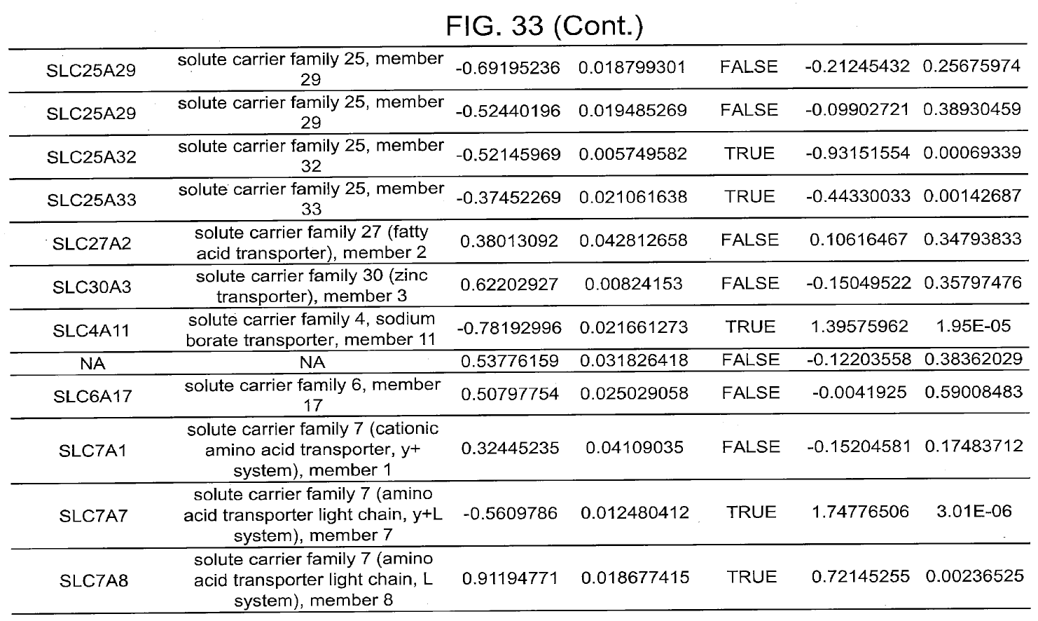

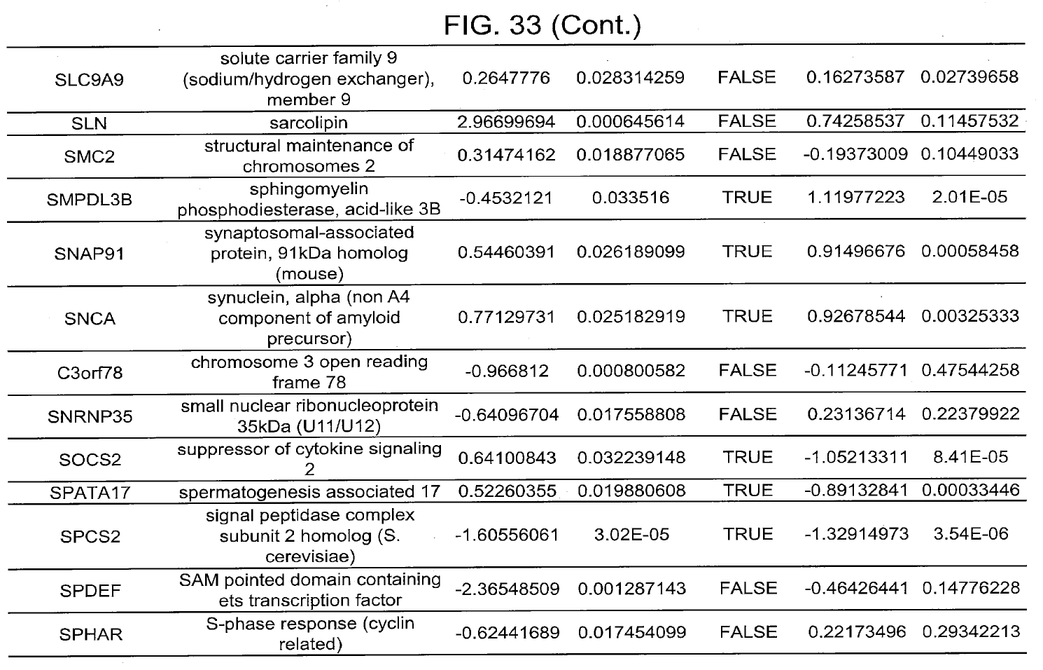

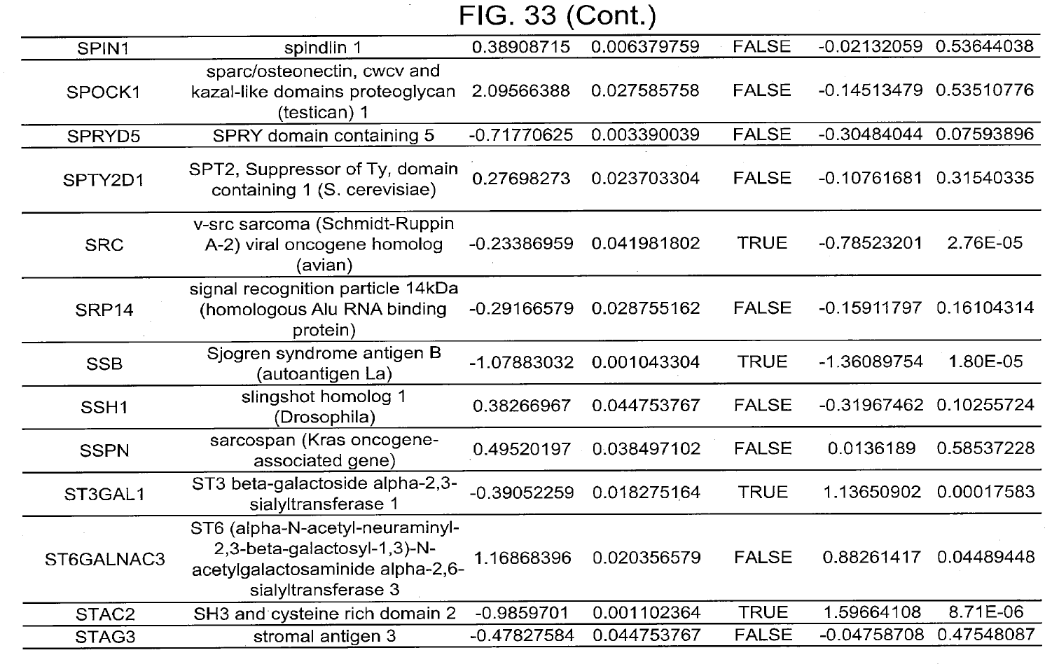

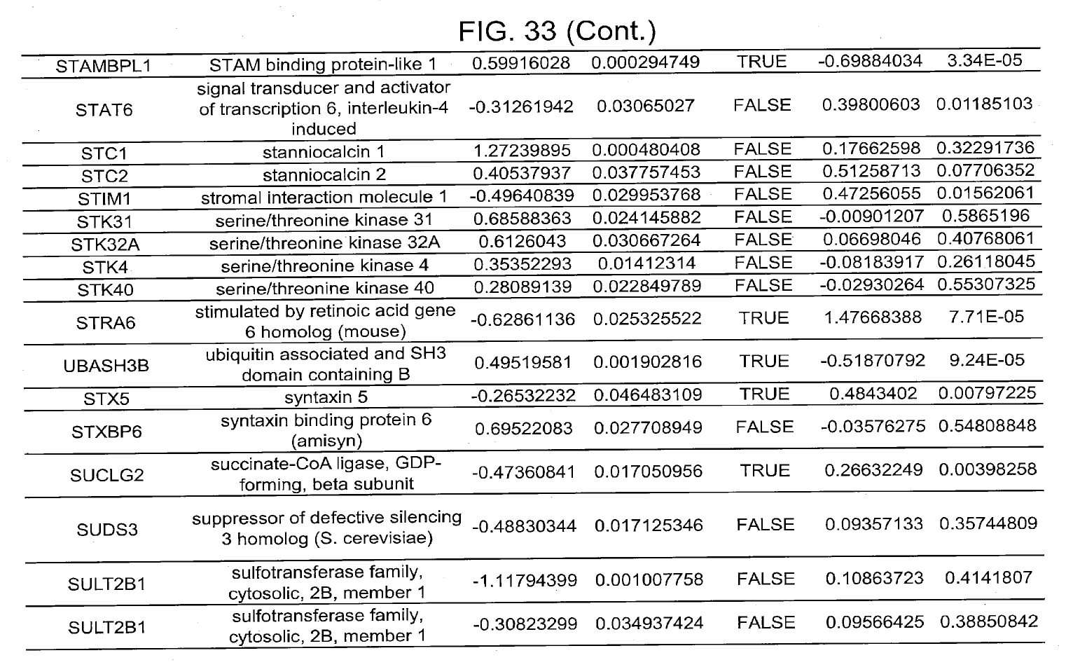

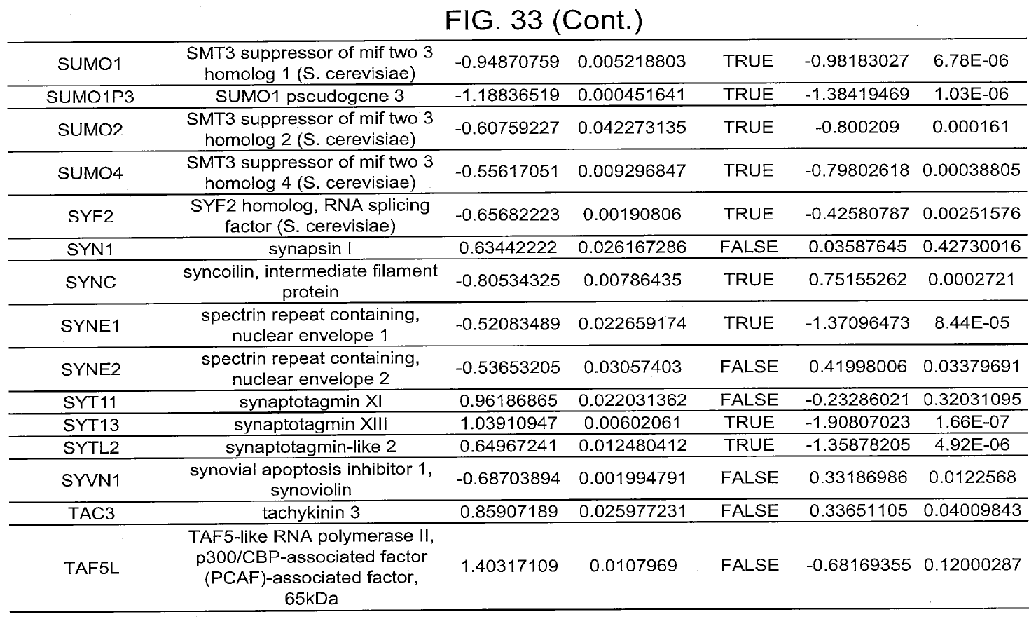

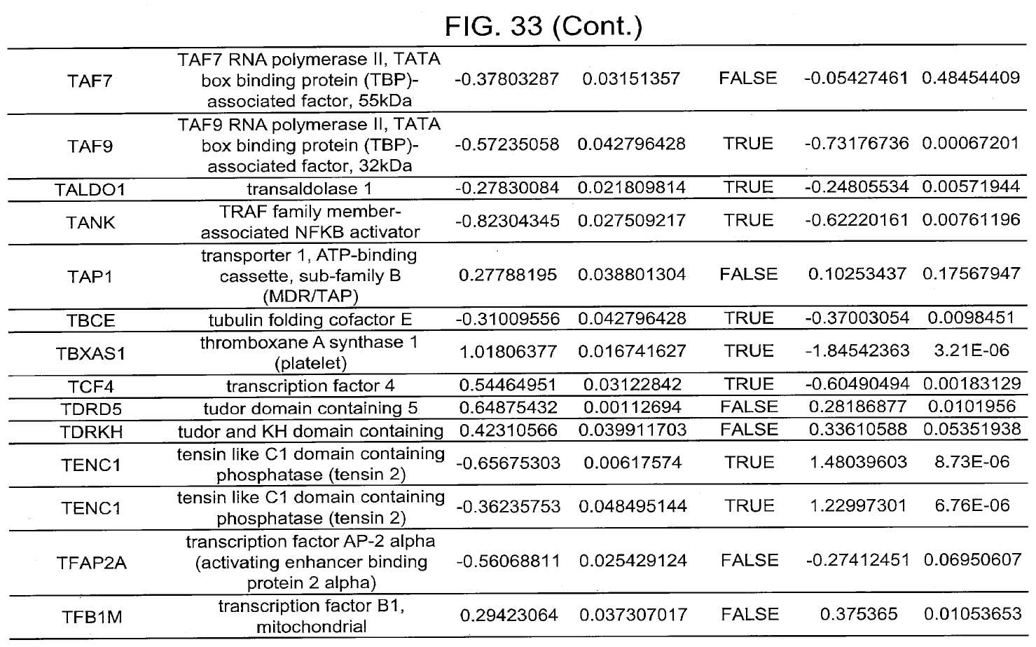

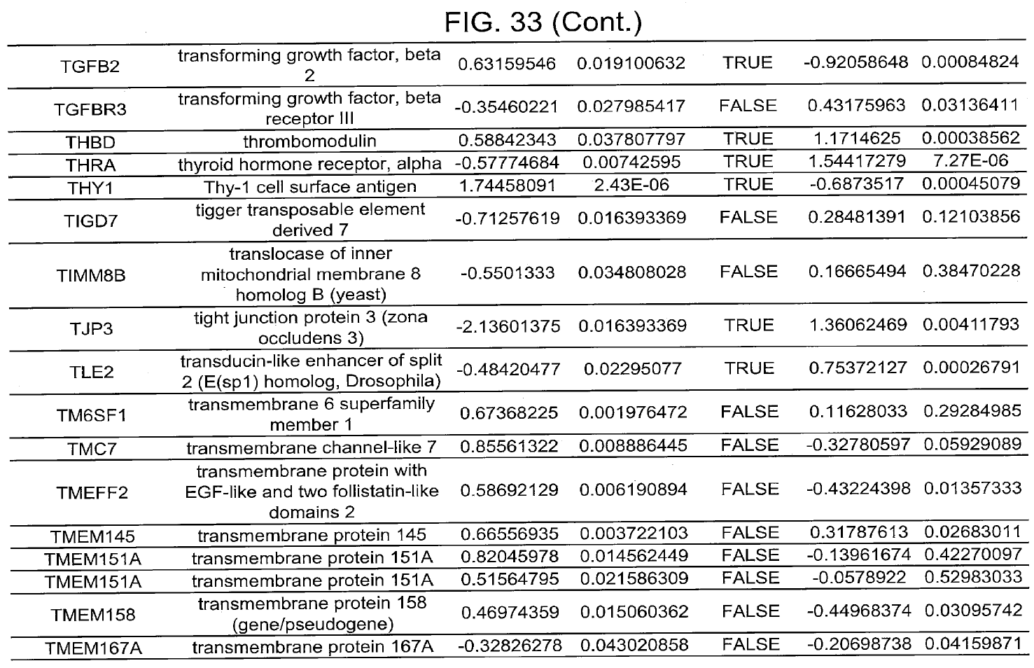

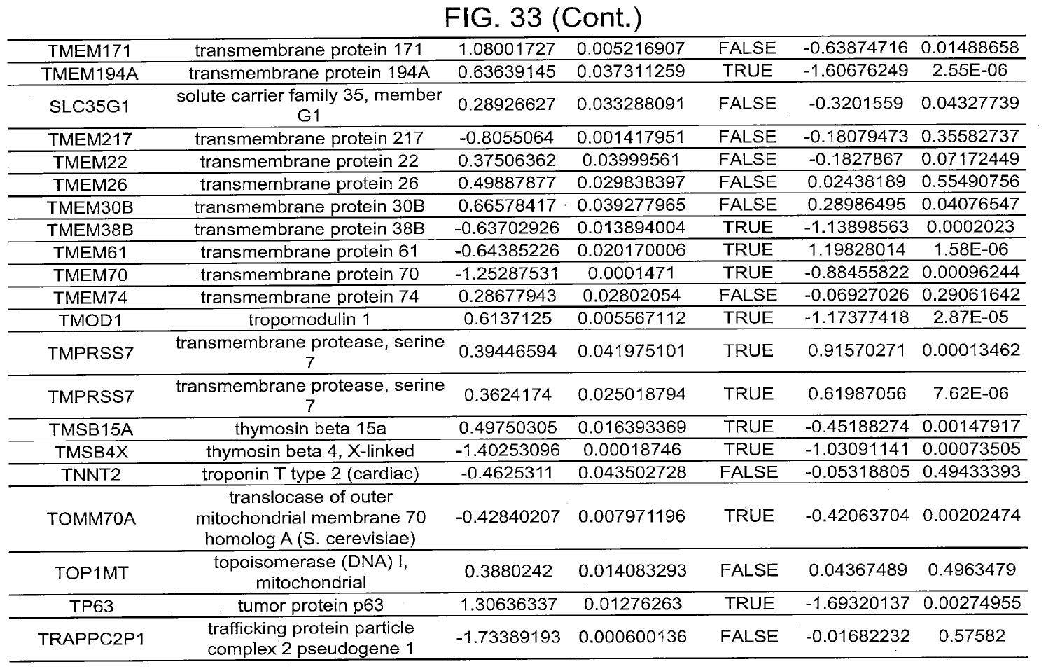

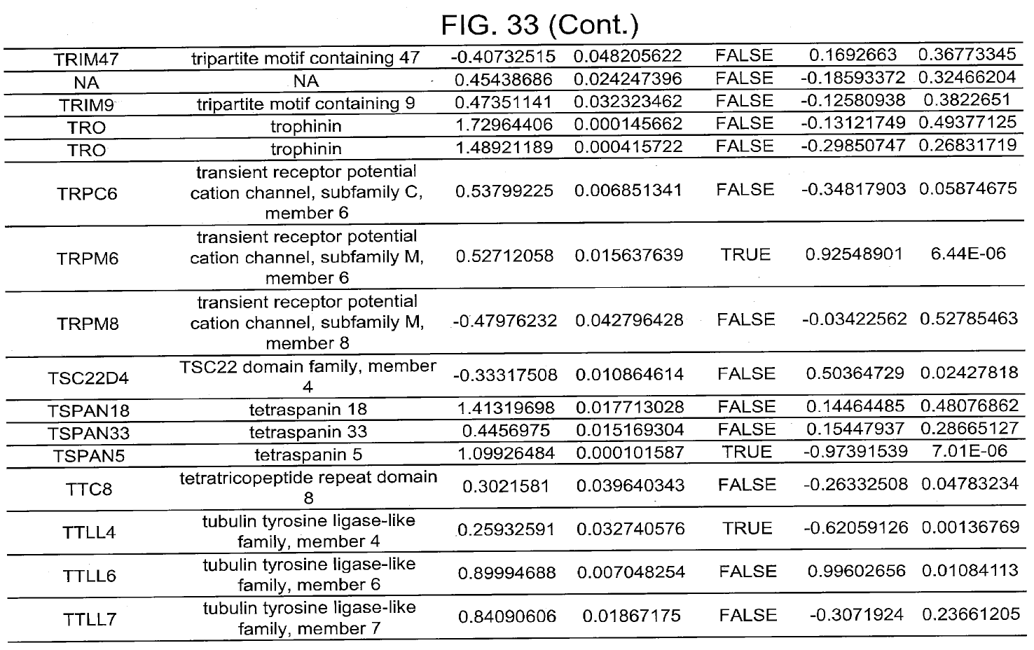

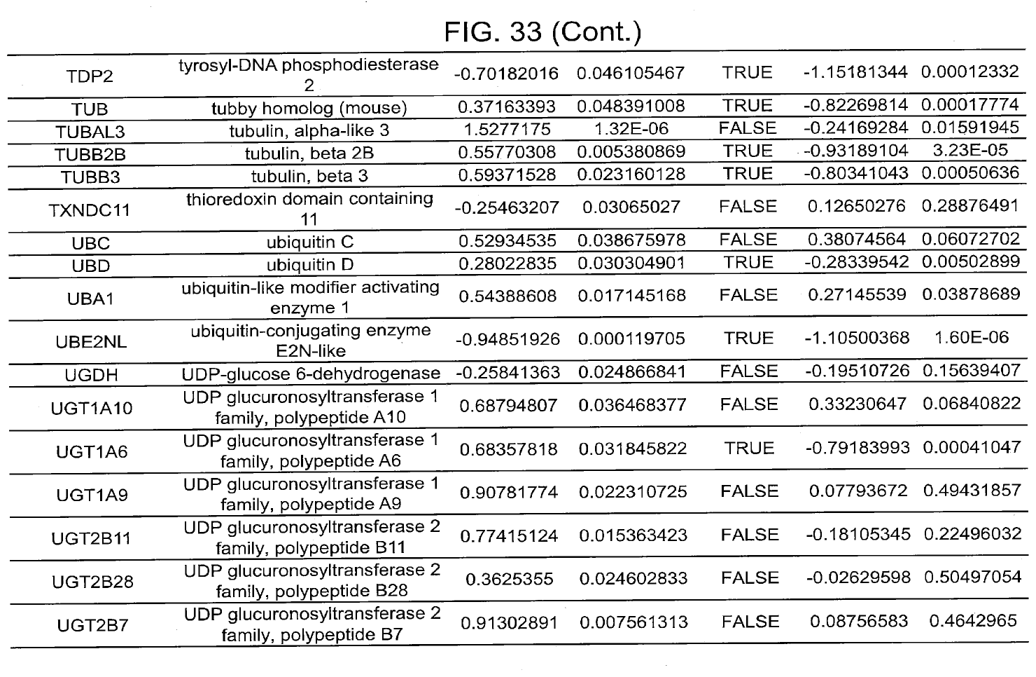

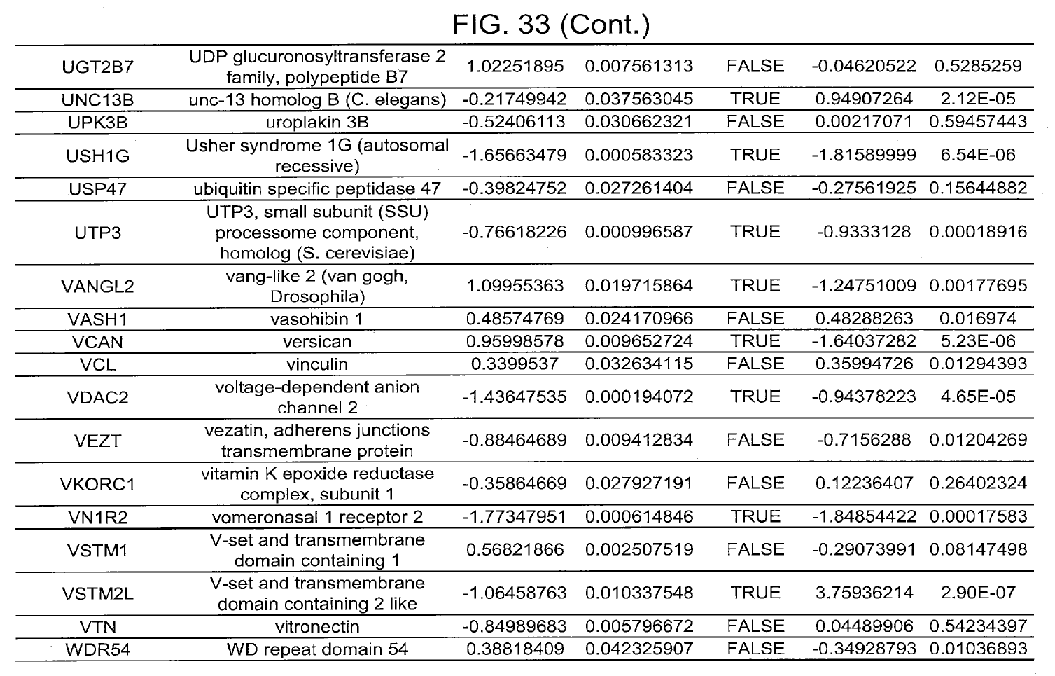

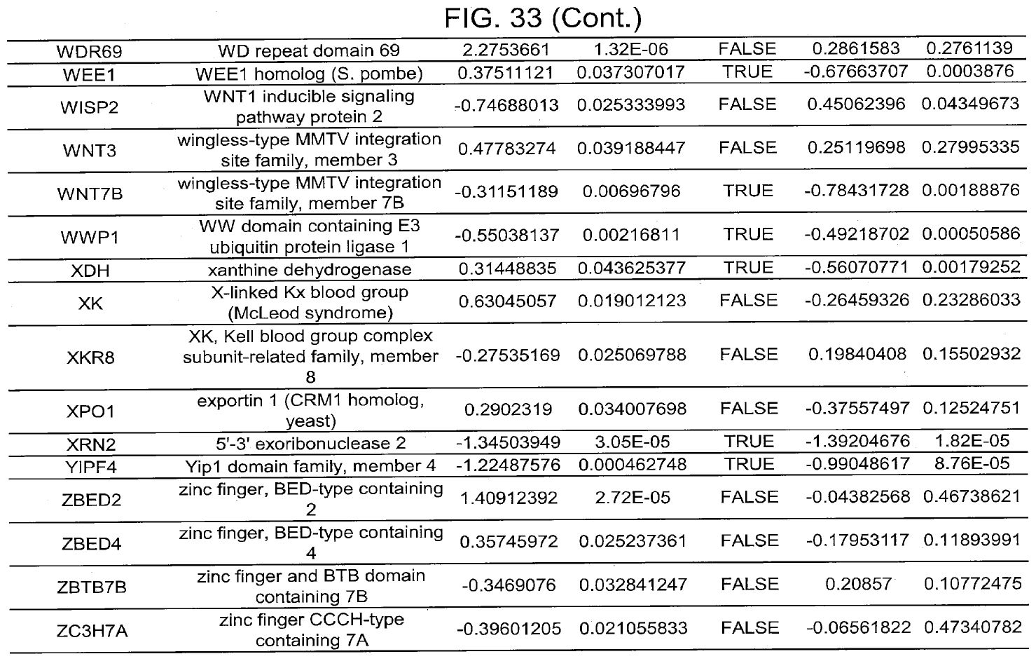

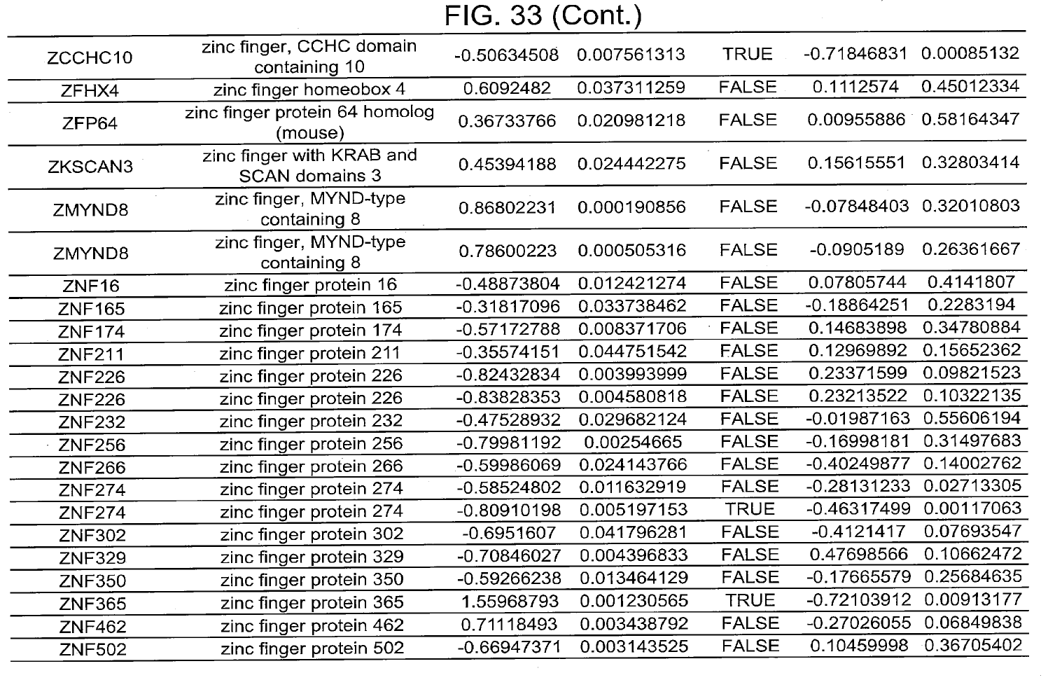

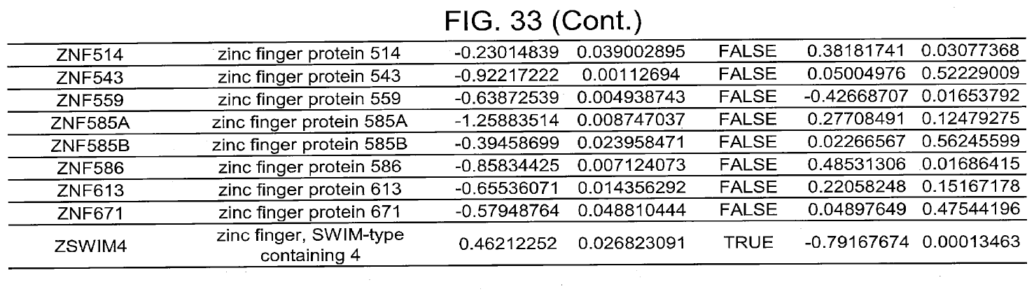

FIG. 33 is a supplemental dataset of genes affected significantly by GLP Depletion (24 h treatment with 100 nM Dex) was prepared. The dataset lists all genes for which expression was significantly (q≤0.05) different for shGLP cells treated with dex vs. noninfected and shNS-expressing cells treated with dex. Column E represents loge fold change in expression in dex-treated cells, caused by GLP depletion. In column E, positive loge fold change values indicate that the gene expression is up-regulated upon GLP depletion (i.e., GLP negatively regulates the expression of the gene). Inversely, negative loge fold change values indicate that the gene expression is down-regulated upon GLP depletion (i.e., GLP positively regulates the expression of the gene). Column G indicates whether the gene was also significantly hormone-regulated (fold change≥1.5-fold, q≤0.01). Column H represents loge fold change in expression for these genes upon hormone treatment (non infected and shNS-expressing cells treated with 100 nM dex for 24 h vs. untreated samples). In column H, positive loge fold change values indicate that the gene expression is up-regulated by hormone treatment whereas negative log₂ fold change values indicate that the gene expression is down-regulated by hormone treatment.

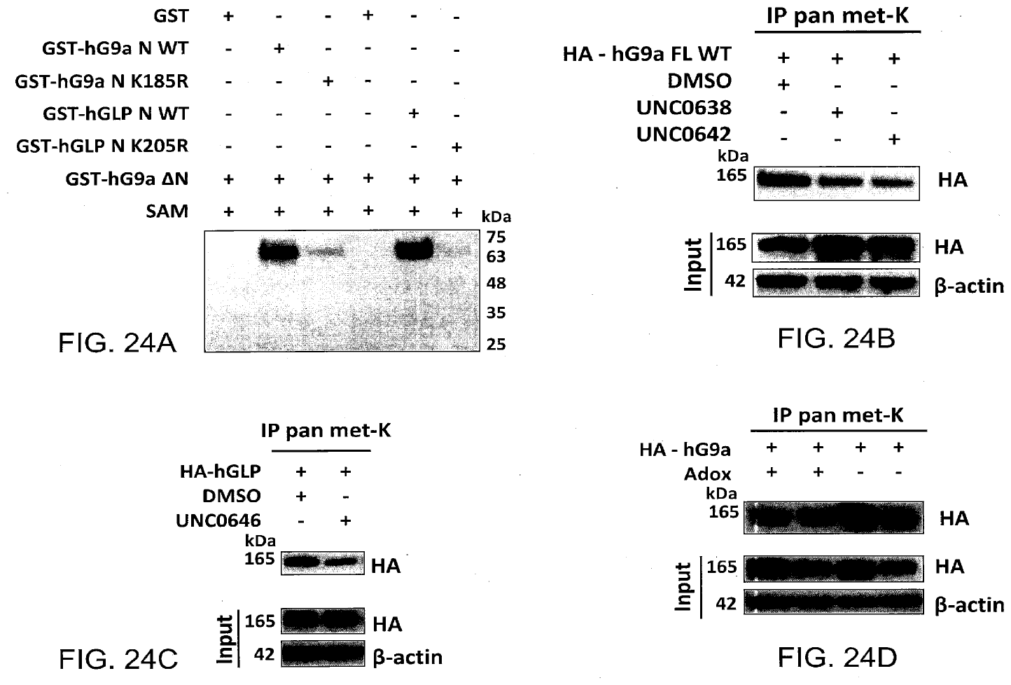

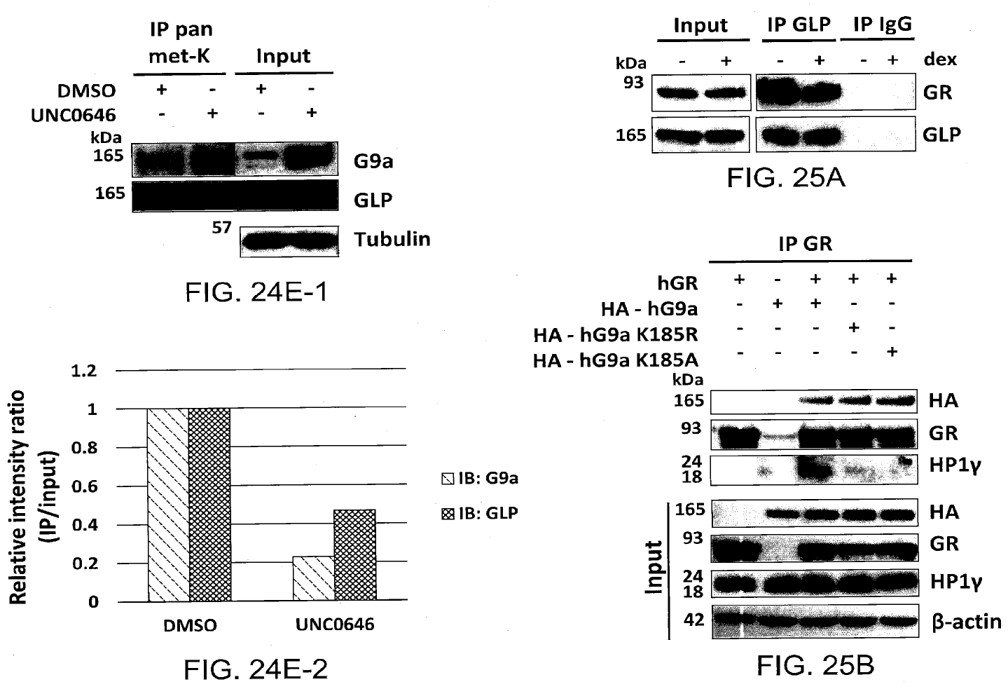

The results as shown in FIG. 24A highlight identification of G9a and GLP methylation sites in vitro. N-terminal domains of hG9a, hGLP wild type or mutants were incubated with GST-hG9a ΔN in the presence of [methyl-³H] SAM. Reaction products were analyzed by SDS-PAGE followed by fluorography.

G9a methylation in cells is reduced by G9a/GLP methyltransferase inhibitors as highlighted in the results shown in FIG. 24B. Cos-7 cells were transfected with a plasmid encoding full length HA-hG9a and treated with 2 μM UNC0638, UNC0642 or vehicle DMSO for 24 h. Lysates were immunoprecipitated with pan met-K antibody and immunoblotted with HA antibody (top). Expression of HA-G9a and β-actin (loading control) in the unfractionated samples (Input) is also shown (bottom).

GLP methylation in cells is reduced by G9a/GLP methyltransferase inhibitor as highlighted in the results shown in FIG. 24C. Cos-7 cells transfected with a plasmid encoding full length HA-hGLP were treated with 2 μM G9a/GLP methyltransferase inhibitor UNC0646 or vehicle DMSO for 24 h. Lysates were immunoprecipitated with pan met-K antibody and immunoblotted with HA antibody.

G9a methylation in cells is reduced by a general SAM-dependent methylation inhibitor as shown in FIG. 24D. Cos-7 cells were transfected with a plasmid encoding full length HA-hG9a and treated with 40 μM adenosine dialdehyde (Adox) or vehicle DMSO for 24 h. Lysates were immunoprecipitated with pan met-K antibody and immunoblotted with HA antibody (top). Expression of HA-G9a and β-actin (loading control) in the unfractionated samples (Input) is also shown (bottom).

The results as shown in FIGS. 24E-1 and 24E-2 highlight methylation of endogenous G9a and GLP in A549 cells. A549 cells were treated with 2 μM UNC0646 or vehicle DMSO for 24 h. Lysates were immunoprecipitated with pan met-K antibody and immunoblotted with G9a or GLP antibody (left panels). Expression of G9a, GLP and tubulin (loading control) in the unfractionated extracts is shown at the right (Input). Western-Blot quantification was determined relative to input using ChemiDoc MP (Biorad) to measure chemiluminescence from the immunoblots, and the ratio of the IP signal to the input signal was calculated for each sample.

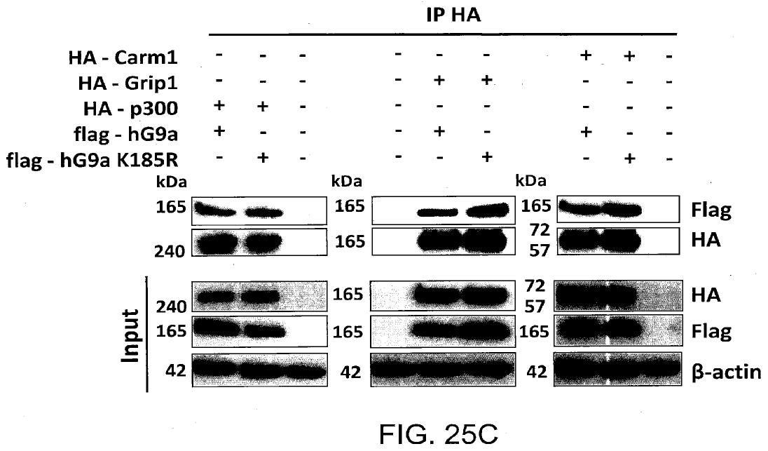

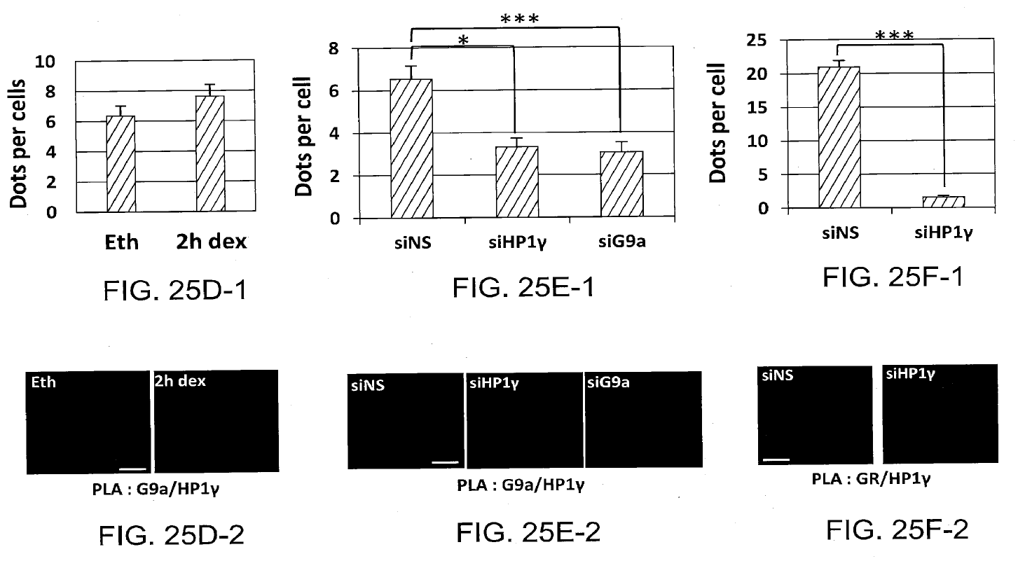

GR interacts with GLP as highlighted in the results as shown in FIG. 25A. Coimmunoprecipitation of endogenous GR with GLP from lysates of A549 cells treated with 100 nM dex or ethanol for 4 h. Immunoprecipitation was performed with anti-GLP or control IgG antibodies and immunoblotted with anti-GR and anti-GLP antibodies.

GR interaction with G9a did not require K185 as highlighted in the results as shown in FIG. 25B. Cos-7 cell were transfected with plasmids encoding hGR and HA-hG9a wild type or K185A or K185R mutants. Lysates were immunoprecipitated with GR antibody and immunoblotted with antibodies against HA, GR and HP1γ. Expression of the indicated proteins in the Input sample is shown below.

G9a methylation site is not required for interaction with coregulators Grip1, Carm1, and p300 as highlighted in the results as shown in FIG. 25C. Cos-7 cells were transfected with plasmids encoding HA-Carm1, HA-Grip 1, or HA-p300, along with a plasmid encoding full length flag-hG9a wild type or K185R mutant. Lysates were immunoprecipitated with HA antibody and immunoblotted with flag or HA antibodies. Expression of HA- and flag- tagged proteins and β-actin (loading control) in the unfractionated extracts is shown at the bottom (Input).

Interaction of endogenous G9a and HP1γ analyzed by PLA as highlighted in the results as shown in FIGS. 25D-1 and 25D-2. A549 cells were treated with 100 nM dex or the equivalent volume of vehicle ethanol (Eth) for 2 h. Representative images are shown with the quantified interactions, which are shown as the mean±SEM of three independent experiments. p-value was determined using a paired t-test and was not significant. Scale bar represents 10 μm.

Validation of the endogenous interaction between G9a and HP1γ analyzed by PLA as highlighted in the results as shown in FIGS. 25E-1 and 25E-2. A549 cells were transfected with non-specific siRNA (siNS) or siRNA against HP1γ (siHP1γ) or G9a (siG9a) and then treated with 100 nM dex for 2 h. Representative images are shown along with the mean±SEM of the quantified data from three independent experiments. p-value was determined using a paired t-test. *p≤0.05, * * *p≤0.001. Scale bar represents 10 μm.

Validation of the endogenous interaction between GR and HP1γ analyzed by PLA as highlighted in the results as shown in FIGS. 25F-1 and 25F-2. A549 cells were transfected with non-specific siRNA (siNS) or siRNA against HP1γ (siHP1γ) and then treated with 100 nM dex for 2 h. The mean±SEM of three independent experiments is shown. p-value was determined using a paired t-test. * * *p≤0.001. Scale bar represents 10 μm.

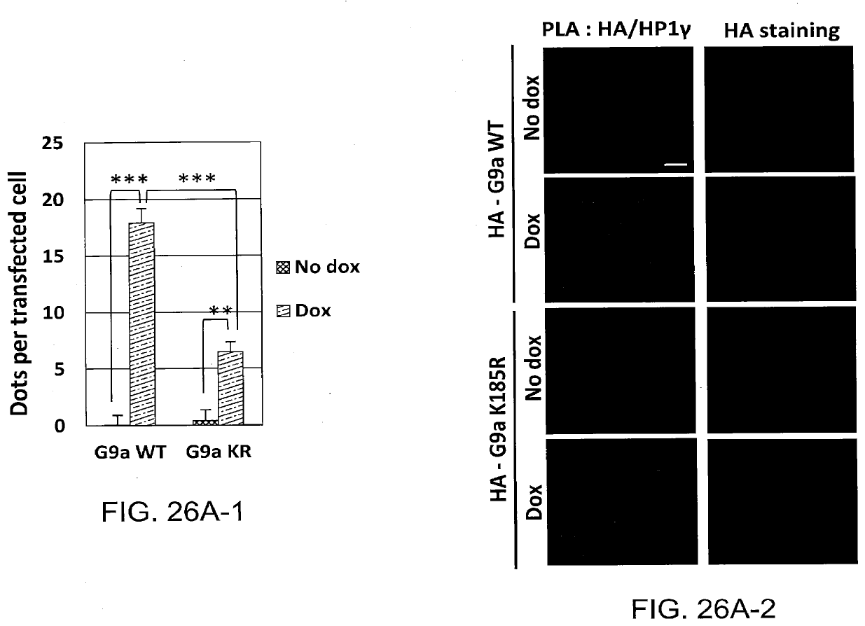

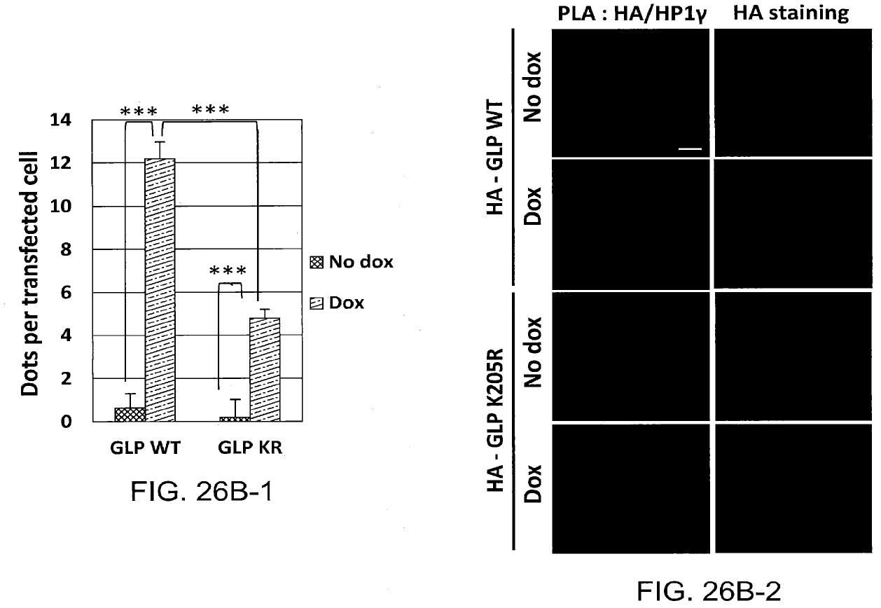

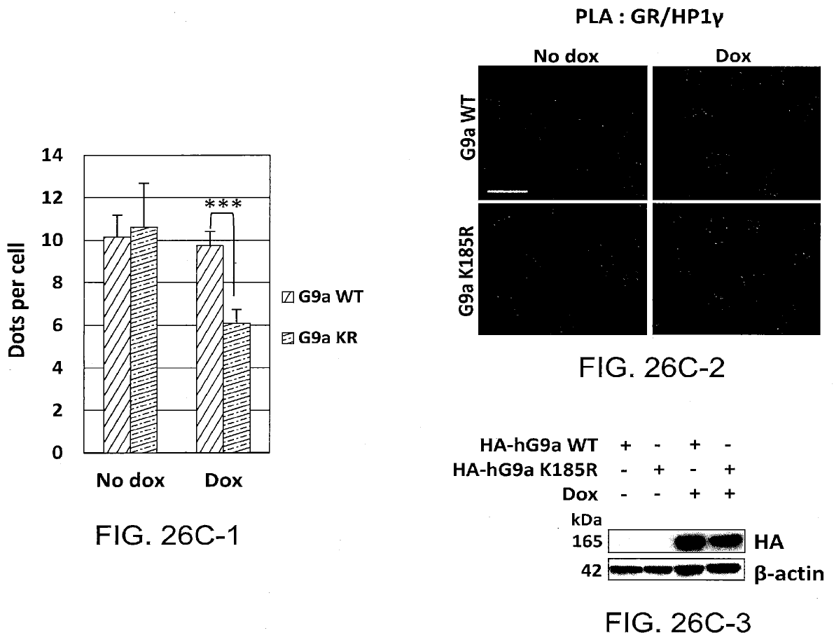

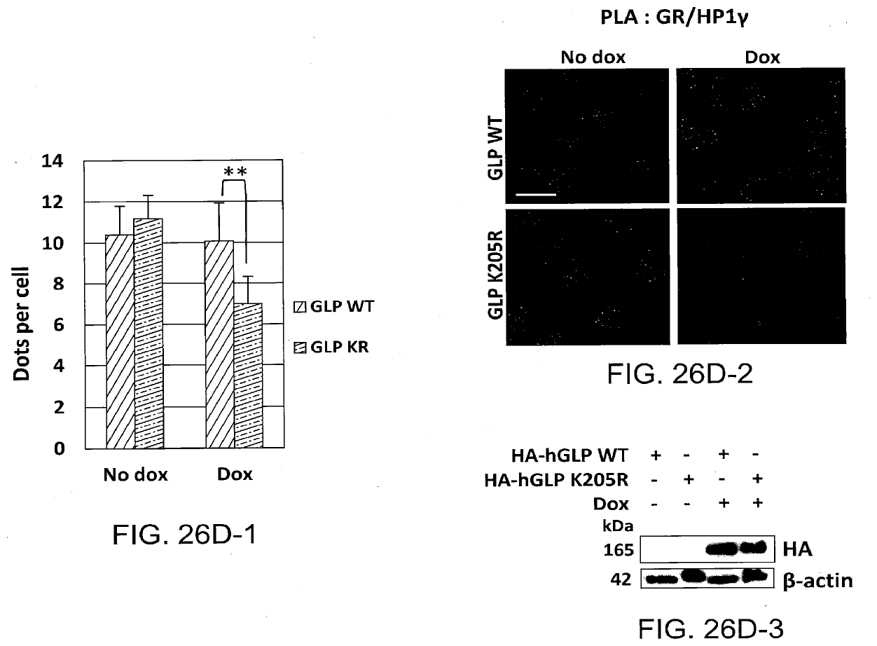

G9a K185 methylation is required for interaction with HP1γ as highlighted in the results as shown in FIGS. 26A-1 and 26A-2. Interactions between HP1γ and G9a wild-type or G9a K185R were analyzed by PLA. A549 rtta cell lines containing a stably-integrated doxycyline-regulated HA-G9a WT or HA-G9a K185R transgene were treated with 10 ng/ml of doxycycline or vehicle DMSO for 24 h prior to and during 2 h of 100 nM dex treatment. PLA was performed with antibodies against HA and HP1γ. Mouse alexa Fluor 488 secondary antibody against the HA primary antibody was added in the reaction during the amplification step in order to stain transfected cell nuclei in green. The detected interactions are indicated by red dots. The nuclei were counterstained with DAPI (blue). The number of interactions detected by Image J analysis is shown as the mean±SEM of three independent experiments. p-value was determined using a paired t-test. **p≤0.01, * * *p≤0.001. Scale bar represents 10 μm.

GLP K205 methylation is required for interaction with HP1γ as highlighted in the results as shown in FIGS. 26B-1 and 26B-2. PLA was conducted as in A on A549 rtta cell lines containing a stably-integrated doxycyline-regulated HA-GLP WT or HA-GLP K205R transgene.

GR-HP1γ interaction is blocked by G9a methylation site mutant as highlighted in the results as shown in FIGS. 26C-1, 26C-2, and 26C-3. Interactions between HP1γ and GR were analyzed by PLA on A549 rtta HA-G9a WT or HA-G9a K185R cells described in A. Cells were treated with 10 ng/ml of doxycycline or vehicle DMSO for 24 h prior to and during 2 h of 100 nM dex treatment. The detected interactions are indicated by red dots. The nuclei were counterstained with DAPI (blue). The number of interactions detected by Image J analysis is shown as the mean±SEM of three independent experiments. p-value was determined using a paired t-test. * * *p≤0.001. A fraction of the cells was analyzed for HA-G9a expression by immunoblot using indicated antibodies. Scale bar represents 10 μm.

GR-HP1γ interaction is blocked by GLP methylation site mutant as highlighted in the results as shown in FIGS. 26D-1, 26D-2, and 26D-3. PLA was conducted as in C on A549 rtta cell lines containing a stably-integrated doxycyline-regulated HA-GLP WT or HA-GLP K205R transgene. **p≤0.01

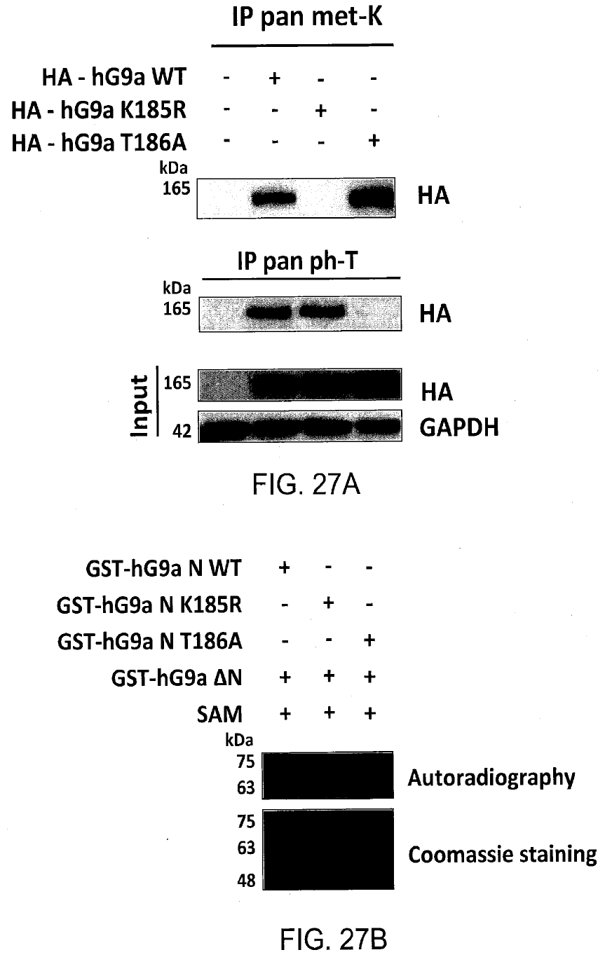

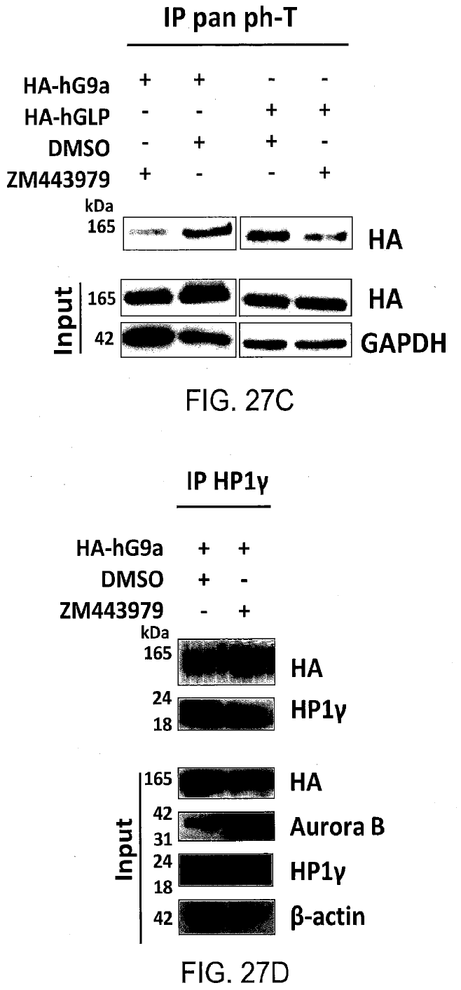

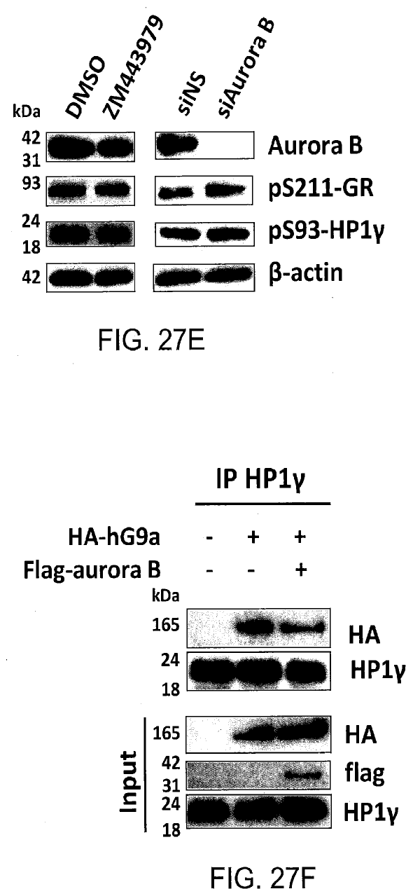

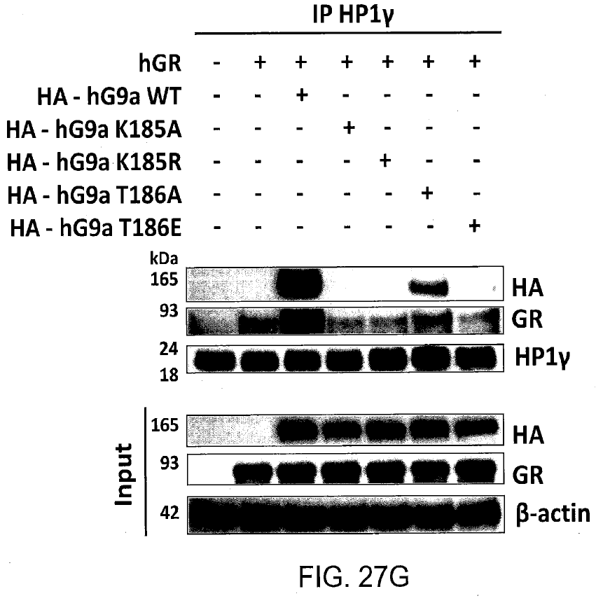

The results as shown in FIG. 27A highlight the effects of PTM site mutations on G9a methylation and phosphorylation. Cos-7 cells were transfected with plasmids encoding full length HA-hG9a wild type, or the K185R or T186A mutants. Lysates were immunoprecipitated with pan met-K or pan ph-T antibody and immunoblotted using antibodies listed.

Phosphorylation site mutation does not prevent G9a methylation in vitro as highlighted in the results as shown in FIG. 27B. N-terminal domain of hG9a wild-type, hG9a K185R or hG9a T186A mutants were incubated in vitro with GST-hG9a ΔN in the presence of [methyl-³H]SAM. Reaction products were analyzed by SDS-PAGE followed by fluorography. The corresponding Coomassi-stained gel is shown as a loading control.

The results as shown in FIG. 27C highlight the effects of Aurora kinase B activity on phosphorylation levels of G9a and GLP. Cos-7 cells were transfected with a plasmid encoding HA-hG9a or HA-hGLP and treated with 2 μM ZM447439 or vehicle DMSO for 24 h. Lysates were immunoprecipitated with pan ph-T antibody and immunoblotted with HA antibody. Expression of the indicated proteins in the Input sample is shown below.

The results as shown in FIG. 27D highlight the effects of Aurora kinase B activity on interaction of G9a with HP1γ. Cos-7 cells were transfected with a plasmid encoding HA-hG9a and treated with 2 μM ZM447439 or vehicle DMSO for 24 h as indicated. Lysates were immunoprecipitated with HP1γ antibody and immunoblotted with indicated antibodies. Expression of the indicated proteins in the Input sample is shown below.

102371 Aurora kinase B inhibition has no effect of phosphorylation of GR or HP1γ as highlighted in the results as shown in FIG. 27E. A549 cells were treated with 2 μM ZM447439 or vehicle DMSO for 24 h as indicated, or transfected with siRNA against Aurora kinase B (siAuroraB) or non-specific siRNA (siNS). Whole-cell extracts were analyzed with the listed antibodies by immunoblot.

The results as shown in FIG. 27F highlight the effects of Aurora kinase B overexpression on HP1γ-G9a interaction. Cos-7 cells were transfected with plasmids encoding HA-hG9a and Flag-aurora kinase B as indicated. Lysates were immunoprecipitated with HP1γ antibody and immunoblotted with the indicated antibodies. Expression of the indicated proteins in the Input sample is shown below.

The results as shown in FIG. 27G highlight the effects of G9a methylation and phophorylation site mutations on HP1γ interaction with G9a and GR. Cos-7 cell were transfected with plasmids encoding hGR and full length HA-hG9a wild type or with the indicated mutations. Lysates were immunoprecipitated with HP1γ antibody and immunoblotted with antibodies against HA, GR and HP1γ. Expression of the indicated proteins in the Input sample is shown below.

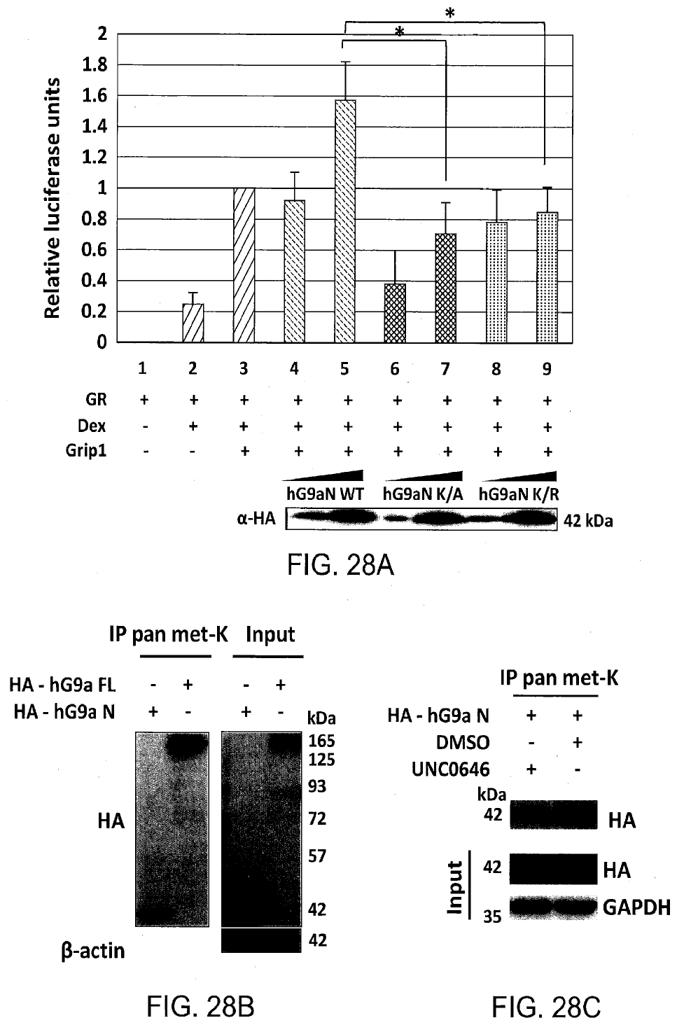

The results as shown in FIG. 28A highlight the role of K185 in the coactivator activity of the N-terminal fragment of G9a. Transient reporter gene assays were performed as in FIG. 4A with plasmids encoding HA-labeled hG9aN WT, hG9aN K185A, or hG9aN K185R (150 and 400 ng) as indicated. Relative luciferase units are normalized to sample 3 and represent mean±SEM for six independent experiments. p-value was calculated using a paired t-test. *p≤0.05.

The results as shown in FIG. 28B highlight methylation of N-terminal G9a fragment in cells. Cos-7 cells were transfected with plasmids encoding full length HA-hG9a or HA-hG9a N; lysates were immunoprecipitated with pan met-K antibody and immunoblotted with HA antibody (left). Immunoblot of HA-tagged proteins (top right) and β-actin (bottom right) in Input samples is shown for comparison.

G9a/GLP methyltransferase inhibitors reduce methylation of the N-terminal G9a fragment as highlighted in the results as shown in FIG. 28C. Cos-7 cells were transfected with a plasmid encoding HA-hG9a N and treated with 2 μM UNC0646 or vehicle DMSO for 24 h. Lysates were immunoprecipitated with pan met-K antibody and immunoblotted with HA antibody (top). Expression of the indicated proteins in the Input sample is shown below.

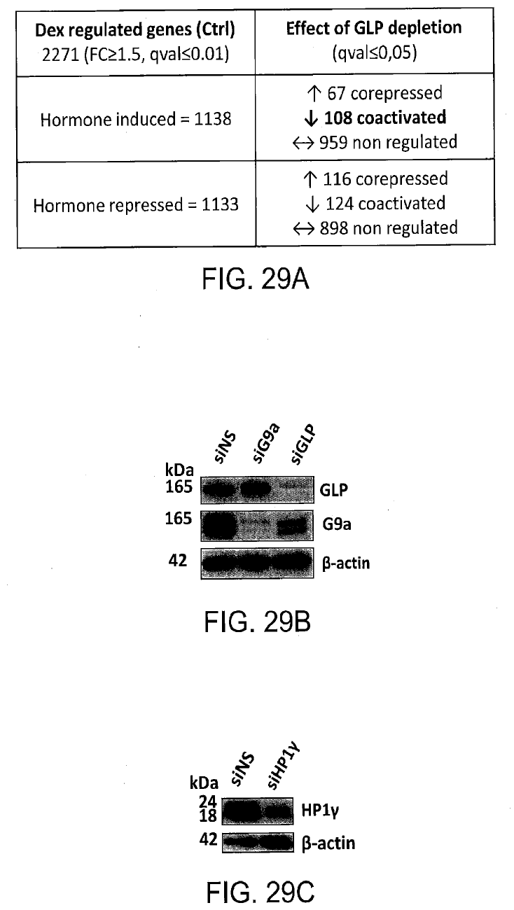

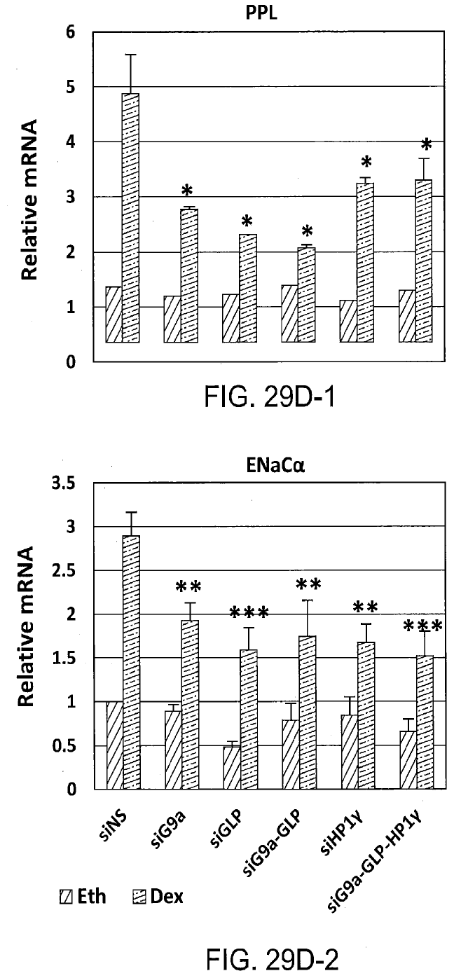

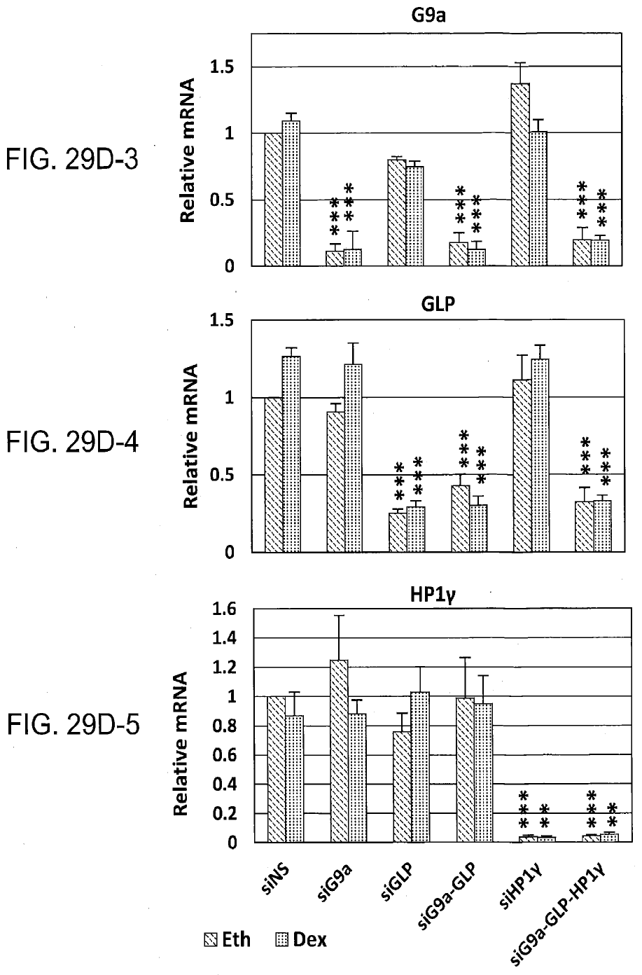

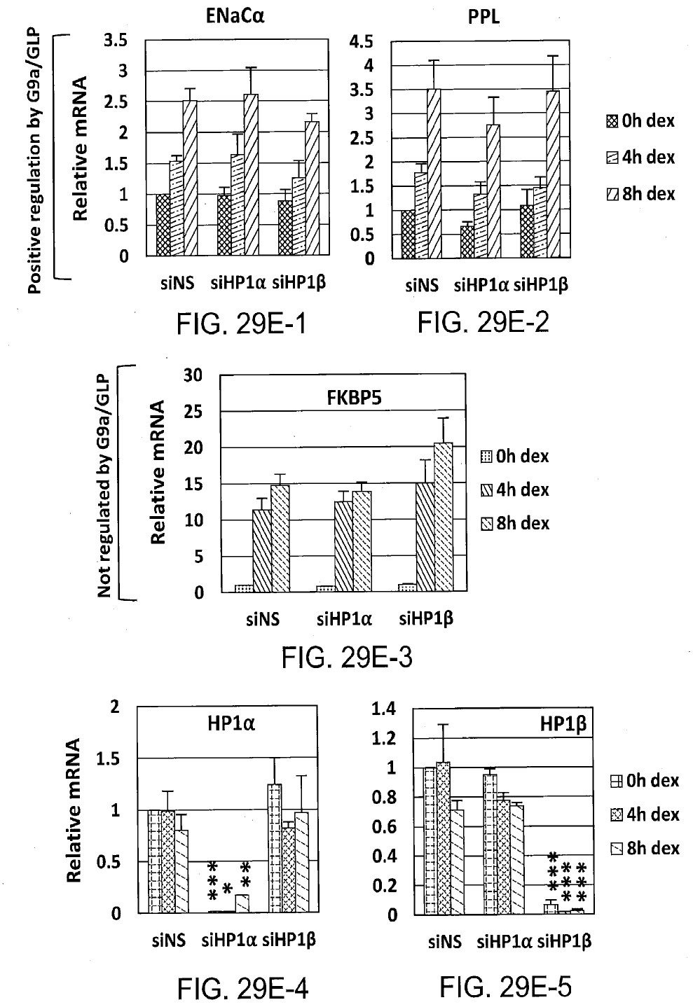

FIG. 29A is a table summarizing bioinformatics analysis of microarray results for effect of GLP depletion on the dex-regulated gene set. Number of hormone induced and repressed genes are shown on the left. These sets are subdivided on the right according to the effect of GLP depletion on the dex-regulated level of mRNA, as indicated by the arrows. Bold type indicates the 108 dex-induced genes that were coactivated by GLP and are further analyzed in FIG. 21C.

The results as shown in FIG. 29B highlight depletion of G9a and GLP. Cells were transfected with siRNA as indicated in FIGS. 21D-1, 21D-2, 21D-3, 21D-4, 21D-5, 21D-6, 21D-7, and 21D-8. Immunoblot shows G9a, GLP and β-actin protein levels in cell extracts from A549 cells after 8 h of dex treatment.

The results as shown in FIG. 29C highlight depletion of HP1γ. Cells were transfected with siRNA as indicated in FIG. 5E. Immunoblot shows HP1γ and β-actin protein levels in cell extracts from A549 cells after 8 h of dex treatment.

The results as shown in FIGS. 29D-1, 29D-2, 29D-3, 29D-4, and 29D-5 highlight the effects of depleting combinations of G9a, GLP, and HP1γ. A549 cells transfected with non-specific siRNA (siNS) or with SMART-pool siRNA targeting G9a (siG9a), GLP (siGLP), HP1γ (siHP1γ), or combinations of siRNAs as indicated, and treated for 8 hours with ethanol (eth) or 100 nM dex. mRNA levels for the indicated genes were measured by reverse transcriptase followed by qPCR and normalized to β-actin mRNA levels. Results shown are mean±SEM for five independent experiments. p-value was calculated using a paired t-test. *p≤0.05, **p≤0.01, * * *p≤0.001.

Depletion of HP 1α or HP1β has no effect on dex-regulated gene expression as highlighted in the results as shown in FIGS. 29E-1, 29E-2, 29E-3, 29E-4, and 29E-5. A549 cells transfected with non-specific siRNA (siNS) or with SMART-pool siRNA targeting HP1α (siHP1α) or HP1β (siHP1β) were treated with 100 nM dex for the indicated times (Oh dex indicates ethanol treatment for 8 h). mRNA levels for the indicated genes were measured by reverse transcriptase followed by qPCR and normalized to β-actin mRNA levels. Results shown are mean±SEM for four independent experiments. p-value was calculated using a paired t-test. *p≤0.05, **p≤0.01, * * *p≤0.001.

Dex induces HP1γ recruitment specifically to GBR of G9a/GLP-dependent GR target genes as highlighted in the results as shown in FIGS. 30A-1, 30A-2, 30A-3, 30A-4, 30A-5, and 30A-6. CUP was performed on A549 cells treated with 100 nM dex (darker bars) or ethanol (light bars) for 4 h, using antibodies against GR (upper panels) or HP1γ (lower panels); immunoprecipitated DNA was analyzed by qPCR using the indicated primers for the CDH16 gene (left) or ENaCαgene (right). Results are normalized to input chromatin and shown as mean±SD of triplicate PCR reactions performed with DNA samples from a single experiment, and are representative of two independent experiments. Arrows in diagrams represents transcription start sites (TSS); numbers indicate distance of amplified sequences from the TSS; GBR, GR binding region.

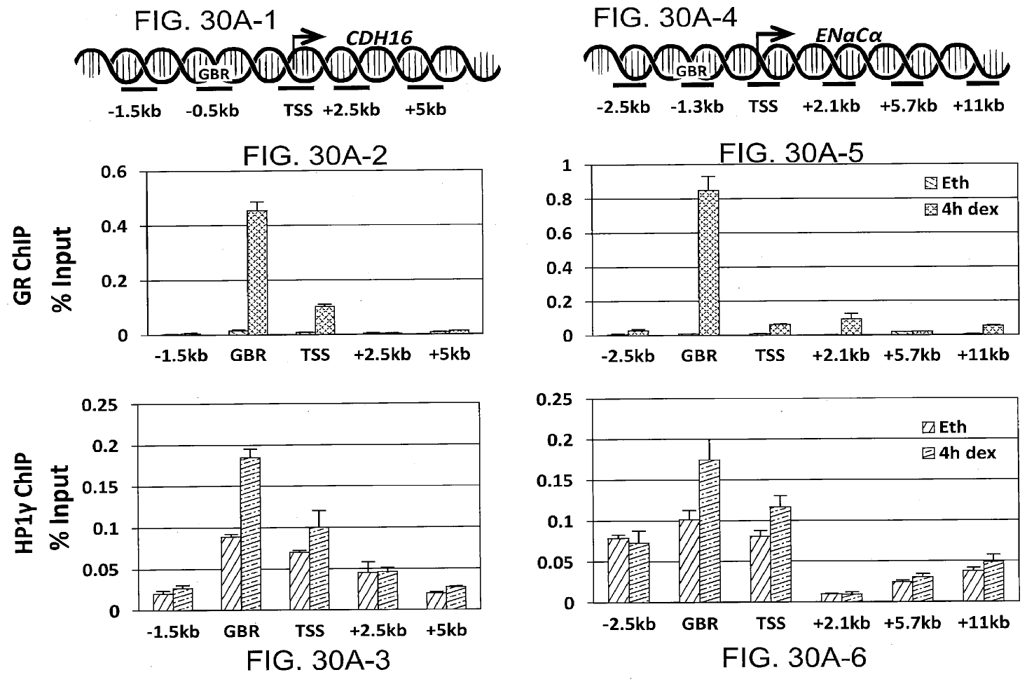

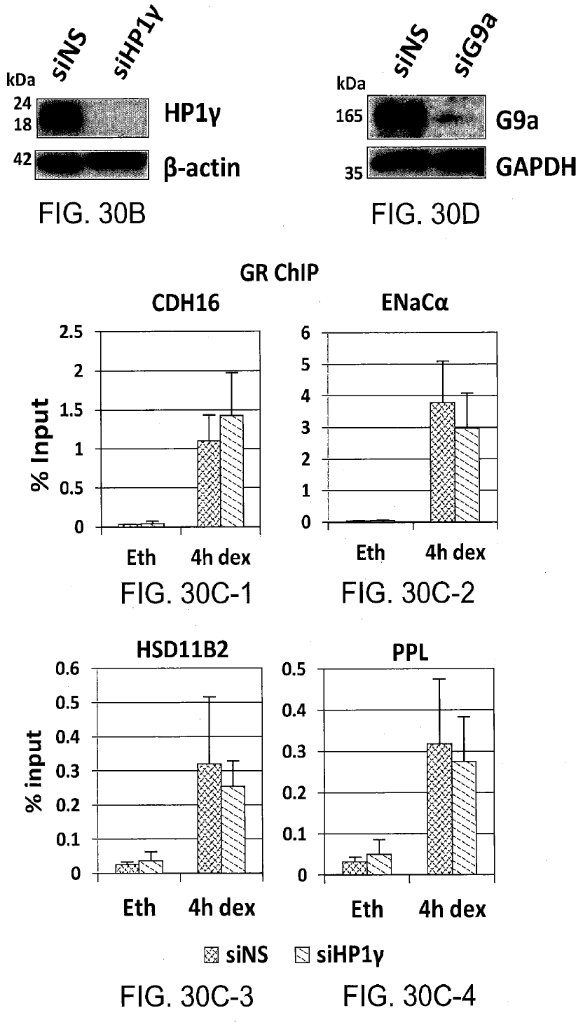

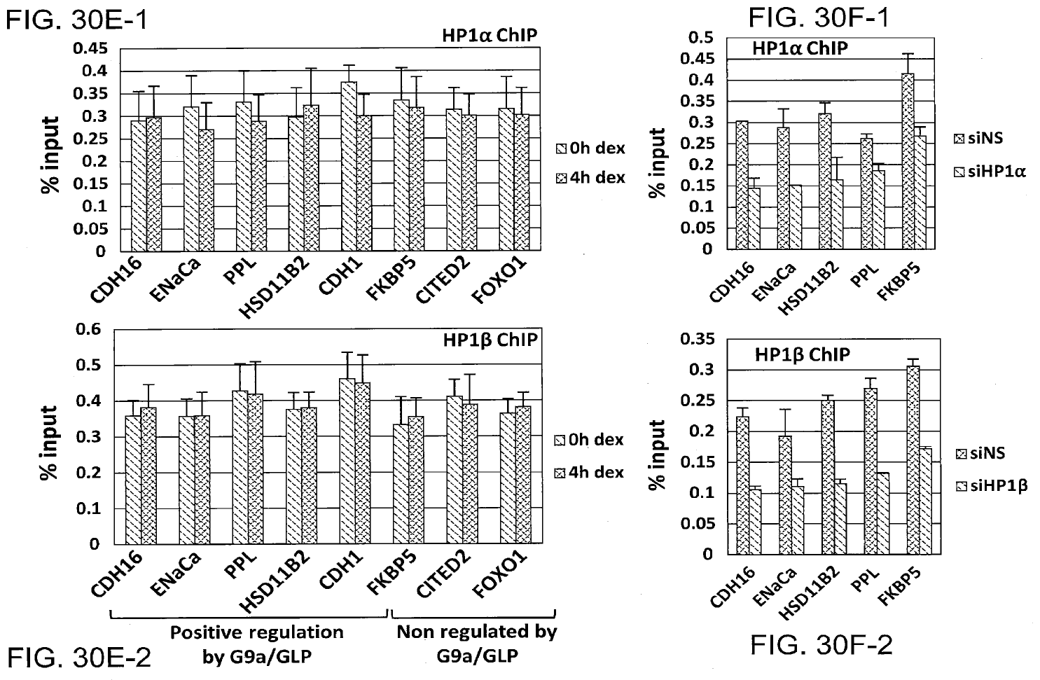

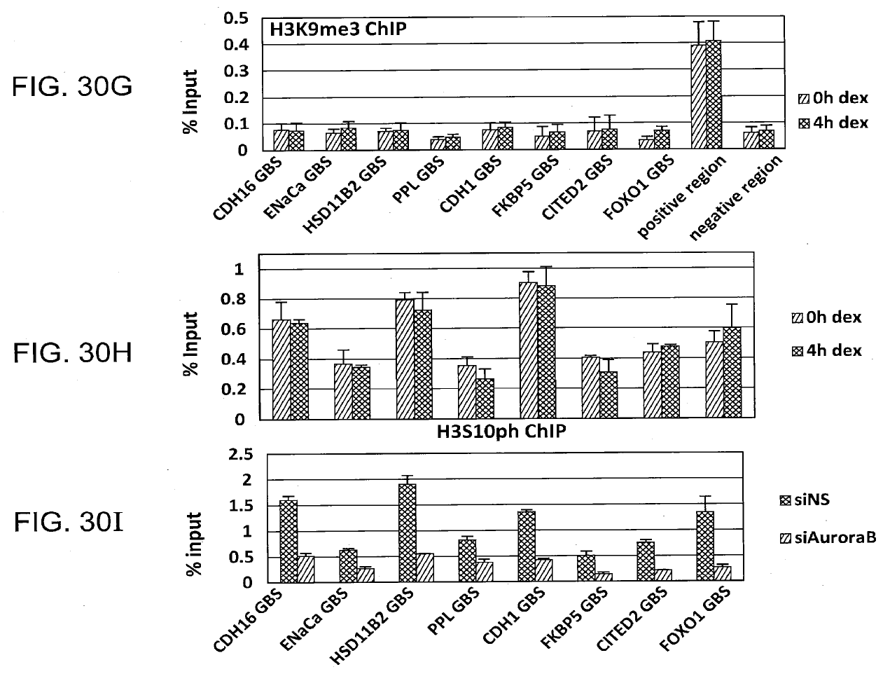

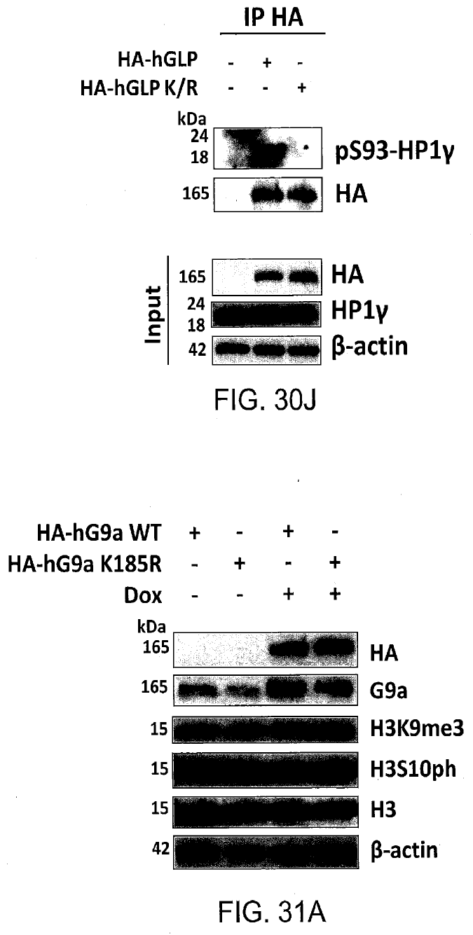

The results as shown in FIG. 30B highlight depletion of HP1γ. Immunoblot showing HP1γ and β-actin protein level in cell extracts from A549 cells transfected with the indicated siRNA and then treated for 4 h with dex.

HP1γ depletion has no effect on GR binding to GBR of target genes as highlighted in the results as shown in FIGS. 30C-1, 30C-2, 30C-3, and 30C-4. ChiP with GR antibodies was performed on A549 cells transfected with non-specific siRNA (siNS, dark bars) or HP1γ siRNA (siHP1γ, light bars) and treated with ethanol (Eth) or 100 nM dex for 4 h. Immunoprecipitated DNA was analyzed by qPCR using primers that amplify the GBR of the indicated GR target genes. Results shown are mean±SEM for four independent experiments. p-values calculated using a paired t-test were not significant for siNS versus siHP1γ samples from dex-treated cells.

The results as shown in FIG. 30D highlight depletion of G9a. Immunoblot showing G9a and GAPDH protein level in cell extracts from A549 cells transfected with the indicated siRNA and then treated for 4 h with dex.

HP1α or HP1β are not recruited to GBR of GR target genes in response to dex as highlighted in the results as shown in FIGS. 30E-1 and 30E-2. A549 cells were treated with 100 nM dex or ethanol for 4 h. ChIP was performed with antibody against HP1a (upper panel) or HP1β (lower panel), and immunoprecipitated DNA was analyzed by qPCR using primers that amplify the GBRs associated with the indicated GR target genes. Results are normalized to input chromatin and shown as mean±SEM for three independent experiments. p-values calculated using a paired t-test were not significant for samples treated with dex versus ethanol.

The results as shown in FIGS. 30E-1 and 30E-2 highlight the validation of HP1α or HP1β ChIP signals by siRNA depletion. A549 cells were transfected with non-specific siRNA (siNS, dark blue) or with SMART-pool siRNA targeting HP1α (siHP1α) or HP1β (siHP1β), and treated with 100 nM dex for 4 h. ChIP was performed with HP1α or HP1β antibodies, and immunoprecipitated DNA was analyzed by qPCR using primers that amplify the GBRs associated with the indicated GR target genes. Results are normalized to input chromatin and shown as mean±SD of triplicate PCR reactions performed with DNA samples from a single experiment, and is representative of two independent experiments.

Dex does not increase H3K9me3 on GBR of GR target genes as highlighted in the results as shown in FIG. 30G. A549 cells were treated with 100 nM dex or ethanol for 4 h. ChIP was performed with antibody against H3K9me3, and immunoprecipitated DNA was analyzed by qPCR using primers that amplify the GBRs associated with the indicated GR target genes or positive and negative control regions identified from previous H3K9me3 ChIP-seq of A549 cells. Results are normalized to input chromatin and shown as mean±SEM for three independent experiments. p-values calculated using a paired t-test were not significant for samples treated with dex versus ethanol.

Dex does not increase H3S10ph on GBR of GR target genes as highlighted in the results as shown in FIG. 30H. Experiments were performed as in G with H3S10ph antibody.

The results as shown in FIG. 30I highlight the validation of H3S10ph ChIP signals by siRNA depletion. A549 cells were transfected with non-specific siRNA (siNS, light bars) or with SMART-pool siRNA targeting aurora kinase B (siAuroraB) (dark bars), and treated with 100 nM dex for 4 h. ChIP was performed with antibodies against H3S10ph, and immunoprecipitated DNA was analyzed by qPCR using primers that amplify the GBRs associated with the indicated GR target genes. Results are normalized to input chromatin and shown as mean±SD of triplicate PCR reactions performed with DNA samples from a single experiment, and is representative of two independent experiments.

GLP K205 methylation is required for interaction with phospho-S93-HP1γ as highlighted in the results as shown in FIG. 30J. Cos-7 cells were transfected with plasmids encoding full length HA-hGLP wild type or K205R mutant. Lysates were immunoprecipitated (IP) with HA antibody and immunoblotted with antibodies against phospho-S93-HP1γ or HA. Expression of HA-tagged GLP, HP1γ and β-actin (loading control) in the unfractionated extracts is shown at the bottom (Input).

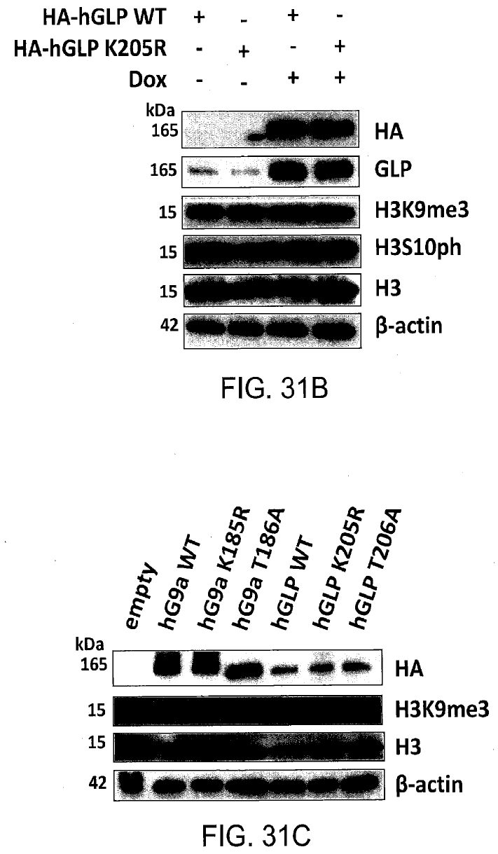

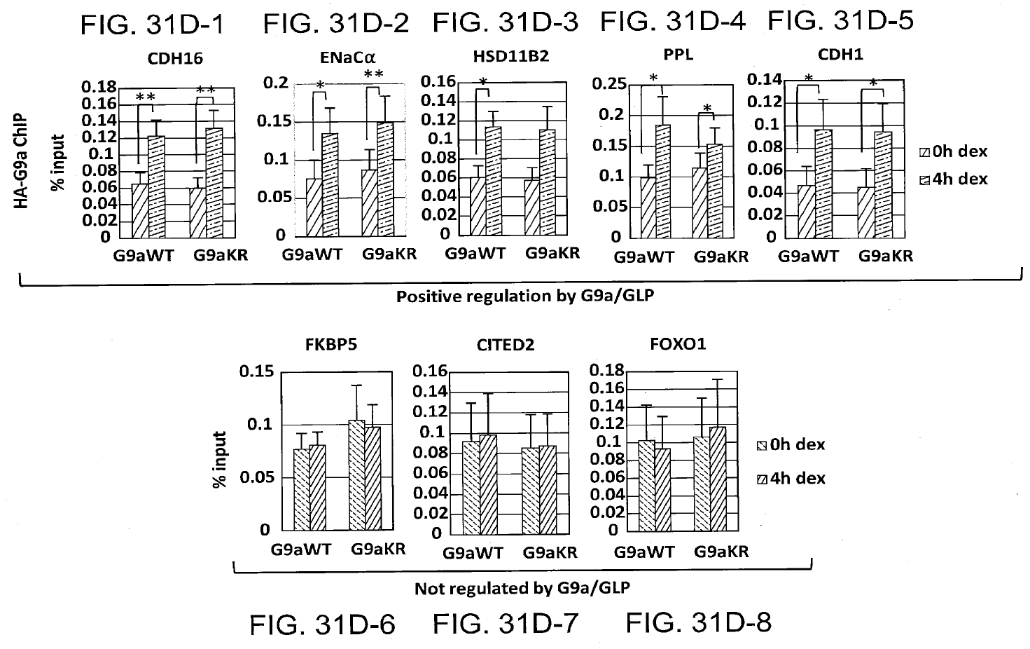

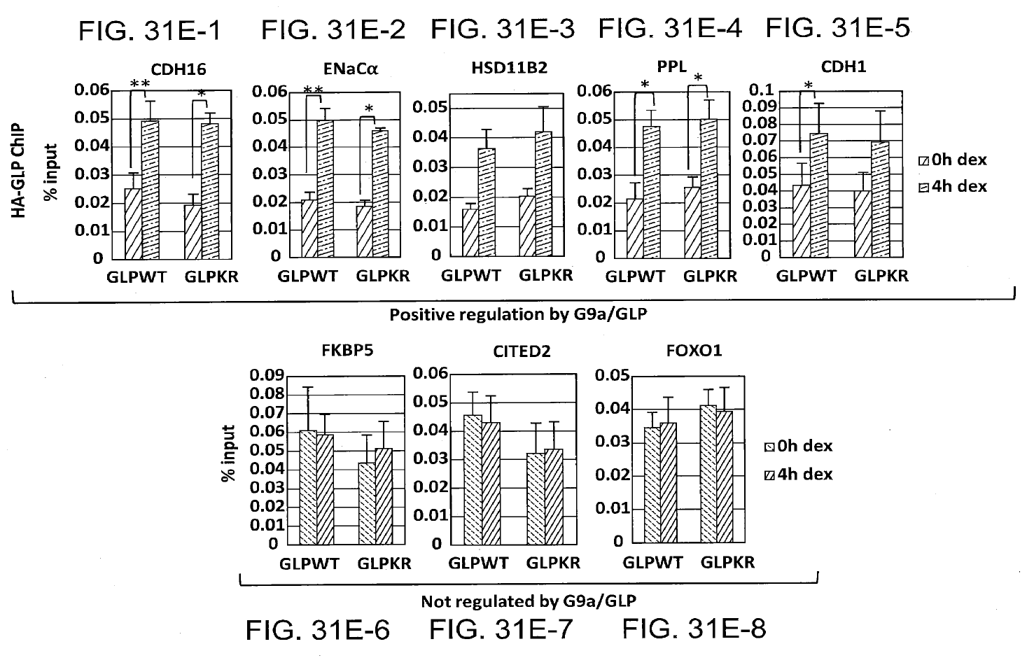

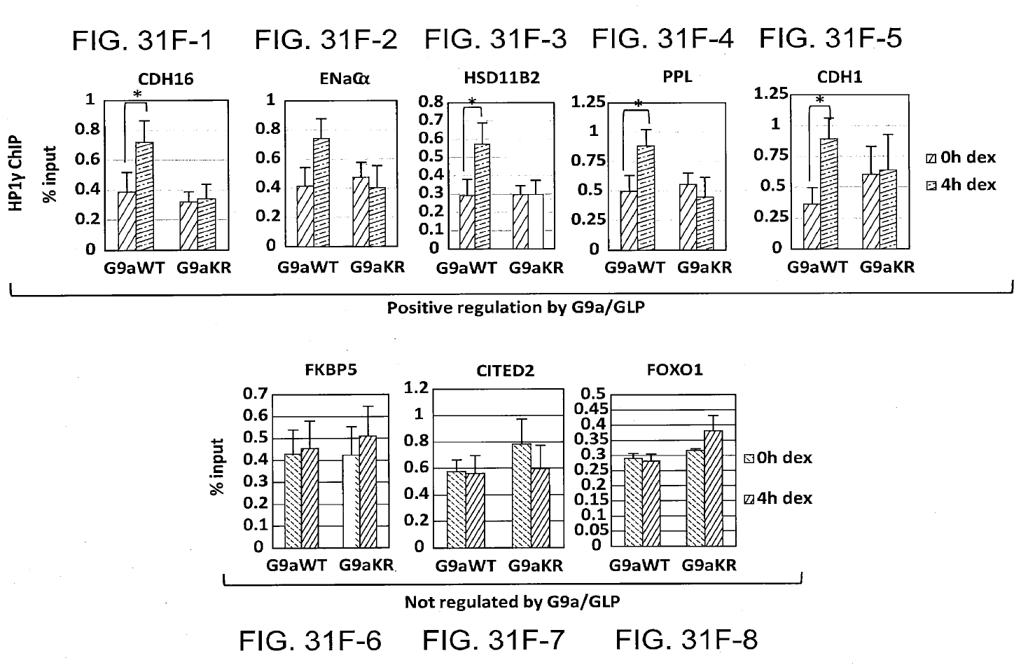

G9a methylation site mutation does not affect cellular levels of H3K9me3 or H3S10ph as highlighted in the results as shown in FIG. 31A. A549 rtta cell lines containing a stably-integrated doxycyline-regulated G9a WT or K185R transgene were treated with 10 ng/ml of doxycycline or DMSO vehicle for 24 h prior to and during 4 h of 100 nM dex treatment. A fraction of the cells was analyzed for G9a expression and histone modifications by immunoblot using indicated antibodies.

GLP methylation site mutation does not affect cellular levels of H3K9me3 or H3S10ph as highlighted in the results as shown in FIG. 31B. A549 rtta cell lines containing a stably-integrated doxycyline-regulated GLP WT or K205R transgene were treated with 50 ng/ml of doxycycline or DMSO vehicle for 24 h prior to and during 4 h of 100 nM dex treatment. A fraction of the cells was analyzed for GLP expression and histone modifications by immunoblot using indicated antibodies.

G9a or GLP PTM site mutations do not affect cellular levels of H3K9me3 as highlighted in the results as shown in FIG. 31C. Cos-7 cells were transfected with plasmids encoding full length HA-hG9a or HA-hGLP wild type or K/R and T/A mutants. Whole-cell extracts were analyzed for expression of HA, H3K9me3, H3 and β-actin by immunoblot.

Methylation site mutation does not affect G9a recruitment to GR target genes as highlighted in the results as shown in FIGS. 31D-1, 31D-2, 31D-3, 31D-4, 31D-5, 31D-6, 31D-7, and 31D-8. ChIP was performed on A549 cells from A using HA antibody, and immunoprecipitated DNA was analyzed by qPCR using primers specific for the GBRs associated with the indicated genes. Results are normalized to input chromatin and shown as mean±SEM for three independent experiments. p-value was calculated using a paired t-test. *p≤0.05, **p≤0.01.

Methylation site mutation does not affect GLP recruitment to GR target genes as highlighted in the results as shown in FIGS. 31E-1, 31E-2, 31E-3, 31E-4, 31E-5, 31E-6, 31E-7, and 31E-8. ChIP was performed on A549 cells from B using HA antibody, and immunoprecipitated DNA was analyzed by qPCR using primers specific for the GBRs associated with the indicated genes. Results are normalized to input chromatin and shown as mean±SEM for three independent experiments. p-value was calculated using a paired t-test. *p≤0.05, **p≤0.01.

Methylation of G9a is required for recruitment of HP1γ to GBR of GR target genes as highlighted in the results as shown in FIGS. 31F-1, 31F-2, 31F-3, 31F-4, 31F-5, 31F-6, 31F-7, and 31F-8. ChIP was performed on A549 cells from A using HP1γ antibody and immunoprecipitated DNA was analyzed by qPCR using primers specific for the GBRs associated with the indicated genes. Results are normalized to input chromatin and shown as mean±SEM for three independent experiments. p-value was calculated using a paired t-test. *p≤0.05.

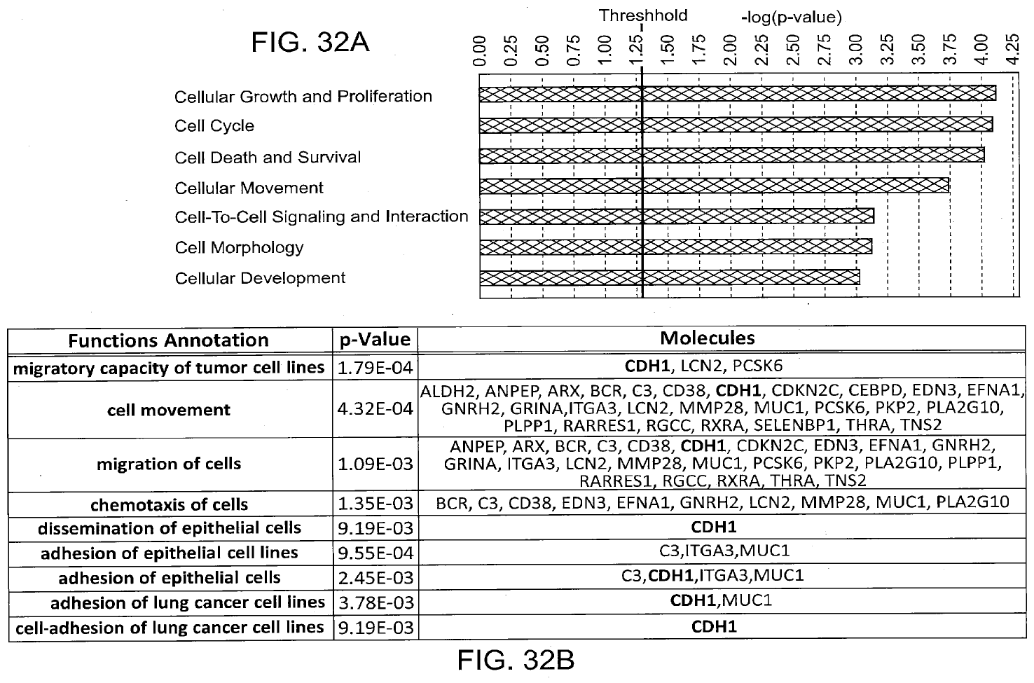

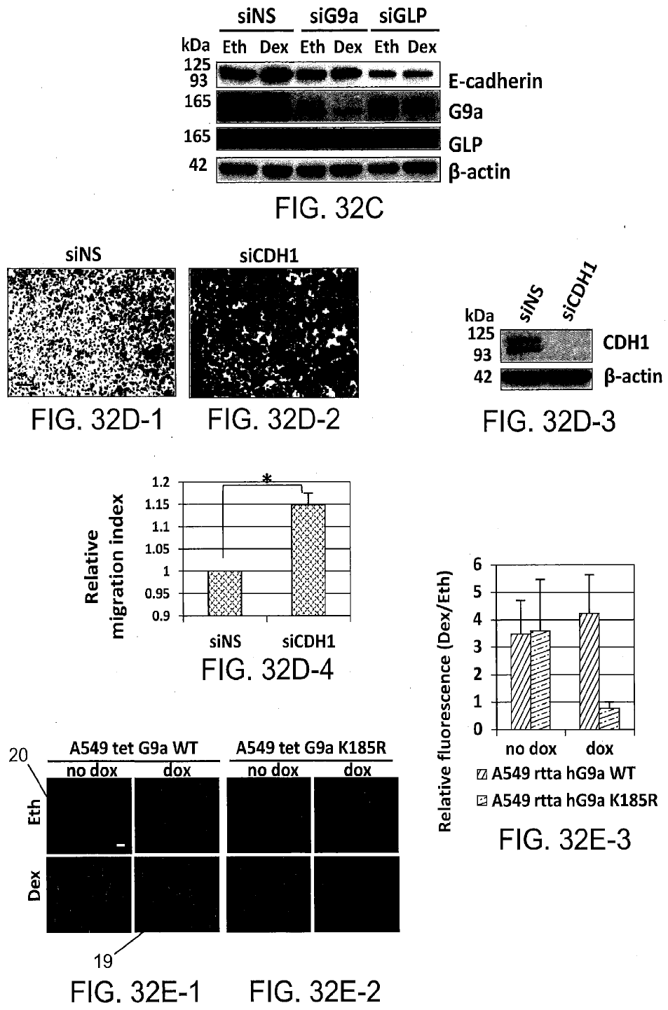

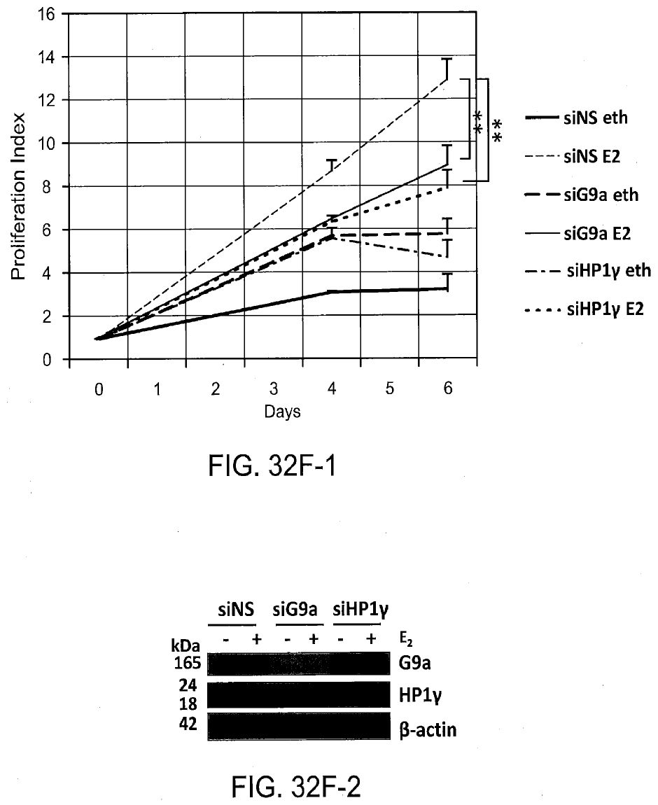

FIG. 32A is an Ingenuity Pathway Analysis (Version 01-07) on cellular functions of the 108 dex-activated genes that are coactivated by GLP in A549 cells (from FIG. 21C). The top categories are shown, and the threshhold for significance is indicated by the vertical orange line or the black open box.

As highlighted in results as shown in FIG. 32B, the enriched categories linked with cell movement from the analysis in A are shown, along with the p-value for the enrichment and the identities of genes included in each category. Highlighted gene is CDH1 (light gray) which was chosen for further analysis.

The results as shown in FIG. 32C highlight the effects of G9a and GLP depletion on dex regulation of E-cadherin expression. A549 cells transfected with non-specific siRNA or SMART-pool siRNA targeting G9a (siG9a) or GLP (siGLP) were treated with 100 nM dex or ethanol for 24 h. Immunoblotting was conducted with indicated antibodies.

The results as shown in FIGS. 32D-1, 32D-2, 32D-3, and 32D-4 highlight the effects of E-cadherin depletion on A549 cell migration. A549 cells were transfected with non-specific siRNA (siNS) or SMART-pool siRNA targeting E-cadherin (siCDH1). Cell migration was analyzed using Transwell migration assays. Migratory cells on the bottom of the polycarbonate membrane were stained. Representative images are shown (top panel). Scale bar represents 100 μm. Then, dye extracted from the cells was quantified at OD 560 nm. Relative migration index is shown as the mean±SEM of four independent experiments (right bottom panel). p-value was determined using a paired t-test. *p≤0.05. A fraction of the cells used for the Transwell migration assays were analyzed for E-cadherin expression by immunoblot using CDH1 antibody. β-actin is used as a loading control.

G9a methylation is required for dex induction of E-cadherin protein expression as highlighted in the results as shown in FIGS. 32E-1, 32E-2, and 32E-3. A549 rtta cell lines containing a stably-integrated doxycyline-regulated G9a WT or K185R transgene were treated with 10 ng/ml of doxycycline or vehicle DMSO for 24 h prior to and during 24 h of dex treatment. E-cadherin expression was analyzed by immunofluorescence (green 19). The nuclei were counterstained with DAPI (blue 20). Representative images are shown. Scale bar represents 10 μm. The ratios of E-cadherin expression in cells treated with dex versus ethanol, as determined by image analysis of at least 1500 cells per sample, are shown as the mean±SEM of three independent experiments.

G9a and HP1γ are required for estrogen-enhanced proliferation of MCF-7 breast cancer cells as highlighted in the results as shown in FIGS. 32F-1 and 32F-2. Cells transfected with the indicated siRNA were grown in medium containing 5% charcoal-stripped serum and free of phenol red for the indicated times with 10 nM estradiol (E2) or equivalent volume of ethanol. Results are from a single experiment which is representative of two independent experiments. Mean±SD, n=3 biological replicates, **p≤0.01. A fraction of the cells was analyzed for G9a, HP1γ and β-actin expression.

### Results

G9a and GLP Methylation are Required for Recruitment of HP1γ to a Complex with GR

To study possible effects of G9a and GLP methylation in cells, we first confirmed sites of G9a methylation and identified sites of GLP methylation. The sequence in the N-terminal domain of human G9a (hG9a) containing the methylation site is highly conserved with hGLP (FIG. 17A). Purified N-terminal domains of hG9a and hGLP or the mutant version with substitutions for the putative methylated lysines (K185R and K205R respectively) were incubated with [³H-methyl]S-adenosylmethionine (SAM) and a recombinant hG9a C-terminal fragment (amino acids 735-1210, hG9a ΔN) containing the enzymatic activity. Fluorography showed that N-terminal fragments of both hGLP and hG9a are methylated by hG9a ΔN (FIG. 24A). Substitution of K185 of hG9a or K205 of hGLP with arginine strongly decreased methylation. These data indicate that hG9a methylates hG9a and hGLP primarily on K185 and K205, respectively, in vitro.

In order to determine if G9a and GLP are methylated in cells, we found a pan-methyllysine antibody (developed to recognize methyllysine on a variety of methylated proteins) that did not recognize an unmethylated recombinant hG9a N-terminal fragment (amino acids 1-280) but interacted strongly with the G9a N-terminal fragment after in vitro methylation by hG9a ΔN (FIG. 17B-1, left panel). In contrast, the same N-terminal hG9a fragment with a K185R mutation was not recognized by the pan-methyllysine antibody after incubation in the methylation reaction, confirming K185 as the methylation site. Using the same approach, we found that hGLP is also auto-methylated on K205 (FIG. 17B-2, right panel). The N-terminal fragments of both G9a and GLP were methylated by the C-terminal fragment of either G9a or GLP (FIGS. 17B-1, 17B-2). Thus, while intramolecular auto-methylation is possible, G9a and GLP methylation can occur in-trans.

The pan-methyllysine antibody also recognized (by immunoprecipitation or immunoblot) wild type full length hG9a transiently expressed in Cos-7 cells, but not full length hG9a with the K185R mutation (FIG. 17C, left panel), confirming that G9a in cells is methylated on K185. Similarly, full length hGLP transiently expressed is methylated on the K205 (FIG. 17C, right panel). In addition, the signal from this antibody was strongly decreased when cells expressing wild type hG9a or hGLP were treated with small molecule inhibitors (UNC0646, UNC0638, UNC0642) specific for G9a and GLP methyltransferase activity [19, 20] (FIGS. 17D, 24B and 24C), or treated with the general SAM-dependent methylation inhibitor adenosine dialdehyde (Adox) (FIG. 24D), confirming that the signal detected on G9a and GLP in cells by the pan-methyllysine antibody is due to methylation. We also detected methylation of endogenous G9a and GLP in A549 human lung adenocarcinoma cells (FIGS. 17E-1, 17E-2), which were the primary cells used for G9a and GLP functional analyses in this study; in multiple experiments there was no consistent change in the G9a or GLP methylation level in response to dexamethasone (dex), the synthetic GR agonist used in this study. When A549 cells were treated with G9a/GLP methyltransferase inhibitor UNC0646, the endogenous level of G9a and GLP increased, but the proportion of G9a and GLP that was methylated decreased substantially (FIGS. 24E-1, 24E-2). The decreased methylation signal further validates the methylation of endogenous G9a and GLP, while the increased levels of G9a and GLP indicate that methylation somehow influences G9a and GLP protein production or turnover, but additional experiments are required to test the latter possibilities.

To explore the role of G9a/GLP methylation in binding to GR and coregulators HP1γ, GRIP1, p300, and CARM1 in the context of GR signaling, we first performed co-immunoprecipitation experiments with wild type and methylation site mutants of G9a and GLP. GR interacts in a hormone-independent manner with G9a via its N-terminal domain [4] and also with GLP (FIG. 25A). Mutation of the methylation site (K185) did not affect GR binding to G9a as determined by co-immunoprecipitation (FIG. 25B), indicating that G9a methylation is not involved in its interaction with GR. Similarly, mutation of the G9a methylation site did not affect its previously described interaction with coregulators GRIP1, p300, and CARM1 [4, 11] (FIG. 25C). It was previously shown that G9a methylation is essential for its interaction with HP1 γ [17] Likewise, when wild-type G9a or GLP or the corresponding methylation site point mutants were over-expressed in Cos-7 cells, the methylation site mutations almost eliminated co-immunoprecipitation of G9a and GLP with HP1γ (FIGS. 18A, 18B). Interestingly, GR also co-precipitated with HP1γ in these experiments, but only very weakly unless wild-type G9a or GLP was co-expressed (FIGS. 18A, 18B, 25B), indicating that the automethylation site is important for the formation of a ternary complex (GR-G9a/GLP-HP1γ), with either G9a or GLP binding HP1γ via the methylated lysine site and binding GR through a different site.

Importantly, we confirmed these observations for the endogenous proteins in A549 cells using proximity ligation assay technology (PLA). With this technique, protein-protein interactions are visualized by immunofluorescence, where each red dot represents a single molecular complex [21b]. HP1γ interacted with G9a in nuclei of A549 cells in a hormone independent manner (FIGS. 25D-1, 25D-2); depletion of either protein with siRNA eliminated most of the signal, validating the interaction and the antibodies used to detect it (FIGS. 25E-1, 25E-2). Moreover, we established stable cell lines where expression of wild-type or K/R mutant G9a or GLP (containing an N-terminal HA-tag) are doxycycline inducible. In this system, HP1γ interacted significantly less with G9a/GLP methylation site mutants than with wild-type G9a/GLP (FIGS. 26A-1, 26A-2, 26B-1, 26B-2). Moreover, HP1γ also associated with GR, and this interaction was highly dependent on treatment of cells with dex (FIGS. 18C-1, 18C-2, 18C-3), presumably due to the nuclear localization of GR caused by dex; depletion of HP1γ further validated the detection of the complex by PLA (FIGS. 25F-1, 25F-2). The dex-induced GR-HP1γ interaction was also inhibited by the depletion of G9a or GLP (FIGS. 18D-1, 18D-2, 18D-3, 18D-4, 18D-5), thus validating the ternary complex GR-G9a/GLP-HP1γ. Depletion of GLP also caused depletion of G9a protein (FIGS. 118D-1, 18D-2, 18D-3, 18D-4, 18D-5), since the stability of G9a protein depends on the presence of GLP [22b]. Therefore, while G9a is clearly required for the association between GR and HP1γ, we cannot conclude whether GLP is also directly involved. GR-HP1γ interaction in PLA was also strongly decreased when cells were treated with G9a/GLP methyltransferase inhibitor UNC0646 (FIGS. 18E-1, 18E-2, 18E-3), consistent with our observation that G9a/GLP methylation is crucial for GR-G9a/GLP-HP1γ ternary complex formation. Moreover, over-expression of the methylation site mutant of G9a or GLP (but not over-expression of wild type G9a or GLP) inhibited the GR-HP1γ interaction (FIGS. 26C-1, 26C-2, 26C-3, 26D-1, 26D-2, 26D-3). Thus G9a and/or GLP nucleates a ternary complex with GR and HP1γ, and methylation of G9a K185 or GLP K205 is required for their interactions with HP1γ.

### G9a and GLP Phosphorylation by Aurora Kinase B Antagonizes HP1γ Recognition

Since aurora kinase B (also known as AURKB) was previously shown to phosphorylate G9a at T186 in a cell-free reaction[17b], we tested whether this occurred in cells. Using an approach similar to that described above for detecting methylation, we validated a pan-phosphothreonine antibody to detect G9a phosphorylation at T186 in cells. The pan-phosphothreonine antibody recognized over-expressed wild type G9a but not the T186A mutant in immunoprecipitation and immunoblot experiments, thus indicating that G9a is phosphorylated on T186 in Cos-7 cells (FIG. 19A, left panel). Likewise, we demonstrated for the first time that GLP is phosphorylated in cells on T206 (FIG. 19A, right panel). Depletion of aurora kinase B from Cos-7 cells with siRNA strongly decreased the phosphorylation detected by immunoprecipitation with the pan-phosphothreonine antibody followed by immunoblot with antibody against the HA epitope-labeled G9a or GLP (FIG. 19B, upper panel), confirming that aurora kinase B phosphorylates G9a and GLP in cells. Using the same detection strategy, we demonstrated that endogenous G9a and GLP are phosphorylated in A549 cells, in a hormone independent manner (FIGS. 17E-1, 17E-2). Interestingly, inhibition of G9a and GLP phosphorylation by depleting aurora kinase B from cells increased the interaction between HP1γ and G9a or GLP (FIG. 19B, lower panels). Consistent with this result, inhibition of aurora kinase B kinase activity with a specific inhibitor (ZM447439) decreased G9a and GLP phosphorylation signals (FIG. 27C) and increased the interaction between HP1γ and G9a (FIG. 27D). However, inhibition of Aurora kinase B activity did not affect GR or HP1γ phosphorylation (FIG. 27E). Overexpression of aurora kinase B had the opposite effect, decreasing the HP1γ-G9a interaction (FIG. 27F). Furthermore, we found that mutations of either the methylation site (K185) or phosphorylation site (T186) of G9a inhibited co-immunoprecipitation of G9a and GR with HP1γ (FIG. 27G). A phospho-mimic mutation T186E prevented co-immunoprecipitation of G9a and GR with HP1γ, confirming the effect of the phosphorylation on the binding of HP1γ to G9a. Also, we observed that the T186A mutation decreased the interaction between G9a and HP1γ, presumably because unmodified T186 is part of the recognition sequence of HP1γ. As a control, we showed that the methylation site mutation did not prevent phosphorylation of G9a in cells, and the phosphorylation site mutation did not prevent methylation (FIG. 27A). Similarly, in cell free methylation reactions the phosphorylation site mutation did not prevent G9a methylation (FIG. 27B). We conclude that G9a or GLP phosphorylation by aurora kinase B in cells prevents HP17 recognition.

### G9a and GLP Coactivator Function Requires HP1γ and is Regulated by Automethylation and Phosphorylation

As G9a and GLP PTMs occur in the N-terminal domain that is required for the coactivator function, we investigated the role of G9a and GLP PTMs in the regulation of their coactivator function, first using transient luciferase reporter genes. As shown previously [11b], G9a is not a very effective coactivator for steroid receptors by itself but acts cooperatively with coactivator GRIP1. Thus, when GR and coregulator GRIP1 were overexpressed by transient transfection of CV-1 cells, dex-induced expression of a GR-regulated reporter gene was enhanced by coexpression of full-length G9a (FIG. 20A, bars 4-5). In contrast the K185A and K185R mutants of full-length G9a were significantly less active (FIG. 20A, bars 6-9), although mutant and wild type hG9a were expressed at similar levels. Similar results were obtained when the N-terminal fragment of hG9a (amino acids 1-280 with wild type sequence or substitutions for K185) was used instead of full length G9a (FIG. 28A), consistent with our previous finding that this N-terminal fragment is necessary and sufficient for G9a coactivator function in these transient reporter gene assays [12b]. Thus, the lysine at residue 185 is required for the full coactivator function of G9a in this assay. Likewise, in the same system, dex-induced expression of the GR-regulated reporter gene was enhanced by coexpression of full-length GLP (FIG. 20B, bars 4-5), indicating that GLP, as well as G9a, can act as a coactivator of GR. In contrast, the K205R mutant of GLP is less active (FIG. 20B).

If K185 methylation is necessary for G9a coactivator function, then we would expect that the N-terminal fragment must be methylated in order to function as a coactivator; but G9a catalytic activity is localized in the C-terminal domain, suggesting that methylation of the N-terminal fragment would need to occur in trans. We found that the N-terminal fragment of G9a is indeed methylated when over-expressed in Cos-7 cells, but at a lower efficiency compared with over-expressed full length G9a (FIG. 28B), and treatment of the cells with the G9a/GLP inhibitor UNC0646 decreased methylation of the N-terminal fragment (FIG. 28C) as well as full length G9a (FIG. 17D). This result indicates that G9a and GLP can be methylated in trans in cells. Consistent with this, methyltransferase assays in vitro with G9a and GLP fragments also demonstrated that methylation of G9a or GLP can happen in trans (FIGS. 17B-1, 17B-2).

Since phosphorylation of G9a on T186 or GLP on T206 inhibits binding to HP17 (FIG. 19A), we next studied the impact of G9a and GLP phosphorylation on its coactivator function. In transient luciferase reporter gene assays the coactivator function of G9a and GLP, in cooperation with GRIP1, was significantly enhanced by the specific aurora kinase enzyme inhibitor ZM447439 (FIGS. 20C, 20D, bars 6-7 in comparison to bars 4-5). This finding further supports the roles of G9a/GLP PTMs and HP1γ in G9a and GLP coactivator function.

To characterize the effect of these PTMs on the endogenous target genes that are induced by dex-activated GR, we used gene expression microarray profiling to identify genes that require G9a and GLP for activation by dex and GR. The subset of GR target genes positively regulated by G9a in A549 cells was already identified by comparing cells expressing shRNA against G9a (shG9a) with cells expressing a non-specific shRNA (shNS) [4b]. A similar analysis with shGLP was performed in parallel with the previously published shG9a analysis and is reported shown in FIG. 33. As indicated above (FIGS. 18D-1, 18D-2, 18D-3, 18D-4, 18D-5), both GLP and G9a were depleted by shGLP in the samples analyzed by microarray (FIG. 21A). We identified 1254 genes for which mRNA level was significantly different (no fold cutoff was imposed) in the 24 h dex-treated shGLP cells vs. the dex-treated shNS control cells (FIG. 21B). The expression of 2271 genes was significantly changed by at least 1.5 fold after 24 h of dex treatment, and 415 of the total 2271 dex-regulated set of genes also belonged to the GLP-regulated gene set (FIG. 21B). By comparison, 122 of the 2271 dex-regulated genes were also significantly regulated by G9a [4b], and the majority of the G9a-regulated gene set overlapped with the GLP-regulated gene set. Of the 415 genes significantly regulated by dex and GLP, 240 (116+124 in the table) were repressed by dex and 175 (67+108 in the table) were activated by dex (FIG. 29A, right panels). Interestingly, from the 175 genes that were activated by dex and significantly regulated by GLP, 108 were induced less upon GLP depletion, indicating a putative coactivator function for GLP on these genes (FIG. 29A and FIG. 21C, darker bars). Moreover, the great majority among these 108 genes that required GLP for their dex-induced expression also required G9a for optimal dex-induced expression (FIG. 21C, lighter bars), as indicated by the negative fold change in expression due to GLP or G9a depletion (by comparing gene expression profiles in the dex-treated cells expressing shNS and the dex-treated shGLP or shG9a cells). Even if they were not always significantly regulated by G9a, the effect of G9a depletion in the previous microarray analysis [4b] was in the same direction as that for GLP depletion. However, there were a few GR target genes that were strongly dependent on GLP as a coactivator for their dex-induced expression, but were affected little or not at all by depletion of G9a (FIG. 21C). This demonstrates that although G9a and GLP largely supported the same genes, there was a smaller number of GR target genes that required GLP but not G9a for dex-induced expression.

As validation of the microarray results, quantitative RT-PCR showed that depletion of G9a or GLP by siRNAs (FIG. 29B) significantly decreased dex-induced expression of specific G9a- and GLP-dependent GR target genes (FIGS. 21D-1, 21D-2, 21D-3, 21D-4, 21D-5), but had little or no effect on dex-induced expression of genes that do not require G9a or GLP (FIGS. 21D-6, 21D-7, 21D-8). The GR target genes selected for validation and further mechanistic studies included three genes that were significantly dependent on GLP for dex-induced expression in the microarray analysis of 24 h-dex-treated cells (CDH1, CDH16, and PPL), one gene that was not quite significant in the above shGLP microarray but required GLP significantly after shorter periods of dex treatment (HSD11B2), and one gene that was previously shown to be G9a-dependent for dex-induced expression (ENaCα, also called SCNN1A) [4]; three GR target genes that were not dependent on G9a or GLP for dex-induced expression were also chosen, to serve as controls in various functional studies. In addition to these properties, these genes were selected because of strong response to dex, making it easier to observe effects of coregulator depletion, and well-documented GR binding sites associated with them [23b].

As we previously demonstrated that methylation of G9a K185 and GLP K205 facilitates recruitment of HP1γ (FIG. 18), we next analyzed the importance of HP1γ for dex-induced expression of endogenous GR target genes that are positively regulated in A549 cells by G9a or GLP. We depleted HP1γ using a pool of four siRNAs (FIG. 29C) and measured mRNA levels of the same eight endogenous GR target genes. Dex-induced levels of mRNAs for the G9a- and GLP-dependent genes, CDH16, ENaCα, PPL, HSD11B2 and CDH1 were significantly reduced by HP12γ depletion (FIGS. 21E-1, 21E-2, 21E-3, 21E-4, 21E-5), indicating a positive regulatory effect of HP1γ. However, induction of mRNAs from G9a- and GLP-independent GR target genes, FKBP5, FOXO1 and CITED2, by dex was not affected by HP1γ depletion (FIGS. 21E-6, 21E-7, 21E-8). Depletion of pairs or all three of the G9a, GLP and HP1γ coregulators did not have a greater effect than individual depletion of any of them indicating that these coregulators all function in the same pathway (FIGS. 29D-1, 29D-2, 29D-3, 29D-4, 29D-5).

As HP1γ is part of the HP1 family of proteins, we analyzed the involvement of the other two family members, HP1α and HP1β, in the dex-induced expression of these genes. Depletion of HP1α or HP1β did not affect the dex-induced expression of the G9a/GLP-dependent or G9a/GLP-independent GR target genes (FIGS. 29E-1, 29E-2, 29E-3, 29E-4, 29E-5). These results indicate that endogenous HP1γ is selectively required for full induction by dex of the endogenous GR target genes that are positively regulated by G9a and GLP and thus is required for G9a and GLP coactivator function.

To explore the role of G9a and GLP phosphorylation on G9a and GLP coactivator function, we analyzed the expression of the same eight endogenous GR target genes after treatment of the A549 cells with ZM447439 inhibitor. We observed significant increases of dex-induced CDH16, ENaCα, PPL, HSD11B2 and CDH1 mRNA levels in comparison to cells not treated with the inhibitor (FIGS. 21F-1, 21F-2, 21F-3, 21F-4, 21F-5). However, induction of mRNAs for the G9a- and GLP-independent GR target genes, FKBP5, FOXO1 and CITED2, by dex was not significantly altered by inhibition of the kinase activity of aurora kinase B (FIGS. 21F-6, 21F-7, 21F-8). As G9a phosphorylation is reduced by inhibition of aurora kinase B in cells, we conclude that the selective increase in the dex-induced expression of GR target genes that required G9a, GLP and HP1γ as coactivators is due to enhanced binding of HP1γ to G9a and/or GLP. HP1γ is recruited to GR binding regions associated with G9a/GLP-dependent GR target genes and facilitates recruitment of RNA polymerase II

G9a is selectively recruited to GR binding regions (GBR) associated with GR target genes that require G9a for their dex-induced expression [4b]. To test whether the GR-G9a-HP1γ complex we observed by coimmunoprecipitation and PLA assay (FIGS. 18A, 18B, 18C-1, 18C-2, 18C-3, 18D-1, 18D-2, 18D-3, 18D-4, 18D-5, 18E-1, 18E-2, 18E-3) forms on the GBR associated with G9a/GLP-dependent GR target genes, we tested for dex-induced occupancy of HP1γ on GBR associated with the same G9a/GLP-dependent and G9a/GLP-independent GR target genes that were analyzed above for expression. In chromatin immunoprecipitation (ChIP) analyses we observed dex-induced HP1γ occupancy on the GBRs closely associated with the CDH16 and ENaCα genes which are G9a/GLP-dependent GR target genes (FIGS. 30A-1, 30A-2, 30A-3, 30A-4). However, little or no dex-induced enhancement of HP1γ occupancy was observed at other sites in and around the CDH16 and ENaCα genes, except for a modest enhancement at the transcription start sites (TSS) where some GR occupancy was also observed. Dex-induced enhancement of HP1γ occupancy was also observed on GBRs associated with three other genes (PPL, HSD11B2 and CDH1) that require G9a and GLP for their dex-induced expression (FIG. 22A, left panel, darker bars). Importantly, when HP1γ was depleted with a pool of four siRNAs (FIG. 30B), hormone-induced HP1γ occupancy at the GBRs of all five of these G9a/GLP-dependent GR target genes was abolished (FIG. 22A-1, lighter bars), validating the specificity of the HP1γ ChIP enrichment using this antibody. GR occupancy at the GBRs of the G9a/GLP-dependent GR target genes was not affected by HP1γ depletion (FIGS. 30C-1, 30C-2, 30C-3, 30C-4).

In contrast to the G9a- and GLP-dependent GR target genes, no dex-induced enhancement of HP1γ occupancy was observed at GBRs associated with the FKBP5, CITED2 and FOXO1 genes (FIG. 22A-2, darker bars), which do not require G9a or GLP for their dex-induced expression (FIGS. 21D-1, 21D-2, 21D-3, 21D-4, 21D-5, 21D-6, 21D-7, 21D-8) and exhibit no dex-induced occupancy of G9a [4b] (FIGS. 31D-1, 31D-2, 31D-3, 31D-4, 31D-5, 31D-6, 31D-7D-8) on the associated GBRs. It is interesting to note that some HP1γ occupancy was observed on most of the above eight GBRs even in the absence of dex, as indicated by the reduction in the ChIP signal observed in the cells treated with ethanol (the vehicle for dex) after HP1γ depletion (FIGS. 22A-1, 22A-2, lighter bars). Similarly, higher-than-background HP1γ ChIP signals on some non-GBR sites associated with the CHD16 and ENaCα genes in ethanol-treated cells indicate constitutive HP1γ occupancy (FIGS. 30A-1, 30A-2, 30A-3, 30A-4, 30A-5, 30A-6). Thus, HP1γ occupancy was observed on all of the eight GBRs (and some other sites in and around these genes) prior to dex treatment, but was enhanced after dex treatment only on the GBRs of G9a/GLP-dependent GR target genes (FIG. 22A).

Since dex-induced occupancy of HP1γ (FIGS. 22A-1, 22A-2) corresponded to dex-induced occupancy of G9a [4b] (FIGS. 31D-1, 31D-2, 31D-3, 31D-4, 31D-5, 31D-6, 31D-7, 31D-8), we tested whether G9a is required for dex-induced HP1γ occupancy on the GBRs of the GR target genes. Indeed, depletion of G9a using a pool of four siRNAs (FIG. 30D) essentially eliminated the dex-dependent HP1γ recruitment specifically on GBRs of GR target genes that are positively regulated by G9a and GLP (FIG. 22B); in contrast, the constitutive, non-dex-inducible HP1γ occupancy observed on the GBRs of the G9a/GLP-independent GR target genes (FIG. 22A-2 panel) was not affected by G9a depletion (FIG. 22B).

As it was previously shown that HP1α and HP1β, in addition to HP1γ, bind methylated G9a [16b], we analyzed HP1α and HP1β recruitment on the GBRs of the GR target genes previously studied. There was no dex-induced enrichment of HP1α or HP1β on the GBRs of the G9a/GLP-dependent or G9a/GLP-independent GR target genes (FIGS. 30E-1, 30E-2). However, when HP1α or HP1β was depleted with a pool of four siRNAs, their occupancy at the GBRs decreased, showing there was some constitutive occupancy and validating the ChIP signals from the antibodies used (FIGS. 30E-1, 30E-2).

A similar PTM switch (adjacent methylation and phosphorylation sites) exists on histone H3, i.e. H3K9me3 recruits HP1γ and H3S10ph opposes this effect [24b, 25b]. Since these histone H3 PTMs could also affect the expression of the GR target genes, we analyzed H3K9me3 and H3S10ph levels at the GBR associated with the GR target genes of interest. ChIP experiments showed that H3K9me3 levels at these GBR were near background levels and did not increase with dex treatment (FIG. 30G). A region with high H3K9me3 occupancy served as a positive control. H3S10ph levels varied at the different GR binding sites but also did not change with dex treatment (FIG. 30H). Depletion of Aurora kinase B reduced the signals at all of these sites and thus validated the ChIP signal (FIG. 30I). Since H3K9me3 and H3S10ph were not increased by dex, they are not responsible for the dex-dependent binding of HP1γ to these sites or the dex-induced expression of these genes.

To study the role of G9a/GLP methylation in HP1γ recruitment to GBR of G9a/GLP-dependent GR target genes, we established stable cell lines where expression of wild-type or K/R mutant G9a or GLP (containing an N-terminal HA-tag) are doxycycline inducible (FIGS. 31A, 31B). We first validated the fact that overexpression of G9a/GLP wild-type, K/R and T/A mutants does not have any impact on total cellular H3K9me3 or H3S 10ph levels (FIGS. 31A-31C). In ChIP experiments using HA antibody, mutation of the methylation site did not reduce dex-induced G9a and GLP occupancy on the GBRs of G9a/GLP-dependent GR target genes (FIGS. 31D-31E). As expected, there was no dex-induced G9a and GLP occupancy on the GBRs of G9a/GLP-independent GR target genes. Dex-dependent HP1γ recruitment observed in cell lines over-expressing wild type G9a was eliminated in the cell lines that over-express the unmethylatable mutant G9a (FIGS. 31F-1, 31F-2, 31F-3, 31F-4, 31F-5, 31F-6, 31F-7, 31F-8), indicating that methylation of this lysine is a prerequisite for dex-induced HP1γ occupancy on the GBRs of G9a/GLP-dependent GR target genes. Altogether these results indicate that dex-induced HP1γ recruitment requires G9a/GLP methylation and is specific for the subset of GR target genes where G9a is recruited by GR and is required as a coactivator. In contrast, the constitutive HP1γ occupancy does not require G9a.

To explore the mechanism by which HP1γ contributes to dex-induced expression of G9a/GLP-dependent target genes, we used possible clues from previous reports that HP1γ is phosphorylated by Pim-1 and PKA [26b, 27b], that phosphorylation of HP1γ on S93 impaired its repression activity [26b, 27b], that HP1γ interacts with RNA polymerase II [6b], and that phospho-S93-HP1γ interacts with RNA polymerase II that is phosphorylated on S5 of the C-terminal repeat domain [27b]. We observed that wild-type G9a or GLP, but not the unmethylatable mutants, co-immunoprecipitated with phospho-S93-HP1γ (FIGS. 22C and 30J). In ChIP experiments, occupancy of phospho-S93-HP1γ on GBRs of G9a/GLP-dependent GR target genes (but not on G9a/GLP-independent GR target genes) was significantly induced by dex, and the dex-induced ChIP signal was eliminated by depletion of HP1γ (FIG. 22D). G9a was previously reported to be important for dex-induced RNA polymerase II occupancy of TSS associated with G9a-dependent GR target genes [12b]; and we observed that dex-induced occupancy by RNA polymerase II at TSS of G9a/GLP-dependent GR target genes (but not at a G9a/GLP-independent GR target gene) was significantly reduced and essentially eliminated by depletion of HP1γ (FIGS. 22E-1, 22E-2, 22E-3, 22E-4). Thus, recruitment of HP1γ by G9a or GLP methylation facilitates recruitment of RNA polymerase II to the TSS for efficient transcriptional activation.

### G9a and GLP Methylation and Coactivator Function Drive Dex-Induced Inhibition of Cell Migration

A pathway analysis of the genes from the microarray data that require GLP for their dex-induced expression indicated that genes involved in cell movement were enriched (FIGS. 32A, 32B), including CDH1, which encodes E-cadherin, a key component of adherens junctions. Loss of E-cadherin expression is important for epithelial-mesenchymal-transition and increased cell motility [28b]. Quantitative RT-PCR analysis confirmed that depletion of GLP significantly decreased dex-induced expression of CDH-1 mRNA after 8 h of dex treatment, and G9a depletion produced a similar although not significant trend (FIGS. 21D-1, 21D-2, 21D-3, 21D-4, 21D-5, 21D-6, 21D-7, 21D-8), indicating that G9a and GLP act as coactivators for this gene. Likewise, 24 h of dex treatment significantly increased E-cadherin protein expression at the plasma membrane (FIGS. 23A-1, 23A-2). However, G9a or GLP depletion largely prevented dex enhancement of E-cadherin expression, as indicated by quantification of the staining with Image J software (FIGS. 23A-1, 23A-2) and by immunoblot analysis (FIG. 32C). Since E-cadherin inhibits cell migration (FIGS. 32D-1, 32D-2, 32D-3, 32D-4), we analyzed the effect of G9a/GLP depletion and dex on cell migration by the transwell migration assay. There was a significant decrease of migration in cells incubated with dex for 24 h compared to ethanol-treated cells (FIG. 23B). However, depletion of G9a or GLP by siRNA significantly prevented repression of cell migration by dex (FIGS. 23B-1, 23B-2, 23B-3). In order to determine the impact of G9a methylation on this phenotype, we used the stable cell line where G9a expression (wild-type or K185 mutant) is doxycycline inducible (FIG. 32C). Dex treatment decreased migration of A549 cells as previously demonstrated, and similar dex inhibition of migration was observed in cells overexpressing wild type G9a (FIGS. 23D-1, 23D-2, 23D-3). In contrast, overexpression of G9a K185R significantly prevented the dex-induced decrease in migration and in fact caused increased cell migration after dex treatment, suggesting that the overexpressed mutant version of G9a has a dominant-negative effect, suppressing the activity of endogenous G9a and interfering with the dex-induced decrease in migration. Consistent with these results, analyses of the E-cadherin expression by western-blot (FIG. 23C) or immunofluorescence (FIGS. 32E-1, 32E-2, 32E-3) showed that there is little or no dex-induced increase of E-cadherin gene expression after overexpression of G9a K185R. These findings further demonstrate that methylation of G9a and subsequent recruitment of HP1γ are involved in the regulation of cell migration, an important function in normal cell biology, EMT, and cancer metastasis in many systems. In addition, in another experimental model the estrogen-dependent proliferation of MCF-7 breast cancer cells was dependent on G9a and HP1γ (FIGS. 32F-1, 32F-2). Since HP1γ is critical for the coactivator activity of G9a, this implicates the coactivator activity of G9a in estrogen-dependent proliferation of breast cancer cells.

### Discussion

PTMs Provide a Switch that Regulates G9a and GLP Coactivator Function

A growing list of transcriptional coregulators has been associated with both gene activation and gene repression [1b, 3b, 5b], and indeed TFs that recruit these coregulators also activate or repress different subsets of their direct target genes (i.e. those genes that are regulated by the TFs and coregulators and are associated with regulatory sites where the TFs/coregulators bind). Thus far very little is known about the factors that dictate whether TFs and coregulators act positively or negatively on each of their direct target genes. A relevant observation is that TFs and coregulators act in a gene-specific manner, e.g. different direct target genes of the same TF have distinct mechanisms of transcriptional activation, as indicated by the fact that they require different sets of transcriptional coregulators [4b, 5b, 29b]. These observations lead to our working hypothesis that each gene has a unique regulatory environment that specifies which coregulators are required and is determined by several factors, including but perhaps not limited to: the specific DNA sequence to which the TF binds, which can alter the conformation of the TF [30b, 31b]; the DNA sequence surrounding the TF binding site, which dictates which other TFs may bind with their associated coregulators; the status of various cellular signaling pathways and the presence or absence of their effecter proteins (some of which make PTMs which may regulate DNA binding and activity of various TFs and coregulators) on regulatory sites associated with specific genes; and the local chromatin conformation which may also dictate which coregulators are required for the appropriate chromatin remodeling events needed to achieve gene regulation.

Here, using a model system of glucocorticoid-regulated gene transcription, we investigated the mechanism that controls transcriptional activation by two specific coregulators, G9a and GLP. G9a and GLP are two important, ubiquitous, and essential coregulators that have been implicated in many mammalian physiological processes. We demonstrated here that GLP acts in a gene-specific manner as a coregulator for GR: GLP facilitates glucocorticoid activation of some GR target genes and glucocorticoid repression of others, while a third subset of GR target genes is regulated by the hormone independently of GLP, as was already described for G9a [4b]. Furthermore, there is substantial overlap in the dex-induced genes that are negatively affected by depletion of G9a or GLP, but a few GR target genes were regulated by GLP and not G9a, showing that these two proteins support the regulation of highly similar but not identical gene sets (FIGS. 21A, 21B, 21C, 21D-1, 21D-2, 21D-3, 21D-4, 21D-5, 21D-6, 21D-7, 21D-8, 21E-1, 21E-2, 21E-3, 21E-4, 21E-5, 21E-6, 21E-7, 21E-8, 21F-1, 21F-2, 21F-3, 21F-4, 21F-5, 21F-6, 21F-7, 21F-8). We show here that two specific PTMs shared by G9a and GLP provide a molecular switch that regulates the ability of G9a and GLP to function as coactivators (FIG. 23E). It is interesting to speculate that regulation of the coactivator function of G9a and GLP may have an effect on the decision as to whether G9a and GLP function as coactivator or corepressor on a given gene to which they are recruited. However, further work is required to address this issue.

### HP1γ Recruitment by G9a and GLP is Regulated by PTMs and is Required for G9a and GLP Coactivator Function

We demonstrated here that GLP is methylated on K205 and phosphorylated on T206 by aurora kinase B in a sequence of amino acids with high homology to the similarly modified region of G9a. The formation of the G9a/GLP-HP1γ complex in cells is regulated by G9a/GLP methylation and phosphorylation, as indicated by co-immunoprecipitation of over-expressed proteins and by PLA using endogenous proteins in A549 cells (FIGS. 17A, 17B-1, 17B-2, 17C, 17D, 17E-1, 17E-2, 17E-3, 18A, 18B, 18C-1, 18C-2, 18C-3, 18D-1, 18D-2, 18D-3, 18D-4, 18D-5, 18E-1, 18E-2, 18E-3, 19A, 19B). G9a or GLP binding to HP1γ requires lysine methylation of K185 in G9a or K205 in GLP and is inhibited by threonine phosphorylation (T186 or T206); furthermore, both G9a and GLP can nucleate a ternary complex with HP1γ and GR.

Comparison of dex-induced gene expression for the G9a/GLP-dependent and the G9a/GLP-independent GR target genes served as an internally controlled experimental system to demonstrate the gene-specific nature of the G9a/GLP coactivator pathway and the role of the G9a/GLP PTMs in controlling their coactivator function (FIG. 23E). There was a consistent contrast in the roles of all components of the G9a/GLP coactivator pathway in mediating dex-induced expression of the G9a/GLP-dependent and G9a/GLP-independent genes. Depletion of G9a, GLP, and HP1γ, and use of an Aurora kinase B inhibitor all had distinct effects on the G9a/GLP-dependent versus G9a/GLP-independent genes (FIGS. 21A, 21B, 21C, 21D-1, 21D-2, 21D-3, 21D-4, 21D-5, 21D-6, 21D-7, 21D-8, 21E-1, 21E-2, 21E-3, 21E-4, 21E-5, 21E-6, 21E-7, 21E-8, 21F-1, 21F-2, 21F-3, 21F-4, 21F-5, 21F-6, 21F-7, 21F-8). Similarly, dex-induced occupancy by G9a, GLP, HP1γ, S93-phosphorylated HP1γ, and RNA polymerase II was consistently different on G9a/GLP-dependent versus G9a/GLP-independent GR target genes (FIGS. 22A-1, 22A-2, 22B, 22C, 22D-1, 22D-2, 22D-3, 22D-4, 22D-5, 22D-6, 22E-1, 22E-2, 22E-3, 22E-4, 31A, 31B, 31C, 31D-1, 31D-2, 31D-3, 31D-4, 31D-5, 31D-6, 31D-7, 31D-8, 31E-1, 31E-2, 31E-3, 31E-4, 31E-5, 31E-6, 31E-7, 31E-8, 31F-1, 31F-2, 31F-3, 31F-4, 31F-5, 31F-6, 31F-7, 31F-8). Thus, multiple experimental comparisons of the roles of multiple components of the G9a/GLP coactivator pathway in dex-induced expression of these two groups of GR target genes provide compelling, well-controlled evidence for the importance of the PTMs of G9a and GLP in controlling their coactivator function for a specific subset of GR target genes. Furthermore, the fact that the mechanisms of G9a coactivator and corepressor functions are distinct and utilize different domains of G9a and GLP, along with the selective recruitment of G9a, GLP and HP1γ only to GR target genes where they are required as coactivators, provides very strong evidence to validate our conclusion that G9a, GLP and HP1γ are acting directly as coactivators on these genes, rather than by some indirect mechanism in which G9a, GLP and HP1γ are acting as corepressors (e.g. repressing a gene that encodes a repressor of the GR target genes).

In addition, these findings demonstrate that the molecular mechanism of coactivator function of G9a and GLP involves recruitment of HP1γ. Thus, HP1γ functions as a coactivator on these genes after dex treatment, mediating the coactivator function of G9a and GLP (FIGS. 21A, 21B, 21C, 21D-1, 21D-2, 21D-3, 21D-4, 21D-5, 21D-6, 21D-7, 21D-8, 21E-1, 21E-2, 21E-3, 21E-4, 21E-5, 21E-6, 21E-7, 21E-8, 21F-1, 21F-2, 21F-3, 21F-4, 21F-5, 21F-6, 21F-7, 21F-8), by facilitating the recruitment of RNA polymerase II (FIGS. 22A-1, 22A-2, 22B, 22C, 22D-1, 22D-2, 22D-3, 22D-4, 22D-5, 22D-6, 22E-1, 22E-2, 22E-3, 22E-4, 23E). While the HP1 family of proteins (α, β, and γ) are primarily known for their roles in gene repression, HP1γ in particular has also been shown to function as a coactivator for regulation of specific genes [18b]. Consistent with that, even though they interact with methylated G9a [16b], HP1α or HP1β do not function as coactivators for regulation of G9a/GLP-dependent or independent GR target genes (FIGS. 29E-1, 29E-2, 29E-3, 29E-4, 30E-1, 30E-2, 30E-1, 30E-2), in contrast to HP1γ (FIGS. 21E-1, 21E-2, 21E-3, 21E-4, 21E-5, 21E-6, 21E-7, 21E-8, 22A-1, 22A-2, 22B, 22C, 22D-1, 22D-2, 22D-3, 22D-4, 22D-5, 22D-6, 22E-1, 22E-2, 22E-3, 30A-1, 30A-2, 30A-3, 30A-4, 30A-5, 30A-6).

In a previous report [4b] we concluded that the methyltransferase activity of G9a was required for its corepressor activity but not for its coactivator activity, based on an experiment where we pretreated A549 cells with a G9a/GLP-specific methyltransferase inhibitor for 1 hour prior to dex treatment. The data reported here show that self or reciprocal methylation by G9a and GLP is required for coactivator function which obviously contradicts the previous conclusion. The explanation lies in the length of pretreatment with the G9a/GLP-specific methyltransferase inhibitor. The 24-hour pretreatment with the inhibitor used in the current study is required to substantially reduce the methylation of G9a K185 and GLP K205 and thus inhibit G9a/GLP coactivator function. Thus, the 1-hour inhibitor pre-treatment used in the previous study [4b] was sufficient to prevent new methylation of histone H3K9, which is required for G9a/GLP corepressor function; but the 1-hour pretreatment was not sufficient to reduce the N-terminal methylation of G9a and GLP and thus did not significantly affect the coactivator function.

### G9a and GLP Coactivator Function Regulates Cell Migration of a Lung Cancer Cell Line

The biological function of the two PTMs on G9a and GLP, and of the regulation of their interaction with HP1y, has not been previously addressed. Using the A549 lung cancer cell model, we demonstrated that G9a and GLP mediate glucocorticoid repression of cell migration by cooperating with GR to induce the expression of target genes such as CDH1 (which encodes E-cadherin) that are involved in cell migration (FIGS. 23A-1, 23A-2, 23B-1, 23B-2, 23B-3, 23C, 23D-1, 23D-2, 23D-3, 23E). Furthermore, methylation of G9a on K185 is directly involved in this process. Indeed, overexpressed G9a K185R acts as a dominant-negative, preventing dex-induced expression of E-cadherin and the resulting dex repression of cell migration (FIGS. 23A-1, 23A-2, 23B-1, 23B-2, 23B-3, 23C, 23D-1, 23D-2, 23D-3, 23E, 32A, 32B, 32C, 32D-1, 32D-2, 32D-3, 32D-4, 32E-1, 32E-2, 32E-3, 32F-1, 32F-2). These findings directly implicate the methylation of G9a and the resulting coactivator function of G9a in cell migration and thus demonstrate a specific biological regulatory function for G9a/GLP PTMs in the GR signaling pathways. The fact that reduction of CDH1 expression is a critical part of the mechanism of epithelial-mesenchymal transition, which is involved in many developmental processes as well as tumor progression [28b], suggests that the coactivator function of G9a and GLP may play critical roles in these developmental and pathogenic processes.

Possible Mechanisms Regulating G9a and GLP PTMs, and their Implications

Regulation of the methylation and phosphorylation status of G9a and GLP modulates glucocorticoid regulation of the specific subset of GR target genes (among all the genes regulated by GR) that require G9a and GLP as coactivators. In effect, this provides a mechanism for modulating the hormone response. Since G9a and GLP are controlled by this dual-PTM switch and also serve as coregulators for many different TFs, it seems likely that the same PTM switch controls positive gene regulation by G9a and GLP much more broadly than just with steroid hormone receptors. Furthermore, since the same methylation/phosphorylation switch regulates binding of HP1 proteins to histone H3 (at methylated lysine 9), we speculate that a similar switch mechanism will be found to control positive versus negative gene regulation by other coregulators and TFs, controlling many others biological functions.

Based on current knowledge there are many potential pathways to regulate the addition or removal of these two PTMs on G9a and GLP. Methylation could be regulated by controlling the intramolecular or intermolecular interaction of the N-terminal methylation site with the C-terminal regions of G9a or GLP containing the methyltransferase activity. Indeed, in vitro methylation experiments with G9a and GLP fragments indicate that trans-methylation as well as intramolecular auto-methylation is possible (FIGS. 17A, 17B-1, 17B-2, 17C, 17D, 17E-1, 17E-2, 17E-3), suggesting similar mechanisms in cells since G9a and GLP heterodimerize. In addition, there are many different potential enzymes to test for G9a and GLP demethylation. JMJD1A, LSD1/KDM1, PHF8, KMD4A, and KMD7A can all demethylate H3K9 [32b-35b], which has almost the same local amino acid sequence context (ARKS) as the G9a and GLP methylation sites (ARKT), suggesting that these enzymes may also demethylate G9a and GLP.

In addition, the protein level and activity of aurora kinase B are regulated in many ways. Transcription of the aurora kinase B gene is regulated by the cell cycle [36b, 37b] and by transcription factors such as c-Myc, p53, and ETS-1 [38b, 39b]. Aurora kinase B activity is regulated by multiple protein-protein interactions, and by phosphorylation and dephosphorylation [36]. Stability of the protein [37b] and mRNA [40b] is also regulated. G9a has been shown to regulate proliferation and differentiation of skeletal muscle cells, regulating the cell cycle by two different mechanisms, serving as a corepressor for some genes and as a coactivator for other genes [41b], suggesting a possible complex interaction with the regulation of methylation and phosphorylation of G9a and/or GLP in this context. It will be important to explore the many possible regulatory mechanisms for the G9a and GLP PTMs, including the identity and regulation of G9a/GLP demethylases, and which of the many aurora kinase B regulatory mechanisms identified in the context of the cell cycle may apply to its function as a modulator of G9a and GLP coactivator activity. In addition, since G9a [10b, 42b] and aurora kinase B [37b, 38b, 43b] are both over-expressed in many different types of cancer, it is important to ask whether the gene targets that require G9a as a coactivator, as a corepressor, or both are involved in the transformed phenotype.

The following publication is incorporated in its entirety by reference herein: Poniard, Coralie, et al. “A post-translational modification switch controls coactivator function of histone methyltransferases G9a and GLP.” EMBO reports (2017): e201744060.

### EXAMPLE 3

### Inhibition of Aurora Kinase B Potentiates Glucocorticoid Activity in B-Cell Acute Lymphoblastic Leukemia

Glucocorticoids (GCs) are used in combination chemotherapies as front-line treatment for lymphoid cancers, including B -cell acute lymphoblastic leukemia (B-ALL). Although effective, many patients relapse and become resistant to chemotherapy, and GCs in particular. Why these patients relapse is not clear. We took a comprehensive, functional genomics approach to identifying sources of GC resistance that could be targeted to restore sensitivity. We compared results from a genome-wide shRNA screen to identify genes that affect growth and GC-sensitivity in B-ALL to misexpressed genes in relapsed patients. We identified cell cycle genes, including AURKB, as sources of relapse. AURKB restrains the activity of the glucocorticoid receptor by phosphorylating specific coregulators, EHMT1/2. Inhibition of AURKB catalytic activity enhanced the GC-regulation of cell death genes, resulting in potentiation of GC cytotoxicity in cell-line and patient B-ALL specimens. These results validate a functional genomic approach to the design of combination chemotherapeutics for relapsed patients and demonstrate how transcription can be tailored by inhibiting pathways that impinge on coregulators.

Glucocorticoid (GCs), including dexamethasone (dex) and prednisone (pred), are a component of front-line combination chemotherapies used to treat lymphoid cancers (Granner et al., 2015). In children with B-cell acute lymphoblastic leukemia (B-ALL), the response to GCs alone is highly correlated with the response to treatment overall, suggesting that GCs may be the key component in treatment efficacy (Inaba and Pui, 2010; Klumper et al., 1995; Lönnerholm et al., 2009; Mullighan et al., 2011). GCs work by binding to the glucocorticoid receptor (GR), a ligand-activated transcription factor, which then translocates to the nucleus, associates with DNA, and regulates genes (Yamamoto, 1985). Regulation of genes by GR is essential to the cytotoxicity of GCs (Smith and Cidlowski, 2010). Although effective, about 10% of children with B-ALL do not respond to GC-based combination chemotherapy or develop resistance upon relapse (Terwilliger and Abdul-Hay, 2017). Until the advent of CarT cells, few options have been available for relapsed patients, and their prognosis is poor.

Because CarT cells are not an option for all patients, and relapse can still occur (Fischer et al., 2017), understanding the sources of relapse to identify new treatments has been intensely studied. Genome-wide sequencing studies identifying mutations associated with relapse (Mullighan et al., 2011) revealed the importance of transcription factors, and also transcriptional coregulators known to associate with GR, including CREBBP/P300. Work from our lab showed that the genes most frequently mutated modulate the sensitivity of B-ALL cells to GCs (Kruth et al., 2017). Genetic deletion of TBL1XR1, another GR coregulator, in treatment refractory patients also blunts the sensitivity of B-ALL to GCs, underscoring the importance of coregulators in treatment efficacy. Despite these efforts, genetic lesions explain a small fraction of GC resistance (Madhusoodhan et al., 2016).

Another potential source of resistance to GCs is misexpression of genes. Three studies have compared the gene expression of patients at diagnosis to those at relapse in children with B-ALL. Each study identified tens of misexpressed genes that were most prominently related to cell cycle and replication (e.g. PTTG1, CDC20), apoptosis (BIRCS, HRK), and DNA repair (FANC genes) (Bhojwani et al., 2006; Hogan et al., 2011; Staal et al., 2010). Analyses in the three studies differed substantially, making comparison difficult, and resulting in identification of different misexpressed genes. A meta-analysis of data from all three studies identified ˜1,500 up and down regulated genes by non-parametric rank product testing (Chow et al., 2017). Integration of misexpression with other data, including DNA methylation and copy number variation, yielded higher confidence hits. These include cell cycle, WNT, and MAP kinase cascades, including the B-cell receptor pathways. Nonetheless, few functional links between gene misexpression and GC resistance have been established, thwarting development of therapies to overcome resistance.

One method used to overcome resistance is by potentiating GCs. Stronger GCs, such as deacylcortivazol, which bind GR with higher affinity and induce stronger gene activation, have been developed. However, because GCs have many other physiological roles, high doses result in acute and long-term life-threatening side-effects (Inaba and Pui, 2010; Ness et al., 2011), preventing their use in chemotherapy. Recently, we used functional genomics methods to identify strategies for potentiating GCs, but specifically in the tissue of interest. By integrating the transcriptional response of B-ALL samples with a shRNA screen of ˜5,600 genes, we identified a role for GCs in B-cell developmental programs. Inhibiting a node in the B-cell receptor signaling network, the lymphoid-restricted PI3Kδ, potentiated GCs even in some resistant patient samples (Kruth et al., 2017). Although this combination would be expected to have few side effects, it does not specifically target sources of relapse that would attenuate GC function.

In this study we extended our functional genomics approach to all genes in order to elucidate mechanisms of GC resistance and identify therapeutic targets. We first combined available data sets of gene expression at diagnosis and relapse in children with B-ALL to identify misexpressed genes and pathways associated with relapse. We integrated this data with a comprehensive genome-wide shRNA screen to identify genes that affect growth and sensitivity. This approach revealed the importance of specific GR coregulators and cell-cycle proteins, including AURKB. We showed that inhibition of AURKB potentiates GCs in B-ALL cell lines and patient samples by enhancing the activity of specific GR transcriptional coactivators EHMT2 (aka G9a), EHMT1 (aka GLP), and CBX3 (aka HP1γ).

### Methods

### Cell Culture—Cell Lines

Nalm6 and pre-B 697 cells were purchased from American Type Culture Collection (ATCC) and Deutsche Sammlung von Mikroorganismen and Zellkulturen (DSMZ), respectively and maintained in RPMI 1640 medium containing L-Glutamine and supplemented with 10% fetal bovine serum (FBS) at 37° C. and 5% CO2. HEK293T cells were purchased from Clonetech and maintained in Dulbecco modified Eagle medium supplemented with 10% FBS at 37° C. and 5% CO2. All cells were screened for mycoplasma contamination. Dexamethasone (Sigma-Aldrich) was dissolved in ethanol. ZM447439 (Tocris) and AZD2811 (Selleckhem) were dissolved in dimethylsulfoxyde.

### Cell Culture—Patients Samples

Bone marrow and peripheral blood samples from ALL patients were acquired in compliance with the Institutional Review Board regulations of each institution. Informed consent for cell banking was obtained from all human subjects.

Primary ALL cells (LAX7R and LAX56) were established from mouse xenografts derived from patient samples (Adam et al., 2017; Hsieh et al., 2013). They were cultured in 24 well tissue-culture plates on irradiated OP-9 cells with growth medium containing MEM-α with 20% heat-inactivated fetal bovine serum, and 100 IU/ml penicillin and 100 μg/ml streptomycin. On day 3 and day 5 after the addition of drugs, ALL cells were collected, and apoptosis was assessed using PE Annexin V Apoptosis Detection Kit with 7-AAD.

### Aggregated Processing of Affymetrix Arrays at Diagnosis and Relapse for Childhood B-ALL

Three studies have previously compared gene expression at diagnosis and relapse in children with ALL (both B-ALL and T-ALL) (Bhojwani et al., 2006; Hogan et al., 2011; Staal et al., 2010). Two of these studies (Hogan et al., 2011; Staal et al., 2010) further classified patients by time of relapse, either early (<3 years) or late (>3 years). We downloaded these data sets (GSE3920, GSE18497, GSE28460) from the Gene Expression Omnibus (https://www.ncbi.nlm.nih.gov/geo/) and imported them into R using GEOquery (Davis and Meltzer, 2007). We first examined the background correction and normalization and found that GSE18497 and GSE28460 looked to be properly processed, however the expression levels of different patients were heterogeneous for GSE3920. We therefore downloaded the raw intensities for GSE39820 and renormalized the samples using rma (from the affy package) (Gautier et al., 2004). We then separated the B-ALL from other (e.g. T-ALL) samples and performed downstream analysis on these samples. Because samples were run on different Affymetrix platforms, we performed differential gene expression analysis on each separately. This was done by setting up a contrast matrix to compare samples from diagnosis and relapse (Supplementary Dataset 1), as well as to compare expression at diagnosis for patient who eventually relapsed early versus late (Supplementary Dataset 4). Differential expression was then tested using limma (Ritchie et al., 2015). For each contrast (diagnosis versus relapse and early versus late) in each data set, a fold change and p-value was calculated. The fold changes across samples were combined by simply averaging them. p-values were combined by two methods: the Fisher and Stauffer methods. After generating a combined p-value for each gene, a multiple testing correction was applied by calculating a qvalue (Storey and Tibshirani, 2003). The Fisher and Stauffer methods were largely consistent (80% qvalue <0.05), and thus the Fisher qvalues were used for downstream analysis.

Next-Generation shRNA Screen

The shRNA constructs for the next-generation knockdown screen were designed and synthesized as previously described (Kampmann et al., 2013, 2014). The screen was performed largely as described (Kampmann et al., 2014), and previously implemented by our group (Kruth et al., 2017). The details and modifications are described below.

The shRNAs are synthesized in 13 sub-libraries that correspond to genes grouped by function or biological process. The 13 libraries were mixed in equimolar amounts to ensure equal representation of each library and shRNA. 293T cells were seeded on two poly-L-lysine coated 15-cm plates and grown to 70% confluence. Cells were then transfected with 32 μg of pooled shRNA libraries and 32 μg of pooled 3rd generation lentiviral packaging constructs (VSV-G, RSV, MDL, Addgene #s: 12259, 122532, and 12251, respectively) using Mirus LT1 transfection reagent. The supernatant containing virus was harvested after 48 hours and then 0.45 μM filtered.

NALM6 cells were grown in RPMI +10% FBS at 37° C., 5% CO2 in 1L spinner flasks to a density of no more than 3 million cells/ml. Prior to infection, cells were spun down, then resuspended in growth medium at a concentration of 3 million cells/ml and plated into 6-well plates. Virus was diluted 1:20 and added 1:1 to the cells along with 8 μg/ml polybrene and spinfected (1000 rpm, 2 hours, 33° C.). Cells were then resuspended and allowed to recover in a 1L spinner flask in growth medium. After two days, cells were treated with 0.5 μg/ml puromycin for three days to select for infected cells, washed in PBS, and allowed to recover. Infected cells were then counted as mCherry positive by flow cytometry. We obtained an average of 900 cells infected with each shRNA in the screen at this point.

Cells were grown in spinner flasks until we had a sufficient number to begin treatment. At this point the culture was divided into five cultures: T0, our infection control; TF, our growth control; and R1-3, our three repeats of dex treated. 500 million cells of T0 were immediately spun down and stored at −80° C. 1L each of TF and R1-3 were grown to a density of 2 million cells/ml in spinner flaks, and then treated with 35 nM dex in 0.1% ethanol, a concentration chosen to achieve 50% death, for 3 days. TF cells were mock treated with 0.1% ethanol. After three days, cells were spun down, washed with PBS, and resuspended in growth medium to a density of ˜500,000 cells/ml and allowed to recover. Recovery was very rapid in the spinner flask, and some cells were discarded prior to the next treatment. Treatment was then repeated 2 times, for a total of three treatments. TF, and R1-3 were then spun down and either stored in aliquots at -80° C., or genomic DNA was immediately harvested.

Genomic DNA was harvested from 500 million T0, TF, and R1-3 cells using Qiagen Blood Maxi kit (#51194). Genomic DNA was then digested overnight with 10 U/mg PvuII restriction enzyme overnight and run on a 0.8% agarose gel. A slice of gel encompassing the 1.6 kb expected size was excised, and DNA purified using Qiagen gel extraction kits (28706). shRNA cassettes were then amplified and barcodes introduced using 25 rounds of PCR and the following primers as shown in Table 8

TABLE 8

| Sample | Primer | Index | Sequence | SEQ ID NO: # |
|---|---|---|---|---|
| T0 | oMK483 | CTTGTA | aatgatacggcgaccaccgaGATCGGAAGAG | SEQ ID NO: 86 |
| | | | CACACGTCTGAACTCCAGTCAC | |
| | | | CTTGTACTCTAGATGACTGACCCCT | |
| | | | TG | |
| TF | oMK484 | GCCAAT | aatgatacggcgaccaccgaGATCGGAAGAG | SEQ ID NO: 87 |
| | | | CACACGTCTGAACTCCAGTCAC | |
| | | | GCCAATCTCTAGATGACTGACCCCT | |
| | | | TG | |
| R1 | oMK485 | AGTTCC | aatgatacggcgaccaccgaGATCGGAAGAG | SEQ ID NO: 88 |
| | | | CACACGTCTGAACTCCAGTCAC | |
| | | | AGTTCCCTCTAGATGACTGACCCCT | |
| | | | TG | |
| R2 | oMK486 | TAGCTT | aatgatacggcgaccaccgaGATCGGAAGAG | SEQ ID NO: 89 |
| | | | CACACGTCTGAACTCCAGTCAC | |
| | | | TAGCTTCTCTAGATGACTGACCCCT | |
| | | | TG | |
| R3 | oMK487 | TTAGGC | aatgatacggcgaccaccgaGATCGGAAGAG | SEQ ID NO: 90 |
| | | | CACACGTCTGAACTCCAGTCAC | |
| | | | TTAGGCCTCTAGATGACTGACCCCT | |
| | | | TG | |

PCR products were then run on 12% polyacrylamide gels, with the bands @ 273 bp excised and extracted from the gel by electroelution. The DNA was then cleaned and concentrated using a MinElute PCR Purification Kit from Qiagen. The libraries were quantified by Bioanalyzer, mixed into one pool, and sequenced via Illumina HiSeq 2500 to a depth of ˜160 million reads/sample (˜320 reads/shRNA).

Values for the effect of gene depletion on growth and sensitivity were generated using the Glmap suite of tools (gimap.ucsf.edu). These tools generate a value gamma (γ) that is the ratio of TF/T0 and represents the effect of depletion on growth, and rho (ρ) that is the ratio of Rs/TF. The confidence for each value is tested in two way, by Mann-Whitney and Kolmogorov-Smirnov tests against a set of control RNAs. Tables for the results of the screen (Supplementary Dataset 2) are available on-line.

The genes with a significant effect on dex sensitivity were in good accord with a previous, more limited screen (Kruth et al., 2017) (Wilcoxon signed rank test, p-value=0.2). The effect on growth, however shows more divergence between the data sets (Wilcoxon signed rank test, p-value=1.5e-5), likely due to the difference in growth conditions (spinner vs. still flasks), which caused significantly faster growth (doubling at 24 vs 36 hours).

### Gene Expression Analysis After Coregulator Depletion

NALM6 cells were depleted of either EHMT1, EHMT2, or NCOA2 using lentiviral delivered shRNAs described above. Uninfected NALM6 cell and non-specific shRNA (shSCR) infected cells were used as controls. After selection with 2 μg/ml Puromycin for three days, cells were allowed to recover, and grown in RPMI+10% FBS (Atlanta Biologicals) to a density of 1 million cells/ml then plated in 6 well plates, 3 million cells/well. Cells were treated with 1 μM dexamethasone for 4 hours and then spun down @ 400 g for 5 minutes. Cell pellets were resuspended in 700 μl Trizol (or Qiazol) and stored at −80° C. Total RNA was isolated using the miRNAeasy kit (Qiagen) and sent to the UCLA Neuroscience Genomics Core where samples were labeled, hybridized to Illumina HT12 v4 gene expression microarrays and scanned. Three biological repeats of each condition were performed.

Arrays were processed using R/Bioconductor. Raw intensities were transformed, normalized, and background corrected using the lumi package (Du et al., 2008). Contrasts were set up, and differential expression tested using limma (Ritchie et al., 2015). To identify which genes were regulated by dex for each condition we performed pairwise tests of the shRNA knockdown for each coregulator to the shSCR control. We then tested which genes were regulated by dex differently upon coregulator knockdown by pairwise tests of the fold change for each coregulator depletion compared to the fold change after dex treatment for the shSCR control. The effect of coregulator knockdown on the expression level of each gene was tested by comparing each shRNA knockdown in the presence of dex to the control in the presence of dex. Code for the above analyses will be provided upon request.

### Immunoprecipitation and Immunoblots

Cells were treated as indicated, and cell extracts were prepared in RIPA buffer (50 nM Tris-HCl, pH 8, 150 mM NaCL, 1 mM EDTA, 1% NP-40, and 0.25% deoxycholate) supplemented with protease inhibitor tablets (Roche Molecular Biochemicals) and phosphatase inhibitors (1 mM NaF, 1 mM Na3VO4, and 1 mM β-glycerophosphate). Protein extracts were incubated overnight at 4° C. with shaking with 1 μg of antibodies against EHMT2 (Sigma G6919), EHMT1 (Millipore 09-078), or pan phospho-threonine (Millipore AB1607). Protein A/G plus agarose beads (Santa Cruz sc-2003) were added, and the mixture was incubated 2 h at 4° C. The immunoprecipitates were separated on SDS-PAGE. Immunoblotting was conducted with primary antibodies against EHMT2 (Sigma G6919), β-actin (Sigma A5441), EHMT1 (Millipore 09-078), CBX3 (Abcam ab10480), pan-methyllysine (Abcam ab23366), GR (Santa Cruz sc-8992), cleaved Caspase 3 (Cell Signaling 9664S), cleaved Caspase 7 (Cell Signaling 8438S) or cleaved PARP1 (Cell Signaling 9541S). Secondary antibodies from Promega were used for chemiluminescence detection using ECL prime detection reagent (Amersham) according to the manufacturers' instructions.

### Cell Death Assays

Cells were plated in CellStar low evaporation lid 96-well round-bottom plate at a density of 100,000 cells/ml. Directly after plating, cells were treated in triplicate with serial dilutions of dexamethasone or vehicle control (0.1% ethanol). After 72 h, cell viability of each well was analyzed in duplicate using the Presto Blue Assay Reagent (Life Technologies). Fluorescence was measured and data were analyzed with Prism6 software.

### Chromatin Immunoprecipitation

ChIP experiments were performed according to previously described protocols (Poulard et al., 2017) with antibodies against GR (Santa Cruz sc-8992X) or CBX3-S93p (ab45270). Results are expressed relative to the signal obtained from input chromatin.

### Real-Time RT-qPCR

RNA was isolated using TRIzol (Invitrogen) according to the manufacturer's instructions. Reverse transcription reaction was performed using Superscript III (ThermoFisher) according to specifications with 1 μg of total RNA as template. Quantitative PCR amplification of the resulting cDNA was performed on a Roche LightCycler 480 using SYBR green I master mix (Roche). mRNA levels were normalized to the level of b-actin mRNA.

### Figure Legends

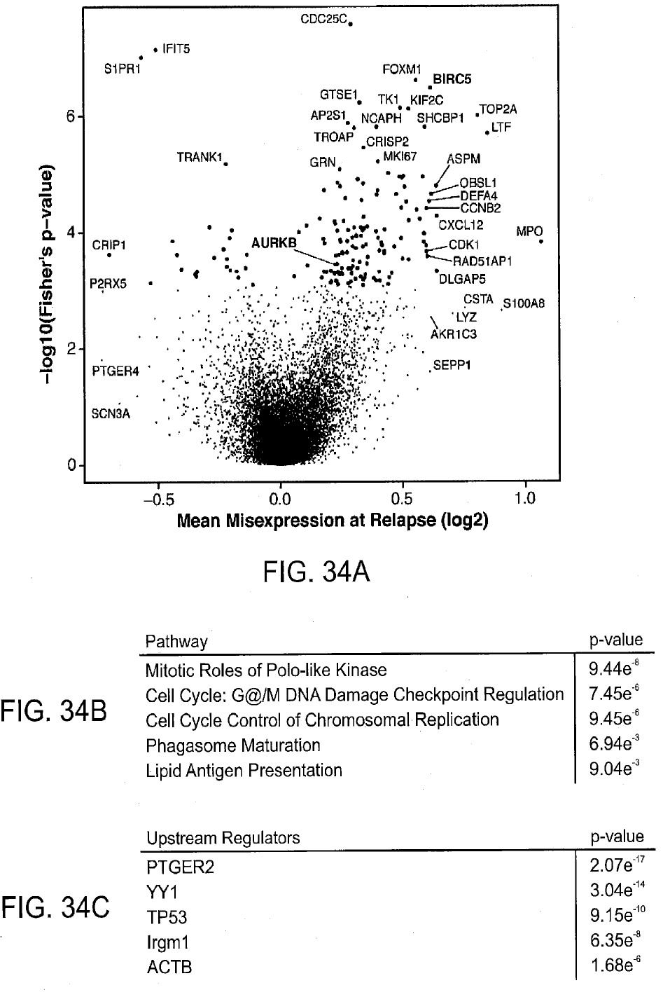

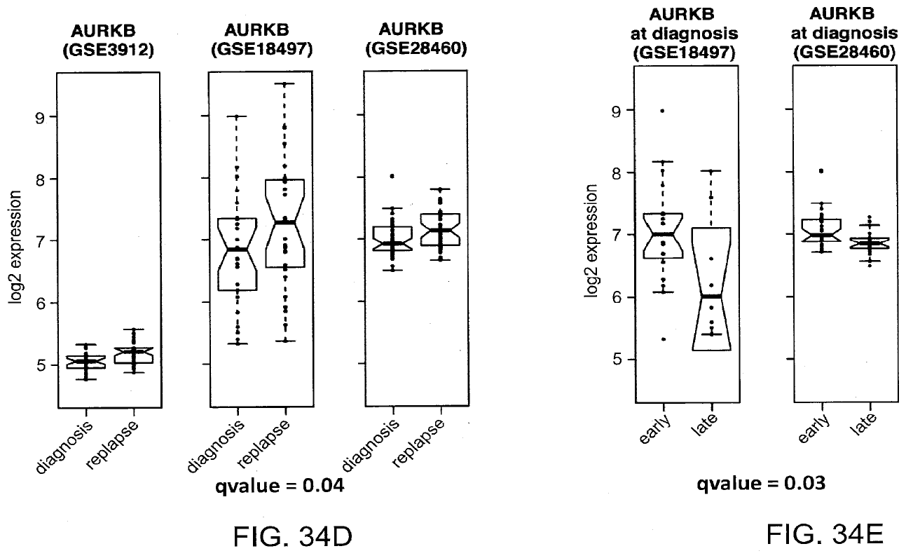

FIGS. 34A, 34B, 34C, 34D, 34E show genes differentially expressed in B-ALL at relapse versus diagnosis. FIG. 34A shows three studies collecting paired RNA samples from B-ALL patients at diagnosis and relapse (GSE3912, GSE18497, GSE28460) were combined. A fold-change and p-value for each gene were first calculated for each data set. The fold changes were then averaged, and the p-values combined using Fisher's method to generate the volcano plot. Genes with a qvalue≤0.1 are colored red 21. Outlying genes are labeled. FIG. 34B shows an Ingenuity pathway analysis of misexpressed genes indicates that cell cycle genes are highly enriched. FIG. 34C shows an upstream analysis of misexpressed genes shows an enrichment for prostaglandin signaling, specifically through PTGER2. FIG. 34D shows that AURKB is overexpressed upon relapse. Boxplots depict the relative expression of AURKB in B-ALL patient blood samples taken sequentially at diagnosis and relapse for the three different studies. Notches in boxplots represent a 95% confidence interval. As shown in FIG. 34E and using the two indicated databases, AURKB expression levels were compared in samples taken at diagnosis for B-ALL patients stratified according to length of time from diagnosis to relapse: patients who relapsed within 36 months, or patients who relapsed beyond 36 months from diagnosis.

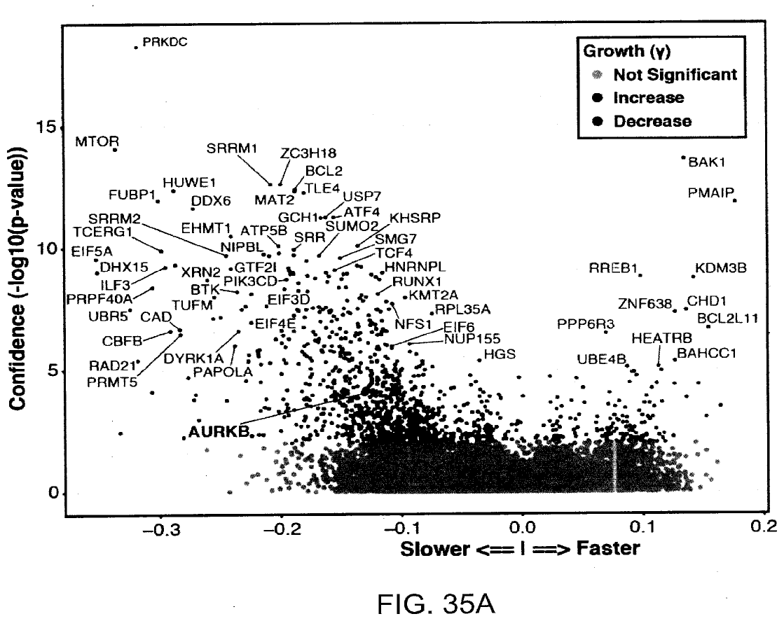

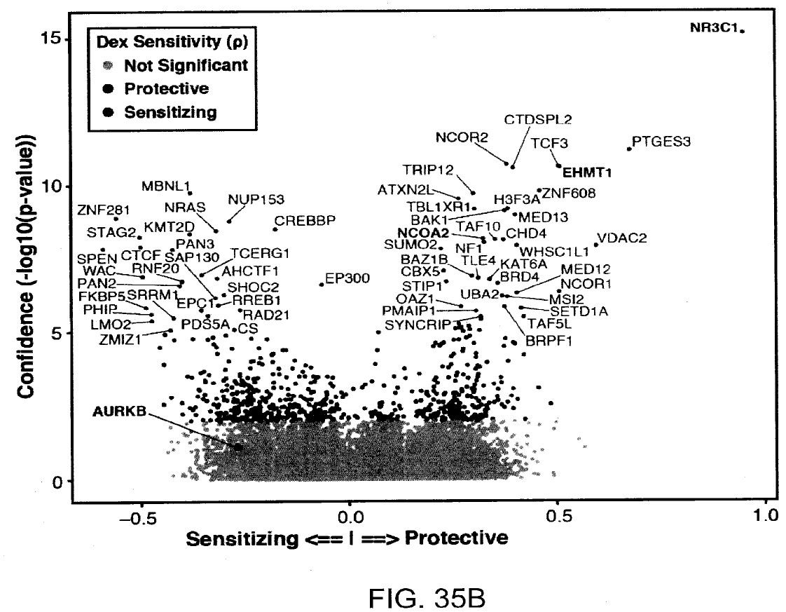

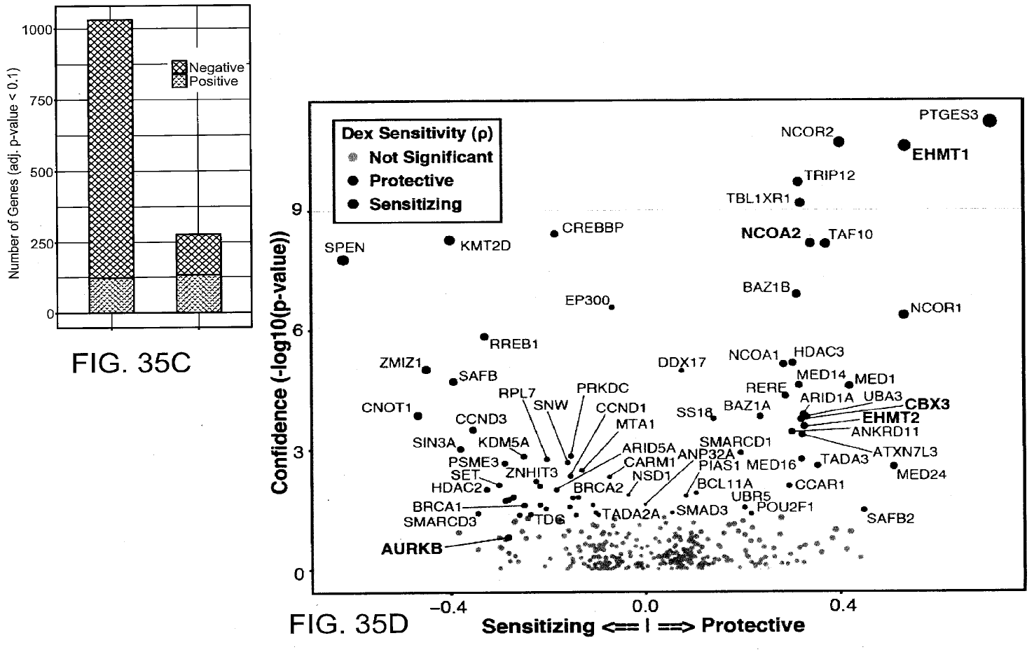

FIGS. 35A, 35B, 35C, 35D show genes that affect growth and sensitivity to dex in the B-ALL cell line NALM6. FIG. 35A shows that the gamma or growth values are calculated by averaging the enrichment of each shRNA in the cell population at the end of the growth period (TF) versus at initial infection (T0). Confidence values (p-values) are calculated by a Mann-Whitney test comparing enrichment of shRNAs vs thousands of control shRNAs. Green or light gray points indicate slower growth upon knockdown, purple or draker gray points represent faster growth upon knockdown. FIG. 35B shows that Rho or dex sensitivity values are calculated for each gene as the effect of knockdown on the average enrichment of shRNAs upon dex treatment (R1-R3) versus growth control (TF). Confidence values (p-values) are calculated by a Mann-Whitney test comparing enrichment of shRNAs vs thousands of control shRNAs. Green points are genes that sensitize cells to dex when knocked down, whereas purple points are those that render cells more resistant when knocked down. FIG. 35C shows a stacked bar chart representing the total number of genes that significantly (qvalue≤0.1) affect growth or dex sensitivity. Green represents slower growth or increased dex sensitivity, and purple represent faster growth or decreased dex sensitivity. FIG. 35D shows a volcano plot of the effect of coregulator knockdown on dex sensitivity, as in B. Coregulators were compiled from NURSA (https://nursa.org) or the literature.

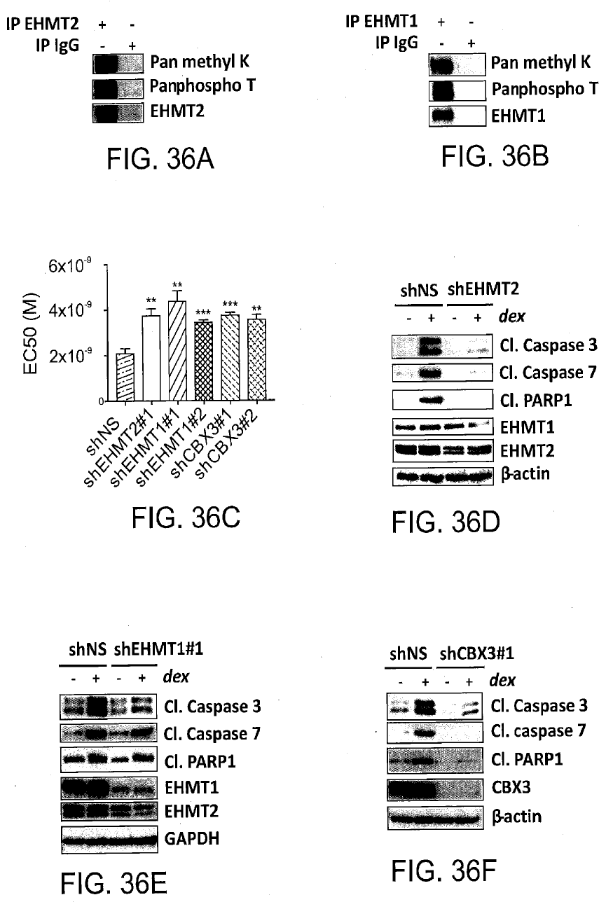

FIGS. 36A, 36B, 36C, 36D, 36E, 36F show that EHMT2/EHMT1/CBX3 facilitate GC-induced cell death. As shown in FIGS. 36A and 36B, methylation and phosphorylation of EHMT2 (FIG. 36A) and EHMT1 (FIG. 36B) in NALM-6 cells was analyzed by immunoprecipitation with control IgG, anti-EHMT2 antibody (A), or anti-EHMT1 antibody (FIG. 36B), followed by immunoblot with the indicated antibodies. As shown in FIG. 36C, NALM-6 cells expressing shRNA against EHMT2 (shEHMT2), EHMT1 (shEHMT1), CBX3 (shCBX3) or a non-specific sequence (shNS) were treated with two-fold dilutions of dex for 72 h. Cell survival was measured by a fluorescence metabolic assay. EC50s were calculated as the concentration at which half the cells remained alive, compared to vehicle controls. Error bars depict the SEM of 4 independent experiments and p-value was calculated to compare each coregulator depletion to shNS using a paired t-test **p≤0.01, * * *p≤0.001. (D-F) NALM-6 cells depleted or not for EHMT2 (FIG. 36D), EHMT1 (FIG. 36E), or CBX3 (FIG. 36F) were treated with 100 nM dex (+) or ethanol (−) for 24 h, and the indicated proteins were examined by western-blot.

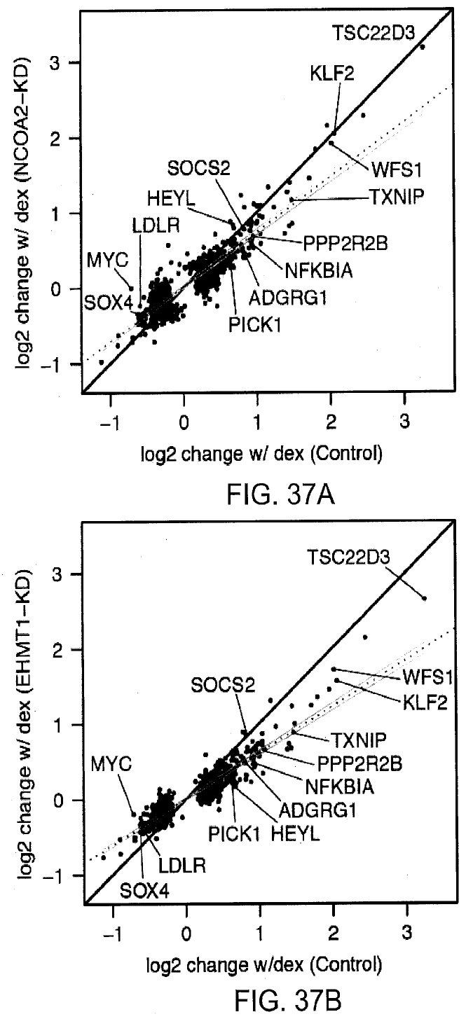

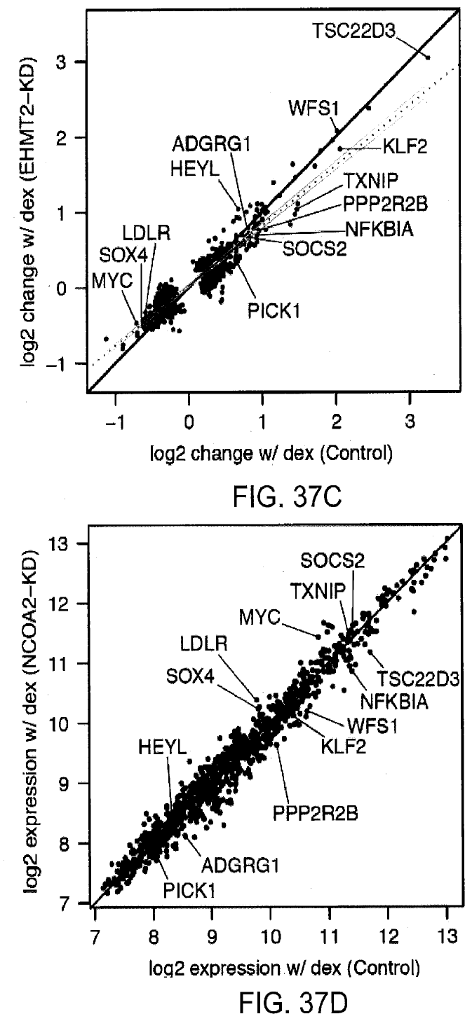

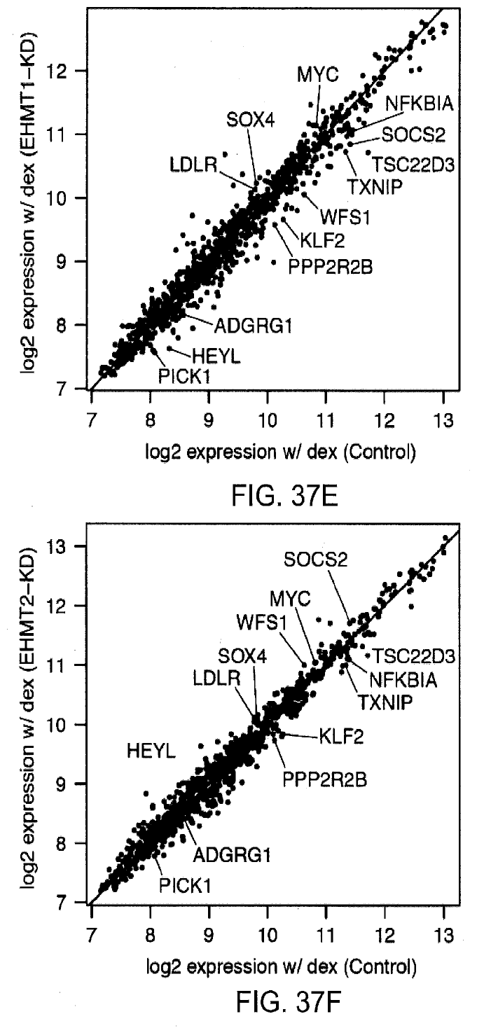

FIGS. 37A, 37B, 37C, 37D, 37E, and 37F show that each coregulator supports GC regulation of a subset of GR target genes. EHMT1, EHMT2, and NCOA2 were knocked down, treated with dexamethasone for 4 hours, then run on Illumina microarrays. FIGS. 37A, 37B, 37C show genes that were significantly regulated in the control (scrambled shRNA) or knockdown in response to dex were then plotted. Each point represents the log2 change in expression after dex exposure for the control (x-axis) or knockdown (y-axis) for each gene. The dashed line represents the linear least-squared regression fit to the points, and the flanking curved lines a 99% confidence interval about that line. Red or gray dots are genes that do not fit (p-value≤0.01) a slope of 1 (solid line), which would represent no change. (FIGS. 37D, 37E, and 37F) The expression level in dex-treated cells for genes that are significantly regulated under any condition are plotted for the control (x-axis) versus the knockdown (y-axis). A line of slope 1 (solid line) representing no change in regulation is plotted for each comparison. Genes that are significantly different (p-value≤0.01) are shown in red. Genes referred to in the text are labeled.

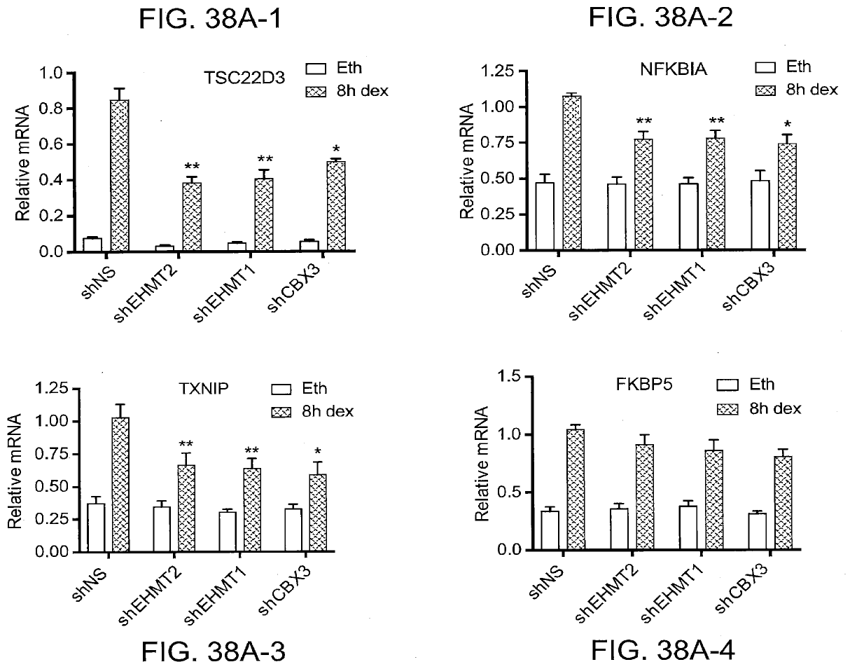

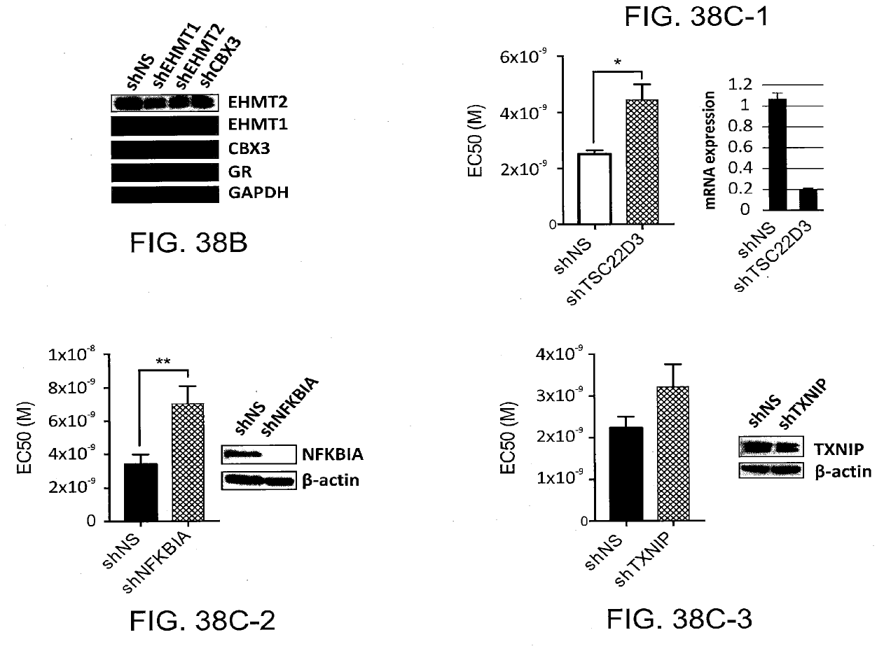

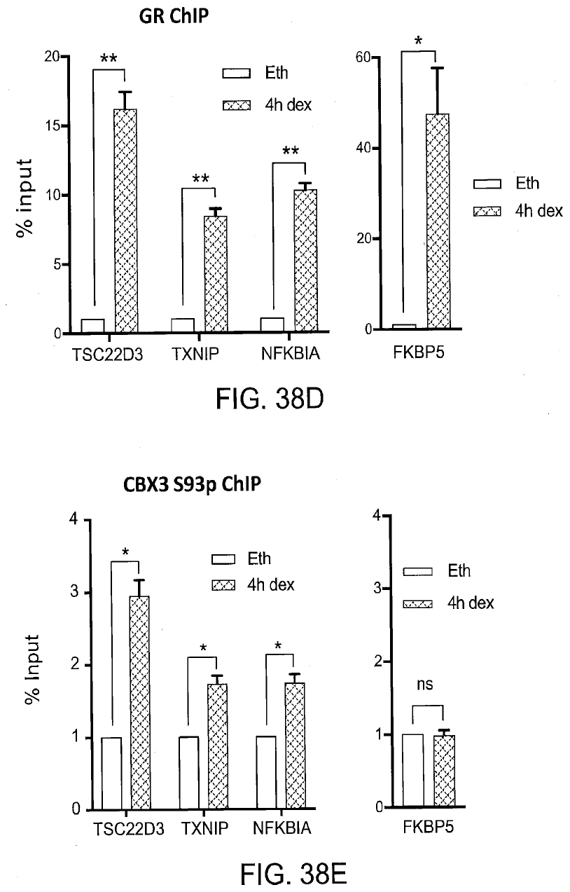

FIGS. 38A-1, 38A-2, 38A-3, 38A-4, 38B, 38C-1, 38C-2, 38C-3, 38D, and 38E show that EHMT2, EHMT1 and CBX3 are coactivators for a subset of GR target genes. As shown in FIGS. 38A-1, 38A-2, 38A-3, 38A-4, NALM-6 cells expressing shRNA against EHMT2, EHMT1, CBX3 or a non-specific sequence (shNS) were treated for 8 h with 100 nM dex or equivalent volume of vehicle ethanol. mRNA levels for the indicated GR target genes were measured by RT-qPCR and normalized to α-actin mRNA levels. Results shown are mean±SEM for three independent experiments. p-value was calculated using a paired t-test comparing results for each shRNA to shNS, *p≤0.05, **p≤0.01. For FKBP5, p-values for each shRNA compared to shNS were not significant. FIG. 38B is an immunoblot showing EHMT2, EHMT1, GR, CBX3 and GAPDH protein levels in extracts from NALM-6 cells analyzed in A. As shown in FIG. 38C, NALM-6 cells expressing shRNA against TSC22D3, NFKBIA, TXNIP, or a non-specific sequence (shNS) were treated with two-fold dilutions of dex for 72 h. Cell survival was measured by a fluorescence metabolic assay. An EC50 was calculated as the concentration at which half the cells remained alive, compare to vehicle controls. Error bars depict the SEM of 4 independent experiments and p-values were calculated to compare each coregulator depletion to shNS using a paired t-test *p≤0.05, **p≤0.01. Insets show immunoblots or mRNA levels (bar graphs) measured by RT-qPCR and normalized to (3-actin mRNA levels. As shown in FIGS. 38D and 38E, CBX3 is selectively recruited to EHMT2/EHMT1-dependent GR target genes in response to dex. NALM-6 cells were treated with 100 nM dex or ethanol for 4 h. ChIP was performed with antibody against GR (FIG. 38D) or CBX3 phosphorylated at S93 (CBX3-S93p) (FIG. 38E), and immunoprecipitated DNA was analyzed by qPCR using primers that amplify the GBRs associated with the indicated GR target genes. Results are normalized to input chromatin and shown as mean±SEM for three independent experiments. P-value was calculated using a paired t-test, *p≤0.05, **p≤0.01; ns, not significant.

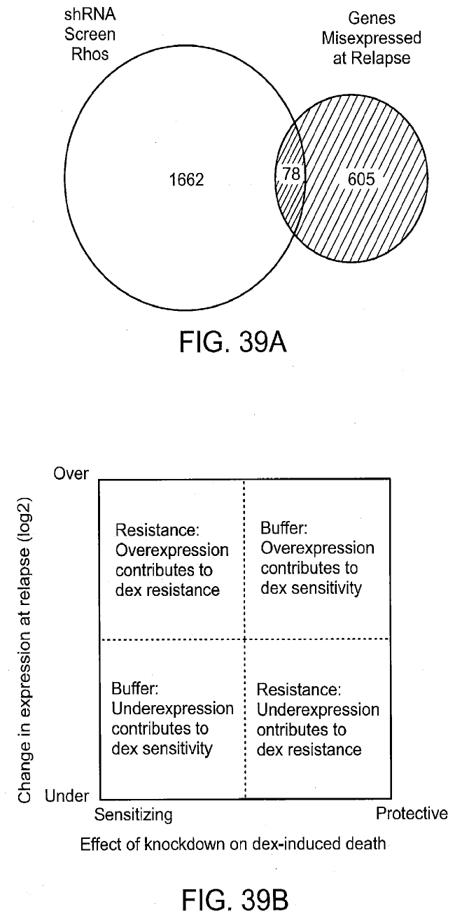

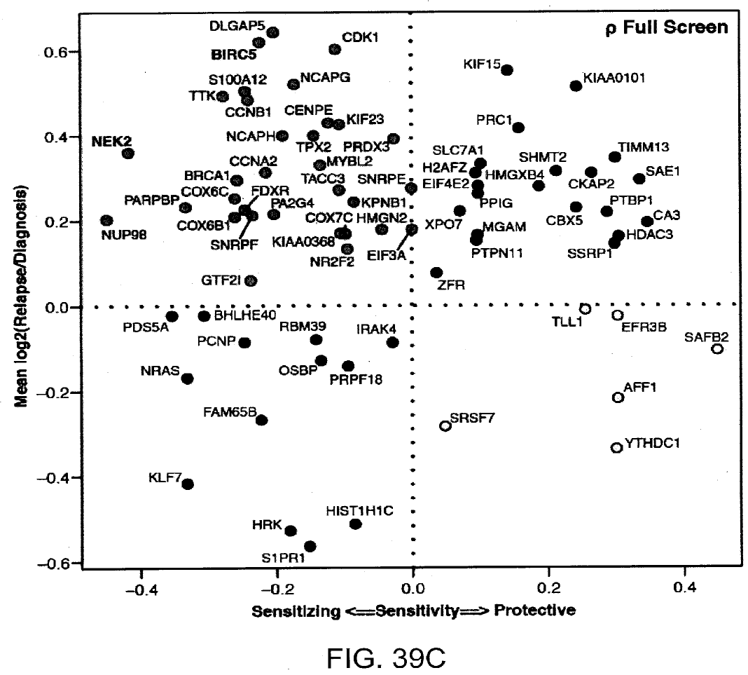

FIGS. 39A, 39B, 39C, 39D show overexpression of cell cycle genes is functionally linked to dex resistance in B-ALL. As shown in FIG. 39A, the intersection of genes that affect the dex sensitivity (rho) of NALM6 cells (purple 24, p-value≤0.05) and genes misexpressed at relapse in B-ALL (Yellow 25, p-value≤0.05). FIG. 39B is the plotting of the effect of gene depletion on dex-induced death versus the change in expression at relapse identifies genes specifically associated with dex-resistance at relapse. FIG. 39C shows overexpression of genes at relapse that increase dex-sensitivity when depleted (orange 22) contributes to dex resistance. Reduced expression at relapse of genes that decrease dex-sensitivity when depleted (yellow 23) can also contributes to dex resistance. The misexpression of other genes (purple, green) increase dex sensitivity. FIG. 39D shows relapse-resistance genes are associated with cell cycle and DNA damage (Ingenuity Pathway Analysis).

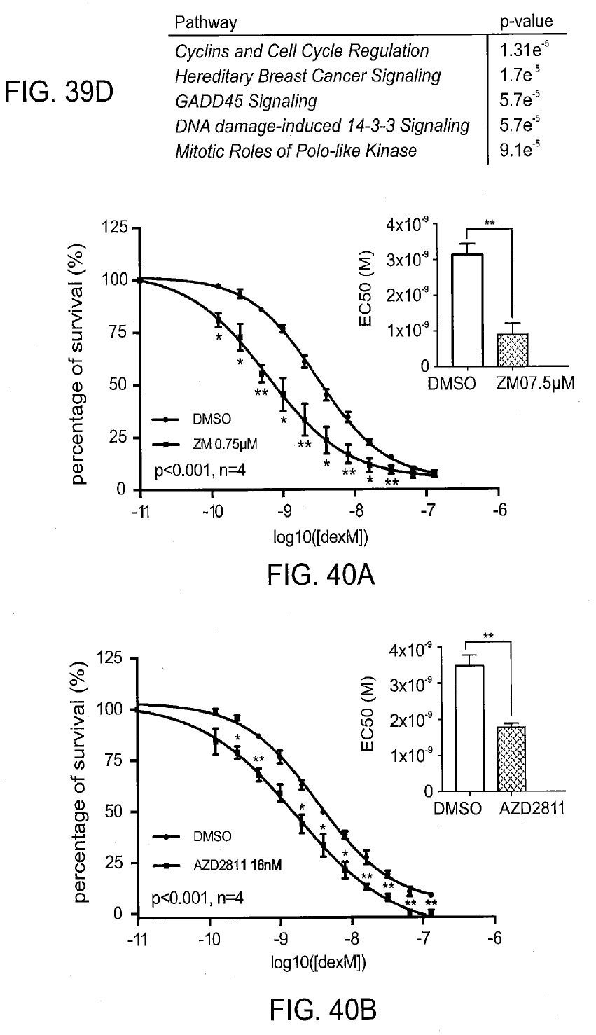

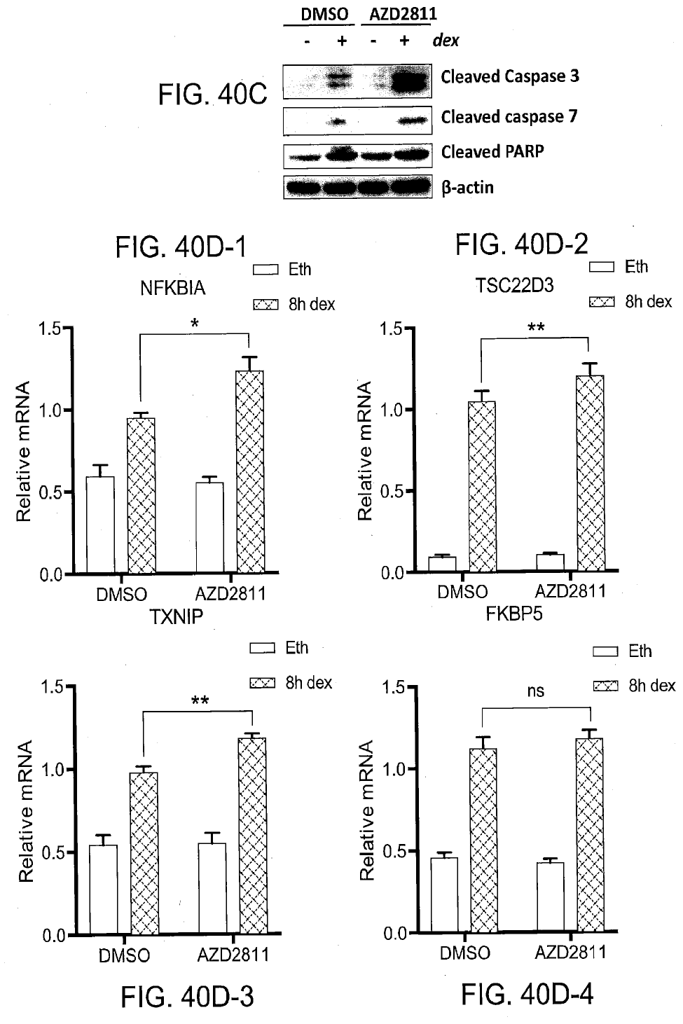

FIGS. 40A, 40B, 40C, 40D-1, 40D-2, 40D-3, and 40D-4 show Aurora kinase B inhibitors sensitizing NALM6 cells to GC-induced cell death. As shown in FIGS. 40A and 40B, NALM6 cells were treated with the indicated dex concentration in addition to 0.75 μM ZM447439 (FIG. 40A), 16 nM AZD2811 (FIG. 40B), or equivalent volume of vehicle DMSO for 72 h, and cell survival was measured by a fluorescence metabolic assay. In each condition, the value measured with dex was normalized to the fluorescence value measured with ethanol. Percentage of survival is shown as the mean±SEM of 4 independent experiments and p-values for individual dex concentrations were calculated using a paired t-test. *p≤0.05, **p≤0.01, * * *p≤0.001. F-test comparing the two curves: p≤0.001. Insets show the EC50s that were calculated as the concentration at which half the cells remained alive, compare to vehicle controls. Error bars depict the SEM of 4 independent experiments and p-values were calculated to compare AURKB inhibitor treatment to DMSO using a paired t-test ** p≤0.01, * * *p≤0.001. As shown in FIGS. 40C, NALM6 cells were pretreated for 24 h with DMSO or 16 nM AZD2811, and then ethanol (−) or 100 nM dex (+) was added for an additional 24 h. The indicated proteins were then examined by immunoblot. As shown in FIGS. 40D-1, 40D-2, 40D-3, and 40D-4, NALM6 cells pre-treated with AZD2811 (16 nM) or DMSO for 24 h, were then treated for 8 h with 100 nM dex or ethanol. mRNA levels for the indicated GR target genes were measured by RT-qPCR and normalized to β-actin mRNA levels. Results shown are mean±SEM for three independent experiments. p-value was calculated using a paired t-test, *p≤0.05, **p≤0.01, ns, not significant.

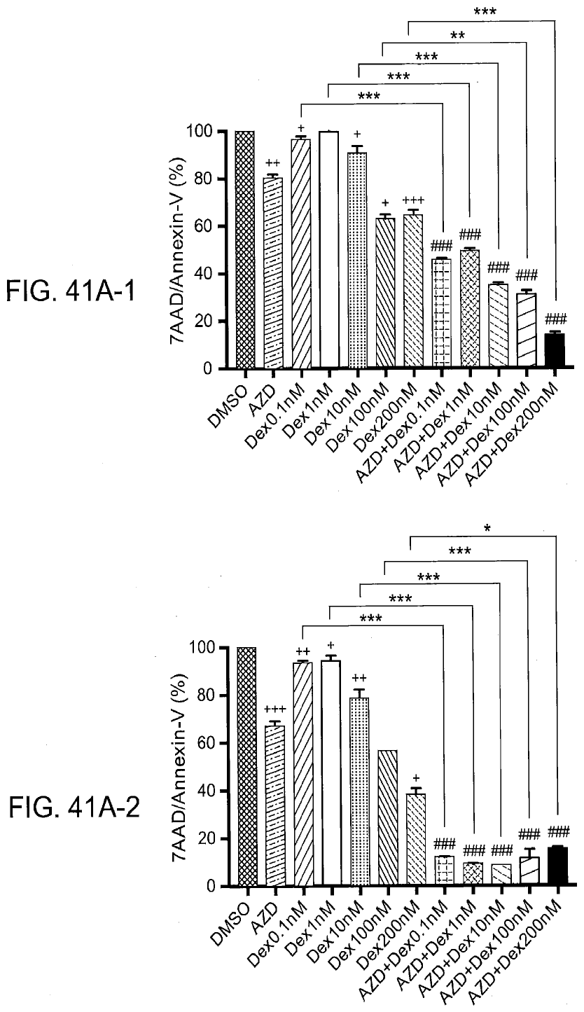

FIGS. 41A-1, 41A-2, 41B-1, and 41B-2 show that AURKB inhibition enhances GC-induced death of primary B-ALL cells from relapsed patients. As shown FIGS. 41A-1, 41A-2, 41B-1, and 41B-2, LAX7R (FIGS. 41A-1 and 41A-2) or LAX56 (FIGS. 41B-1 and 41B-2) primary human B-ALL cells were co-cultured with OP-9 feeder cells for 3 days (left) or 5 days (right) in the presence of the indicated drugs, and cell survival was determined by staining with Annexin/7AAD. p-value was calculated using a paired t-test, and different symbols were used to indicate p-values between different groups.+indicates statistical significance (+p≤0.05, ++p≤0.01 and + + + p≤0.001) between Dexamethasone or AZD2811 treated group and DMSO control group; # indicates statistical significance (#p≤0.05, ##p≤0.01 and # # # p≤0.001) between AZD2811+Dexamethasone combination group and AZD2811 group; * indicates statistical difference (*p≤0.05, **p≤0.01, * * *p≤0.001) between combination group and respective Dexamethasone treated groups. Values shown are mean and SD for 3 biological replicates, which are representative of 3 independent experiments.

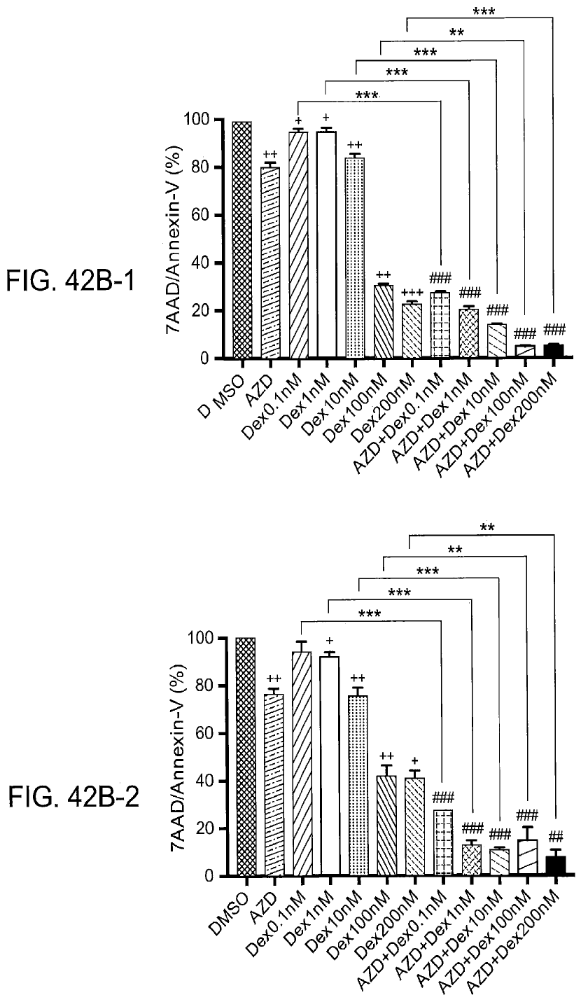

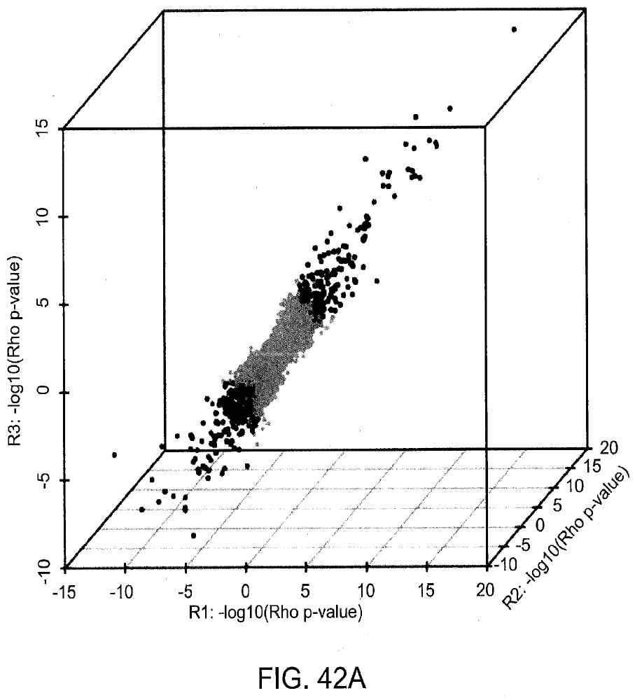

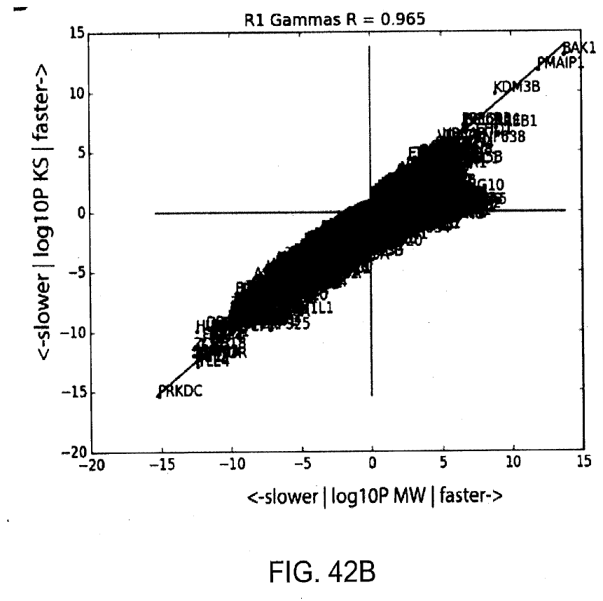

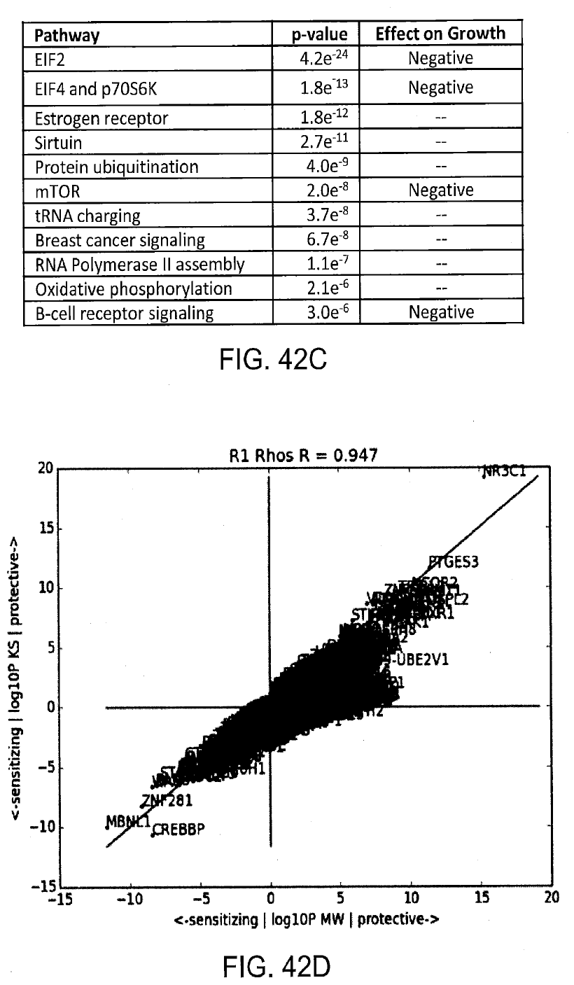

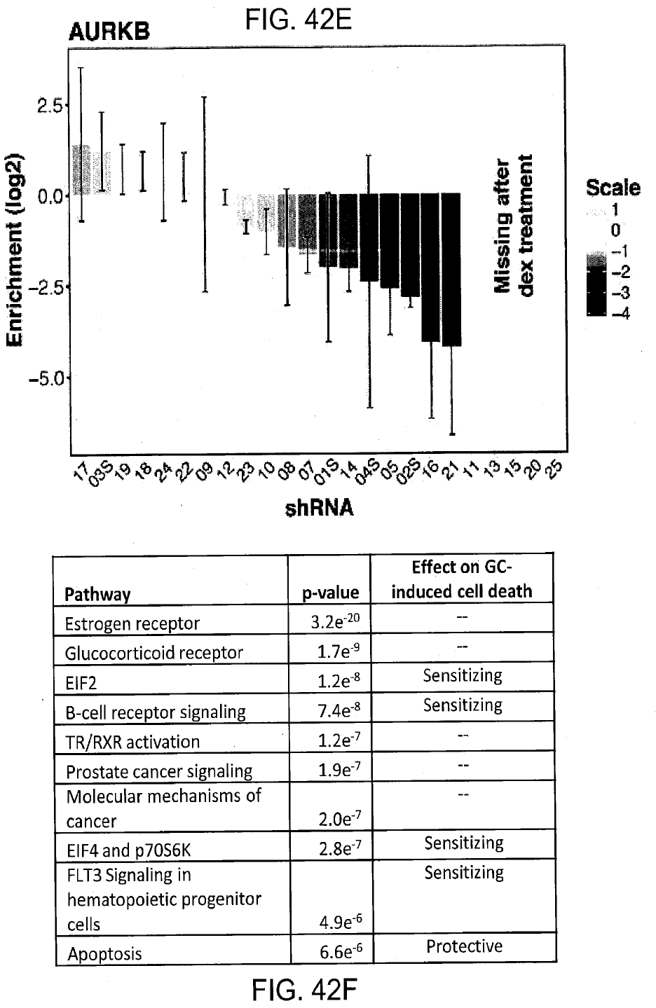

FIGS. 42A, 42B, 42C, 42D, 42E, and 42F show the results of the full next generation shRNA screen are sensitive and consistent despite dropout of some shRNAs. FIG. 42A is a plot of the p-values for each of the three biological replicates for Rho (change in dexamethasone sensitivity). The tight bundling of the points (gray) shows that the replicates are very consistent. Each point represents one gene, colored or darker gray points show significant changes for knockdown versus control across all replicates (qvalue≤0.1). FIGS. 42b and 42D are representative plots for the results of calculating the significance for each gene. On the x-axis are the (-log10) p-values for each gene calculated by the Mann-Whitney test, and the y-axis by the Kolmogorov-Smirnov test. For both Gamma (FIG. 42B, growth) and Rho (FIG. 42D, dex sensitivity) a subset of genes were calculated significant by the Mann-Whitney test (y˜0, x>0), but not the Kolmogorov-Smirnov test. FIG. 42C shows genes that significantly (qvalue≤0.1) affect the growth of NALM6 cells were analyzed using Ingenuity Pathway Analysis (Qiagen). If protein depletion has a consistent effect on a pathway, the effect is scored as either “Positive” or “Negative”. If depletion of the genes included in the pathway are not predicted to have a consistent effect on the pathway, it receives no score (—). FIG. 42E is an example of one of these genes, AURKB, shows that most shRNAs sensitize NALM6 cells to dex, and are depleted in the dex-treated cell population. Despite a clear trend, the low significance appears to be due to shRNAs that fall out of the screen during dex treatment. FIG. 42F shows genes that significantly (qvalue≤0.1) affect the dexamethasone sensitivity of NALM6 cells were analyzed using Ingenuity Pathway Analysis (Qiagen). If protein depletion has a consistent effect on a pathway, the effect is scored as either “Sensitizing” or “Protective”. For example, and consistent with our previous study, depletion of B-cell receptor components has a consistently sensitizing effect on NALM6 cells. If depletion of the genes included in the pathway are not predicted to have a consistent effect on the pathway, it receives no score (—).

FIGS. 43A, 43B-1, 43B-2, 43C-1, 43C-2, 43D-1, 43D-2, 43D-3, 43D-4 are validation of shRNAs on EHMT2 and EHMT1. As shown in FIGS. 43A, 43B-1, 43B-2, 43C-1, and 43C-2, NALM-6 cells expressing shRNAs against EHMT2 (shEHMT2, A), EHMT1 (shEHMT1, B), CBX3 (shCBX3, C) or a non-specific sequence (shNS) were treated with the indicated dex concentration for 72 h, and cell survival was measured by a fluorescence metabolic assay. In each condition, the intensity measured with dex was normalized to the fluorescence intensity measured with ethanol. Percentage of survival is shown as the mean±SEM of 4 individual experiments and p-values for results at individual dex concentrations were calculated using a paired t-test. *p≤0.05, **p≤0.01, * * *p≤0.001. A F-test was also calculated to compare the two curves: p≤0.001. Inset shows immunoblot of depletion by the relevant shRNA. The corresponding EC50 was presented in FIG. 3C. As shown in FIGS. 43D-1, 43D-2, 43D-3, and 43D-4, pre-B 697 cells expressing shRNA against EHMT2, EHMT1, or shNS were treated and analyzed as in A. EC50s were calculated as in FIG. 36C. Immunoblots show EHMT2 and EHMT1 depletion.

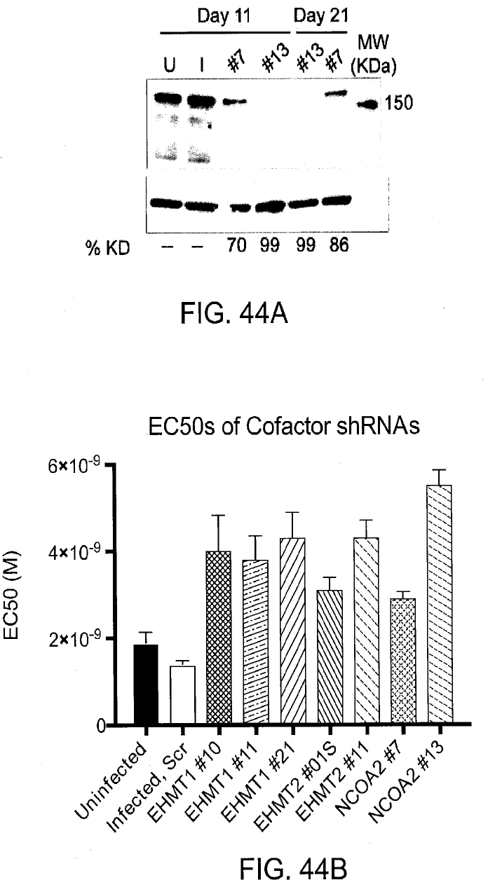

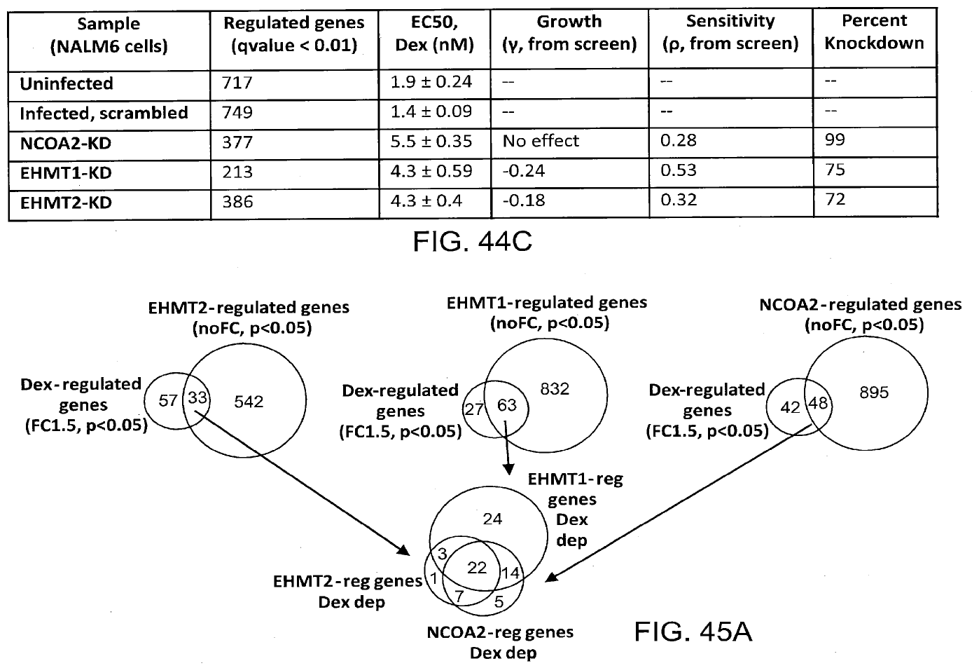

As shown in FIGS. 44A, 44B, and 44C, shRNAs directed against EHMT1, EHMT2, and NCOA2 induce durable depletion of proteins and decreased sensitivity to dex. shRNAs that were most enriched in the screen were individually cloned into a lentiviral packaging vector (pMK1221), then packaged into virus. NALM6 cells were infected with each virus, and the depletion of the target protein was monitored versus actin control for each coregulator. As shown in FIG. 44A, shRNA #13 directed against NCOA2 produces almost complete depletion of the protein, even after 3 weeks. FIG. 44B shows changes in sensitivity to dex upon coregulator depletion were measured in NALM6 cells. Cells were grown in 96-well plates and treated with two-fold dilutions of dexamethasone for 3 days. Cell survival was measured by PrestoBlue. An EC50 was calculated as the concentration at which half the cells remained alive, compare to vehicle controls. Error bars depict the standard deviation of at least 3 replicates. FIG. 44C is statistics for NALM6 cell populations infected with coregulator shRNAs or scrambled control shRNA that were analyzed by microarray.

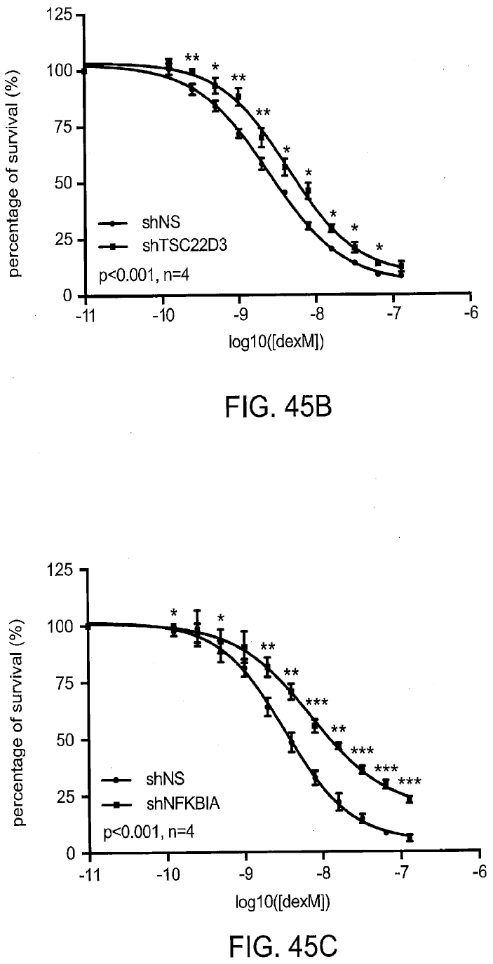

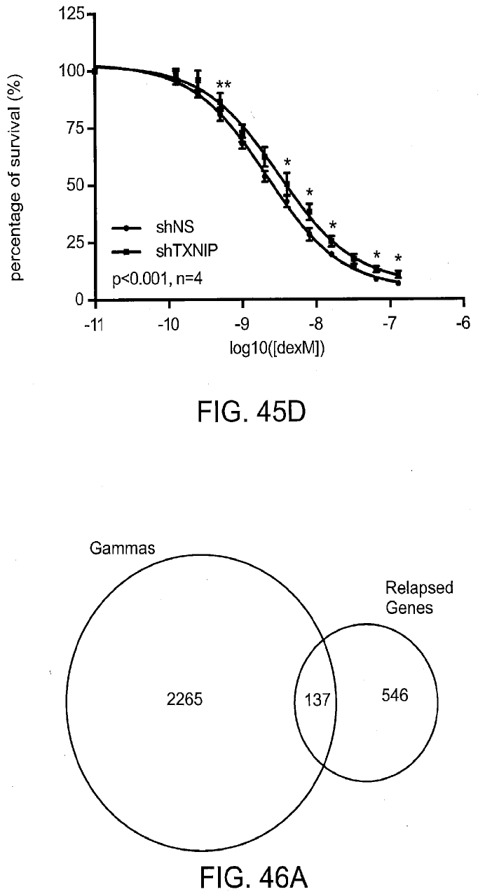

FIGS. 45A, 45B, 45C, 45D show that EHMT1/EHMT2/NCOA2 are coactivators for a subset of endogenous target genes. At the top of FIG. 45A, the small gray Venn diagram represents the total number of dex-regulated genes from the microarray analysis (q-value≤0.05 and at least 1.5-fold increase or decrease) for NALM-6 cells expressing shNS and treated with 1 μM dex for 4 h compared with ethanol. Large blue Venn diagram (left) represents the number of EHMT2-regulated genes with significantly different expression (q-value≤0.05, no fold-change cutoff) in dex-treated cells expressing shEHMT2 versus shNS. Large orange Venn diagram (middle) represents the number of EHMT1-regulated genes with significantly different expression in dex-treated cells expressing shEHMT1 versus shNS. Large green Venn diagram (right) represents the number of NCOA2-regulated genes with significantly different expression in dex-treated cells expressing shNCOA2 versus shNS. Overlap areas indicate the number of genes shared between sets. At the bottom, the genes from the intersections of the three top diagrams are overlapped. As shown in FIGS. 45B, 45C, 45D, NALM-6 cells expressing shRNA against TSC22D3, NFKBIA, TXNIP, or a non-specific sequence (shNS) were treated with the indicated dex concentration for 72 h, and cell survival was measured by a fluorescence metabolic assay. In each condition, the intensity measured with dex was normalized to the fluorescence intensity measured with ethanol. Percentage of survival is shown as the mean±SEM of 4 individual experiments and the p-value was calculated using a paired t-test. *p≤0.05, **p≤0.01, * * *p≤0.001. F-test: p≤0.001. The corresponding EC50 was presented in FIGS. 38C-1, 38C-2, 38C-3.

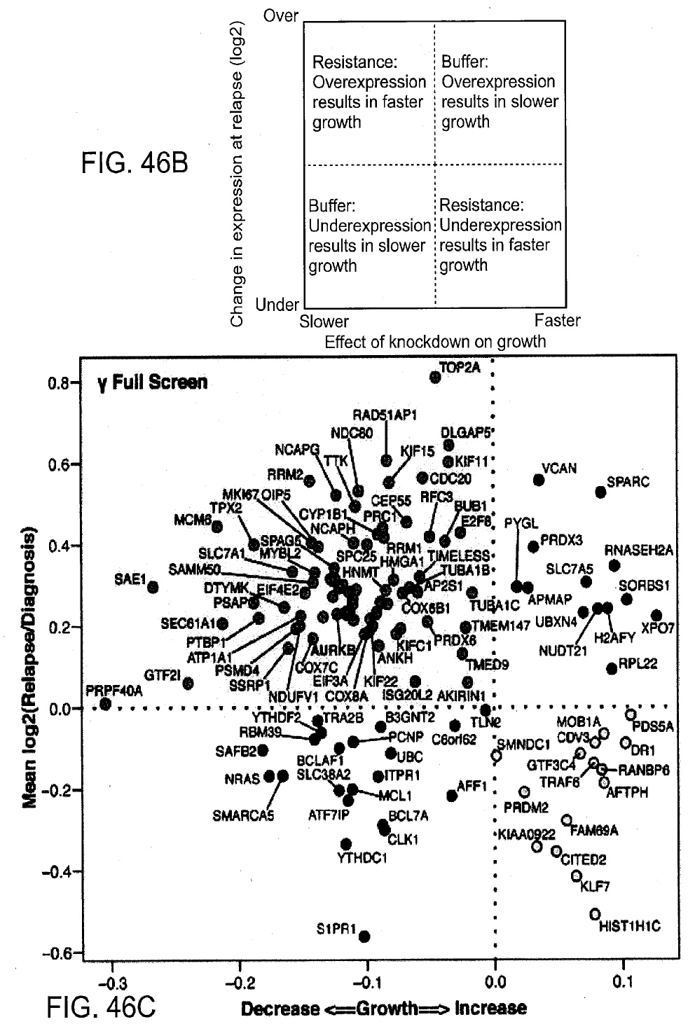

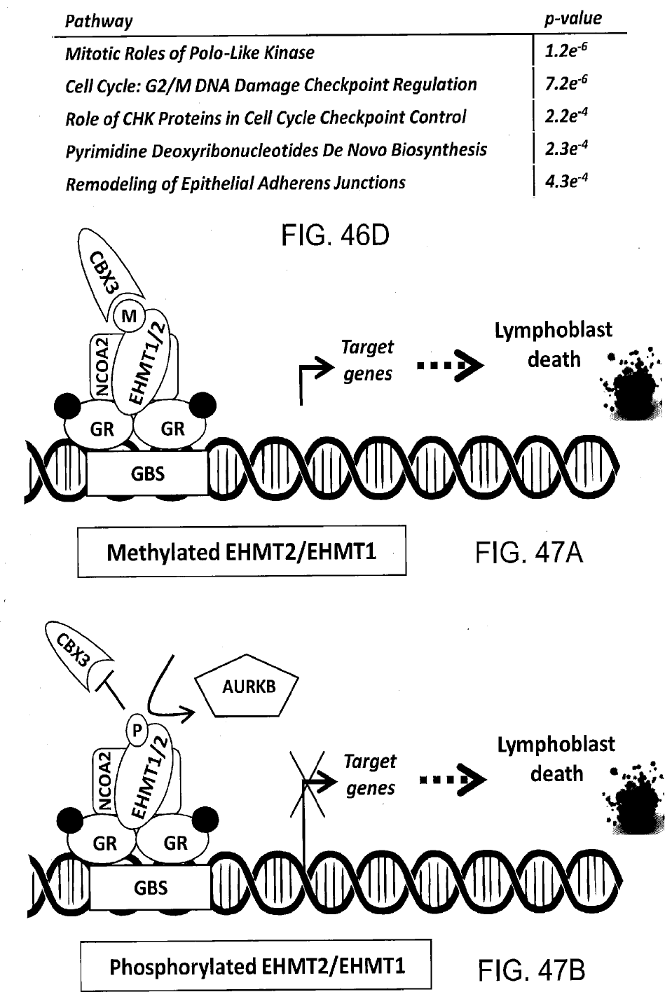

FIGS. 46A, 46B, 46C, and 46D show the identification of resistance genes based on misexpression and effect on cell growth (Gamma). FIG. 46A shows genes misexpressed at relapse (Yellow 29, p-value≤0.05) are compared to those with a significant (Gammas, Blue 28, p-value≤0.05) effect on the growth of NALM6 cells. FIG. 46B is a schematic for how resistance genes are identified. Genes that slow growth when depleted may increase proliferation when overexpressed at relapse (orange 26). Similarly, genes that increase growth when depleted cause an increase in growth when underexpressed (yellow 27). As shown in FIG. 46C, of the 137 genes that are misexpressed and cause a growth phenotype, 101 can be classified as relapse genes (orange and yellow). Two sets of genes may buffer this growth effect. Genes that are overexpressed at relapse and increase growth when depleted (purple 30) may suppress increased growth at relapse. Similarly, genes that decrease growth when depleted likely decrease growth when they are underexpressed at relapse (green). (D) Misexpressed genes with an effect on growth from Table 9 were analyzed by Ingenuity Pathway Analysis (Qiagen) to identify pathways affected.

TABLE 9
Misexpressed genes with an effect on growth.

| | Gamma | Gamma | Relapse/ | Fisher's |
|---|---|---|---|---|
| Symbol | phenotype | p-value | diagnostic (log2) | p-value |
| ACSBG1 | −0.09 | 4.94E−04 | 0.23 | 5.13E−03 |
| AFTPH | 0.08 | 5.12E−03 | −0.19 | 9.13E−03 |
| AKIRIN1 | −0.02 | 2.68E−02 | 0.06 | 8.46E−03 |
| ANKH | −0.09 | 3.59E−02 | 0.15 | 7.96E−03 |
| AP2S1 | −0.06 | 6.98E−03 | 0.28 | 1.33E−06 |
| ATP1A1 | −0.15 | 6.81E−04 | 0.22 | 2.44E−03 |
| AURKA | −0.12 | 5.90E−03 | 0.3 | 1.76E−04 |
| AURKAIP1 | −0.15 | 1.47E−02 | 0.25 | 2.67E−03 |
| AURKB | −0.12 | 7.15E−04 | 0.23 | 3.60E−04 |
| BUB1 | −0.04 | 4.66E−04 | 0.4 | 3.11E−03 |
| C14ORF2 | −0.1 | 3.71E−02 | 0.21 | 3.20E−03 |
| CBX5 | −0.12 | 4.31E−05 | 0.23 | 7.42E−03 |
| CDC20 | −0.06 | 3.14E−02 | 0.56 | 4.18E−05 |
| CDC25B | −0.02 | 3.08E−02 | 0.24 | 1.83E−03 |
| CDCA8 | −0.12 | 1.65E−03 | 0.23 | 2.17E−02 |
| CDK1 | −0.2 | 2.31E−02 | 0.6 | 2.16E−04 |
| CDV3 | 0.08 | 9.40E−03 | −0.09 | 8.75E−03 |
| CEP55 | −0.09 | 3.62E−03 | 0.44 | 1.18E−02 |
| CHEK1 | −0.15 | 4.58E−02 | 0.38 | 9.64E−03 |
| CITED2 | 0.05 | 6.96E−04 | −0.36 | 2.68E−03 |
| COX6B1 | −0.05 | 2.74E−03 | 0.21 | 6.13E−03 |
| COX7C | −0.14 | 2.63E−02 | 0.17 | 5.78E−03 |
| COX8A | −0.1 | 1.20E−02 | 0.18 | 6.65E−03 |
| CSF3R | −0.11 | 2.87E−02 | 0.21 | 3.45E−03 |
| CYP1B1 | −0.11 | 2.20E−02 | 0.4 | 1.94E−05 |
| DLGAP5 | −0.04 | 3.27E−03 | 0.64 | 4.74E−04 |
| DNMT1 | −0.11 | 2.92E−06 | 0.25 | 1.53E−03 |
| DR1 | 0.1 | 2.24E−04 | −0.09 | 1.65E−02 |
| DTYMK | −0.17 | 1.49E−06 | 0.24 | 5.28E−04 |
| E2F8 | −0.03 | 2.79E−02 | 0.43 | 1.63E−03 |
| EIF3A | −0.1 | 8.83E−04 | 0.18 | 5.57E−03 |
| EIF4E2 | −0.15 | 2.42E−04 | 0.28 | 1.65E−03 |
| ERH | −0.13 | 5.82E−04 | 0.27 | 2.02E−02 |
| FAM69A | 0.06 | 9.96E−03 | −0.28 | 1.68E−02 |
| FBL | −0.09 | 2.67E−03 | 0.25 | 1.43E−02 |
| GMPS | −0.12 | 1.11E−04 | 0.29 | 2.19E−03 |
| GTF2I | −0.24 | 5.91E−10 | 0.06 | 5.75E−04 |
| GTF3C4 | 0.07 | 5.56E−03 | −0.12 | 3.53E−03 |
| H2AFZ | −0.13 | 6.75E−07 | 0.31 | 3.81E−03 |
| HIST1H1C | 0.08 | 3.14E−04 | −0.51 | 3.01E−03 |
| HMGA1 | −0.08 | 2.52E−02 | 0.31 | 6.43E−04 |
| HMGXB4 | −0.07 | 3.60E−03 | 0.28 | 5.87E−03 |
| HNMT | −0.09 | 2.39E−03 | 0.28 | 3.18E−03 |
| IDH2 | −0.11 | 1.73E−03 | 0.29 | 4.83E−03 |
| ISG20L2 | −0.06 | 3.01E−02 | 0.06 | 1.94E−02 |
| KIAA0922 | 0.03 | 5.51E−03 | −0.35 | 5.23E−04 |
| KIF11 | −0.04 | 4.84E−03 | 0.6 | 1.72E−04 |
| KIF15 | −0.08 | 5.42E−03 | 0.55 | 2.01E−03 |
| KIF22 | −0.1 | 1.30E−02 | 0.2 | 4.84E−03 |
| KIFC1 | −0.08 | 2.51E−03 | 0.18 | 1.31E−04 |
| KLF7 | 0.06 | 3.25E−02 | −0.42 | 9.95E−04 |
| MCM6 | −0.22 | 1.28E−06 | 0.44 | 7.93E−04 |
| MKI67 | −0.14 | 3.73E−06 | 0.4 | 6.12E−06 |
| MOB1A | 0.08 | 3.01E−02 | −0.07 | 1.07E−02 |
| MRPS15 | −0.13 | 1.07E−05 | 0.22 | 6.69E−03 |
| MRPS18B | −0.09 | 2.89E−02 | 0.26 | 2.33E−04 |
| MYBL2 | −0.14 | 1.41E−04 | 0.33 | 1.85E−03 |
| NCAPG | −0.12 | 2.89E−02 | 0.52 | 3.02E−05 |
| NCAPG2 | −0.11 | 2.91E−02 | 0.26 | 5.56E−03 |
| NCAPH | −0.1 | 4.70E−05 | 0.4 | 1.57E−06 |
| NDC80 | −0.11 | 8.07E−03 | 0.53 | 6.30E−05 |
| NDUFA13 | −0.09 | 4.47E−03 | 0.23 | 9.86E−03 |
| NDUFV1 | −0.15 | 3.87E−03 | 0.2 | 3.51E−03 |
| OIP5 | −0.14 | 2.76E−04 | 0.39 | 2.53E−03 |
| PDS5A | 0.11 | 2.26E−04 | −0.02 | 6.09E−03 |
| PLK4 | −0.07 | 2.16E−02 | 0.38 | 6.21E−04 |
| POLE2 | −0.12 | 4.32E−03 | 0.31 | 1.18E−02 |
| PRC1 | −0.09 | 1.88E−04 | 0.41 | 2.06E−03 |
| PRDM2 | 0.02 | 9.71E−03 | −0.21 | 8.48E−03 |
| PRDX6 | −0.02 | 3.78E−02 | 0.19 | 8.42E−03 |
| PRPF40A | −0.31 | 3.64E−09 | 0.01 | 1.07E−02 |
| PSAP | −0.19 | 9.45E−10 | 0.25 | 2.24E−04 |
| PSMD4 | −0.16 | 5.27E−08 | 0.19 | 7.80E−03 |
| PTBP1 | −0.18 | 2.34E−09 | 0.22 | 2.34E−03 |
| PTPN13 | −0.02 | 6.96E−03 | 0.03 | 8.76E−03 |
| RAD51AP1 | −0.08 | 1.81E−02 | 0.6 | 2.62E−04 |
| RANBP6 | 0.08 | 1.96E−02 | −0.16 | 1.52E−02 |
| RFC3 | −0.07 | 7.98E−03 | 0.45 | 2.80E−03 |
| RPN2 | −0.08 | 2.64E−02 | 0.25 | 5.07E−04 |
| RRM1 | −0.05 | 4.86E−02 | 0.42 | 1.51E−04 |
| RRM2 | −0.14 | 4.31E−02 | 0.55 | 1.01E−03 |
| SAE1 | −0.27 | 1.03E−03 | 0.29 | 5.14E−04 |
| SAMM50 | −0.14 | 7.04E−04 | 0.3 | 7.54E−03 |
| SEC61A1 | −0.21 | 3.18E−04 | 0.2 | 4.03E−03 |
| SHMT2 | −0.13 | 6.96E−03 | 0.31 | 1.15E−04 |
| SLC7A1 | −0.16 | 1.46E−06 | 0.33 | 1.48E−02 |
| SMARCC1 | −0.12 | 3.41E−05 | 0.22 | 6.60E−05 |
| SMNDC1 | 1.00E−03 | 1.18E−04 | −0.12 | 8.61E−03 |
| SPAG5 | −0.13 | 3.50E−05 | 0.34 | 1.70E−03 |
| SPC25 | −0.09 | 4.86E−03 | 0.42 | 2.80E−03 |
| SSRP1 | −0.16 | 1.62E−07 | 0.14 | 7.71E−03 |
| TIMELESS | −0.06 | 5.50E−05 | 0.32 | 8.01E−03 |
| TMED9 | −0.03 | 8.66E−03 | 0.13 | 6.92E−03 |
| TMEM147 | −0.07 | 7.39E−03 | 0.19 | 1.45E−02 |
| TOP2A | −0.05 | 3.78E−02 | 0.81 | 9.88E−07 |
| TPX2 | −0.19 | 5.10E−08 | 0.4 | 1.60E−03 |
| TRAF6 | 0.08 | 2.69E−02 | −0.14 | 4.15E−04 |
| TUBA1B | −0.07 | 1.14E−03 | 0.29 | 9.03E−04 |
| TUBA1C | −0.02 | 1.74E−02 | 0.28 | 1.83E−03 |
| TUBB | −0.11 | 4.15E−03 | 0.28 | 4.26E−03 |
| TUBG1 | −0.11 | 1.92E−02 | 0.24 | 7.68E−03 |

Table 9 shows misexpressed genes with an effect on growth. Genes that are misexpressed upon relapse have the potential to be deleterious if they have an effect on growth. We identify these genes as having their expression change significantly from diagnosis to relapse (Fisher p-value≤0.01) and causing a significant effect on the growth (gamma) of NALM-6 cells (Gamma p-value≤0.05). Genes whose knockdown makes cells die or grow more slowly would be predicted to increase growth if overexpressed. Thus, genes that are overexpressed upon relapse (positive Relapse/Diagnostic) and cause cells to grow more slowly (Gamma phenotype<0) may cause greater proliferative potential. By the same logic, genes that are underexpressed upon relapse (negative Relapse/Diagnostic) and make cells grow faster upon knockdown (Gamma phenotype>0) may also cause greater proliferative potential.

FIGS. 47A and 47B show models for regulation of dex-induced genes involved in B-ALL cell death by GR, EHMT2, EHMT1, CBX3, and AURKB. Left, GR recruits NCOA2/EHMT2/EHMT1. Methylated EHMT2/EHMT1 recruit CBX3, which recruits RNA polymerase II to activate transcription of cell death genes and promotes lymphoblast death. Right, Phosphorylation of EHMT2/EHMT1 by Aurora kinase B prevents CBX3 recruitment, reduces death gene activation by GC, and reduces leukemia cell death.

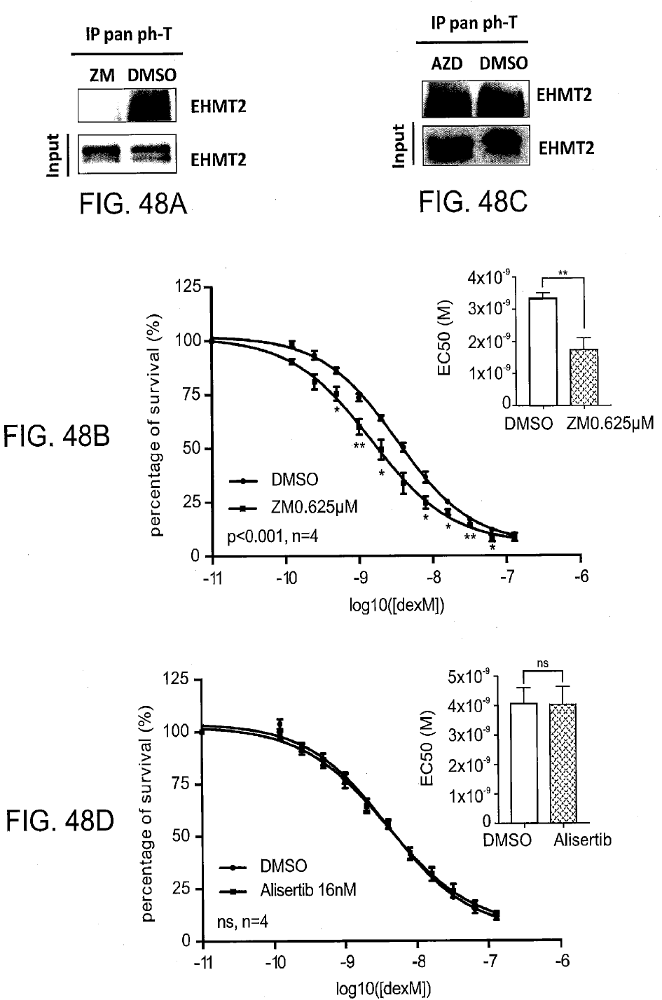

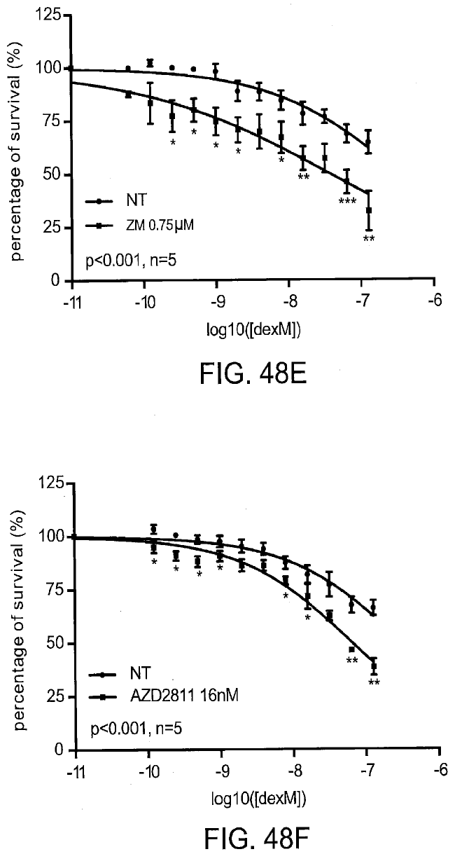

FIGS. 48A, 48B, 48C, 48D, 48E, and 48F show the effect of AURKB inhibitors on EHMT2 phosphorylation and dex-induced cell death. FIG. 48A shows phosphorylation of EHMT2 in NALM-6 cells treated with 0.75 μM ZM447439 or DMSO for 24 h was analyzed by immunoprecipitation with pan ph-T antibody, followed by immunoblot with EHMT2 antibodies. As shown in FIG. 48B, NALM-6 cells were treated with 0.625 μM ZM447439 or DMSO for 72 h, and cell survival was measured by a fluorescence metabolic assay. In each condition, the value measured with dex was normalized to the fluorescence value measured with ethanol. Percentage of survival is shown as the mean±SEM of 4 independent experiments and p-values for individual dex concentrations were calculated using a paired t-test. *p≤0.05, **p≤0.01, * * *p≤0.001. F-test comparing the two curves: p≤0.001. EC50s were calculated as the concentration at which half the cells remained alive, compare to vehicle controls. Error bars depict the SEM of 4 independent experiments and p-value was calculated to compare ZM treatment to DMSO using a paired t-test **p≤0.01, * * *p≤0.001. FIG. 48C shows phosphorylation of EHMT2 in NALM-6 cells treated with 16 nM AZD2811 or DMSO for 24 h was analyzed as in A. As shown in FIG. 48D, NALM-6 cells were treated with the indicated dex concentration in addition to Alisertib (16 nM) or DMSO for 72 h, and cell survival was measured and analyzed as in B. F-test calculated was not significant (ns). EC50s were calculated as the concentration at which half the cells remained alive, compare to vehicle controls. Error bars depict the SEM of 4 independent experiments and p-value was calculated to compare alisertib treatment to DMSO using a paired t-test. Result was not significant. (E-F) RCH-ACV cells were treated with the indicated dex concentration in addition to 0.75 μM ZM447439 (FIG. 48E), 16 nM AZD2811 (FIG. 48F), or DMSO for 72 h, and cell survival was measured by a fluorescence metabolic assay. In each condition, the value measured with dex was normalized to the fluorescence value measured with ethanol. Percentage of survival is shown as the mean±SEM of 5 individual experiments and the p-value for each dex concentration was calculated using a paired t-test. *p≤0.05, **p≤0.01, * * *p≤0.001. F-test comparing the two curves: p≤0.001.

### Results

Transcriptome Analysis of Paired Diagnostic/Relapsed B-ALL Samples Identifies Cell Cycle Genes Associated with Relapse

Previously, three groups measured mRNA levels of B-ALL cells from paired diagnostic/relapse samples using Affymetrix arrays. Each study, containing data from 27 to 49 B-ALL patients, reported misexpressed genes related to cell cycle and replication (e.g. PTTG1, CDC20), apoptosis (BIRCS, HRK), and DNA repair (FANC genes) (Bhojwani et al., 2006; Hogan et al., 2011; Staal et al., 2010). A recent meta-analysis of these three studies (Chow et al., 2017) identified hundreds more genes misexpressed at relapse, however some originally identified genes were not found to be significant.

To increase the power and the number of misexpressed genes identified, we performed a combined analysis of these three paired diagnostic/relapsed data sets. Because one data set (GSE3912) was performed on a platform (Affymetrix U133A) different from the other two (GSE18497 and GSE28460; Affymetrix U133 Plus 2.0), we processed each separately. Increased statistical power was achieved by identifying genes that are consistently significant across all data sets using the Fisher and Stouffer methods, which calculates a combined p-value from the three individual p-values. We thereby identified 197 significantly misexpressed genes (qvalue≤0.1), the vast majority of which (169) are overexpressed (FIG. 34A). Subsequently, by combining all data into a single data set, we were able to identify a much larger number of significantly misexpressed genes than in the individual studies due to increased sample numbers, with nonetheless reasonable confidence levels. Of 683 top hits (p-value≤0.01), most (502) were again overexpressed (Supplementary Dataset 1).

Similar to previous analyses (McDonald et al., 2017; Rodriguez-Fraticelli et al., 2018), the top categories of genes misexpressed at relapse are cell cycle and replication (FIG. 34B), with cell cycle genes (e.g. CCNB2, CDK1) consistently overexpressed at relapse across all studies. Other genes, such as BIRCS (FIG. 34A), increase survival by opposing apoptosis (Fulda, 2009). These categories are dominated by overexpressed genes. Underexpressed genes are not related to cell cycle, but instead immune cell function (e.g. Toll-like receptor signaling and IL-1 signaling). These data suggest that relapse results from an increase in proliferation and survival and a decrease in immune-cell characteristics. Another pathway significantly enriched among the genes misexpressed at relapse indicates activation of prostaglandin signaling through PTGER2 (FIG. 34C). The significance of this finding suggests a strong link between PTGER2 and relapse that has not been reported previously.

Genome-Wide Identification of Genes that Influence Sensitivity to Dex-Induced Cell Death

To identify the factors that affect glucocorticoid sensitivity and to determine the impact of genes misexpressed in relapsed B-ALL, we measured the effect of every protein coding gene in the genome on growth and dex sensitivity using a next-generation shRNA screen. The screen targeted >20,000 protein coding genes with an average of 25 shRNAs per gene delivered by lentivirus, which allowed not only identification of high confidence hits, but also a quantitative measurement of the contribution of each gene to growth and dex sensitivity. We performed the screen in NALM6 cells, which has a dex response consistent with primary B-ALL tumor samples (Kruth et al., 2017). The screen was performed as described previously, except in spinner flasks rather than still tissue culture flasks (Kampmann et al., 2014; Kruth et al., 2017). From this we generated five libraries: T0, which was harvested immediately as an infection control; TF, our growth control which was treated with vehicle (ethanol) through the experiment; and three pools (R1, R2, and R3) that were each treated independently with three rounds of 35 nM dex. Libraries were then sequenced and processed as described (Kampmann et al., 2014). The dex-treated biological repeats (R1-3) showed excellent concordance (FIG. 42A).

Hundreds of genes affected either growth or sensitivity to dex (Supplementary Dataset 2). The effect of each gene on growth (y score) was calculated as the enrichment of cells containing the shRNA cassette for a particular gene measured in the mock treated replicate at the end of the experiment (TF) versus immediately after infection (TO). Mann-Whitney and Kolmogorov-Smirnov tests calculated whether the distribution of enrichments for shRNAs for a given gene is different from that for the control shRNAs. Although the tests in general agreed well, a cohort of genes that protected against dex-induced cell death exhibited greater significance by the Mann-Whitney test (FIG. 42B). This is likely because many of these genes affect cell growth and had shRNAs drop out during growth, and the Mann-Whitney test is less sensitive to a smaller number of shRNAs. We identified 1015 genes (qvalue≤0.1) that affected growth, with the majority (898 genes) impeding growth when knocked down (FIGS. 35A, 35C). Depletion of components involved in translation, mTOR signaling, and energy production had a negative growth impact (FIG. 42C). The B-cell receptor pathway was also important likely because signaling through this pathway enhances growth and survival of NALM6 cells (Kruth et al., 2017).

Fewer genes affected the dex-sensitivity of NALM6 cells. The effect of each gene on dex sensitivity (ρ score) is calculated as the enrichment of cells containing the shRNA cassette for a particular gene after dex treatment (R1-3) compared to growth control (TF). Importantly, depletion of GR (NR3C1), the sole target of dex, was the most protective, highest confidence hit in the screen (FIG. 35B). At the other end of the spectrum, MBNL1, was the most significant sensitizing hit. Similar to γ, the Mann-Whitney and Kolmogorov-Smirnov tests for p were largely similar but did not agree for a cohort of genes, again likely because of shRNA drop out during dex treatment (FIG. 42D). This dropout likely caused an underestimation of the significance of genes that affect growth or sensitivity. For example, most shRNAs for AURKB (a focus of the study) were depleted in the dex-treated cell populations compared with the untreated control (FIG. 42E). However, five AURKB shRNAs were present in the control but absent from the dex-treated population, presumably because they rendered cells extremely sensitive to dex. Their loss from the population reduced statistical power for AURKB (p-value=0.15) even though it had a substantial phenotype value (ρ=−0.30) (Supplementary Dataset 2).

Overall, we identified 132 genes (qvalue≤0.1) that contribute to GC-induced cell death in NALM6 cells (positive ρ, e.g. NR3C1 which is GR and EHMT1/2), and 140 that restrain dex sensitivity (negative ρ, e.g. EP300, MBNL1) (FIGS. 35B, 35C). Knockdown of genes in the estrogen and glucocorticoid signaling pathways had a significant impact on dex-induced cell death (FIG. 42F). However, because ESR1 (encoding estrogen receptor a) depletion did not have any effect on dex-sensitivity, the ER pathway appears to be significant because it shares many components with GR, especially transcriptional coregulators. As in the previous partial shRNA screen (Kruth et al., 2017), knockdown of components of B-cell receptor signaling (e.g. SYK, PI3Kδ) and translational machinery (EIF pathways) also sensitized cells to dex. This concordance validates the results of the current screen.

### Specific Nuclear Receptor Coregulators Affect Dex Sensitivity

Because nuclear receptor coregulators were identified prominently in the screen and are important for transcriptional regulation, we further examined the results for this class of proteins. Of 337 nuclear receptor coregulators identified on-line and in the literature (https://www.nursa.org/nursa/index.jsf) (Bakker et al., 2017; Kininis and Kraus, 2008; Petta et al., 2016), about one quarter (78) affected GC-induced cell death (FIG. 35D), using a more relaxed cutoff (p-value≤0.05). Depletion of 34 coregulators reduced sensitivity to dex (e.g. PTGES3, EHMT1), indicating that these coregulators contribute to GC-induced cell death. The most prominent hit was PTGES3 (aka p23), a chaperone that serves both as a coregulator for GR and as an enzyme producing the prostaglandin PGE2 (Tanioka et al., 2000). Depletion of the other 44 coregulators sensitized NALM6 cells to dex (e.g. CREBBP, KMT2D), indicating that they restrain GC-induced cell death. We therefore hypothesized that specific coregulators would cooperate with GR to regulate genes involved in GC-induced cell death.

### An Intact GR-EHMT1/2-CBX3 Complex is Required for Full Dex Potency

In our previous work, we established the interplay of EHMT1/2, CBX3, and AURKB in A549 cells (Poulard et al., 2017). EHMT1 (aka GLP) and EMHT2 (aka G9a) form a heterodimeric pair that automethylate and directly associate with GR. Automethylation forms a binding interface for CBX3 (aka HP1γ), which is required for full coactivator activity of the complex. AURKB opposes methylation and interaction of CBX3 by phosphorylating EHMT1/2, reducing the activity of GR on a subset of genes. Methylation and phosphorylation of EHMT2 and EHMT1 were found in Nalm6 cells by immunoprecipitation with antibodies against EHMT2 or EHMT1 followed by immunoblot with previously validated antibodies (Poulard et al., 2017) that recognize any protein containing methylated lysine (pan methyl K) or phosphorylated threonine (pan phospho T) (FIGS. 36A, 36B).

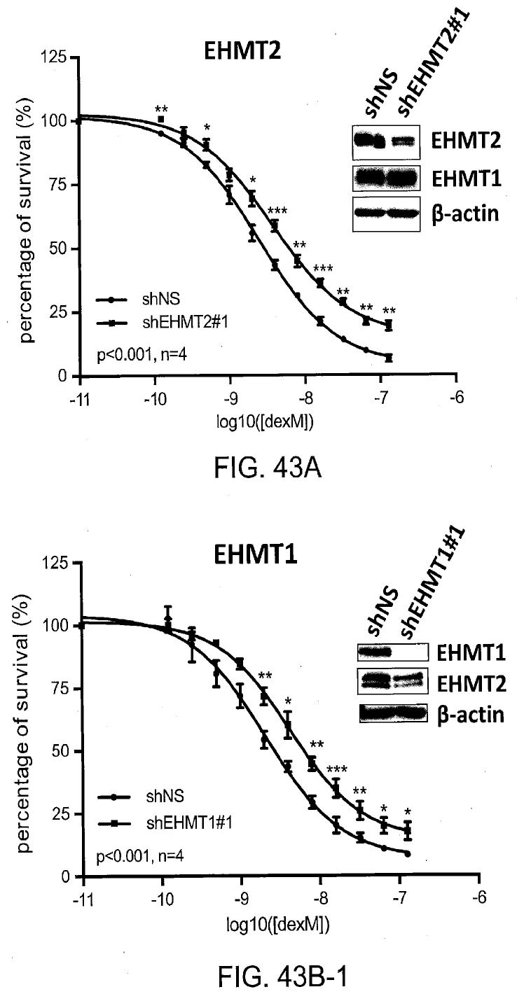

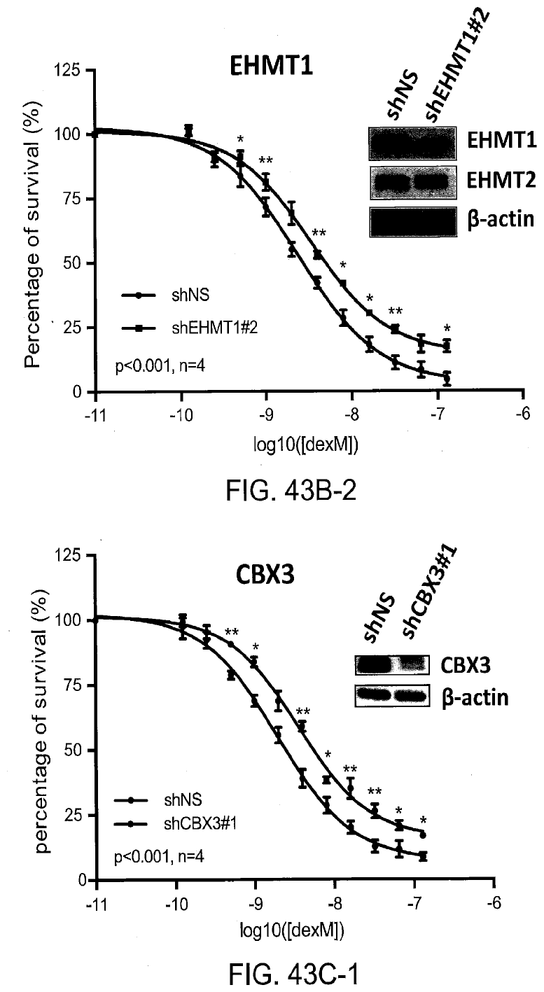

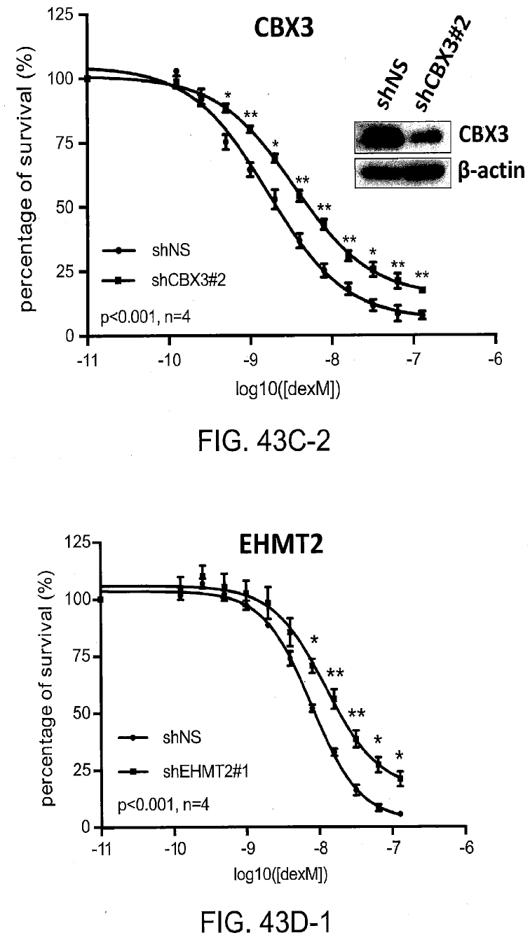

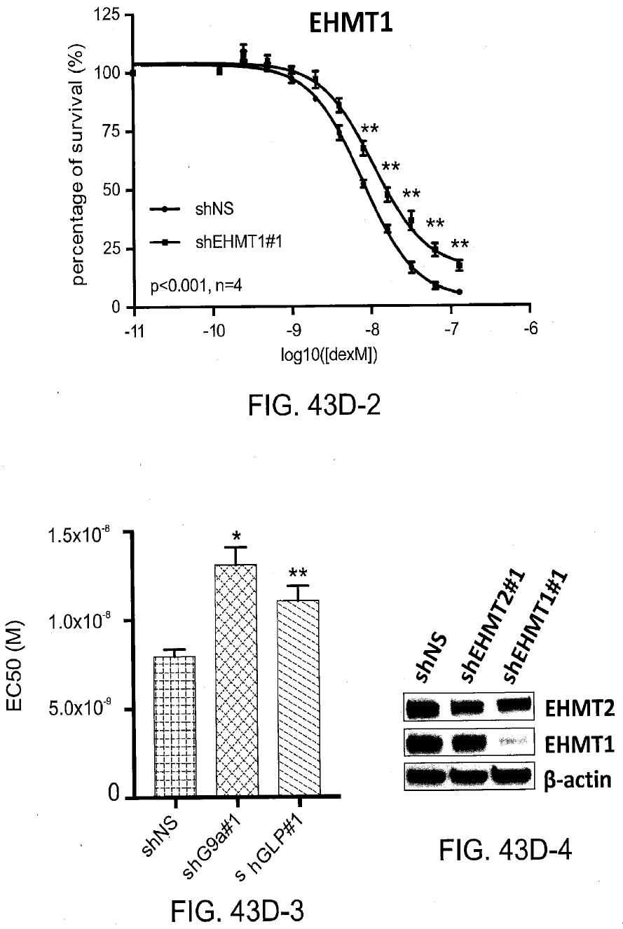

Data from the shRNA screen revealed that EHMT2, EHMT1, and CBX3 had strong positive ρ values (EHMT2, +0.33, EHMT1, +0.53, CBX3, +0.32) indicating that these proteins contribute to dex sensitivity (FIGS. 35B, 35D). To assay the importance of the intact GR-EHMT1/2-CBX3 complex, NALM-6 cells were then depleted of EHMT2, EHMT1 or CBX3 by lentiviral vectors expressing shRNAs (FIGS. 43A, 43B, 43C inset panels). Cells were exposed to increasing dex concentrations for 72 h, and cell survival was measured by a fluorescence metabolic assay. Cells depleted of EHMT2, EHMT1 or CBX3 were more resistant to dex than cells expressing a control non-specific shRNA (shNS) (FIGS. 36C, 43A, 43B, 43C). Another dex-sensitive B-ALL cell line, (pre-B 697) also became less sensitive to dex when EHMT2 or EHMT1 was depleted (FIG. 43D), indicating that this mechanism is not limited to the NALM6 cell line. Decreased Caspase 3, 7, and Poly(ADP-Ribose) Polymerase 1 (PARP1) cleavage in EHMT1/2 or CBX3 depleted cells after 24 hours of dex treatment confirmed attenuation of apoptosis rather than simply growth (FIGS. 36D, 36E, 36F).

### Depletion of Coregulators Generally Attenuates Dex Regulation and Causes Misregulation of Specific GC-Regulated Genes

To understand mechanistically how coregulator depletion affects dex sensitivity, we measured the effect on dex-regulation of genes. We chose three coregulators to deplete; EHMT1, EHMT2, and NCOA2 (aka GRIP1/SRC2/TIF2) which is known to cooperate with EHMT1 and EHMT2 as a coactivator (Bittencourt et al., 2012; Lee et al., 2006; Poulard et al., 2017). GR recruits EHMT1 and EHMT2 as well as NCOA2, to regulate genes. As with EHMT1/2, we generated lentiviral shRNA expression vectors for NCOA2, infected NALM6 cells, and then tested cells for depletion by western-blot and dex sensitivity (FIGS. 44A, 44B, 44C). Using the most effective shRNA for each gene, we treated five NALM6 samples (uninfected, scrambled, NCOA2-KD, EHMT2-KD, EHMT1-KD) with 1 μM dex or control (0.01% ethanol) for four hours, isolated RNA and then analyzed the samples on Illumina HT12 v4 arrays, and calculated differential gene expression (R/Bioconductor, lumi/limma packages) (Supplementary Dataset 3).

Depletion of each coregulator resulted in fewer genes significantly regulated by dex (qvalue<0.01) (FIG. 44C). Much of this effect is due to a general attenuation of dex-regulation of genes. Linear of regression of the dex-induced fold change coregulator-depleted versus control infected cells (shSCR) for each gene indicated that depletion significantly attenuated both activation and repression of genes by 25-35%, on average (FIGS. 37A, 37B, 37C). Depletion of EHMT1 had the most pronounced effect (FIG. 37B), likely because of a concomitant reduction in EHMT2 protein level (Tachibana et al., 2005). The majority of genes appeared to fit this trend (r2=0.84-0.9) indicating that ablation of any of these coregulators attenuates most dex-regulated genes, with some genes exhibiting a significant change in regulation (FIGS. 37A, 37B, 37C, red dots). Some cell-death effector genes did not follow this trend (TP53INP1, DDIT4, RCAN1) and appeared to be unaffected by cofactor depletion. Other effector genes are reliant on specific coregulators, including MYC and RAG1, which are unaffected by depletion of EHMT2. Thus, these coregulators have both general and gene-specific effects on dex-regulation of genes that contribute to B-ALL cell death.

Another potential mechanism of resistance is through a failure to achieve proper mRNA levels of effector genes after exposure to dex. We therefore also compared the dex-induced mRNA levels in the coregulator-depleted cells versus the dex-induced mRNA levels of control cells (FIGS. 37D, 37E). From 305 (EMHT2-KD) to 482 (NCOA2-KD) genes were significantly different from controls after dex administration (FIGS. 37D, 37E, 37F, red dots). Most of these genes overlapped with the genes that failed to be properly regulated by dex upon coregulator depletion (FIGS. 37A, 37B, 37C), and even with a more stringent 1.5-fold change cutoff for the dex effect there was considerable overlap among the subsets of dex-regulated genes that require each coregulator (FIG. 45A) This analysis revealed misexpression of potential effector genes, including TSC22D3 (aka GILZ), TXNIP, and NFKBIA, which were identified in the pathway analysis (FIG. 42F) and have been implicated in cell death and survival pathways (Bruscoli et al., 2015; Chen et al., 2011; Fuchs, 2010; Schmidt et al., 2004). The upregulation of these potential effector genes by dex is impaired by depletion of some, but not all, coregulators. Similarly, some repressed effector genes, including MYC and SOX4, fail to be fully repressed, and mRNA levels remain aberrantly high. Thus coregulators have both general and gene-specific effects on the dex-induced expression level of effector genes, as well general and specific effects on regulation of effector genes.

We then sought to confirm these effector genes, and test whether coregulator depletion affected their regulation. Using quantitative reverse transcriptase-PCR (RT-qPCR) we measured the effect of coregulator depletion on expression and dex-regulation of TSC22D3, TXNIP, and NFKBIA. Depletion of EHMT2, EHMT1, or CBX3 by shRNAs (FIG. 38B) significantly decreased expression of these three GR target genes after 8 h of dex treatment, but had no significant effect on dex-induced expression of FKBPS, a gene that did not require EHMT2 and EHMT1 (FIG. 38A). Importantly, depletion of TSC22D3, NFKBIA, and TXNIP decreased sensitivity to dex-induced cell death (FIGS. 38C, 45B, 45C, 45D). Thus, in NALM6 cells EHMT2, EHMT1 and CBX3 cooperate as coactivators for GC-induced expression of GC effector genes involved in cell death.

We also tested whether these three effector genes are targets of the GR-EHMT1/2-CBX3 complex. We identified the GR binding regions for TSC22D3, TXNIP and NFKBIA from our previously published chromatin immunoprecipitation followed by deep sequencing (ChIP-seq) data in NALM-6 cells (Kruth et al., 2017). We first validated dex-induced binding of GR using ChIP-qPCR (FIG. 38D) and then showed that CBX3 is recruited to these sites upon treatment with dex (FIG. 38E). Importantly, CBX3 is not recruited to the FKBPS genes, whose dex regulation is not dependent on EHMT1/2 (FIG. 38E). This indicates that recruitment of the full complex to GR-EHMT1/2-CBX3 complex accompanies regulation of dex effector genes.

Having established the importance of coregulators in dex-mediated cell death, we then asked whether misexpression of coregulators could be a source of resistance in relapsed B-ALL.

### AURKB is a Relapse-Resistance Gene

To identify genes that might be causative for resistance, we overlapped the set of misexpressed genes at relapse (683 top hits with p-value≤0.01, from the Supplementary Dataset 1) with the sets of genes with significant effects of growth (γ) (FIG. 35A) or dex sensitivity (ρ) (FIG. 35B) from the shRNA screen. Using a relaxed cutoff (p-value=0.05), 137 misexpressed genes also had a significant positive or negative effect on growth or survival (FIG. 46A). Genes overexpressed at relapse should show decreased growth when knocked down (FIG. 46B orange shaded, negative γ score in the shRNA screen). Similarly, genes underexpressed at relapse should show increased growth when depleted (FIG. 46B yellow shaded, positive p score). Of the 137, 101 fit these two categories (FIG. 46C, Table 9), with cell cycle genes significantly overrepresented (FIG. 46D, Table 9). In addition to genes previously identified (e.g. CCNB2, CDK1) (Bhojwani et al., 2006; Chow et al., 2017; Hogan et al., 2011; Staal et al., 2010), AURKB was overexpressed at relapse (FIG. 34D), more highly expressed in patients that relapsed within 3 years of diagnosis compared with those that relapsed later (FIG. 34E), and decreased proliferation when depleted (FIG. 35A). In addition to AURKB, other epigenetic (e.g. DNMT1, H2AZ, CBXS, PRDM2) factors and transcription factors (e.g. PRDM2, KLF7, and CITED2) were also implicated in increasing growth upon relapse.

We also identified 78 genes that are misexpressed at relapse and affect GC sensitivity (p) in our shRNA screen (FIGS. 39A, 39BB). Of the 78 overlapped genes, 40 genes could be termed resistance-relapse genes as their misexpression is functionally linked to dex resistance in B-ALL (FIG. 39C, Table 10). As examples, overexpression of specific cell cycle (BIRCS, CDK1, CCNB2, and NEK2) and DNA repair genes (BRCA1, PARPBP) increased resistance to dex (FIG. 39D, Table 10). There is no known link between these proteins and GR function, and thus they represent new potential targets for combination therapy. Some, including CDK1 (Aleem and Arceci, 2015) and TOP2A, are already targets. Inhibition of TOP2A with anthracyclines (e.g doxorubicin) is already included as a component of some standard and relapsed therapy. However, because inhibitors to these proteins have been disappointing, showing off-target effects or resulting in secondary malignancies (Asghar et al., 2015; Pendleton et al., 2014), we sought a cell-cycle component that would be more specific.

Surprisingly, we found very few coregulators with significant effects on dex-sensitivity that were misexpressed upon relapse. However, moving one regulatory level out from GR-bound coregulators we found AURKB. Depletion of AURKB inhibited cell proliferation (γ=−0.12, Table 9) and sensitized NALM6 cells to dex (ρ=−0.30). Importantly, AURKB was significantly overexpressed at relapse in all three data sets that we analyzed (FIGS. 34A, 34D). Further, AURKB is overexpressed at diagnosis in patients who relapse within 36 months compared with patients who relapse later (FIG. 34E), indicating that it may be both a prognostic indicator and a cause of relapse. We have previously shown that AURKB negatively regulates GR-regulated transcription by phosphorylating EMHT1 and 2 (Poulard et al., 2017), and we confirmed that EHMT1 and 2 are phosphorylated in Nalm6 cells (FIGS. 36A, 36B). Phosphorylation of EHMT1/2 blocks binding of CBX3 to EHMT1/2, which is required for full regulation of subsets of GR-regulated genes. The effect of depletion on dex-sensitivity from the screen (FIGS. 35B, 35D), with EHMT1/2 and CBX3 depletion being desensitizing and AURKB depletion being sensitizing, was consistent with this mechanism. This suggested that AURKB is a resistance-relapse gene that can be targeted.

### AURKB Inhibitor Enhances GC Sensitivity of B-ALL Cell Lines and Patient-Derived Xenografts in Culture

According to our model, AURKB blunts dex cytotoxicity in B-ALL by phosphorylating EHMT1/2, which interferes with recruitment of CBX3 (FIGS. 47A, 47B). Thus, inhibition of AURKB should sensitize NALM6 cells to dex by enhancing dex regulation of effector genes. To test this, we used two AURKB specific inhibitors, ZM447439 (Ditchfield et al., 2003; Girdler et al., 2006) and AZD2811 (also called AZD1152-HQPA) (Floc'h et al., 2017; Mortlock et al., 2007; Wilkinson et al., 2007). Inhibiting AURKB is itself toxic to NALM6 cells, consistent with our screen data and presumably due to cell cycle inhibition (Goldenson and Crispino, 2015). Inhibition of AURKB with ZM447439 reduced phosphorylation of EHMT2 (FIG. 48A) and significantly sensitized NALM6 cells to dex-induced apoptosis, with 0.75 □M ZM447439 (FIG. 7A) having a stronger effect than 0.625 μM (FIG. 48B). A similar result is observed using AZD2811, a more potent and specific inhibitor of AurKB. Like ZM447439, AZD2811 decreased EHMT2 phosphorylation (FIG. 48C) and enhanced dex-induced cell death (FIG. 48B). In contrast Alisertib, an Aurora kinase A-specific inhibitor, did not have any effect on cell survival (FIG. 48D). AZD2811 also increased cleavage of apoptotic markers in dex-treated NALM6 (FIG. 40C). In addition to NALM6 cells, AURKB inhibitors also enhanced the sensitivity of RCH-ACV, a dex-resistant B-ALL cell line (FIG. 48E, 48F). Importantly, AZD2811 enhanced dex-induced expression of dex-effector genes that utilize EHMT2, EHMT1 and CBX3, but not the EHMT2/EHMT1-independent FKBPS gene (FIGS. 40D-1, 40D-2, 40D-3, and 40D-4). Thus, the effect of the AurKB inhibitor on cell survival involves its selective regulation of EHMT2/EHMT1-dependent GR target genes.

We then tested the combination of AZD2811 and dex in two patient-derived xenograft lines derived from relapsed of B-ALL, LAX7R and LAX56 (Adam et al., 2017; Hsieh et al., 2013). LAX7R and LAX56 have been shown to be resistant to both dex and vincristine, another component of standard B-ALL combination chemotherapy. Treatment of these cells with 16 nM AZD2811 alone for 3 (FIGS. 41A-1, 41A-2, 41B-1, 41B-2, left) or 5 days (FIGS. 41A-1, 41A-2, 41B-1, 41B-2, right) reduced cell survival to 70-80%, compared with the vehicle-treated control. Treatment with 100-200 nM Dex alone reduced survival to 20-60%, whereas lower concentrations of dex had little if any effect on cell survival. In contrast, in combination with 16 nM AZD2811, even 0.1 nM dex reduced cell survival to 10-40%, and lower survival was achieved with higher dex concentrations. Thus, AZD2811 dramatically enhanced the dex sensitivity of these two dex-resistant primary B-ALL lines.

### Discussion

### Identification of Sources of GC Resistance

In B-ALL, treatment resistance arises when cancer cells escape the selective pressure of chemotherapy. However, because the genetic backgrounds of patients differ, as do the developmental stages of the disease, the routes to relapse across all patients are varied. Accordingly, single nucleotide polymorphism (SNP) studies have associated individual genes (IL15, ELMO1, DGKB) (Yang et al., 2009) with treatment resistance and relapse. Nonetheless, some common themes have emerged. Relapse or resistance is correlated with mutations in the transcriptional machinery, including transcription factors, such as Ikaros (IKZF1), and coregulators, such as CBP/P300, BTG1, and TBL1XR1 (Jones et al., 2014; Mullighan et al., 2011; van Galen et al., 2010). Indeed, the SNP studies have also implicated a GR coregulator, NCOA3, in treatment resistance (Yang et al., 2009). In our previous work we used functional genomics to determine that most genetic lesions would have a significant effect on GC sensitivity specifically (Kruth et al., 2017). In this study we use functional genomics to show that, surprisingly, the selective pressure of chemotherapy does not cause widespread misexpression of the transcriptional machinery directly, but rather pathways that result in an attenuated transcriptional and cytotoxic response to GCs.

One expected source of resistance under selective pressure was loss of GR itself and its associated coregulators. As noted, several known GR coregulators that have an effect on dex sensitivity are mutated in some resistant B-ALL patients. GR can also be mutated in resistant B-ALL (Mullighan et al., 2011), although it is not common. Surprisingly, neither the expression level of GR nor its associated coregulators appear to be a major source of resistance. Knockdown of both EMHT1 and EHMT2 had a significant effect on cell growth, suggesting that their activity in maintaining epigenetic marks and chromatin state may be too important to select against. Similarly, our previous work revealed a potential role for GR in overall B-cell development (Kruth et al., 2017). Although knockdown has little effect on growth, GR may be important in early B-cell specification and development. Another coregulator example is PTGES3, which from our shRNA screen is the most essential coregulator for GR (FIG. 35D). Analysis of the relapse misexpressed genes (using Ingenuity Pathway Analysis, Qiagen) indicated a strong signature for the presence of PTGER2 signaling (FIG. 34C). This reveals a contradictory role for PGE2 in treating B-ALL. PGE2 has been shown to be toxic to B-ALL cells (Giordano et al., 1997; Soleymani Fard et al., 2012), while at the same time protecting B-ALL from DNA-damage induced cell death (Naderi et al., 2015; Naderi et al., 2013). Thus, although augmenting PTGES3 or administration of PGE2 would likely enhance GCs, it would be protective against damaging agents, such as doxorubicin.

In addition to having pleiotropic effects on cells, many coregulators are required for GR activity. These include well-studied coregulators that interact with GR, including the NCOAs (aka SRC/p160), NCORs, and TBL1XR1, as well as EHMT1/2, studied here. Direct inhibition of these coregulators would thus likely not enhance sensitivity to dex. There are some exceptions to this, including CBP/P300, HDAC2, and CARM1, that sensitize cells to dex when knocked down, indicating that they restrain GR function. Although these too are not misexpressed upon relapse, they could nonetheless be inhibited to enhance dex sensitivity. That said, inhibition of CBP/P300 is also likely to have pleiotropic effects as these coregulators are expressed in all tissues and associate with dozens of TFs (Goodman and Smolik, 2000; Vo and Goodman, 2001). An intriguing target identified in our shRNA screen is HDAC2 of the NuRD complex. Knockdown of HDAC2 and several other NuRD associated proteins (MTA1, SPEN, MBD2/3, GATAD2B) (Lai and Wade, 2011) sensitized cells to dex. Specific inhibitors to HDAC2 exist that have been shown to have (Stubbs et al., 2015) therapeutic value in B-ALL as a monotherapy. Though these would be predicted to synergize with dex in B-ALL, inhibition of HDAC2 activity has been shown to reduce GC-sensitivity in the lungs (Ito et al., 2006). Instead, a more effective strategy might be to inhibit pathways that impinge on GR coregulators, modulating specific activities.

In our previous work we screened ˜35% of the known protein coding genes (Kruth et al., 2017). Absent from this, but detected here with our full genome shRNA screen, were genes that were not classified as cancer genes (e.g. CBX3, PTGES3, CHD3/4) that nonetheless impinge on GR gene-regulation relevant to B-ALL cell death. Intersecting this data with aggregated data from relapsed patients enabled us to identify factors misexpressed at relapse that are associated with dex resistance. This integration revealed what had been proposed, that upregulation of cell cycle genes is not only associated with relapse, but also a source of glucocorticoid resistance specifically (Bhojwani et al., 2006). The activity of GR is cell cycle dependent, being most active in G1/S, but with reduced activity in G2/M (Hsu and DeFranco, 1995; Hsu et al., 1992). Although CDKs have been shown to modify GR (Krstic et al., 1997; Kumar and Calhoun, 2008), whether this accounts for the cell-cycle dependent activity is not clear. CDK1 exhibits significantly higher expression levels in relapsed patients, and blunts GC activity according to our screen. Inhibitors for CDK1 are under development, but are generally not specific, and have not been as clinically effective as hoped. Also fitting these criteria was AURKB, which we had identified in our previous work as a modulator of GR function through phosphorylation of EHMT1/2.

### Targeting AURKB Sensitizes B-ALL Cells to GC Cell Death

AURKB is a component of the chromosomal passenger complex (CPC), composed of BIRC5 (aka Survivin), CDCA8 (Borealin), and INCENP. The CPC has important roles at all stages of mitosis, from spindle formation through cytokinesis (D'Avino and Capalbo, 2015; Goldenson and Crispino, 2015). Each member of the complex has an effect on either the growth or survival of NALM6 cells. Interestingly, both BIRC5 and AURKB can be classified as resistance-relapse genes, as they are overexpressed upon relapse and enhance sensitivity when knocked down (FIGS. 34A, 35B, Table 10). This raises the question of whether the BIRC5 and AURKB act independently form the other CPC components when overexpressed to render B-ALL specifically resistant to dex.

TABLE 10
Relapse-resistance genes.

| | Rho | Rho | Relapse/ | Fisher's |
|---|---|---|---|---|
| Symbol | phenotype | p-value | diagnostic (log2) | p-value |
| AFF1 | 0.3 | 1.59E−03 | −0.22 | 2.14E−03 |
| BIRC5 | −0.23 | 3.00E−02 | 0.62 | 3.31E−07 |
| BRCA1 | −0.26 | 4.01E−02 | 0.29 | 9.83E−03 |
| CCNA2 | −0.22 | 2.22E−02 | 0.31 | 3.78E−03 |
| CCNB1 | −0.24 | 1.07E−03 | 0.48 | 1.98E−03 |
| CDK1 | −0.11 | 1.81E−03 | 0.6 | 2.16E−04 |
| CENPE | −0.12 | 9.47E−03 | 0.43 | 2.16E−04 |
| COX6B1 | −0.26 | 9.28E−03 | 0.21 | 6.13E−03 |
| COX6C | −0.26 | 2.37E−02 | 0.25 | 1.06E−02 |
| COX7C | −0.1 | 6.95E−03 | 0.17 | 5.78E−03 |
| DLGAP5 | −0.2 | 3.55E−02 | 0.64 | 4.74E−04 |
| EFR3B | 0.3 | 4.10E−02 | −0.03 | 3.83E−03 |
| EIF3A | −0.05 | 2.39E−03 | 0.18 | 5.57E−03 |
| FDXR | −0.24 | 4.11E−02 | 0.21 | 4.68E−04 |
| GTF2I | −0.24 | 7.35E−03 | 0.06 | 5.75E−04 |
| HMGN2 | −1.30E−03 | 1.26E−02 | 0.18 | 1.48E−02 |
| KIAA0368 | −0.11 | 4.51E−02 | 0.17 | 8.58E−03 |
| KIF23 | −0.11 | 2.42E−02 | 0.42 | 2.26E−03 |
| KPNB1 | −0.09 | 4.77E−04 | 0.24 | 2.46E−04 |
| MYBL2 | −0.14 | 3.07E−02 | 0.33 | 1.85E−03 |
| NCAPG | −0.17 | 2.47E−02 | 0.52 | 3.02E−05 |
| NCAPH | −0.19 | 2.69E−04 | 0.4 | 1.57E−06 |
| NEK2 | −0.42 | 2.56E−02 | 0.36 | 4.59E−03 |
| NR2F2 | −0.1 | 1.04E−02 | 0.13 | 2.11E−03 |
| NUP98 | −0.45 | 1.62E−02 | 0.2 | 1.84E−02 |
| PA2G4 | −0.2 | 2.77E−02 | 0.21 | 1.22E−03 |
| PARPBP | −0.34 | 3.24E−02 | 0.23 | 2.54E−04 |
| PRDX3 | −0.03 | 4.27E−02 | 0.39 | 7.83E−03 |
| S100A12 | −0.25 | 4.24E−03 | 0.5 | 6.08E−03 |
| SAFB2 | 0.45 | 3.58E−02 | −0.11 | 6.88E−03 |
| SNRPE | −1.90E−03 | 3.37E−02 | 0.27 | 6.19E−03 |
| SNRPF | −0.25 | 1.65E−03 | 0.22 | 4.26E−03 |
| SRSF7 | 0.05 | 3.70E−02 | −0.29 | 7.19E−03 |
| TACC3 | −0.11 | 1.51E−02 | 0.27 | 4.12E−03 |
| TLL1 | 0.25 | 4.69E−02 | −0.01 | 9.93E−03 |
| TOP2A | −0.13 | 4.79E−02 | 0.81 | 9.88E−07 |
| TPX2 | −0.15 | 4.12E−02 | 0.4 | 1.60E−03 |
| TTK | −0.28 | 4.34E−03 | 0.49 | 1.42E−03 |
| TUBB | −0.25 | 4.61E−02 | 0.28 | 4.26E−03 |
| YTHDC1 | 0.3 | 1.01E−02 | −0.34 | 4.77E−04 |

Table 10 shows relapse-resistance genes. Resistance genes are defined as causing a significant effect on the sensitivity of NALM-6 cells to dexamethasone (Rho p-value≤0.05) and expression changes significantly from diagnosis to relapse (Fisher p-value≤0.01). Genes whose knockdown makes cells more sensitive to dex would be predicted to cause resistance if overexpressed. Thus, genes that are overexpressed upon relapse (positive Relapse/Diagnostic) and make cells more sensitive (Rho phenotype<0) are relapse-resistance genes. By the same logic, genes that are underexpressed upon relapse (negative Relapse/Diagnostic) and make cells more resistant upon knockdown (Rho phenotype>0) are also relapse-resistance genes.

This functional genomics approach thus flagged BIRCS/AURKB as a promising potential therapeutic target and allowed us to make a mechanistic connection to suppression of GR activity. Instrumental to this discovery was mechanistic work performed in an unrelated system. Consistent with our previous work in A549 lung adenocarcinoma cells, the screen showed that GR coregulators EHMT2, EHMT1, and CBX3 contribute to GR activity, whereas AURKB restrains it. Inhibition of AURKB reduced phosphorylation of EHMT1/2, thus enhancing interaction of CBX3 with methylated EHMT1/2 and potentiating dex-induced expression and regulation of the subset of GR target genes requiring EHMT2, EHMT1, and CBX3 (FIGS. 47A, 47B). These include genes that contribute to dex-induced cell death (e.g. TSC22D3, TXNIP, NFKBIA) in cell lines and patient samples cultured in vitro, some of which are quite resistant to dex alone. This illustrates how functional genomic studies in relevant systems can allow researchers to lift therapeutic targets from the vast reservoir of detailed basic discovery in cell-based systems and model organisms.

Thus, we define here a novel role for AURKB in regulating GC-induced cell death, through its regulation of EHMT2/EHMT1 coactivator function with GR. This activity is completely separate from the role of AURKB in promoting cell cycle progression. In fact, as discussed above, regulation of gene expression by GC is greatly reduced during mitosis (Hsu and DeFranco, 1995; Hu et al., 1994), and thus the regulation of GR activity by AURKB is likely to be during non-mitotic phases of the cell cycle. Ultimately, the enhanced GC-induced cell death by AURKB inhibitors may result from a combination of its inhibition of cell cycle progression and its mechanistically separate enhancement of GC-induced expression of EHMT2/EHMT1-dependent GR target genes. Thus, the use of AURKB inhibitors to augment GC potency may represent a novel therapeutic strategy for addressing relapsed B-ALL, as well as other hematologic malignancies where acquired resistance to dex is a cause of patient relapse.

While this study ultimately focused on the regulation of GR coregulators by AURKB, data from the shRNA screen will be an invaluable resource for researchers in the leukemia and steroid hormone receptor fields who are looking to functionally validate genes identified through correlative studies, such as SNPs, QTLs, differential gene expression, and mutational studies.

Glucocorticoids (GCs) are used in combination chemotherapies as front-line treatment for lymphoid cancers, including B-cell acute lymphoblastic leukemia (B-ALL). Although effective, many patients relapse and become resistant to chemotherapy, and GCs in particular. Why these patients relapse is not clear. We took a comprehensive, functional genomics approach to identifying sources of GC resistance that could be targeted to restore sensitivity. We compared results from a genome-wide shRNA screen to identify genes that affect growth and GC-sensitivity in B-ALL to misexpressed genes in relapsed patients. We identified cell cycle genes, including AURKB, as sources of relapse. AURKB restrains the activity of the glucocorticoid receptor by phosphorylating specific coregulators, EHMT1/2. Inhibition of AURKB catalytic activity enhanced the GC-regulation of cell death genes, resulting in potentiation of GC cytotoxicity in cell-line and patient B-ALL specimens. These results validate a functional genomic approach to the design of combination chemotherapeutics for relapsed patients and demonstrate how transcription can be tailored by inhibiting pathways that impinge on coregulators.

### EXAMPLE 4

### Demethylase Inhibitors for Sensitizing Hematologic Malignancies to Glucocorticoid Therapy

Synthetic glucocorticoid (GC) analogues are first-line drugs used to treat many hematologic cancers because they induce cell death by a mechanism that is specific to the lymphoid cell lineage. While many patients respond favorably to these drugs, the cancers for many patients are resistant to these drugs or develop resistance. Long-term, high dose GC treatments cause serious adverse side-effects. As previously disclosed, we reported that 1) specific GC-inducible genes in a B-ALL leukemia cell line are required for efficient GC-induced cell death; 2) specific transcriptional coactivators (G9a, GLP, HP1y) are required for the GC-induced expression of these death genes; 3) post-translational modifications (phosphorylation and methylation) of G9a and GLP control the ability of these three coregulators to facilitate GC activation of the genes that promote cell death; 4) inhibitors of Aurora kinase B reduce phosphorylation of G9a and GLP, enhance the activity or these three coactivators, enhance GC activation of the genes that promote cell death, and thereby enhance the GC sensitivity of the B-ALL leukemia cell line. Here we report that specific protein demethylase inhibitors enhance the methylation of G9a and GLP, the coactivator activity of G9a, GLP, and HP1y, GC activation of the genes that promote leukemia cell death, and the sensitivity of a B-ALL cell line to GC-induced cell death. Our results add the protein demethylase inhibitors to Aurora kinase B (AURKB) inhibitors as potential new therapeutic options for treating patients with hematologic malignancies that are normally treated with GC. Particularly, demethylase and AURKB inhibitors may be particularly attractive for treating patients who have become resistant to GC.

### Defining the Mechanism by Which Coregulators G9a and GLP Function as Coactivators for the Glucocorticoid Receptor

GC activate the glucocorticoid receptor (GR), a hormone regulated transcription factor which activates and represses specific genes in cells. GR binds in a DNA sequence-specific manner to sites in the genome that serve as enhancer and silencer elements that are physically associated with and control the expression of specific genes. GR recruits specific coregulator proteins to these DNA binding sites, and the coregulators perform a complex set of functions that modulate local chromatin conformation and regulate the formation of an active transcription complex on the transcription start site of the associated GR target genes (1). Several hundred coregulators have been identified, indicating a high level of complexity in the process of transcriptional regulation. The actions of coregulators are gene-specific, i.e. each specific coregulator is required only for a subset of the genes that are regulated by a specific DNA- binding transcription factor (such as GR) (2-6). This finding suggests that individual coregulators may be associated with genes belonging to a specific physiological pathway. For example, glucocorticoids regulate many different physiological pathways, including inflammation, bone remodeling, and metabolism of glucose, lipids and proteins. Recent studies with GR and other transcription factors have in fact demonstrated that specific coregulators are preferentially required for genes involved in selected physiological responses among multiple pathways that are regulated by a given transcription factor (5-7).

Coregulators that help to activate genes are called coactivators, and those that help to repress genes are called corepressors. In fact, many coregulators can cooperate with a specific transcription factor to act as coactivator on some genes and corepressor on other genes in the same cell line or cell type (2-4). However, very little is known about the factors that dictate whether a coregulator activates or represses transcription when recruited to a specific gene. Two coregulators that are central to this application, G9a (EHMT2) and GLP (EHMT1), are excellent examples of this dual coactivator/corepressor activity (3,6). They are highly homologous histone H3 lysine 9 (H3K9) methyltransferases that are responsible for the majority of the H3K9 monomethylation (H3K9me1) and dimethylation (H3K9me2) found in mammalian cells (8). These histone modifications are highly associated with the regulatory regions of inactive genes, and G9a and GLP are well known to function as corepressors. However, our lab, followed by others, demonstrated that G9a and GLP function also as coactivators (6,9-11). For example, when GC regulated gene expression was examined in A549 lung adenocarcinoma cells depleted of either G9a or GLP, we found that G9a and GLP helped GR to activate some genes and helped GR to repress other genes, but there were many other GR target genes that were activated or repressed similarly in the presence or absence of G9a and GLP (3,6). The corepressor activity was previously shown to involve the C-terminal methyltransferase activity and other domains in the central region of the polypeptide chains of G9a and GLP. In contrast, the coactivator activity involves the N-terminal region, which binds directly to GR and also contains an activation function (6,10).

We have recently identified the mechanism by which G9a and GLP function as coactivators and also showed that a pair of adjacent post-translational modifications in the N-terminal coactivator region of G9a and GLP regulate the coactivator function (6). The findings, performed in the A549 lung adenocarcinoma cell line, are briefly summarized here. Both G9a and GLP can be methylated (by either G9a or GLP) on a lysine residue in the amino acid sequence ARKT found within the N-terminal coactivator domain (K185 for G9a and K205 for GLP). They can also be phosphorylated on the adjacent T residue (T186 in G9a and T206 in GLP) by Aurora kinase B (AURKB). Methylated G9a and GLP bind to HP1 γ and form a ternary complex GR-G9a/GLP- HP1 γ. Phosphorylation by AURKB prevents HP1γ binding to G9a and GLP (FIG. 1).

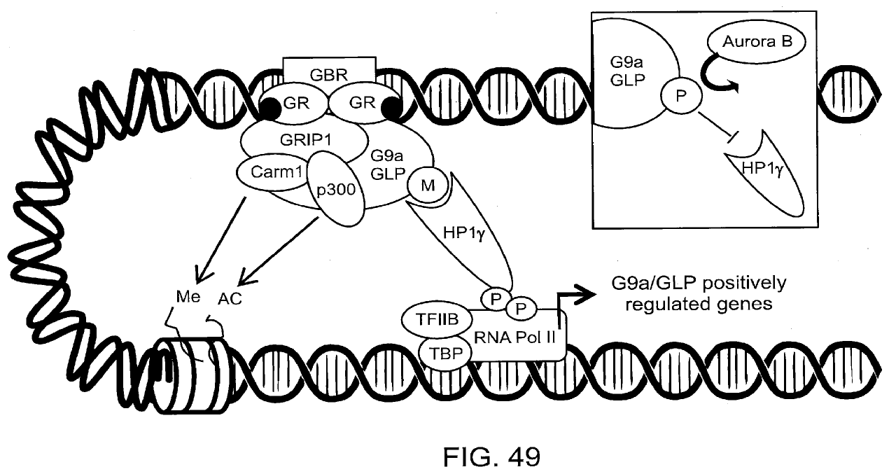

GR target genes that require G9a and GLP for their GC-induced expression also require HP1 γ, while GR target genes that do not require G9a and GLP also do not require HP1γ for their GC-induced expression (6). Thus different subsets of GC-activated genes have different coregulator requirements for G9a, GLP, and HP1γ. Inhibitors of AURKB, as expected, enhance the GC-induced expression of GR target genes that require G9a, GLP, and HP1γ, because they enhance the interaction of HP1γ with G9a and GLP; in contrast, the GC induced expression of GR target genes that are independent of G9a, GLP, and HP1γ are not affected by AURKB inhibitors (6). GR, G9a, GLP, and HP1γ all assemble in a hormone-dependent manner on GR binding sites (GBS) associated with GR target genes that require G9a as a coactivator; but G9a, GLP, and HP1γ do not occupy GR target genes that do not require these coregulators. The GC- induced occupancy of HP1γ is eliminated when G9a is depleted from cells (6). All of these data are consistent with the model that GC-activated GR recruits G9a and GLP to specific GBS, and if G9a/GLP is methylated, they recruit HP1γ as well. But phosphorylation of G9a and GLP blocks binding of HP1γ and thus blocks activation of G9a/GLP-dependent target genes by GC (FIG. 49). The coactivator function of G9a/GLP requires HP1y.

### Figure Descriptions

FIG. 49 is a graphical depiction showing that G9a/GLP coactivator activity is regulated by methylation and phosphorylation. FIG. 49 further highlights that: (1) Methylation of G9a and GLP (self-methylation) recruits HP1γ, which facilitates recruitment of RNA pol II, to activate G9a/GLP-dependent GR target genes and; (2) Phosphorylation of G9a and GLP (by Aurora kinase B) prevents HP1γ recruitment, thereby inhibiting dex-induced expression of the G9a/GLP-dependent GR target genes.

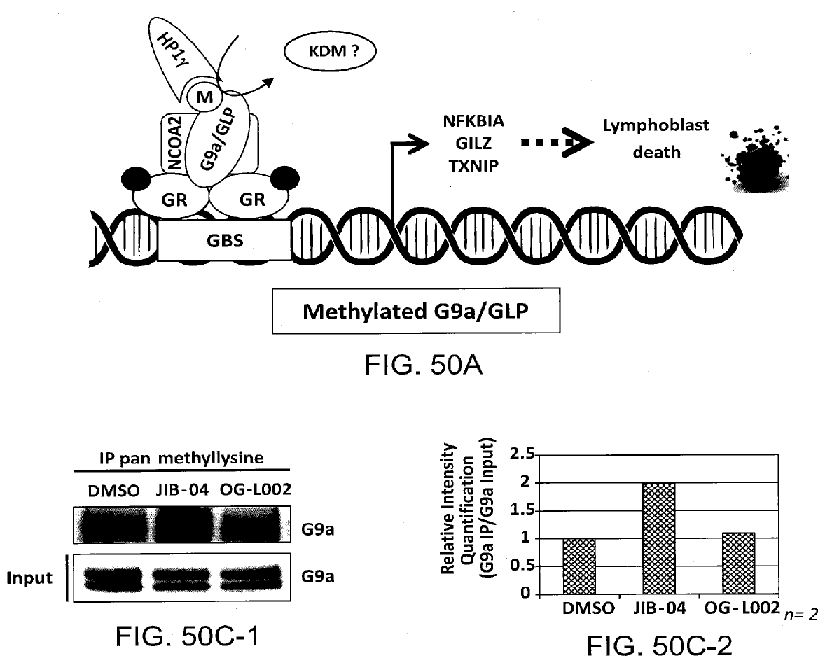

FIGS. 50A, 50B, 50C-1, 50C-2, and 50D show that the Jumonji family lysine demethylases (KDM) demethylate G9a/GLP in B-ALL cells. FIG. 50A shows a graphical depiction showing the effect of demethylation. As highlighted in FIG. 50B, there are two KDM families. The removal of G9a/GLP methylation by KDMs (Lysine demethylases) inhibits G9a/GLP coactivator activity, induction of apoptosispromoting genes by GC, and lymphoblast cell death. FIGS. 50C-1 and 50C-2 shows th effect of the different KDM inhibitors.

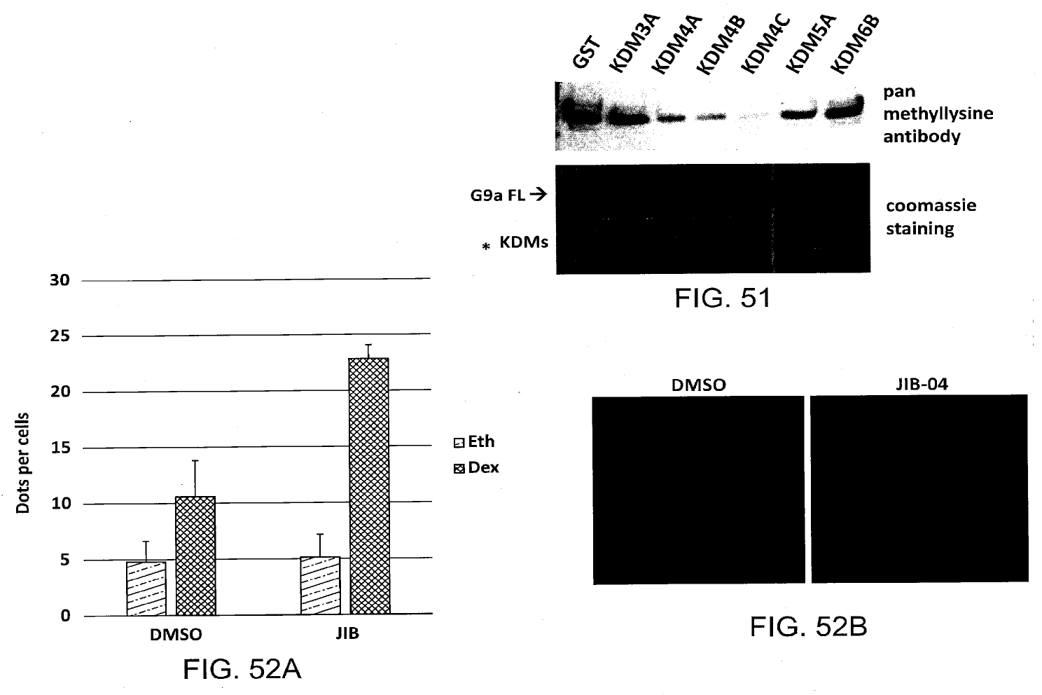

FIG. 51 shows that KDM4 family demethylates G9a. Using an in vitro demethylase assays, recombinant G9a was allowed to self-methylate with S-adenosyl methionine and then incubated with recombinant demethylases. Methylation status was assessed by western blot. As shown in FIG. 51, KDM4 family demethylases are among the group of demethylases inhibited by JIB-04.

FIGS. 52A and 52B show that JIB-04 inhibitor enhancing GR-G9a-HP1γ complex formation. FIGS. 52A and 52B show results from proximity ligation assays in A549 cells that detects the interaction between GR and HP1γ.

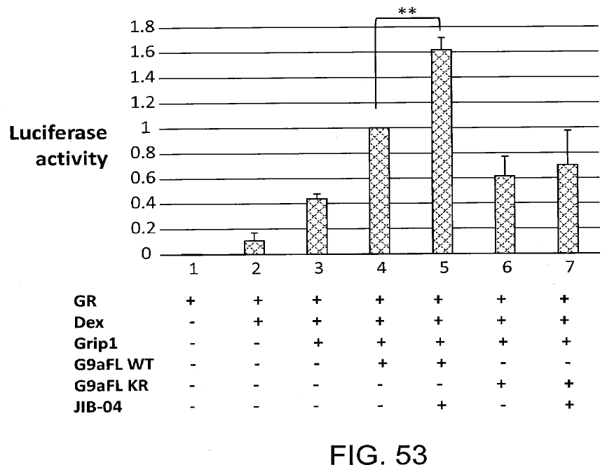

FIG. 53 shows JIB-04 inhibitor enhancing G9a coactivator function. Particularly, JIB-04 inhibitor treatment enhances G9a coactivator activity with GR in a transient reporter gene assay.

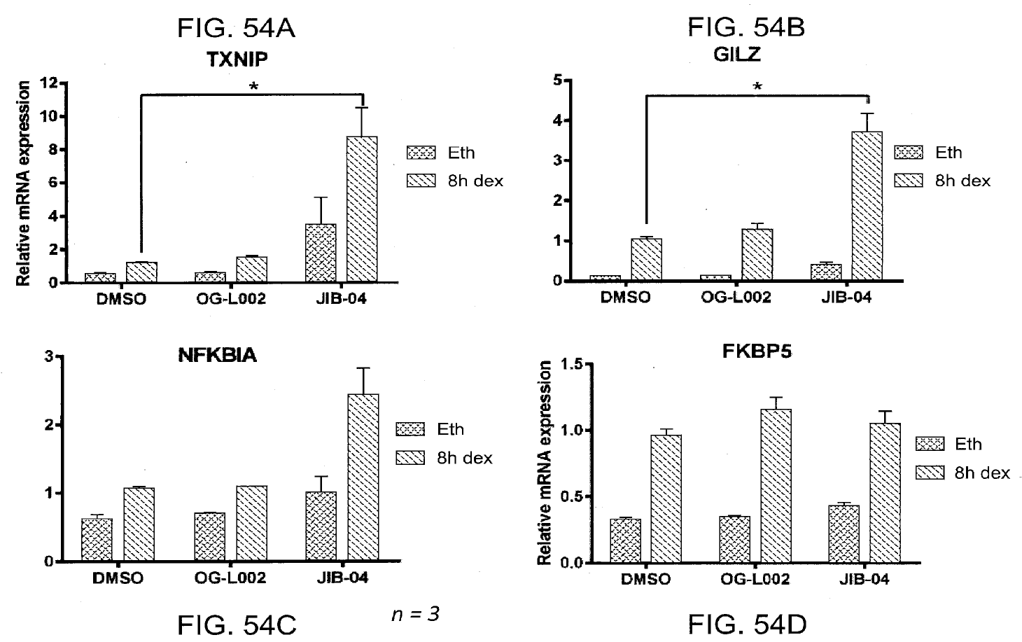

FIGS. 54A, 54B, 54C, and 54D shows JIB-04 enhancing G9a coactivator function. FIGS. 54A, 54B, and 54C highlight the GR Target genes that require G9a and GLP as coactivators. FIG. 54D shows the GR Target genes that do not require G9a and GLP as coactivators.

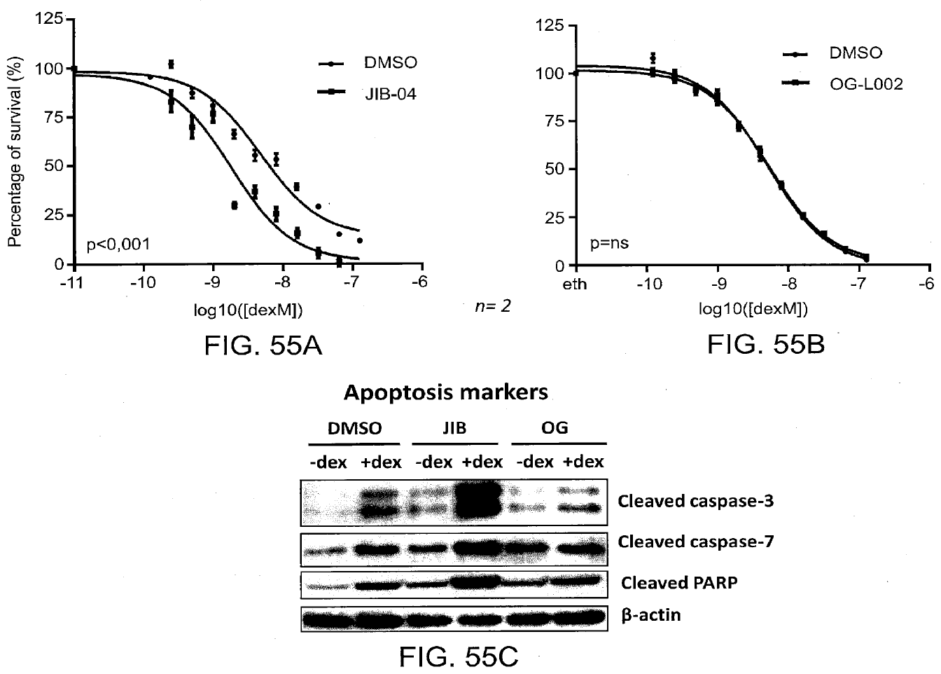

FIGS. 55A, 55B, and 55C show JIB-04 enhances GC-induced death of B-ALL cell line NALM6.

### Results

### Sensitivity and Resistance of Hematologic Malignancies to GC-Induced Cell Death

Synthetic GC analogues such as dexamethasone (dex) and prednisone (or prednisolone) are first-line drugs used to treat many hematologic cancers, including B-cell and T-cell ALL, non-Hodgkins Lymphoma (NHL), Hodgkins Lymphoma, chromic lymphocytic leukemia (CLL), and multiple myeloma, because they induce cell death by a mechanism that is specific to the lymphoid cell lineage, particularly immature lymphoid cells from which many hematologic cancers are derived. While many patients respond favorably to these drugs there are also severe problems associated with their use (12,13). The cancers for many patients are resistant to these drugs, or they develop resistance during or after the treatment.

In pediatric B-cell acute lymphoblastic leukemia (B-ALL) first-line treatment of patients generally involves combinations of GC with other standard chemotherapeutic drugs. A variety of resistance mechanisms have been identified, and some of these have helped to elucidate parts of the mechanism involved in GC-induced cell death, which surprisingly is still poorly understood. The most well-described mechanism is regulation of BCL2 family member genes. GC typically activate the pro-apoptotic BCL2L11 (aka BIM) and repress the anti-apoptotic BCL2 (23). Epigenetic silencing of BIM (14), and enhanced expression of anti-apoptotic BCL2 (15) has been identified as a resistance mechanism. Other gene sets that are predictive of early response have been identified (12) and some of these are GC-regulated genes and suggest that MAPK, NFκB, and carbohydrate metabolism pathways are major pathways upregulated by GC (16). Upregulation of CASP1 in some resistant leukemias leads to proteolytic cleavage and thus inactivation of GR (17). Reduced expression of components of ATP-dependent chromatin remodeling complexes was associated with GC-resistance (18). Treatment with glycolysis inhibitors enhances sensitivity of some leukemia cell lines and patient-derived samples to GC (19). Silencing of MCL1 expression by GC was implicated in GC-induced cell death (20). Activating RAS pathway mutations have been implicated in resistance to GC (21), and enhanced MAPK signaling has been associated with GC resistance (22). EMP1, identified as a poor prognostic factor, regulates GC resistance along with cell proliferation, migration and adhesion (23). Loss of BTG1 expression has been implicated in reduced expression of the gene encoding GR (24). GC-induced gene GILZ is important for regulation of B cell proliferation and GC-induced apoptosis (25). Mutation of TBL1XR1 or depletion of its expression interfered with GC signaling by inhibiting GR association with target genes (26). Unfortunately, despite our understanding of these genetic mechanisms, few targets have been identified that are amenable to small molecule intervention that can specifically enhance glucorticoid sensitivity in lymphoid malignancies.

Options for treatments of resistant disease are severely limited. In addition, long-term treatment with high doses of GC result in serious adverse side-effects, including osteoporosis hyperglycemia, hyperlipidemia, insulin resistance, muscle wasting, and obesity. Thus, novel treatments based on an enhanced understanding of GC-induced cell death and the mechanisms of resistance are clearly needed.

### Specific Lysine Demethylase Inhibitors Enhance the Sensitivity of B-ALL Cells to GC-Induced Cell Death

As described above we validated a molecular model for the mechanism of G9a/GLP coactivator function with GR in A549 lung adenocarcinoma cells (FIG. 49). We have now demonstrated the same molecular mechanism in the NALM6 B-ALL cell line (FIG. 50A). We have already demonstrated that G9a and GLP are required for GC- induced expression of a subset of GR target genes, including several genes (TXNIP, GILZ, and NFKBIA) that contribute to GC-induced cell death. Our molecular model for leukemia cells (FIG. 50A) suggests that GC induction of these genes could be enhanced by increasing methylation of G9a and GLP, which should lead to enhanced interaction of G9a/GLP with HP1γ, enhanced activation of the cell death genes, and enhanced sensitivity of B-ALL cells to GC- induced cell death. Therefore, we tested the effect of lysine demethylase (KDM) inhibitors.

There are two distinct families of KDM with distinct catalytic mechanisms (FIG. 50B). We tested one inhibitor for each family: OG-L002 inhibits the two members of the LSD family of KDM, and JIB-04 inhibits several members of the large JmjC family of KDM, including members of the KDM2, KDM3, and KDM4 subfamilies. We propose that there is an equilibrium between the enzymes that methylate G9a and GLP (in this case they methylate themselves) and the KDM enzymes to maintain a moderate level of G9a/GLP methylation (FIGS. 50C-1, 50C-2), and that inhibition of the KDMs that normally demethylate G9a and GLP (currently unknown) will increase the level of G9a/GLP methylation and thereby enhance the coactivator activity of G9a/GLP, the GC activation of the cell death genes, and GC-induced cell death in B-ALL cells. Thus, as a first step to identify the G9a/GLP-specific demethylases, we treated NALM6 cells with the two inhibitors named above. OG-L002 had no effect on the level of G9a methylation, but JIB-04 increased the level of G9a methylation in NALM6 cells (FIG. 50D).

To identify specific JmjC demethylases that can demethylate G9a/GLP we used a cell free assay with recombinant G9a and recombinant KDMs. Recombinant G9a was allowed to self-methylate and then incubated with various recombinant JmjC KDMs. Three KDM4 subfamily members (which are among those inhibited by JIB-04) demethylated G9a, while several other KDMs catalyzed little or no demethylation of G9a (FIG. 51). Additional work is needed to identify the specific KDM(s) that demethylate G9a/GLP in B-ALL cells.

Since JIB-04 enhanced G9a methylation level in NALM6 cells (FIG. 50D), it should enhance the formation of a GR-G9a-HP1γ complex, and indeed using the Proximity Ligation Assay method in A549 cells treated with the synthetic GC dexamethasone (dex) JIB-04 enhanced the dex-induced interaction between GR and HP1γ (FIGS. 52A, 52B), indicating the formation of a GR-G9a-HP1γ complex. Enhanced formation of the GR-G9a-HP1γ complex should enhance the coactivator activity of G9a for GR, and indeed in a transient reported gene assay in CV-1 cells with a luciferase reporter gene controlled by a glucocorticoid response element (GRE) JIB-04 enhanced dex-induced expression of the reporter gene by GR, the coactivator GRIP1 and wild type G9a; in contrast, when a mutant G9a with the methylation site mutated from lysine to arginine was used, JIB-04 had no effect (FIG. 53). Wild type and mutant G9a were expressed at the same level. This demonstrates that the effect of JIB-04 is dependent on the methylation status of G9a and thus works by enhancing the methylation level of G9a. Similarly, with endogenous GR gene targets in NALM6 cells, JIB-04 (but not OG-L002) enhanced the dex- induced expression of three G9a/GLP-dependent GR target genes (TXNIP, GILZ, and NFKBIA) that contribute to cell death (FIGS. 54A, 54B, 54C). In contrast, JIB-04 had no effect on dex-induced expression of FKBPS, which is independent of G9a and GLP (FIG. 54D). This demonstrates the specificity of JIB-04 for genes that require G9a and GLP as coactivators for GR: JIB-04 is not causing a global increase in gene expression or in dex-induced expression of all GR target genes, but is only enhancing dex-induced expression of genes that require methylated G9a/GLP as coactivators for GR.

Since JIB-04 enhances dex-induced expression of genes that promote cell death, we also tested whether it would have an effect on GC-induced death of NALM6 cells. Indeed JIB-04 enhanced GC-induced death of NALM6 cells treated for 72 hours with various dex concentrations, while OG-L002 had no effect (FIGS. 55A, 55B). In addition, 24-hour treatment with JIB-04, but not OG-L002, enhanced dex-induced cleavage of caspase-3, caspase-7, and PARP1, which are markers of apoptosis (FIG. 55C).

### Applications

Lysine demethylase (KDM) inhibitors can be used to enhance GC-sensitivity of leukemia cells. Two possible scenarios are envisioned for use of KDM inhibitors in the first line of treatment in combination with the standard regimens of drugs. First, use of KDM inhibitors can be used to reduce the dose of GC used in treatment, thus reducing side-effects caused by GC. Second, KDM inhibitors can be used with the currently prescribed concentrations of GC to enhance the level of cell death achieved.

KDM inhibitors can potentially be used to reverse resistance of at least some leukemias which failed initial round of treatment, presumably in combination with other chemotherapeutic drugs.

KDM inhibitors may be used in combination with AURKB inhibitors; the ability of AURKB inhibitors to enhance GC sensitivity in B-ALL cells was previously disclosed (related USC disclosure 2017-134). AURKB inhibitors would limit G9a/GLP phosphorylation, and KDM inhibitors would enhance G9a/GLP methylation, both of which should contribute to enhanced sensitivity of B-ALL cells to GC-induced cell death.

Since the levels of G9a, GLP, HP1γ, AURKB, and the KDMs influence the sensitivity of ALL cells to GC-induced cell death, the levels of these proteins in leukemia cells may serve as a predictor of the sensitivity to GC-induced cell death. Further tests will be required to establish whether these proteins will serve as effective predictors of outcome for particular subsets of patients.

It is especially noteworthy that the actions of G9a, GLP, and HP1γ as coactivators for GR are gene-specific. They are required for some dex-induced GR target genes but not other dex-induced GR target genes. G9a and GLP also function as corepressors for some GR target genes that are repressed by GR in response to dex. Since the coactivator activity of G9a and GLP is located in their N-termini, while the corepressor activity is located in their C-terminal regions, we speculate that the N-terminal methylation and phosphorylation of G9a and GLP described here will not affect the ability of G9a/GLP to serve as corepressors for GR. This means that KDM and AURKB inhibitors will only affect the dex-regulated expression of the subsets of GR target genes that require G9a/GLP as coactivators and will thus limit certain side effects. Thus, the fact that G9a and GLP are gene-specific in their actions and support specific dex-regulated physiological pathways (leukemia cell death in this case) among the many physiological pathways regulated by GC, makes G9a and GLP and the post-translational modifications that regulate them attractive potential targets for therapeutic intervention.

### EXAMPLE 5

### In Vivo Testing

We test the efficacy of AurkB inhibitors on these primary patient-derived leukemia cell lines both in culture and in Patient Derived Xenograft (PDX) models. Sensitive and resistant primary patient-derived cell lines are grown in both model systems under the following conditions: 1) no treatment; 2) AurkB inhibitor alone; 3) chemotherapy+GC; 4) AurkB inhibitor+chemotherapy+GC. In culture, we monitor cell survival as a function of time. In mouse PDX system we will monitor tumor volume, animal health, and animal weight. In both systems, we will monitor AurkB protein and mRNA levels at the beginning and end of the treatment regimen. This will allow us to correlate efficacy of the treatments with AurkB expression.

### Inhibitors of Aurora Kinase B And Lysine Demethylases Enhance GC-Induced Cell Death in Primary B-ALL And T-ALL Tumor Lines In Vitro And in Mouse Xenograft Models.

Primary B-ALL and T-ALL tumor lines from relapsed patients in mouse xenografts have been established.

Commercially available AURKB inhibitor AZD2811 (aka AZD1152-hQPA) is used in vitro. We obtained the proprietary nanoparticle formulation of AZD2811 (AZD2811NP) for use in vivo. AZD2811NP is approved for clinical trials (NCT03217838), as a cell cycle inhibitor.

JIB-0420,21, a commercially available inhibitor of a few subfamilies of the JmjC lysine demethylase family is used in vitro and in vivo. JIB-04 effectively enhanced dex-induced death of Nalm6 B-ALL cells. For xenograft studies we will follow the dosing protocols used previously for this inhibitor.

### Inhibition of Aurkb or Lysine Demethylases Overcomes GC Resistance In Vitro In Primary ALL

Primary ALL cells are co-cultured on irradiated OP9 cells to support long-term survival and proliferation of ALL cells24 and treated with the following drug regimens: vehicle control, AZD2811 (16, 24, and 32 nM), JIB-04 (0.3, 0.5, 1.0 μM), Dex (0.1, 1, and 10 nM) and combinations of AZD2811+Dex and JIB-04+Dex. Concentrations specified are based on our prior data. Viability (Annexin V/7AAD) and cell cycle distribution (BrdDU) is determined by flow cytometry. As potential future clinical markers, we assess drug effects on known targets of AurkB and JIB-04: levels of phosphorylated G9a/GLP and H3S10 and methylated G9a/GLP and histone H3K9 will be compared to total levels of G9a, GLP, and histone H3. We also monitor apoptosis markers (cleaved caspases 3 and 7 and cleaved PARP1). After these initial single and double agent tests, we use the optimal concentrations of each agent to test the combination of Dex+AZD2811+JIB-04. We also test additional combinations of AZD2811 or JIB-04 with the typical ALL chemotherapy regimen (Vincristine, Dex, and L-asparaginase, i.e. VDL). Synergy between AZD2811+Dex or JIB-04+Dex is tested by varying concentrations and analyzing the data with Combosyn software (Chou-Talalay algorithm). Tumor lines is selected from a B-ALL and T-ALL tumor line bank. We select ten primary lines representing various karyotypes and test each primary line in triplicate to get an initial indication of which karyotypes respond to the treatments. If primary samples of one karyotype did not respond or exhibit large variation in response, we test additional tumor lines for that karyotype.

### Preclinical Evaluation of Aurkb Inhibitors and Lysine Demethylase Inhibitors With Dex in Primary ALL Xenograft Models.

The ADZ2811 naoparticle formulation (AZD2811NP) monotherapy is used here showed inhibition of acute myeloid leukemia cell line HL60 growth in vivol8. The JIB-04 lysine demethylase inhibitor was previously effective in mouse xenograft models of lung, breast and glioblastoma cancers at dosages of 5-50 mg/kg. Here, we test AZD2811NP and JIB-04 in xenograft models of primary ALL in combination with Dex.

Combination of Dex with AZD2811NP or JIB-04. NSG mice are injected with luciferase-labelled primary ALL cells to allow real-time monitoring of progression of ALL. Bioluminescent imaging is used to assess reduction of leukemia burden faster than and in addition to assessing survival. Engraftment is confirmed by flow cytometry measurement of human CD45/ human CD19 in the peripheral blood (greater than 1%).

4 weekly i.v. monotherapy doses of 25 mg/kg of AZD2811NP extended survival significantly in a B-ALL xenograft model using a tumor line from a relapsed patient. Dex is administered i.p. daily 5 days per week for 4 weeks along with the weekly injections of AZD2811NP. We use 5 mice per condition, treated as follows: 1) Saline, 2) AZD2811NP, 3) 10, 20, or 40 mg/kg/day Dex, 4) combined AZD2811NP and Dex.

For JIB-04, we are conducting monotherapy experiments to determine effects and tolerated doses, using published studies as guide. From these results, we chose 1-2 doses of JIB to test in combination with the 3 Dex dosages specified above.

Tumor burden is analyzed weekly by bioluminescent imaging of live mice. Survival is analyzed by Kaplan Meier analysis. We initially tested one of the B-ALL tumor lines from relapsed patients which have shown promising results in vitro. We subsequently test additional B-ALL cell lines and T-ALL cell lines that responded favorably to the combined AZD2811NP+Dex treatment in vitro.

Biomarker. Decreased phosphorylation of G9a/GLP and histone H3S10 (for AZD2811) and increased methylation of G9a/GLP and histone H3K9 (for JIB-04) serve as markers of the effectiveness, measured by western blot and compared with total G9a/GLP and H3. Apoptosis markers (cleaved caspases 3 and 7 and cleaved PARP1) is examined. Mice are subjected to peripheral blood drawings after the 1st and last day of treatment. These markers are assessed in future clinical trials.

Toxicity. We test for bone marrow (BM) exhaustion, via blood counts (CBC) on peripheral blood and the BM using a Hemanalyzer to assess white and red blood cells and platelets. Effects on non-hematopoietic tissues in immunocompetent mice (C57/BL6) is followed long-term via histological assessment of femurs (BM), lung, small and large guts, liver and spleen.

Statistical Analysis. In vitro experiments are performed with triplicate samples; statistical analysis is conducted. At least 3 independent experiments for each tumor line is performed. For mouse studies, the CHLA Biostatistical Core supervises statistical analysis, which will be carried out using a 2-tailed Student's t-test or 2-way ANOVA with Bonferroni's post-tests. Survival is analyzed by Kaplan-Meier with multiple testing adjustments. A sample size of 5 mice have 80% power to detect an effect size of 2.024 in difference of marker expressions or in difference of survival, at a two-sided significance level of 0.05. To increase robustness, in vivo experiments are performed with female and repeated with male mice. Testing 3 different ALL samples per karyotype ensures generalizable findings.

### The AZD2811 and JIB-04 Inhibitors Enhance Dex-Induced Cell Death in Cell Line Models of Other Hematologic Malignancies.

Many other hematologic malignancies, in addition to ALL, are normally treated with GC, and GC resistance is also associated with relapse in these patients. Dex induction of genes promote cell deaths using the same mechanism we have demonstrated in ALL i.e. that is AZD2811 and JIB-04 enhances Dex sensitivity by enhancing the coactivator function of G9a/GLP.

The following cell lines are tested: T-ALL: [non-Hodgkins Lymphoma (NHL), Hodgkins Lymphoma, chromic lymphocytic leukemia (CLL), and multiple myeloma]. Cell survival assays are performed as described above. Cells are incubated with various concentrations of AZD2811 and JIB-04 for 72 hours to determine the maximum tolerated concentrations of these inhibitors. Optimal inhibitor concentrations are combined with various Dex concentrations. Apoptosis markers are also examined after 24-hour incubations. Statistical analyses for cell survival assays are conducted. Western blots are repeated for at least 3 independent experiments.

### The Regulatory Mechanisms Controling G9a/GLP Phosphorylation and Methylation.

G9a/GLP coactivator activity, which is critical for GC-induced leukemia cell death, is regulated by automethylation and phosphorylation by AURKB. During mitosis AURKB activity is regulated by kinases and phosphatases. Similar mechanisms regulate AURKB activity during interphase when transcriptional regulation by GR occurs. JIB-04, which inhibits demethylation of G9a/GLP, targets several members of the large JmjC demethylase family. We define the specific G9a/GLP demethylase(s), allowing for future development of more specific inhibitors. Studies are conducted with NALM6 B-ALL cells. Successful identification of these regulatory mechanisms define additional potential sites of therapeutic intervention.

Determining the effects of upstream regulators of AURKB on G9a/GLP phosphorylation and Dex-induced ALL cell death. Aurora kinases play crucial roles in mitosis, and their cellular levels and activity are highly cell cycle regulated, peaking in G2/M28,29. However, since gene regulation by GR occurs during non-mitotic cell cycle phases30,31, we expect that AurkB regulation of G9a is separate from its function in mitosis. A specific role for AurkB regulating transcription during interphase is a novel finding. Experiments we have conducted so far are on unsynchonized cell populations, where AurkB inhibitors may indirectly influence GC regulation of gene expression by altering cell cycle distribution. To eliminate this variable, we block Nalm6 cells in G1/G0 using low serum conditions and then test effects of AurkB inhibitors on GC regulation of gene expression compared with proliferating cells. FACS analysis monitors cell cycle profiles. During mitosis, Chk1 phosphorylates and activates AurkB, EB1 protects AurkB from dephosphorylation, and phosphatases PP1 and PP2A dephosphorylate AurkB33. We deplete and inhibit these factors to test effects on G9a/GLP phosphorylation, using immunoprecipitation of G9a and GLP, followed by western blot with pan phosphothreonine antibodies. The phosphorylated active form of AURKB and total AURKB is also monitored. We also monitor Dex-induced expression of genes that require G9a/GLP as coactivators for GR; GC-induced genes that do not require G9a/GLP will be used as specificity controls. These target genes are previously defined. Finally cell survival assays are conducted in cells where the AURKB regulators have been depleted or inhibited; unsynchronized cells depleted of AURKB regulators or treated with inhibitors of these regulators are incubated with various Dex concentrations for 72 hours before assessing survival.

Identify G9a/GLP demethylases. We tested one inhibitor for each of the two demethylase families: OG-L002 inhibits LSD1 and 234; JIB-04 inhibits some members of the JmjC family, including members of KDM2, KDM3, and KDM4 subfamilies. We propose that inhibition of KDMs that demethylate G9a/GLP (currently unknown) will increase G9a/GLP methylation levels and thereby enhance G9a/GLP coactivator activity, GC activation of cell death genes, and Dex-induced cell death. Each demethylase is depleted with two different lentiviral-delivered shRNAs (depletion confirmed by western blot); effects on G9a/GLP methylation levels will be tested by G9a/GLP immunoprecipitation, followed by western blot with pan-methyllysine antibodies. To confirm we over-express candidate demethylases using lentiviral vectors to look for reduced methylation of G9a/GLP. When they become available, specific inhibitors of individual demethylases will also be tested. Candidate demethylases are also be tested (by depletion, inhibition, or over-expression) for effects on Dex-induced expression of G9a/GLP-dependent and independent genes, and on Dex-induced cell death and Dex-induced apoptosis markers, as described above.

The results allow us to plan specific clinical trials with AZD2811 in relapsed B-ALL and T-ALL patients. AZD2811NP has already been approved for clinical trials as a cell cycle inhibitor, which will facilitate our use of this agent. JIB-04 has been involved in several preclinical studies, but FDA approval will be needed before this agent can be moved into clinical trials.

Cited References for Example 1:
1a. Mullighan C G, Zhang J, Kasper L H, et al. CREBBP mutations in relapsed acute lymphoblastic leukaemia. Nature. 2012; 471(7337):235-239.
2a. Mullighan C G, Phillips L A, Su X, et al. Genomic analysis of the clonal origins of relapsed acute lymphoblastic leukemia. Science. 2008; 322(5906):1377-1380.
3a. Chan L N, Chen Z, Braas D, et al. Metabolic gatekeeper function of B-lymphoid transcription factors. Nature. 2017; 542(7642):479-483.
4a. Ma X, Edmonson M, Yergeau D, et al. Rise and fall of subclones from diagnosis to relapse in pediatric B-acute lymphoblastic leukaemia. Nature Communications. 2015; 6:6604.
5a. Jing D, Bhadri V A, Beck D, et al. Opposing regulation of BIM and BCL2 controls glucocorticoid-induced apoptosis of pediatric acute lymphoblastic leukemia cells. Blood. 2014;125(2):273-283.
6a. Langmead B, Salzberg S L. Fast gapped-read alignment with Bowtie 2. Nat Meth. 2012; 9(4):357-359.
7a. Quinlan A R, Hall I M. BEDTools: a f3lexible suite of utilities for comparing genomic features. Bioinformatics. 2010; 26(6):841-842.
8a. Feng J, Liu T, Qin B, Zhang Y, Liu X S. Identifying ChIP-seq enrichment using MACS. Nat Protoc. 2012; 7(9):1728-1740.
9a. Kampmann M, Bassik M C. Integrated platform for genome-wide screening and construction of high-density genetic interaction maps in mammalian cells. 2013.
10a. Kampmann M, Bassik M C, Weissman J S. Functional genomics platform for pooled screening and generation of mammalian genetic interaction maps. Nat Protoc. 2014; 9(8):1825-1847.
11 a. Pfaffl M W. A new mathematical model for relative quantification in real-time RT-PCR. Nucleic Acids Research. 2001; 29(9):e45.
12a. Roberts K G, Li Y, Payne-Turner D, et al. Targetable kinase-activating lesions in Ph-like acute lymphoblastic leukemia. N. Engl. J. Med. 2014; 371(11):1005-1015.
13a. Maude S L, Tasian S K, Vincent T, et al. Targeting JAK1/2 and mTOR in murine xenograft models of Ph-like acute lymphoblastic leukemia. Blood. 2012; 120(17):3510-3518.
14a. Tasian S K, Li Y, Ryan T, et al. In Vivo Efficacy of PI3K Pathway Signaling Inhibition for Philadelphia Chromosome-Like Acute Lymphoblastic Leukemia. Blood. 2013; 122(21):2672-2672.
15a. Arroyo A G, Yang J T, Rayburn H, Hynes R O. Differential requirements for alpha4 integrins during fetal and adult hematopoiesis. Cell. 1996; 85(7):997-1008.
16a. Schebesta M, Heavey B, Busslinger M. Transcriptional control of B-cell development. Current Opinion in Immunology. 2002; 14(2):216-223.
17a. Ma Q, Jones D, Springer T A. The chemokine receptor CXCR4 is required for the retention of B lineage and granulocytic precursors within the bone marrow microenvironment. Immunity. 1999; 10(4):463-471.
18a. Burger J A, Montserrat E. Coming full circle: 70 years of chronic lymphocytic leukemia cell redistribution, from glucocorticoids to inhibitors of B-cell receptor signaling. Blood. 2013; 121(9):1501-1509.
19a. Cupps T R, Gerrard T L, Falkoff R J, Whalen G, Fauci A S. Effects of in vitro corticosteroids on B cell activation, proliferation, and differentiation. J. Clin. Invest. 1985; 75(2):754-761.
20a. John S, Sabo P J, Thurman R E, et al. Chromatin accessibility pre-determines glucocorticoid receptor binding patterns. Nature Publishing Group. 2011; 43(3):264-268.
21a. Chodankar R, Wu D-Y, Schiller B J, Yamamoto K R, Stallcup M R. Hic-5 is a transcription coregulator that acts before and/or after glucocorticoid receptor genome occupancy in a gene-selective manner. Proceedings of the National Academy of Sciences. 2014; 111(11):4007-4012.
22a. Bassik M C, Kampmann M, Lebbink R J, et al. A systematic mammalian genetic interaction map reveals pathways underlying ricin susceptibility. Cell. 2013; 152(4):909-922.
23a. Kampmann M, Bassik M C, Weissman J S. Integrated platform for genome-wide screening and construction of high-density genetic interaction maps in mammalian cells. Proc. Natl. Acad. Sci. U.S.A. 2013; 110(25):E2317-E2326.
24a. Kampmann M, Horlbeck M A, Chen Y, et al. Next-generation libraries for robust RNA interference-based genome-wide screens. Proc. Natl. Acad. Sci. U.S.A. 2015; 112(26):E3384-E3391.
25a. Mullighan C G, Phillips L A, Su X, et al. Genomic Analysis of the Clonal Origins of Relapsed Acute Lymphoblastic Leukemia. Science. 2008; 322(5906):1377-1380.
26a. Buchner M, Swaminathan S, Chen Z, Müschen M. Mechanisms of pre-B-cell receptor checkpoint control and its oncogenic subversion in acute lymphoblastic leukemia. Immunol. Rev. 2015; 263(1):192-209.
27a. van Galen J C, Kuiper R P, van Emst L, et al. BTG1 regulates glucocorticoid receptor autoinduction in acute lymphoblastic leukemia. Blood. 2010; 115(23):4810-4819.
28a. Müschen M. Rationale for targeting the pre-B-cell receptor signaling pathway in acute lymphoblastic leukemia. Blood. 2015; 125(24):3688-3693.
29a. Wei G, Twomey D, Lamb J, et al. Gene expression-based chemical genomics identifies rapamycin as a modulator of MCL1 and glucocorticoid resistance. Cancer Cell. 2006; 10(4):331-342.
30a. Piovan E, Yu J, Tosello V, et al. Direct Reversal of Glucocorticoid Resistanceby AKT Inhibition in Acute Lymphoblastic Leukemia. Cancer Cell. 2013; 24(6):766-776.
31a. Riviére J-B, Mirzaa G M, O'Roak B J, et al. De novo germline and postzygotic mutations in AKT3, PIK3R2 and PIK3CA cause a spectrum of related megalencephaly syndromes. Nat Genet. 2012; 44(8):934-940.
32a. Chiron D, Martin P, Di Liberto M, et al. Induction of prolonged early G 1 arrest by CDK4/CDK6 inhibition reprograms lymphoma cells for durable PI3Kδ inhibition through PIK3IP1. Cell Cycle. 2014; 12(12):1892-1900.
33a. Fruman D A, Cantley L C. Idelalisib—A PI3Kδ Inhibitor for B-Cell Cancers. N. Engl. J. Med. 2014; 370(11):1061-1062.
34a. Tasian S K, Teachey D T, Li Y, et al. Potent efficacy of combined PI3K/mTOR and JAK or ABL inhibition in murine xenograft models of Ph-like acute lymphoblastic leukemia. Blood. 2017; 129(2):177-187.
35a. Tallarida R J. Quantitative Methods for Assessing Drug Synergism. Genes & Cancer. 2012; 2(11):1003-1008.
36a. Geng H, Hurtz C, Lenz K B, et al. Self-Enforcing Feedback Activation between BCL6 and Pre-B Cell Receptor Signaling Defines a Distinct Subtype of Acute Lymphoblastic Leukemia. Cancer Cell. 2015; 27(3):409-425.
37a. Silveira A B, Laranjeira A B A, Rodrigues G O L, et al. PI3K inhibition synergizes with glucocorticoids but antagonizes with methotrexate in T-cell acute lymphoblastic leukemia. Oncotarget. 2015; 6(15):13105-13118.
38a. Blind R D, Garabedian M J. Differential recruitment of glucocorticoid receptor phospho-isoforms to glucocorticoid-induced genes. The Journal of Steroid Biochemistry and Molecular Biology. 2008; 109(1-2): 150-157.
39a. Wang Z, Garabedian M J. Modulation of glucocorticoid receptor transcriptional activation, phosphorylation, and growth inhibition by p27Kip1. J. Biol. Chem. 2003; 278(51):50897-50901.
40a. Krstic M D, Rogatsky I, Yamamoto K R, Garabedian M J. Mitogen-activated and cyclin-dependent protein kinases selectively and differentially modulate transcriptional enhancement by the glucocorticoid receptor. Molecular and Cellular Biology. 1997; 17(7):3947-3954.
41a. Galliher-Beckley A J, Cidlowski J A. Emerging roles of glucocorticoid receptor phosphorylation in modulating glucocorticoid hormone action in health and disease. IUBMB Life. 2009; 61(10):979-986.
42a. Mittelstadt P R, Monteiro J P, Ashwell J D. Thymocyte responsiveness to endogenous glucocorticoids is required for immunological fitness. J. Clin. Invest. 2012; 122(7):2384-2394.

43a. Johnson D E, Redner R L. An ATRActive future for differentiation therapy in AML. YBLRE. 2015; 29(4):263-268.
44a. Drexler H, MacLeod R, Nagel S, Dirks W. Guide to Leukemia-Lymphoma Cell Lines on CD. Blood; 2005.
45a. Acosta-Alvear D, Cho M Y, Wild T, Buchholz T J. Paradoxical resistance of multiple myeloma to proteasome inhibitors by decreased levels of 19S proteasomal subunits. eLife. 2015.
46a. Nahar R, Müschen M. Pre-B cell receptor signaling in acute lymphoblastic leukemia. Cell Cycle. 2009; 8(23):3874-3877.

Cited References for Example 1:

Feng, J., Liu, T., Qin, B., Zhang, Y., Liu, X. S., 2012. Identifying ChIP-seq enrichment using MACS. Nat Protoc 7, 1728-1740. doi:10.1038/nprot.2012.101

Kampmann, M., Bassik, M. C., 2013. Integrated platform for genome-wide screening and construction of highdensity genetic interaction maps in mammalian cells, in:. Presented at the Proceedings of the . . . . doi:10.1073/pnas.1307002110/-/DCSupplemental

Kampmann, M., Bassik, M. C., Weissman, J. S., 2014. Functional genomics platform for pooled screening and generation of mammalian genetic interaction maps. Nat Protoc 9, 1825-1847. doi: 10.1038/nprot.2014.103

Langmead, B., Salzberg, S. L., 2012. Fast gapped-read alignment with Bowtie 2. Nat Meth 9, 357-359. doi:10.1038/nmeth.1923

Pfaffl, M. W., 2001. A new mathematical model for relative quantification in real-time RT-PCR. Nucleic Acids Research 29, e45.

Quinlan, A. R., Hall, I. M., 2010. BEDTools: a flexible suite of utilities for comparing genomic features. Bioinformatics 26, 841-842. doi:10.1093/bioinformatics/btq033

Cited References for Example 2:
1b. Tagami T, Madison L D, Nagaya T, Jameson J L (1997) Nuclear receptor corepressors activate rather than suppress basal transcription of genes that are negatively regulated by thyroid hormone. Mol Cell Biol 17: 2642-8
2b. Huang S M, Stallcup M R (2000) Mouse Zac1, a transcriptional coactivator and repressor for nuclear receptors. Mol Cell Biol 20: 1855-67
3b. Rogatsky I, Luecke H F, Leitman D C, Yamamoto K R (2002) Alternate surfaces of transcriptional coregulator GRIP1 function in different glucocorticoid receptor activation and repression contexts. Proc Natl Acad Sci USA 99: 16701-6
4b. Bittencourt D, Wu D Y, Jeong K W, Gerke D S, Herviou L, lanculescu I, Chodankar R, Siegmund K D, Stallcup M R (2012) G9a functions as a molecular scaffold for assembly of transcriptional coactivators on a subset of glucocorticoid receptor target genes. Proc Natl Acad Sci USA 109: 19673-8
5b. Chodankar R, Wu D Y, Schiller B J, Yamamoto K R, Stallcup M R (2014) Hic-5 is a transcription coregulator that acts before and/or after glucocorticoid receptor genome occupancy in a gene-selective manner. Proc Natl Acad Sci USA111: 4007-12
6b. Vakoc C R, Mandat S A, Olenchock B A, Blobel G A (2005) Histone H3 lysine 9 methylation and HP1gamma are associated with transcription elongation through mammalian chromatin. Mol Cell 19: 381-91
7b. Tachibana M, Sugimoto K, Nozaki M, Ueda J, Ohta T, Ohki M, Fukuda M, Takeda N, Niida H, Kato H, et al. (2002) G9a histone methyltransferase plays a dominant role in euchromatic histone H3 lysine 9 methylation and is essential for early embryogenesis. Genes Dev 16: 1779-91
8b. Shankar S R, Bahirvani A G, Rao V K, Bharathy N, Ow J R, Taneja R (2013) G9a, a multipotent regulator of gene expression. Epigenetics 8: 16-22
9b. Benevento M, van de Molengraft M, van Westen R, van Bokhoven H, Kasri N N (2015) The role of chromatin repressive marks in cognition and disease: A focus on the repressive complex GLP/G9a. Neurobiol Learn Mem 124: 88-96
10b. Casciello F, Windloch K, Gannon F, Lee J S (2015) Functional Role of G9a Histone Methyltransferase in Cancer. Front Immunol 6: 487
11b. Lee D Y, Northrop J P, Kuo M H, Stallcup M R (2006) Histone H3 lysine 9 methyltransferase G9a is a transcriptional coactivator for nuclear receptors. J Biol Chem 281: 8476-85
12b. Purcell D J, Jeong K W, Bittencourt D, Gerke D S, Stallcup M R (2011) A distinct mechanism for coactivator versus corepressor function by histone methyltransferase G9a in transcriptional regulation. J Biol Chem 286: 41963-71
13b. Purcell D J, Khalid O, Ou C Y, Little G H, Frenkel B, Baniwal S K, Stallcup M R (2012) Recruitment of coregulator G9a by Runx2 for selective enhancement or suppression of transcription. J Cell Biochem 113: 2406-14
14b. Chaturvedi C P, Hosey A M, Palii C, Perez-Iratxeta C, Nakatani Y, Ranish J A, Dilworth F J, Brand M (2009) Dual role for the methyltransferase G9a in the maintenance of beta-globin gene transcription in adult erythroid cells. Proc Natl Acad Sci USA 106: 18303-8
15b. Lehnertz B, Northrop J P, Antignano F, Burrows K, Hadidi S, Mullaly S C, Rossi F M, Zaph C (2010) Activating and inhibitory functions for the histone lysine methyltransferase G9a in T helper cell differentiation and function. J Exp Med 207: 915-22
16b. Chin H G, Estéve P O, Pradhan M, Benner J, Patnaik D, Carey M F, Pradhan S (2007) Automethylation of G9a and its implication in wider substrate specificity and HP1 binding. Nucleic Acids Res 35: 7313-23
17b. Sampath S C, Marazzi I, Yap K L, Krutchinsky A N, Mecklenbrauker I, Viale A, Rudensky E, Zhou M M, Chait B T, Tarakhovsky A (2007) Methylation of a histone mimic within the histone methyltransferase G9a regulates protein complex assembly. Mol Cell 27: 596-608
18b. Kwon S H, Workman J L (2011) The changing faces of HP1: From heterochromatin formation and gene silencing to euchromatic gene expression: HP1 acts as a positive regulator of transcription. Bioessays 33: 280-9
19b. Liu F, Barsyte-Lovejoy D, Allali-Hassani A, He Y, Herold J M, Chen X, Yates C M, Frye S V, Brown P J, Huang J, et al. (2011) Optimization of cellular activity of G9a inhibitors 7-aminoalkoxy-quinazolines. J Med Chem 54: 6139-50
20b. Liu F, Barsyte-Lovejoy D, Li F, Xiong Y, Korboukh V, Huang X P, Allali-Hassani A, Janzen W P, Roth B L, Frye S V, et al. (2013) Discovery of an in vivo chemical probe of the lysine methyltransferases G9a and GLP. J Med Chem 56: 8931-42
21b. Soderberg O, Gullberg M, Jarvius M, Ridderstrale K, Leuchowius KJ, Jarvius J, Wester K, Hydbring P, Bahram F, Larsson L G, et al. (2006) Direct observation of individual endogenous protein complexes in situ by proximity ligation. Nat Methods 3: 995-1000
22b. Tachibana M, Ueda J, Fukuda M, Takeda N, Ohta T, Iwanari H, Sakihama T, Kodama T, Hamakubo T, Shinkai Y (2005) Histone methyltransferases G9a and GLP form heteromeric complexes and are both crucial for methylation of euchromatin at H3-K9. Genes Dev 19: 815-26
23b. Reddy T E, Pauli F, Sprouse R O, Neff N F, Newberry K M, Garabedian M J, Myers R M (2009) Genomic determination of the glucocorticoid response reveals unexpected mechanisms of gene regulation. Genome Res 19: 2163-71
24b. Fischle W, Tseng B S, Dormann H L, Ueberheide B M, Garcia B A, Shabanowitz J, Hunt D F, Funabiki H, Allis C D (2005) Regulation of HP1-chromatin binding by histone H3 methylation and phosphorylation. Nature 438: 1116-22
25b. Hirota T, Lipp J J, Toh B H, Peters J M (2005) Histone H3 serine 10 phosphorylation by Aurora B causes HP1 dissociation from heterochromatin. Nature 438: 1176-80
26b. Koike N, Maita H, Taira T, Ariga H, Iguchi-Ariga S M (2000) Identification of heterochromatin protein 1 (HP1) as a phosphorylation target by Pim-1 kinase and the effect of phosphorylation on the transcriptional repression function of HP1(1). FEBS Lett 467: 17-21
27b. Lomberk G, Bensi D, Fernandez-Zapico M E, Urrutia R (2006) Evidence for the existence of an HP1-mediated subcode within the histone code. Nat Cell Biol 8: 407-15
28b. Le Bras G F, Taubenslag K J, Andl C D (2012) The regulation of cell-cell adhesion during epithelial-mesenchymal transition, motility and tumor progression. Cell Adh Migr 6: 365-73
29b. Wu D Y, Ou C Y, Chodankar R, Siegmund K D, Stallcup M R (2014) Distinct, genome-wide, gene-specific selectivity patterns of four glucocorticoid receptor coregulators. Nucl Recept Signal 12: e002
30b. Meijsing S H, Pufall M A, So A Y, Bates D L, Chen L, Yamamoto K R (2009) DNA binding site sequence directs glucocorticoid receptor structure and activity. Science 324: 407-10
31b. Thomas-Chollier M, Watson L C, Cooper S B, Pufall M A, Liu J S, Borzym K, Vingron M, Yamamoto K R, Meijsing S H (2013) A naturally occurring insertion of a single amino acid rewires transcriptional regulation by glucocorticoid receptor isoforms. Proc Natl Acad Sci USA 110: 17826-31
32b. Yamane K, Toumazou C, Tsukada Y, Erdjument-Bromage H, Tempst P, Wong J, Zhang Y (2006) JHDM2A, a JmjC-containing H3K9 demethylase, facilitates transcription activation by androgen receptor. Cell 125: 483-95
33b. Shi Y, Lan F, Matson C, Mulligan P, Whetstine J R, Cole P A, Casero R A (2004) Histone demethylation mediated by the nuclear amine oxidase homolog LSD1. Cell 119: 941-53
34b. Horton J R, Upadhyay A K, Qi H H, Zhang X, Shi Y, Cheng X (2010) Enzymatic and structural insights for substrate specificity of a family of jumonji histone lysine demethylases. Nat Struct Mol Biol 17: 38-43
35b. Couture J F, Collazo E, Ortiz-Tello P A, Brunzelle J S, Trievel R C (2007) Specificity and mechanism of JMJD2A, a trimethyllysine-specific histone demethylase. Nat Struct Mol Biol 14: 689-95
36b. Carmena M, Ruchaud S, Earnshaw W C (2009) Making the Auroras glow: regulation of Aurora A and B kinase function by interacting proteins. Curr Opin Cell Biol 21: 796-805
37b. Goldenson B, Crispino J D (2015) The aurora kinases in cell cycle and leukemia. Oncogene 34: 537-45
38b. den Hollander J, Rimpi S, Doherty J R, Rudelius M, Buck A, Hoellein A, Kremer M, Graf N, Scheerer M, Hall M A, et al. (2010) Aurora kinases A and B are up-regulated by Myc and are essential for maintenance of the malignant state. Blood 116: 1498-505
39b. Jha H C, Yang K, El-Naccache D W, Sun Z, Robertson E S (2015) EBNA3C regulates p53 through induction of Aurora kinase B. Oncotarget 6: 5788-803
40b. Jung E, Seong Y, Seo J H, Kwon Y S, Song H (2014) Cell cycle-dependent regulation of Aurora kinase B mRNA by the Microprocessor complex. Biochem Biophys Res Commun 446: 241-7
41b. Rao V K, Ow J R, Shankar S R, Bharathy N, Manikandan J, Wang Y, Taneja R (2016) G9a promotes proliferation and inhibits cell cycle exit during myogenic differentiation. Nucleic Acids Res 44: 8129-43
42b. Huang J, Dorsey J, Chuikov S, Perez-Burgos L, Zhang X, Jenuwein T, Reinberg D, Berger S L (2010) G9a and G1p methylate lysine 373 in the tumor suppressor p53. J Biol Chem 285: 9636-41
43b. Larsen S L, Yde C W, Laenkholm A V, Rasmussen B B, Duun-Henriksen A K, Bak M, Lykkesfeldt A E, Kirkegaard T (2015) Aurora kinase B is important for antiestrogen resistant cell growth and a potential biomarker for tamoxifen resistant breast cancer. BMC Cancer 15: 239
44b. Collins R E, Northrop J P, Horton J R, Lee D Y, Zhang X, Stallcup M R, Cheng X (2008) The ankyrin repeats of G9a and GLP histone methyltransferases are mono- and dimethyllysine binding modules. Nat Struct Mol Biol 15: 245-50
45b. Poulard C, Rambaud J, Le Romancer M, Corbo L (2014) Proximity ligation assay to detect and localize the interactions of ERα with PI3-K and Src in breast cancer cells and tumor samples. Methods Mol Biol 1204: 135-43
46b. Rasband, W. S., ImageJ, U. S. National Institutes of Health, Bethesda, Md., USA, http://imagej.nih.gov/ij/, 1997-2015.

Cited References for Example 3:
Adam, E., Kim, H. N., Gang, E. J., Schnair, C., Lee, S., Khazal, S., Kosoyan, O., Konopleva, M., Parekh, C., Bhojwani, D., et al. (2017). The PI3Kδ Inhibitor Idelalisib Inhibits Homing in an in Vitro and in Vivo Model of B ALL. Cancers (Basel) 9.
Aleem, E., and Arceci, R. J. (2015). Targeting cell cycle regulators in hematologic malignancies. Front Cell Dev Biol 3, 16.
Asghar, U., Witkiewicz, A. K., Turner, N. C., and Knudsen, E. S. (2015). The history and future of targeting cyclin-dependent kinases in cancer therapy. Nat Rev Drug Discov 14, 130-146.
Bakker, E., Tian, K., Mutti, L., Demonacos, C., Schwartz, J. M., and Krstic-Demonacos, M. (2017). Insight into glucocorticoid receptor signalling through interactome model analysis. PLoS Comput Biol 13, e1005825.
Bhojwani, D., Kang, H., Moskowitz, N. P., Min, D. J., Lee, H., Potter, J. W., Davidson, G., Willman, C. L., Borowitz, M. J., Belitskaya-Levy, I., et al. (2006). Biologic pathways associated with relapse in childhood acute lymphoblastic leukemia: a Children's Oncology Group study. Blood 108, 711-717.
Bittencourt, D., Wu, D. Y., Jeong, K. W., Gerke, D. S., Herviou, L., lanculescu, I., Chodankar, R., Siegmund, K. D., and Stallcup, M. R. (2012). G9a functions as a molecular scaffold for assembly of transcriptional coactivators on a subset of glucocorticoid receptor target genes. Proc Natl Acad Sci USA 109, 19673-19678.
Bruscoli, S., Biagioli, M., Sorcini, D., Frammartino, T., Cimino, M., Sportoletti, P., Mazzon, E., Bereshchenko, O., and Riccardi, C. (2015). Lack of glucocorticoid-induced leucine zipper (GILZ) deregulates B-cell survival and results in B-cell lymphocytosis in mice. Blood 126, 1790-1801.
Chen, Z., Lopez-Ramos, D. A., Yoshihara, E., Maeda, Y., Masutani, H., Sugie, K., Maeda, M., and Yodoi, J. (2011). Thioredoxin-binding protein-2 (TBP-2/VDUP1/TXNIP) regulates T-cell sensitivity to glucocorticoid during HTLV-I-induced transformation. Leukemia 25, 440-448.
Chow, Y. P., Alias, H., and Jamal, R. (2017). Meta-analysis of gene expression in relapsed childhood B-acute lymphoblastic leukemia. BMC Cancer 17, 120.
D′Avino, P. P., and Capalbo, L. (2015). New Auroras on the Roles of the Chromosomal Passenger Complex in Cytokinesis: Implications for Cancer Therapies. Front Oncol 5, 221.
Davis, S., and Meltzer, P. S. (2007). GEOquery: a bridge between the Gene Expression Omnibus (GEO) and BioConductor. Bioinformatics 23, 1846-1847.
Ditchfield, C., Johnson, V. L., Tighe, A., Ellston, R., Haworth, C., Johnson, T., Mortlock, A., Keen, N., and Taylor, S. S. (2003). Aurora B couples chromosome alignment with anaphase by targeting BubR1, Mad2, and Cenp-E to kinetochores. J Cell Biol 161, 267-280.
Du, P., Kibbe, W. A., and Lin, S. M. (2008). lumi: a pipeline for processing Illumina microarray. Bioinformatics 24, 1547-1548.
Fischer, J., Paret, C., El Malki, K., Alt, F., Wingerter, A., Neu, M. A., Kron, B., Russo, A., Lehmann, N., Roth, L., et al. (2017). CD19 Isoforms Enabling Resistance to CART-19 Immunotherapy Are Expressed in B-ALL Patients at Initial Diagnosis. J Immunother 40, 187-195.
Floc'h, N., Ashton, S., Taylor, P., Trueman, D., Harris, E., Odedra, R., Maratea, K., Derbyshire, N., Caddy, J., Jacobs, V. N., et al. (2017). Optimizing Therapeutic Effect of Aurora B Inhibition in Acute Myeloid Leukemia with AZD2811 Nanoparticles. Mol Cancer Ther 16, 1031-1040.
Fuchs, O. (2010). Transcription factor NF-κB inhibitors as single therapeutic agents or in combination with classical chemotherapeutic agents for the treatment of hematologic malignancies. Curr Mol Pharmacol 3, 98-122.
Fulda, S. (2009). Inhibitor of apoptosis proteins in hematological malignancies. Leukemia 23, 467-476.
Gautier, L., Cope, L., Bolstad, B. M., and Irizarry, R. A. (2004). affy—analysis of Affymetrix GeneChip data at the probe level. Bioinformatics 20, 307-315.
Giordano, L., Moldwin, R. L., Downie, P. A., Goldberg, A., Gupta, R., Allen, R., Aithal, N. H., Kim, D. H., Le Moine, P. J., and Smith, S. D. (1997). Growth inhibition of B-cell precursor acute lymphoblastic leukemia cell lines by monocytes: a role for prostaglandin E2. Leuk Res 21, 925-932.
Girdler, F., Gascoigne, K. E., Eyers, P. A., Hartmuth, S., Crafter, C., Foote, K. M., Keen, N. J., and Taylor, S. S. (2006). Validating Aurora B as an anti-cancer drug target. J Cell Sci 119, 3664-3675.
Goldenson, B., and Crispino, J. D. (2015). The aurora kinases in cell cycle and leukemia. Oncogene 34, 537-545.
Goodman, R. H., and Smolik, S. (2000). CBP/p300 in cell growth, transformation, and development. Genes Dev 14, 1553-1577.
Granner, D. K., Wang, J. C., and Yamamoto, K. R. (2015). Regulatory Actions of Glucocorticoid Hormones: From Organisms to Mechanisms. Adv Exp Med Biol 872, 3-31.
Hogan, L. E., Meyer, J. A., Yang, J., Wang, J., Wong, N., Yang, W., Condos, G., Hunger, S. P., Raetz, E., Saffery, R., et al. (2011). Integrated genomic analysis of relapsed childhood acute lymphoblastic leukemia reveals therapeutic strategies. Blood 118, 5218-5226.
Hsieh, Y. T., Gang, E. J., Geng, H., Park, E., Huantes, S., Chudziak, D., Dauber, K., Schaefer, P., Scharman, C., Shimada, H., et al. (2013). Integrin alpha4 blockade sensitizes drug resistant pre-B acute lymphoblastic leukemia to chemotherapy. Blood 121, 1814-1818.
Hsu, S. C., and DeFranco, D. B. (1995). Selectivity of cell cycle regulation of glucocorticoid receptor function. J Biol Chem 270, 3359-3364.
Hsu, S. C., Qi, M., and DeFranco, D. B. (1992). Cell cycle regulation of glucocorticoid receptor function. EMBO J 11, 3457-3468.
Hu, J. M., Bodwell, J. E., and Munck, A. (1994). Cell cycle-dependent glucocorticoid receptor phosphorylation and activity. Mol Endocrinol 8, 1709-1713.
Inaba, H., and Pui, C. H. (2010). Glucocorticoid use in acute lymphoblastic leukaemia. Lancet Oncol 11, 1096-1106.
Ito, K., Yamamura, S., Essilfie-Quaye, S., Cosio, B., Ito, M., Barnes, P. J., and Adcock, I. M. (2006). Histone deacetylase 2-mediated deacetylation of the glucocorticoid receptor enables NF-kappaB suppression. J Exp Med 203, 7-13.
Jones, C. L., Bhatla, T., Blum, R., Wang, J., Paugh, S. W., Wen, X., Bourgeois, W., Bitterman, D. S., Raetz, E. A., Morrison, D. J., et al. (2014). Loss of TBL1XR1 disrupts glucocorticoid receptor recruitment to chromatin and results in glucocorticoid resistance in a B-lymphoblastic leukemia model. J Biol Chem 289, 20502-20515.
Kampmann, M., Bassik, M. C., and Weissman, J. S. (2013). Integrated platform for genome-wide screening and construction of high-density genetic interaction maps in mammalian cells. Proc Natl Acad Sci USA 110, E2317-2326.
Kampmann, M., Bassik, M. C., and Weissman, J. S. (2014). Functional genomics platform for pooled screening and generation of mammalian genetic interaction maps. Nat Protoc 9, 1825-1847.
Kininis, M., and Kraus, W. L. (2008). A global view of transcriptional regulation by nuclear receptors: gene expression, factor localization, and DNA sequence analysis. Nucl Recept Signal 6, e005.
Klumper, E., Pieters, R., Veerman, A. J., Huismans, D. R., Loonen, A. H., Mällen, K., Kaspers, G. J., van Wering, E. R., Hartmann, R., and Henze, G. (1995). In vitro cellular drug resistance in children with relapsed/refractory acute lymphoblastic leukemia. Blood 86, 3861-3868.
Krstic, M. D., Rogatsky, I., Yamamoto, K. R., and Garabedian, M. J. (1997). Mitogen-activated and cyclin-dependent protein kinases selectively and differentially modulate transcriptional enhancement by the glucocorticoid receptor. Mol Cell Biol 17, 3947-3954.
Kruth, K. A., Fang, M., Shelton, D. N., Abu-Halawa, O., Mahling, R., Yang, H., Weissman, J. S., Loh, M. L., Müschen, M., Tasian, S. K., et al. (2017). Suppression of B-cell development genes is key to glucocorticoid efficacy in treatment of acute lymphoblastic leukemia. Blood 129, 3000-3008.
Kumar, R., and Calhoun, W. J. (2008). Differential regulation of the transcriptional activity of the glucocorticoid receptor through site-specific phosphorylation. Biologics 2, 845-854.
Lai, A. Y., and Wade, P. A. (2011). Cancer biology and NuRD: a multifaceted chromatin remodelling complex. Nat Rev Cancer 11, 588-596.
Lee, D. Y., Northrop, J. P., Kuo, M. H., and Stallcup, M. R. (2006). Histone H3 lysine 9 methyltransferase G9a is a transcriptional coactivator for nuclear receptors. J Biol Chem 281, 8476-8485.
Lönnerholm, G., Thörn, I., Sundström, C., Frost, B. M., Abrahamsson, J., Behrendtz, M., Heldrup, J., Jacobsson, S., Li, A., Olofsson, T., et al. (2009). In vitro cellular drug sensitivity at diagnosis is correlated to minimal residual disease at end of induction therapy in childhood acute lymphoblastic leukemia. Leuk Res 33, 46-53.
Madhusoodhan, P. P., Carroll, W. L., and Bhatla, T. (2016). Progress and Prospects in Pediatric Leukemia. Curr Probl Pediatr Adolesc Health Care 46, 229-241.
McDonald, E. R., de Weck, A., Schlabach, M. R., Billy, E., Mavrakis, K. J., Hoffman, G. R., Belur, D., Castelletti, D., Frias, E., Gampa, K., et al. (2017). Project DRIVE: A Compendium of Cancer Dependencies and Synthetic Lethal Relationships Uncovered by Large-Scale, Deep RNAi Screening. Cell 170, 577-592.e510.
Mortlock, A. A., Foote, K. M., Heron, N. M., Jung, F. H., Pasquet, G., Lohmann, J. J., Warin, N., Renaud, F., De Savi, C., Roberts, N. J., et al. (2007). Discovery, synthesis, and in vivo activity of a new class of pyrazoloquinazolines as selective inhibitors of aurora B kinase. J Med Chem 50, 2213-2224.
Mullighan, C. G., Zhang, J., Kasper, L. H., Lerach, S., Payne-Turner, D., Phillips, L. A., Heatley, S. L., Holmfeldt, L., Collins-Underwood, J. R., Ma, J., et al. (2011). CREBBP mutations in relapsed acute lymphoblastic leukaemia. Nature 471, 235-239.
Naderi, E. H., Skah, S., Ugland, H., Myklebost, O., Sandnes, D. L., Torgersen, M. L., Josefsen, D., Ruud, E., Naderi, S., and Blomhoff, H. K. (2015). Bone marrow stroma-derived PGE2 protects BCP-ALL cells from DNA damage-induced p53 accumulation and cell death. Mol Cancer 14, 14.
Naderi, E. H., Ugland, H. K., Diep, P. P., Josefsen, D., Ruud, E., Naderi, S., and Blomhoff, H. K. (2013). Selective inhibition of cell death in malignant vs normal B-cell precursors: implications for cAMP in development and treatment of BCP-ALL. Blood 121, 1805-1813.
Ness, K. K., Armenian, S. H., Kadan-Lottick, N., and Gurney, J. G. (2011). Adverse effects of treatment in childhood acute lymphoblastic leukemia: general overview and implications for long-term cardiac health. Expert Rev Hematol 4, 185-197.
Pendleton, M., Lindsey, R. H., Felix, C. A., Grimwade, D., and Osheroff, N. (2014). Topoisomerase II and leukemia. Ann N Y Acad Sci 1310, 98-110.
Petta, I., Dejager, L., Ballegeer, M., Lievens, S., Tavernier, J., De Bosscher, K., and Libert, C. (2016). The Interactome of the Glucocorticoid Receptor and Its Influence on the Actions of Glucocorticoids in Combatting Inflammatory and Infectious Diseases. Microbiol Mol Biol Rev 80, 495-522.
Poulard, C., Bittencourt, D., Wu, D. Y., Hu, Y., Gerke, D. S., and Stallcup, M. R. (2017). A post-translational modification switch controls coactivator function of histone methyltransferases G9a and GLP. EMBO Rep 18, 1442-1459.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47.
Rodriguez-Fraticelli, A. E., Wolock, S. L., Weinreb, C. S., Panero, R., Patel, S. H., Jankovic, M., Sun, J., Calogero, R. A., Klein, A. M., and Camargo, F. D. (2018). Clonal analysis of lineage fate in native haematopoiesis. Nature 553, 212-216.
Schmidt, S., Rainer, J., Ploner, C., Presul, E., Riml, S., and Kofler, R. (2004). Glucocorticoid-induced apoptosis and glucocorticoid resistance: molecular mechanisms and clinical relevance. Cell Death Differ 11 Suppl 1, S45-55.
Smith, L. K., and Cidlowski, J. A. (2010). Glucocorticoid-induced apoptosis of healthy and malignant lymphocytes. Prog Brain Res 182, 1-30.
Soleymani Fard, S., Jeddi Tehrani, M., and Ardekani, A. M. (2012). Prostaglandin E2 induces growth inhibition, apoptosis and differentiation in T and B cell-derived acute lymphoblastic leukemia cell lines (CCRF-CEM and Nalm-6). Prostaglandins Leukot Essent Fatty Acids 87, 17-24.
Staal, F. J., de Ridder, D., Szczepanski, T., Schonewille, T., van der Linden, E. C., van Wering, E. R., van der Velden, V. H., and van Dongen, J. J. (2010). Genome-wide expression analysis of paired diagnosis-relapse samples in ALL indicates involvement of pathways related to DNA replication, cell cycle and DNA repair, independent of immune phenotype. Leukemia 24, 491-499.
Storey, J. D., and Tibshirani, R. (2003). Statistical significance for genomewide studies. Proc Natl Acad Sci U S A 100, 9440-9445.
Stubbs, M. C., Kim, W., Bariteau, M., Davis, T., Vempati, S., Minehart, J., Witkin, M., Qi, J., Krivtsov, A. V., Bradner, J. E., et al. (2015). Selective Inhibition of HDAC1 and HDAC2 as a Potential Therapeutic Option for B-ALL. Clin Cancer Res 21, 2348-2358.
Tachibana, M., Ueda, J., Fukuda, M., Takeda, N., Ohta, T., Iwanari, H., Sakihama, T., Kodama, T., Hamakubo, T., and Shinkai, Y. (2005). Histone methyltransferases G9a and GLP form heteromeric complexes and are both crucial for methylation of euchromatin at H3-K9. Genes Dev 19, 815-826.
Tanioka, T., Nakatani, Y., Semmyo, N., Murakami, M., and Kudo, I. (2000). Molecular identification of cytosolic prostaglandin E2 synthase that is functionally coupled with cyclooxygenase-1 in immediate prostaglandin E2 biosynthesis. J Biol Chem 275, 32775-32782.
Terwilliger, T., and Abdul-Hay, M. (2017). Acute lymphoblastic leukemia: a comprehensive review and 2017 update. Blood Cancer J 7, e577.
van Galen, J. C., Kuiper, R. P., van Ernst, L., Levers, M., Tijchon, E., Scheijen, B., Waanders, E., van Reijmersdal, S. V., Gilissen, C., van Kessel, A. G., et al. (2010). BTG1 regulates glucocorticoid receptor autoinduction in acute lymphoblastic leukemia. Blood 115, 4810-4819.
Vo, N., and Goodman, R. H. (2001). CREB-binding protein and p300 in transcriptional regulation. J Biol Chem 276, 13505-13508.
Wilkinson, R. W., Odedra, R., Heaton, S. P., Wedge, S. R., Keen, N. J., Crafter, C., Foster, J. R., Brady, M. C., Bigley, A., Brown, E., et al. (2007). AZD1152, a selective inhibitor of Aurora B kinase, inhibits human tumor xenograft growth by inducing apoptosis. Clin Cancer Res 13, 3682-3688.
Yamamoto, K. R. (1985). Steroid receptor regulated transcription of specific genes and gene networks. Annu Rev Genet 19, 209-252.
Yang, J. J., Cheng, C., Yang, W., Pei, D., Cao, X., Fan, Y., Pounds, S. B., Neale, G., Treviño, L. R., French, D., et al. (2009). Genome-wide interrogation of germline genetic variation associated with treatment response in childhood acute lymphoblastic leukemia. JAMA 301, 393-403.

Cited References for Example 4:
1c. Wolf I M, Heitzer M D, Grubisha M, DeFranco D B. Coactivators and nuclear receptor transactivation. J Cell Biochem 2008; 104:1580-1586
2c. Rogatsky I, Luecke H F, Leitman D C, Yamamoto K R. Alternate surfaces of transcriptional coregulator GRIP1 function in different glucocorticoid receptor activation and repression contexts. Proc Natl Acad Sci U S A 2002; 99:16701-16706
3c. Bittencourt D, Wu D Y, Jeong K W, Gerke D S, Herviou L, Ianculescu I, Chodankar R, Siegmund K D, Stallcup M R. G9a functions as a molecular scaffold for assembly of transcriptional coactivators on a subset of glucocorticoid receptor target genes. Proc Natl Acad Sci U S A 2012; 109:19673-19678
4c. Chodankar R, Wu D Y, Schiller B J, Yamamoto K R, Stallcup M R. Hic-5 is a transcription coregulator that acts before and/or after glucocorticoid receptor genome occupancy in a gene-selective manner. Proc Natl Acad Sci U S A 2014; 111:4007-4012
5c. Wu D Y, Ou C Y, Chodankar R, Siegmund K D, Stallcup M R. Distinct, genome-wide, gene-specific selectivity patterns of four glucocorticoid receptor coregulators. Nucl Recept Signal 2014; 12:e002
6c. Poulard C, Bittencourt D, Wu D-Y, Hu U, Gerke D S, Stallcup M R. A Post-translational Modification Switch Controls Coactivator Function of Histone Methyltransferases G9a and GLP. EMBO Reports, in press.
7c. Ou C Y, Chen T C, Lee J V, Wang J C, Stallcup M R. Coregulator cell cycle and apoptosis regulator 1 (CCAR1) positively regulates adipocyte differentiation through the glucocorticoid signaling pathway. J Biol Chem 2014; 289:17078-17086
8c. Tachibana M, Sugimoto K, Nozaki M, Ueda J, Ohta T, Ohki M, Fukuda M, Takeda N, Niida H, Kato H, Shinkai Y. G9a histone methyltransferase plays a dominant role in euchromatic histone H3 lysine 9 methylation and is essential for early embryogenesis. Genes Dev 2002; 16:1779-1791
9c. Lee D Y, Northrop J P, Kuo M H, Stallcup M R. Histone H3 lysine 9 methyltransferase G9a is a transcriptional coactivator for nuclear receptors. J Biol Chem 2006; 281:8476-8485
10c. Purcell D J, Jeong K W, Bittencourt D, Gerke D S, Stallcup M R. A distinct mechanism for coactivator versus corepressor function by histone methyltransferase G9a in transcriptional regulation. J Biol Chem 2011; 286:41963-41971
11c. Chaturvedi C P, Hosey A M, Palii C, Perez-Iratxeta C, Nakatani Y, Ranish J A, Dilworth F J, Brand M. Dual role for the methyltransferase G9a in the maintenance of beta-globin gene transcription in adult erythroid cells. Proc Natl Acad Sci U S A 2009; 106:18303-18308
12c. Bhojwani D, Kang H, Menezes R X, Yang W, Sather H, Moskowitz N P, Min D J, Potter J W, Harvey R, Hunger S P, Seibel N, Raetz E A, Pieters R, Horstmann M A, Relling M V, den Boer M L, Willman C L, Carroll W L, Study CsOG, Group DCO, Leukemia GCSGfCAL. Gene expression signatures predictive of early response and outcome in high-risk childhood acute lymphoblastic leukemia: A Children's Oncology Group Study [corrected]. J Clin Oncol 2008; 26:4376-4384
13c. Bhojwani D, Kang H, Moskowitz N P, Min D J, Lee H, Potter J W, Davidson G, Willman C L, Borowitz M J, Belitskaya-Levy I, Hunger S P, Raetz E A, Carroll W L. Biologic pathways associated with relapse in childhood acute lymphoblastic leukemia: a Children's Oncology Group study. Blood 2006; 108:711-717
14c. Bachmann P S, Piazza R G, Janes M E, Wong N C, Davies C, Mogavero A, Bhadri V A, Szymanska B, Geninson G, Magistroni V, Cazzaniga G, Biondi A, Miranda-Saavedra D, Gottgens B, Saffery R, Craig J M, Marshall G M, Gambacorti-Passerini C, Pimanda J E, Lock R B. Epigenetic silencing of BIM in glucocorticoid poor-responsive pediatric acute lymphoblastic leukemia, and its reversal by histone deacetylase inhibition. Blood 2010; 116:3013-3022
15c. Jing D, Bhadri V A, Beck D, Thoms J A, Yakob N A, Wong J W, Knezevic K, Pimanda J E, Lock R B. Opposing regulation of BIM and BCL2 controls glucocorticoid-induced apoptosis of pediatric acute lymphoblastic leukemia cells. Blood 2015; 125:273-283
16c. Tissing W J, den Boer M L, Meijerink J P, Menezes R X, Swagemakers S, van der Spek P J, Sallan S E, Armstrong S A, Pieters R. Genomewide identification of prednisolone-responsive genes in acute lymphoblastic leukemia cells. Blood 2007; 109:3929-3935
17c. Paugh S W, Bonten E J, Savic D, Ramsey L B, Thierfelder W E, Gurung P, Malireddi R K, Actis M, Mayasundari A, Min J, Coss D R, Laudermilk L T, Panetta J C, McCorkle J R, Fan Y, Crews K R, Stocco G, Wilkinson M R, Ferreira A M, Cheng C, Yang W, Karol S E, Fernandez C A, Diouf B, Smith C, Hicks J K, Zanut A, Giordanengo A, Crona D, Bianchi J J, Holmfeldt L, Mullighan C G, den Boer M L, Pieters R, Jeha S, Dunwell T L, Latif F, Bhojwani D, Carroll W L, Pui C H, Myers R M, Guy R K, Kanneganti T D, Relling M V, Evans W E. NALP3 inflammasome upregulation and CASP1 cleavage of the glucocorticoid receptor cause glucocorticoid resistance in leukemia cells. Nat Genet 2015; 47:607-614
18c. Pottier N, Yang W, Assem M, Panetta J C, Pei D, Paugh S W, Cheng C, Den Boer M L, Relling M V, Pieters R, Evans W E, Cheok M H. The SWI/SNF chromatin-remodeling complex and glucocorticoid resistance in acute lymphoblastic leukemia. J Natl Cancer Inst 2008; 100:1792-1803
19c. Hulleman E, Kazemier K M, Holleman A, VanderWeele D J, Rudin C M, Broekhuis M J, Evans W E, Pieters R, Den Boer M L. Inhibition of glycolysis modulates prednisolone resistance in acute lymphoblastic leukemia cells. Blood 2009; 113:2014-2021
20c. Ariës I M, Hansen B R, Koch T, van den Dungen R, Evans W E, Pieters R, den Boer M L. The synergism of MCL1 and glycolysis on pediatric acute lymphoblastic leukemia cell survival and prednisolone resistance. Haematologica 2013; 98:1905-1911
21c. Ariës I M, van den Dungen R E, Koudijs M J, Cuppen E, Voest E, Molenaar J J, Caron H N, Pieters R, den Boer M L. Towards personalized therapy in pediatric acute lymphoblastic leukemia: RAS mutations and prednisolone resistance. Haematologica 2015; 100:e132-136
22c. Jones C L, Gearheart C M, Fosmire S, Delgado-Martin C, Evensen N A, Bride K, Waanders A J, Pais F, Wang J, Bhatla T, Bitterman D S, de Rijk S R, Bourgeois W, Dandekar S, Park E, Burleson T M, Madhusoodhan P P, Teachey D T, Raetz E A, Hermiston M L, Müschen M, Loh M L, Hunger S P, Zhang J, Garabedian M J, Porter C C, Carroll W L. MAPK signaling cascades mediate distinct glucocorticoid resistance mechanisms in pediatric leukemia. Blood 2015; 126:2202-2212
23c. Ariës I M, Jerchel I S, van den Dungen R E, van den Berk L C, Boer J M, Horstmann M A, Escherich G, Pieters R, den Boer M L. EMP1, a novel poor prognostic factor in pediatric leukemia regulates prednisolone resistance, cell proliferation, migration and adhesion. Leukemia 2014; 28:1828-1837
24c. van Galen J C, Kuiper R P, van Emst L, Levers M, Tijchon E, Scheijen B, Waanders E, van Reijmersdal S V, Gilissen C, van Kessel A G, Hoogerbrugge P M, van Leeuwen F N. BTG1 regulates glucocorticoid receptor autoinduction in acute lymphoblastic leukemia. Blood 2010; 115:4810-4819
25c. Bruscoli S, Biagioli M, Sorcini D, Frammartino T, Cimino M, Sportoletti P, Mazzon E, Bereshchenko O, Riccardi C. Lack of glucocorticoid-induced leucine zipper (GILZ) deregulates B-cell survival and results in B-cell lymphocytosis in mice. Blood 2015; 126:1790-1801
26c. Jones C L, Bhatla T, Blum R, Wang J, Paugh S W, Wen X, Bourgeois W, Bitterman D S, Raetz E A, Morrison D J, Teachey D T, Evans W E, Garabedian M J, Carroll W L. Loss of TBL1XR1 disrupts glucocorticoid receptor recruitment to chromatin and results in glucocorticoid resistance in a B-lymphoblastic leukemia model. J Biol Chem 2014; 289:20502-20515
27c. Bavetsias V, Linardopoulos S. Aurora Kinase Inhibitors: Current Status and Outlook. Front Oncol 2015; 5:278
28c. Krenn V, Musacchio A. The Aurora B Kinase in Chromosome Bi-Orientation and Spindle Checkpoint Signaling. Front Oncol 2015; 5:225
29c. Hartsink-Segers S A, Zwaan C M, Exalto C, Luijendijk M W, Calvert V S, Petricoin E F, Evans W E, Reinhardt D, de Haas V, Hedtjärn M, Hansen B R, Koch T, Caron H N, Pieters R, Den Boer M L. Aurora kinases in childhood acute leukemia: the promise of aurora B as therapeutic target. Leukemia 2013; 27:560-568
30c. Tsuboi K, Yokozawa T, Sakura T, Watanabe T, Fujisawa S, Yamauchi T, Uike N, Ando K, Kihara R, Tobinai K, Asou H, Hotta T, Miyawaki S. A Phase I study to assess the safety, pharmacokinetics and efficacy of barasertib (AZD1152), an Aurora B kinase inhibitor, in Japanese patients with advanced acute myeloid leukemia. Leuk Res 2011; 35:1384-1389
31c. Choudary I, Barr P M, Friedberg J. Recent advances in the development of Aurora kinases inhibitors in hematological malignancies. Ther Adv Hematol 2015; 6:282-294
32c. Dennis M, Davies M, Oliver S, D'Souza R, Pike L, Stockman P. Phase I study of the Aurora B kinase inhibitor barasertib (AZD1152) to assess the pharmacokinetics, metabolism and excretion in patients with acute myeloid leukemia. Cancer Chemother Pharmacol 2012; 70:461-469
33c. Goldenson B, Crispino J D. The aurora kinases in cell cycle and leukemia. Oncogene 2015; 34:537-545
34c. Kruth K, Fang M, Shelton D N, Abu-Halawa O, Mahling R, Yang H, Weissman J S, Loh M L, Müschen M, Tasian S K, Bassik M C, Kampmann M, Pufall M A. Suppression of B-cell development genes is key to glucocorticoid efficacy in treatment of acute lymphoblastic leukemia. Blood. 2017 Apr. 19. pii: blood-2017-02-766204. doi: 10.1182/blood-2017-02-766204. [Epub ahead of print]
35c. Girdler F, Gascoigne K E, Eyers P A, Hartmuth S, Crafter C, Foote K M, Keen N J, Taylor S S. Validating Aurora B as an anti-cancer drug target. J Cell Sci 2006; 119:3664-3675
36c. Ditchfield C, Johnson V L, Tighe A, Ellston R, Haworth C, Johnson T, Mortlock A, Keen N, Taylor S S. Aurora B couples chromosome alignment with anaphase by targeting BubR1, Mad2, and Cenp-E to kinetochores. J Cell Biol 2003; 161:267-280
37c. Chou T C. Drug combination studies and their synergy quantification using the Chou-Talalay method. Cancer Res 2010; 70:440-446
38c. Mortlock A A, Foote K M, Heron N M, Jung F H, Pasquet G, Lohmann JJ, Warin N, Renaud F, De Savi C, Roberts N J, Johnson T, Dousson C B, Hill G B, Perkins D, Hatter G, Wilkinson R W, Wedge S R, Heaton S P, Odedra R, Keen N J, Crafter C, Brown E, Thompson K, Brightwell S, Khatri L, Brady M C, Kearney S, McKillop D, Rhead S, Parry T, Green S. Discovery, synthesis, and in vivo activity of a new class of pyrazoloquinazolines as selective inhibitors of aurora B kinase. J Med Chem 2007; 50:2213-2224
39c. Wilkinson R W, Odedra R, Heaton S P, Wedge S R, Keen N J, Crafter C, Foster J R, Brady M C, Bigley A, Brown E, Byth K F, Barrass N C, Mundt K E, Foote K M, Heron N M, Jung F H, Mortlock A A, Boyle F T, Green S. AZD1152, a selective inhibitor of Aurora B kinase, inhibits human tumor xenograft growth by inducing apoptosis. Clin Cancer Res 2007; 13:3682-3688

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A method of treating a hematologic or other malignancy comprising administering to a subject a glucocorticoid and an Aurora Kinase B inhibitor.

2. The method of claim 1, wherein the administering further includes administering a demethylase inhibitor to the subject.

3. The method of claim 1, where the Aurora Kinase B inhibitor is a plurality of Aurora Kinase B inhibitors.

4. The method of claim 2, where the demethylase inhibitor is a plurality of demethylase inhibitors.

5. The method of claim 1, wherein the Aurora Kinase B inhibitor has a half maximal inhibitory concentration (IC₅₀) for inhibiting of Aurora Kinase B of less than about 1 μM.

6. The method of claim 1, wherein the Aurora Kinase B inhibitor includes an isolated antibody capable of specifically binding to and inhibiting Aurora Kinase B.

7. The method of claim 1, wherein the Aurora Kinase B inhibitor includes a small interfering RNA or microRNA-based compound capable of inhibiting expression of Aurora Kinase B.

8. The method of claim 1, wherein the hematologic malignancy is a childhood B-lineage acute lymphoblastic leukemia.

9. The method of claim 1, wherein the hematologic malignancy is resistant to glucocorticoid-mediated cell death.

10. The method of claim 1, wherein the other malignancy is a solid tumor.

11. The method of claim 10, wherein the glucocorticoid and the Aurora Kinase B inhibitor inhibits metastasis of the solid tumor.

12. The method of claim 11, wherein the inhibition of metastasis of the solid tumor is due to inhibition of epithelial-mesenchymal transition of the solid tumor by the glucocorticoid and the Aurora Kinase B inhibitor.

13. The method of claim 11, wherein the inhibition of metastasis of the solid tumor is due to the glucocorticoid and the Aurora Kinase B inhibitor enhancing expression E-cadherin in the solid tumor.

14. The method of claim 10, wherein the solid tumor is resistant to glucocorticoid-mediated cell death.

15. A method of enhancing chemotherapeutic effects of a glucocorticoid in a subject undergoing chemotherapy with the glucocorticoid for a hematologic or other malignancy comprising a step of administering to the subject an amount of an Aurora Kinase B inhibitor effective to enhance chemotherapeutic effects of the glucocorticoid.

16. The method of claim 15, wherein the amount of the Aurora Kinase B inhibitor is effective to enhance efficacy of a reduced dosage of the glucocorticoid as compared to administrating the glucocorticoid without the Aurora Kinase B inhibitor.

17. The method of claim 16, wherein the reduced dosage of the glucocorticoid is effective to reduce side effects associated with glucocorticoid administration.

18. The method of claim 15, wherein the administering further includes administering to the subject an amount of a demethylase inhibitor effective to enhance the chemotherapeutic effect of the glucocorticoid.

19. A method of determining early relapse of hematologic or other malignancies in a subject comprising:
quantifying a concentration or level of expression of Aurora Kinase B in a sample from a subject;
comparing the concentration or level of expression of Aurora Kinase B in the sample to an Aurora Kinase B control; and
identifying the subject as likely to have early relapse of a hematologic and other malignancy when the concentration or level of expression of Aurora Kinase B in the sample is greater than the Aurora Kinase B control.

20-48. (canceled)

49. The method of claim 1, wherein the Aurora Kinase B inhibitor(s) includes at least one of Barasertib (CAS No. 722543-31-9), ZM 447439 (CAS No. 331771-20-1), Danusertib (CAS No, 827318-97-8), AT9283 (CAS No. 896466-04-9), PF-03S14735 (CAS No. 942487-16-3), AMG 900 (CAS No, 945595-80-2), and Cytarabine. (CAS No. 147-94-4).

50.-56. (canceled)


SEQUENCE LISTING (90 sequences)

SEQ ID NO: 1 - DNA, 22 bp, Artificial
  BCL2 Forward Primer
gtggatgactgagtacctgaac

SEQ ID NO: 2 - DNA, 22 bp, Artificial
  BCL2 Reverse Primer
gccaggagaaatcaaacagagg

SEQ ID NO: 3 - DNA, 21 bp, Artificial
  BIM Forward Primer
tgattcttcagatgcccttcc

SEQ ID NO: 4 - DNA, 20 bp, Artificial
  BIM Reverse Primer
aacttgatttctccgcaacc

SEQ ID NO: 5 - DNA, 21 bp, Artificial
  IL7R Forward Primer
ctggagaaagtggctatgctc

SEQ ID NO: 6 - DNA, 20 bp, Artificial
  IL7R Reverse Primer
acatctgggtcctcaaaagc

SEQ ID NO: 7 - DNA, 19 bp, Artificial
  Myc Forward Primer
ggacccgcttctctgaaag

SEQ ID NO: 8 - DNA, 22 bp, Artificial
  Myc Reverse Primer
gtcgaggtcatagttcctgttg

SEQ ID NO: 9 - DNA, 21 bp, Artificial
  PIK3CD Forward Primer
agtggaacaagcatgaggatg

SEQ ID NO: 10 - DNA, 18 bp, Artificial
  PIK3CD Reverse Primer
acttgatggcgaaggagc

SEQ ID NO: 11 - DNA, 22 bp, Artificial
  TXNIP Forward Primer
gatctgaacatccctgataccc

SEQ ID NO: 12 - DNA, 22 bp, Artificial
  TXNIP Reverse Primer
catccatgtcatctagcagagg

SEQ ID NO: 13 - DNA, 20 bp, Artificial
  RPL19 Forward Primer
atcgatcgccacatgtatca

SEQ ID NO: 14 - DNA, 20 bp, Artificial
  RPL19 Reverse Primer
gcgtcgttccttggtcttag

SEQ ID NO: 15 - DNA, 92 bp, Artificial
  PCR amplified fragment of G9a-like gene
  misc_feature (23)..(47): Targeting sequence for shRNA
  misc_feature (57)..(81): Targeting sequence for shRNA
cttgtggaaaggacgaaacaccgaagttcgaggagctagaaatcatattcaagagatatgatctctagcttctcgaacttctttttctgcag

SEQ ID NO: 16 - DNA, 21 bp, Artificial
  ENaC(alpha) -2.5kb Forward Primer
aaactccagtctcccttgagc

SEQ ID NO: 17 - DNA, 20 bp, Artificial
  ENaC(alpha) GBR (-1.3kb) Forward Primer
caccttcagtgcctgctttc

SEQ ID NO: 18 - DNA, 21 bp, Artificial
  ENaC(alpha) TSS Forward Primer
tcaactggaaaggaaccagtc

SEQ ID NO: 19 - DNA, 20 bp, Artificial
  ENaC(alpha) +2.1kb Forward Primer
caacgaaatgacctggcttt

SEQ ID NO: 20 - DNA, 21 bp, Artificial
  ENaC(alpha) +5.7 kb Forward Primer
gaccttttgggagagtgaagg

SEQ ID NO: 21 - DNA, 21 bp, Artificial
  ENaC(alpha) +11 kb Forward Primer
ccggaaattaaagaggagctg

SEQ ID NO: 22 - DNA, 21 bp, Artificial
  CDH16 -1.5 kb Forward Primer
gccaaggtccatacattcctt

SEQ ID NO: 23 - DNA, 22 bp, Artificial
  CDH16 GBR (-0.36 kb) Forward Primer
ttgagctgagcactgaagcatg

SEQ ID NO: 24 - DNA, 21 bp, Artificial
  CDH16 TSS Forward Primer
tggctttccaaagtcaatgag

SEQ ID NO: 25 - DNA, 20 bp, Artificial
  CDH16 +2.5 kb Forward Primer
atctccggagtcctgatgtg

SEQ ID NO: 26 - DNA, 20 bp, Artificial
  CDH16 +5 kb Forward Primer
agtgggtggggtaaggtctc

SEQ ID NO: 27 - DNA, 20 bp, Artificial
  CDH1 GBR (+21kb) Forward Primer
cctgctcatcttctcccaga

SEQ ID NO: 28 - DNA, 21 bp, Artificial
  HSD11B2 GBR (-7.5kb) Forward Primer
tgtaactggtgcgacttggaa

SEQ ID NO: 29 - DNA, 20 bp, Artificial
  HSD11B2 TSS Forward Primer
gggactggacactcaacagg

SEQ ID NO: 30 - DNA, 21 bp, Artificial
  PPL GBR (-7.7kb) Forward Primer
cagcttcacccctgttttgta

SEQ ID NO: 31 - DNA, 22 bp, Artificial
  FKBP5 GBR (+86 kb) Forward Primer
tgtgccagccacattcagaaca

SEQ ID NO: 32 - DNA, 21 bp, Artificial
  FKBP5 TSS Forward Primer
tcccatctagctctggtctca

SEQ ID NO: 33 - DNA, 20 bp, Artificial
  CITED2 GBR (-0.93kb) Forward Primer
agtttgcgtttgcagctctt

SEQ ID NO: 34 - DNA, 20 bp, Artificial
  FOXO1 GBR (-0.2kb) Forward Primer
agatttgggggaacgaagcc

SEQ ID NO: 35 - DNA, 20 bp, Artificial
  H3K9me3 positive region Forward Primer
tcttggagcttgcctttcat

SEQ ID NO: 36 - DNA, 20 bp, Artificial
  H3K9me3 negative region Forward Primer
cagctaatcagcctccttgg

SEQ ID NO: 37 - DNA, 21 bp, Artificial
  ENaC(alpha) -2.5kb Reverse Primer
ccatgctgccttaagctagtg

SEQ ID NO: 38 - DNA, 20 bp, Artificial
  ENaC(alpha) GBR (-1.3kb) Reverse Primer
aggccaggaatgtgtaatcg

SEQ ID NO: 39 - DNA, 21 bp, Artificial
  ENaC(alpha) TSS Reverse Primer
ctcgagctgtgtcctgattct

SEQ ID NO: 40 - DNA, 20 bp, Artificial
  ENaC(alpha) +2.1kb Reverse Primer
ggccccttcgtatattccat

SEQ ID NO: 41 - DNA, 21 bp, Artificial
  ENaC(alpha) +5.7 kb Reverse Primer
ccacacacacaaacctgtgac

SEQ ID NO: 42 - DNA, 21 bp, Artificial
  ENaC(alpha) +11 kb Reverse Primer
tacaggtcaaagagcgtctgc

SEQ ID NO: 43 - DNA, 21 bp, Artificial
  CDH16 -1.5 kb Reverse Primer
ctcctgccattcaataagctg

SEQ ID NO: 44 - DNA, 21 bp, Artificial
  CDH16 GBR (-0.36 kb) Reverse Primer
tgcagccacaccttttcacac

SEQ ID NO: 45 - DNA, 20 bp, Artificial
  CDH16 TSS Reverse Primer
ggcacttgagcaggtaggag

SEQ ID NO: 46 - DNA, 20 bp, Artificial
  CDH16 +2.5 kb Reverse Primer
tgaagcctcaaggaagagga

SEQ ID NO: 47 - DNA, 20 bp, Artificial
  CDH16 +5 kb Reverse Primer
cagggctcaggagctgatac

SEQ ID NO: 48 - DNA, 20 bp, Artificial
  CDH1 GBR (+21kb) Reverse Primer
tgcaccaagaacgctttatg

SEQ ID NO: 49 - DNA, 21 bp, Artificial
  HSD11B2 GBR (-7.5kb) Reverse Primer
ttccaaacaccttgtccccaa

SEQ ID NO: 50 - DNA, 21 bp, Artificial
  HSD11B2 TSS Reverse Primer
ggtggagaactctcccactct

SEQ ID NO: 51 - DNA, 20 bp, Artificial
  PPL GBR (-7.7kb) Reverse Primer
ggccagcacaattttccact

SEQ ID NO: 52 - DNA, 21 bp, Artificial
  FKBP5 GBR (+86 kb) Reverse Primer
gtaaccacatcaagcgagctg

SEQ ID NO: 53 - DNA, 20 bp, Artificial
  FKBP5 TSS Reverse Primer
gggactgcttctcaccatgt

SEQ ID NO: 54 - DNA, 20 bp, Artificial
  CITED2 GBR (-0.93kb) Reverse Primer
aaggtggatctggggacgag

SEQ ID NO: 55 - DNA, 20 bp, Artificial
  FOXO1 GBR (-0.2kb) Reverse Primer
gatggccccgcgaagttaag

SEQ ID NO: 56 - DNA, 20 bp, Artificial
  H3K9me3 positive region Reverse Primer
ttcaatgacctcagcagcag

SEQ ID NO: 57 - DNA, 20 bp, Artificial
  H3K9me3 negative region Reverse Primer
gcctcaagaagctggacatc

SEQ ID NO: 58 - DNA, 21 bp, Artificial
  CDH16 Forward Primer
tcggcagtgggcatccttgta

SEQ ID NO: 59 - DNA, 20 bp, Artificial
  ENaC(alpha) Forward Primer
aacggtctgtccctgatgct

SEQ ID NO: 60 - DNA, 20 bp, Artificial
  HSD11B2 Forward Primer
gacctgaccaaaccaggaga

SEQ ID NO: 61 - DNA, 20 bp, Artificial
  PPL Forward Primer
caggagatcctccaattcca

SEQ ID NO: 62 - DNA, 18 bp, Artificial
  CDH1 Forward Primer
ttcccaactcctctcctg

SEQ ID NO: 63 - DNA, 20 bp, Artificial
  FKBP5 Forward Primer
aggctgcaagactgcagatc

SEQ ID NO: 64 - DNA, 20 bp, Artificial
  CITED2 Forward Primer
gccaggtttaacaactccca

SEQ ID NO: 65 - DNA, 20 bp, Artificial
  FOXO1 Forward Primer
acagttttccaaatggcctg

SEQ ID NO: 66 - DNA, 20 bp, Artificial
  (beta)-actin Forward Primer
ccacactgtgcccatctacg

SEQ ID NO: 67 - DNA, 20 bp, Artificial
  HP1(alpha) Forward Primer
gatgtcatcggcactgtttg

SEQ ID NO: 68 - DNA, 20 bp, Artificial
  HP1(beta) Forward Primer
tttggtttgctctcctctcc

SEQ ID NO: 69 - DNA, 20 bp, Artificial
  HP1(gamma) Forward Primer
aagaggcagagcctgaagaa

SEQ ID NO: 70 - DNA, 20 bp, Artificial
  G9a Forward Primer
atgggtgaagccgtctcgga

SEQ ID NO: 71 - DNA, 20 bp, Artificial
  GLP Forward Primer
gatagcggaaaatggggttt

SEQ ID NO: 72 - DNA, 21 bp, Artificial
  CDH16 Reverse Primer
gcacgctgtctgctggttgat

SEQ ID NO: 73 - DNA, 20 bp, Artificial
  ENaC(alpha) Reverse Primer
ttggtgcagtcgccataatc

SEQ ID NO: 74 - DNA, 20 bp, Artificial
  HSD11B2 Reverse Primer
ccgcatcagcaactacttca

SEQ ID NO: 75 - DNA, 20 bp, Artificial
  PPL Reverse Primer
ctgggaagctctttccctct

SEQ ID NO: 76 - DNA, 19 bp, Artificial
  CDH1 Reverse Primer
aaaccttgccttctttgtc

SEQ ID NO: 77 - DNA, 20 bp, Artificial
  FKBP5 Reverse Primer
cttgcccattgctttattgg

SEQ ID NO: 78 - DNA, 20 bp, Artificial
  CITED2 Reverse Primer
ctggtttgtcccgttcatct

SEQ ID NO: 79 - DNA, 20 bp, Artificial
  FOXO1 Reverse Primer
catccccttctccaagatca

SEQ ID NO: 80 - DNA, 26 bp, Artificial
  (beta)-actin Reverse Primer
aggatcttcatgaggtagtcagtcag

SEQ ID NO: 81 - DNA, 21 bp, Artificial
  HP1(alpha) Reverse Primer
gcacaatacttgggaacctga

SEQ ID NO: 82 - DNA, 20 bp, Artificial
  HP1(beta) Reverse Primer
aacacatgggagccagaaga

SEQ ID NO: 83 - DNA, 22 bp, Artificial
  HP1(gamma) Reverse Primer
tctgtaaatcccttccacttca

SEQ ID NO: 84 - DNA, 20 bp, Artificial
  G9a Reverse Primer
atcttgggtgcctccatgcg

SEQ ID NO: 85 - DNA, 20 bp, Artificial
  GLP Reverse Primer
gtagtcctcaagggctgtgc

SEQ ID NO: 86 - DNA, 80 bp, Artificial
  oMK483 Primer
aatgatacggcgaccaccgagatcggaagagcacacgtctgaactccagtcaccttgtactctagatgactgaccccttg

SEQ ID NO: 87 - DNA, 80 bp, Artificial
  oMK484 Primer
aatgatacggcgaccaccgagatcggaagagcacacgtctgaactccagtcacgccaatctctagatgactgaccccttg

SEQ ID NO: 88 - DNA, 80 bp, Artificial
  oMK485 Primer
aatgatacggcgaccaccgagatcggaagagcacacgtctgaactccagtcacagttccctctagatgactgaccccttg

SEQ ID NO: 89 - DNA, 80 bp, Artificial
  oMK486 Primer
aatgatacggcgaccaccgagatcggaagagcacacgtctgaactccagtcactagcttctctagatgactgaccccttg

SEQ ID NO: 90 - DNA, 80 bp, Artificial
  oMK487 Primer
aatgatacggcgaccaccgagatcggaagagcacacgtctgaactccagtcacttaggcctctagatgactgaccccttg